(19) United States
(12) Patent Application Publication
(10) Pub. No.: US 2025/0109402 A1
(43) Pub. Date: Apr. 3, 2025

(54) COMPOSITIONS AND METHODS FOR TREATMENT OF PAIN

(71) Applicant: Atalanta Therapeutics, Inc., Boston, MA (US)

(72) Inventors: Stefan I. MCDONOUGH, Somerville, MA (US); Corrie GALLANT-BEHM, Wellesley, MA (US); Matthew HASSLER, Boston, MA (US); Daniel CURTIS, Belmont, MA (US); Bruno Miguel Da Cruz GODINHO, Reading, MA (US)

(21) Appl. No.: 18/714,751

(22) PCT Filed: Dec. 1, 2022
(86) PCT No.: PCT/US2022/080764

§ 371 Date: May 30, 2024

Related U.S. Application Data
(60) Provisional application No. 63/284,957, filed on Dec. 1, 2021.

(51) Int. Cl. C12N 15/113

(52) U.S. Cl. CPC C12N 15/1138; C12N 2310/14; C12N 2310/321; C12N 2310/3341

(57) ABSTRACT

The present disclosure provides single- or double-stranded interfering RNA molecules (e.g., siRNA) that target a SCN9A gene. The interfering RNA molecules may contain specific patterns of nucleoside modifications and internucleoside linkage modifications, as pharmaceutical compositions including the same. The siRNA molecules may be branched siRNA molecules, such as di-branched, tri¬branched, ortetra-branched siRNA molecules. The disclosed siRNA molecules may further feature a 5′ phosphorus stabilizing moiety and/or a hydrophobic moiety. Additionally, the disclosure provides methods for delivering the siRNA molecule of the disclosure to the central nervous system of a subject, such as a subject experiencing pain or identified as having a pain disorder.

### TECHNICAL FIELD

This disclosure relates to small interfering RNA (siRNA) molecules, and compositions containing the same, that target RNA transcripts (e.g., mRNA) of a sodium voltage-gated channel alpha subunit 9 (SCN9A) gene. The disclosure further describes methods for the treatment of pain (e.g., chronic or acute pain) by delivering SCN9A-targeting siRNA molecules to the central nervous system of a subject in need.

### BACKGROUND

Pain indications represent a substantial unmet medical need. Among the existing therapeutics for pain, pregabalin and duloxetine have quite limited efficacy, and opioids are effective against some forms of acute or persistent pain but come with severe respiratory, gastrointestinal, and addiction liabilities. Other pharmacological treatments are sometimes used off-label for neuropathic or chronic pain but by and large have weak efficacy and prohibitive side effects. Accordingly, much interest has focused on developing new treatments for pain, particularly on making inhibitors of the Nav1.7 voltage-gated sodium ion channel protein encoded by the voltage-gated sodium channel alpha subunit 9 (SCN9A) gene.

However, Nav1.7 protein has proven difficult to target. One significant difficulty stems from the selectivity required for an Nav1.7 inhibitor to be an effective therapeutic. While Nav1.7 itself is not anticipated to have prohibitive on-target liability to inhibition, among eight other sodium channel paralogs are those governing cellular excitability in brain, cardiac muscle, and skeletal muscle. Since the functional areas of different sodium channels are highly conserved, few small molecule inhibitors have been reported that have meaningful selectivity for Nav1.7 among sodium channel isoforms. Achieving central nervous system penetrance of a small molecule Nav1.7-selective inhibitor has also been challenging.

Accordingly, there remains a need for therapeutics capable of selectively diminishing Nav1.7 activity among other sodium channels in a manner that provides effective relief from various forms of pain.

### SUMMARY OF THE DISCLOSURE

The present disclosure provides compositions and methods for reduction of voltage-gated sodium channel alpha subunit 9 expression by way of small interfering RNA (siRNA)-mediated silencing of sodium voltage-gated channel alpha subunit 9 (SCN9A) transcripts. The compositions and methods provide the benefit of exhibiting high selectivity toward SCN9A over other central nervous system (CNS) genes, including those that encode other sodium channel paralogs.

The siRNA molecules of the disclosure can be used to silence the SCN9A gene, thereby preventing the translation of the corresponding mRNA transcript and reducing SCN9A expression. This reduction of SCN9A levels thus prevents transmission of noxious stimuli that result in pain. The siRNA molecules of the disclosure can be administered to individuals with a pain syndrome or to individuals identified as having a gain-of-function SCN9A mutation. The siRNA molecules of the disclosure can be delivered directly to the CNS or neurons of a subject in need of SCN9A silencing by way of, for example, injection intrathecally, intracerebroventricularly, intrastriatally, intraparenchymally, direct injection into a specific nerve or ganglion (ganglia) (e.g., trigeminal or dorsal root ganglia), intra-cisterna magna injection, such as by catheterization, intravenous injection, subcutaneous injection, or intramuscular injection.

In an aspect, the disclosure provides a siRNA molecule containing an antisense strand and sense strand having complementarity to the antisense strand. The antisense strand has complementarity sufficient to hybridize to a region within an SCN9A mRNA transcript having the nucleic acid sequence of any one of SEQ ID NOs: 385-576 and 961-1152. In some embodiments, the antisense strand has complementarity sufficient to hybridize to a region within an SCN9A mRNA transcript having the nucleic acid sequence of any one of SEQ ID NOs: 385-576. The antisense strand may be, for example, from 10 to 50 nucleotides in length (e.g., from 10 to 45 nucleotides in length, from 10 to 40 nucleotides in length, from 10 to 35 nucleotides in length, from 10 to 30 nucleotides in length, from 10 to 29 nucleotides in length, from 10 to 28 nucleotides in length, from 10 to 27 nucleotides in length, from 10 to 26 nucleotides in length, from 10 to 25 nucleotides in length, from 10 to 24 nucleotides in length, from 10 to 23 nucleotides in length, from 10 to 22 nucleotides in length, from 10 to 21 nucleotides in length, or from 10 to 20 nucleotides in length). In some embodiments, the antisense strand is 10 nucleotides in length, 11 nucleotides in length, 12 nucleotides in length, 13 nucleotides in length, 14 nucleotides in length, 15 nucleotides in length, 16 nucleotides in length, 17 nucleotides in length, 18 nucleotides in length, 19 nucleotides in length, 20 nucleotides in length, 21 nucleotides in length, 22 nucleotides in length, 23 nucleotides in length, 24 nucleotides in length, 25 nucleotides in length, 26 nucleotides in length, 27 nucleotides in length, 28 nucleotides in length, 29 nucleotides in length, 30 nucleotides in length, or more.

In some embodiments of any of the foregoing aspects, the antisense strand has at least 70% (e.g., at least 70%, at least 71%, at least 72%, at least 73%, at least 74%, at least 75%, at least 76, at least 77, at least 78, at least 79, at least 80, at least 81, at least 82, at least 83, at least 84, at least 85, at least 86, at least 87, at least 88, at least 89, at least 90, at least 91, at least 92, at least 93, at least 94, at least 95, at least 96, at least 97, at least 98, at least 99, or 100%) complementarity to a region of 15 contiguous nucleobases within the SCN9A mRNA transcript having the nucleic acid sequence of any one of SEQ ID NOs: 385-576 and 961-1152. In some embodiments, the antisense strand has at least 70% (e.g., at least 70%, at least 71%, at least 72%, at least 73%, at least 74%, at least 75%, at least 76, at least 77, at least 78, at least 79, at least 80, at least 81, at least 82, at least 83, at least 84, at least 85, at least 86, at least 87, at least 88, at least 89, at least 90, at least 91, at least 92, at least 93, at least 94, at least 95, at least 96, at least 97, at least 98, at least 99, or 100%) complementarity to a region of 16 contiguous nucleobases within the SCN9A mRNA transcript having the nucleic acid sequence of any one of SEQ ID NOs: 385-576 and 961-1152. In some embodiments, the antisense strand has at least 70% (e.g., at least 70%, at least 71%, at least 72%, at least 73%, at least 74%, at least 75%, at least 76, at least 77, at least 78, at least 79, at least 80, at least 81, at least 82, at least 83, at least 84, at least 85, at least 86, at least 87, at least 88, at least 89, at least 90, at least 91, at least 92, at least 93, at least 94, at least 95, at least 96, at least 97, at least 98, at least 99, or 100%) complementarity to a region of 17 contiguous nucleobases within the SCN9A mRNA transcript having the nucleic acid sequence of any one of SEQ ID NOs: 385-576 and 961-1152. In some embodiments, the antisense strand has at least 70% (e.g., at least 70%, at least 71%, at least 72%, at least 73%, at least 74%, at least 75%, at least 76, at least 77, at least 78, at least 79, at least 80, at least 81, at least 82, at least 83, at least 84, at least 85, at least 86, at least 87, at least 88, at least 89, at least 90, at least 91, at least 92, at least 93, at least 94, at least 95, at least 96, at least 97, at least 98, at least 99, or 100%) complementarity to a region of 18 contiguous nucleobases within the SCN9A mRNA transcript having the nucleic acid sequence of any one of SEQ ID NOs: 385-576 and 961-1152. In some embodiments, the antisense strand has at least 70% (e.g., at least 70%, at least 71%, at least 72%, at least 73%, at least 74%, at least 75%, at least 76, at least 77, at least 78, at least 79, at least 80, at least 81, at least 82, at least 83, at least 84, at least 85, at least 86, at least 87, at least 88, at least 89, at least 90, at least 91, at least 92, at least 93, at least 94, at least 95, at least 96, at least 97, at least 98, at least 99, or 100%) complementarity to a region of 19 contiguous nucleobases within the SCN9A mRNA transcript having the nucleic acid sequence of any one of SEQ ID NOs: 385-576 and 961-1152. In some embodiments, the antisense strand has at least 70% (e.g., at least 70%, at least 71%, at least 72%, at least 73%, at least 74%, at least 75%, at least 76, at least 77, at least 78, at least 79, at least 80, at least 81, at least 82, at least 83, at least 84, at least 85, at least 86, at least 87, at least 88, at least 89, at least 90, at least 91, at least 92, at least 93, at least 94, at least 95, at least 96, at least 97, at least 98, at least 99, or 100%) complementarity to a region of 20 contiguous nucleobases within the SCN9A mRNA transcript having the nucleic acid sequence of any one of SEQ ID NOs: 385-576 and 961-1152. In some embodiments, the antisense strand has at least 70% (e.g., at least 70%, at least 71%, at least 72%, at least 73%, at least 74%, at least 75%, at least 76, at least 77, at least 78, at least 79, at least 80, at least 81, at least 82, at least 83, at least 84, at least 85, at least 86, at least 87, at least 88, at least 89, at least 90, at least 91, at least 92, at least 93, at least 94, at least 95, at least 96, at least 97, at least 98, at least 99, or 100%) complementarity to a region of 21 contiguous nucleobases within the SCN9A mRNA transcript having the nucleic acid sequence of any one of SEQ ID NOs: 385-576 and 961-1152.

In some embodiments, the antisense strand has at least 70% (e.g., at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100%) complementarity to the region within the SCN9A mRNA transcript having the nucleic acid sequence of any one of SEQ ID NOs: 385-576 and 961-1152.

In some embodiments, the antisense strand has at least 75% complementarity to the region within the SCN9A mRNA transcript having the nucleic acid sequence of any one of SEQ ID NOs: 385-576 and 961-1152. For example, the antisense strand may have at least 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% complementarity to the region within the SCN9A mRNA transcript having the nucleic acid sequence of any one of SEQ ID Nos: 385-576 and 961-1152.

In some embodiments, the antisense strand has at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, or 30 contiguous nucleotides that are fully complementary to a contiguous polynucleotide segment of equal length within the region of the SCN9A RNA transcript having the nucleic acid sequence of any one of SEQ ID NOs: 385-576 and 961-1152.

In some embodiments, the antisense strand has from 10 to 30 contiguous nucleotides (e.g., 10, 11, 12, 13, 14, 15, 16, 17, 18, 19, 20, 21, 22, 23, 24, 25, 26, 27, 28, 29, or 30 contiguous nucleotides) that are fully complementary to a contiguous polynucleotide segment of equal length within the region of the SCN9A RNA transcript having the nucleic acid sequence of any one of SEQ ID NOs: 385-576 and 961-1152.

In some embodiments, the antisense strand has from 12 to 30 contiguous nucleotides (e.g., 10, 11, 12, 13, 14, 15, 16, 17, 18, 19, 20, 21, 22, 23, 24, 25, 26, 27, 28, 29, or 30 contiguous nucleotides) that are fully complementary to a contiguous polynucleotide segment of equal length within the region of the SCN9A RNA transcript having the nucleic acid sequence of any one of SEQ ID NOs: 385-576 and 961-1152.

In some embodiments, the antisense strand has from 15 to 30 contiguous nucleotides (e.g., 15, 16, 17, 18, 19, 20, 21, 22, 23, 24, 25, 26, 27, 28, 29, or 30 contiguous nucleotides) that are fully complementary to a contiguous polynucleotide segment of equal length within the region of the SCN9A RNA transcript having the nucleic acid sequence of any one of SEQ ID NOs: 385-576 and 961-1152.

In some embodiments, the antisense strand has from 18 to 30 contiguous nucleotides (e.g., 18, 19, 20, 21, 22, 23, 24, 25, 26, 27, 28, 29, or 30 contiguous nucleotides) that are fully complementary to a contiguous polynucleotide segment of equal length within the region of the SCN9A RNA transcript having the nucleic acid sequence of any one of SEQ ID NOs: 385-576 and 961-1152.

In some embodiments, the antisense strand has from 18 to 25 contiguous nucleotides (e.g., 18, 19, 20, 21, 22, 23, 24, or 25 contiguous nucleotides) that are fully complementary to a contiguous polynucleotide segment of equal length within the region of the SCN9A mRNA transcript having the nucleic acid sequence of any one of SEQ ID Nos: 385-576 and 961-1152.

In some embodiments, the antisense strand has from 18 to 21 contiguous nucleotides (e.g., 18, 19, 20, or 21 contiguous nucleotides) that are fully complementary to a contiguous polynucleotide segment of equal length within the region of the SCN9A mRNA transcript having the nucleic acid sequence of any one of SEQ ID NOs: 385-576 and 961-1152.

In some embodiments, the antisense strand has 21 contiguous nucleotides that are fully complementary to a contiguous polynucleotide segment of equal length within the region of the SCN9A mRNA transcript having the nucleic acid sequence of any one of SEQ ID NOs: 385-576 and 961-1152.

In some embodiments, the antisense strand has from 21 to 30 contiguous nucleotides (e.g., 21, 22, 23, 24, 25, 26, 27, 28, 29, or 30 contiguous nucleotides) that are fully complementary to a contiguous polynucleotide segment of equal length within the region of the SCN9A RNA transcript having the nucleic acid sequence of any one of SEQ ID Nos: 385-576 and 961-1152.

In some embodiments, the antisense strand has from 24 to 30 contiguous nucleotides (e.g., 24, 25, 26, 27, 28, 29, or 30 contiguous nucleotides) that are fully complementary to a contiguous polynucleotide segment of equal length within the region of the SCN9A RNA transcript having the nucleic acid sequence of any one of SEQ ID NOs: 385-576 and 961-1152.

In some embodiments, the antisense strand has 30 contiguous nucleotides that are fully complementary to a contiguous polynucleotide segment of equal length within the region of the SCN9A RNA transcript having the nucleic acid sequence of any one of SEQ ID NOs: 385-576 and 961-1152.

In some embodiments, the antisense strand has 9 or fewer nucleotide mismatches relative to the region of the SCN9A RNA transcript having the nucleic acid sequence of any one of SEQ ID NOs: 385-576 and 961-1152, optionally wherein the antisense strand contains 8 or fewer, 7 or fewer, 6 or fewer, 5 or fewer, 4 or fewer, 3 or fewer, 2 or fewer, or only 1 mismatch relative to the region of the SCN9A RNA transcript having the nucleic acid sequence of any one of SEQ ID NOs: 385-576 and 961-1152.

In some embodiments of any of the foregoing aspects or embodiments of the disclosure, the region of the SCN9A RNA transcript has the nucleic acid sequence of any one of SEQ ID NOs: 385-576. In some embodiments of any of the foregoing aspects or embodiments of the disclosure, the region of the SCN9A RNA transcript has the nucleic acid sequence of SEQ ID NO: 970 or 1072.

In some embodiments, the antisense strand has a nucleic acid sequence that is at least 85% identical (e.g., 85%, 86%, 87%, 88%, 89%, 90%, 91%, 92%, 93%, 94%, 95%, 96%, 97%, 98%, 99%, or 100% identical) to the nucleic acid sequence of any one of SEQ ID NOs: 1-192 and 577-768. In some embodiments, the antisense strand has a nucleic acid sequence that is at least 85% identical (e.g., 85%, 86%, 87%, 88%, 89%, 90%, 91%, 92%, 93%, 94%, 95%, 96%, 97%, 98%, 99%, or 100% identical) to the nucleic acid sequence of any one of SEQ ID NOs: 1-192. In some embodiments, the antisense strand has a nucleic acid sequence that is at least 85% identical (e.g., 85%, 86%, 87%, 88%, 89%, 90%, 91%, 92%, 93%, 94%, 95%, 96%, 97%, 98%, 99%, or 100% identical) to the nucleic acid sequence of SEQ ID NO: 586 or 688.

In some embodiments, the antisense strand has a nucleic acid sequence that is at least 90% identical (e.g., 90%, 91%, 92%, 93%, 94%, 95%, 96%, 97%, 98%, 99%, or 100% identical) to the nucleic acid sequence of any one of SEQ ID NOs: 1-192 and 577-768. In some embodiments, the antisense strand has a nucleic acid sequence that is at least 90% identical (e.g., 90%, 91%, 92%, 93%, 94%, 95%, 96%, 97%, 98%, 99%, or 100% identical) to the nucleic acid sequence of any one of SEQ ID NOs: 1-192.

In some embodiments, the antisense strand has a nucleic acid sequence that is at least 90% identical (e.g., 90%, 91%, 92%, 93%, 94%, 95%, 96%, 97%, 98%, 99%, or 100% identical) to the nucleic acid sequence of any one of SEQ ID NOs: 586 or 688.

In some embodiments, the antisense strand has a nucleic acid sequence that is at least 95% identical (e.g., 95%, 96%, 97%, 98%, 99%, or 100% identical) to the nucleic acid sequence of any one of SEQ ID NOs: 1-192 and 577-768, optionally wherein the antisense strand has a nucleic acid sequence that is at least 96%, 97%, 98%, or 99% identical to the nucleic acid sequence of any one of SEQ ID NOs: 1-192 and 577-768. In some embodiments, the antisense strand has a nucleic acid sequence that is at least 95% identical (e.g., 95%, 96%, 97%, 98%, 99%, or 100% identical) to the nucleic acid sequence of any one of SEQ ID NOs: 1-192, optionally wherein the antisense strand has a nucleic acid sequence that is at least 96%, 97%, 98%, or 99% identical to the nucleic acid sequence of any one of SEQ ID NOs: 1-192. In some embodiments, the antisense strand has a nucleic acid sequence that is at least 95% identical (e.g., 95%, 96%, 97%, 98%, 99%, or 100% identical) to the nucleic acid sequence of SEQ ID NO: 586 or 688, optionally wherein the antisense strand has a nucleic acid sequence that is at least 96%, 97%, 98%, or 99% identical to the nucleic acid sequence of SEQ ID NO: 586 or 688.

In some embodiments, the antisense strand has the nucleic acid sequence of any one of SEQ ID NOs: 1-192 and 577-768. In some embodiments, the antisense strand has the nucleic acid sequence of any one of SEQ ID NOs: 1-192. In some embodiments, the antisense strand has the nucleic acid sequence of SEQ ID NO: 586 or 688.

In some embodiments, the sense strand has a nucleic acid sequence that is at least 85% identical (e.g., 85%, 86%, 87%, 88%, 89%, 90%, 91%, 92%, 93%, 94%, 95%, 96%, 97%, 98%, 99%, or 100% identical) to the nucleic acid sequence of any one of SEQ ID NOs: 193-384 and 769-960. In some embodiments, the sense strand has a nucleic acid sequence that is at least 85% identical (e.g., 85%, 86%, 87%, 88%, 89%, 90%, 91%, 92%, 93%, 94%, 95%, 96%, 97%, 98%, 99%, or 100% identical) to the nucleic acid sequence of any one of SEQ ID NOs: 193-384. In some embodiments, the sense strand has a nucleic acid sequence that is at least 85% identical (e.g., 85%, 86%, 87%, 88%, 89%, 90%, 91%, 92%, 93%, 94%, 95%, 96%, 97%, 98%, 99%, or 100% identical) to the nucleic acid sequence of SEQ ID NO: 778 or 880.

In some embodiments, the sense strand has a nucleic acid sequence that is at least 90% identical (e.g., 90%, 91%, 92%, 93%, 94%, 95%, 96%, 97%, 98%, 99%, or 100% identical) to the nucleic acid sequence of any one of SEQ ID NOs: 193-384 and 769-960. In some embodiments, the sense strand has a nucleic acid sequence that is at least 90% identical (e.g., 90%, 91%, 92%, 93%, 94%, 95%, 96%, 97%, 98%, 99%, or 100% identical) to the nucleic acid sequence of any one of SEQ ID NOs: 193-384. In some embodiments, the sense strand has a nucleic acid sequence that is at least 90% identical (e.g., 90%, 91%, 92%, 93%, 94%, 95%, 96%, 97%, 98%, 99%, or 100% identical) to the nucleic acid sequence of SEQ ID NO: 778 or 880.

In some embodiments, the sense strand has a nucleic acid sequence that is at least 95% identical (e.g., 95%, 96%, 97%, 98%, 99%, or 100% identical) to the nucleic acid sequence of any one of SEQ ID NOs: 193-384 and 769-960, optionally wherein the sense strand has a nucleic acid sequence that is at least 96%, 97%, 98%, or 99% identical to the nucleic acid sequence of any one of SEQ ID NOs: 193-384 and 769-960. In some embodiments, the sense strand has a nucleic acid sequence that is at least 95% identical (e.g., 95%, 96%, 97%, 98%, 99%, or 100% identical) to the nucleic acid sequence of any one of SEQ ID NOs: 193-384, optionally wherein the sense strand has a nucleic acid sequence that is at least 96%, 97%, 98%, or 99% identical to the nucleic acid sequence of any one of SEQ ID NOs: 193-384. In some embodiments, the sense strand has a nucleic acid sequence that is at least 95% identical (e.g., 95%, 96%, 97%, 98%, 99%, or 100% identical) to the nucleic acid sequence of SEQ ID NO: 778 or 880, optionally wherein the sense strand has a nucleic acid sequence that is at least 96%, 97%, 98%, or 99% identical to the nucleic acid sequence of SEQ ID NO: 778 or 880.

In some embodiments, the siRNA molecule has a sense strand having the nucleic acid sequence of any one of SEQ ID NOs: 193-384 and 769-960. In some embodiments, the siRNA molecule has a sense strand having the nucleic acid sequence of any one of SEQ ID NOs: 193-384. In some embodiments, the siRNA molecule has a sense strand having the nucleic acid sequence of SEQ ID NO: 778 or 880.

In some embodiments, the antisense strand has a structure represented by Formula I, wherein Formula I is, in the 5′-to-3′ direction:

A-B-(A′)ⱼ-C-P²-D-P¹-(C′-P¹)k-C′ Formula I;
wherein A is represented by the formula C-P¹-D-P¹;
each A′ is represented by the formula C-P²-D-P²;
B is represented by the formula C-P²-D-P²-D-P²-D-P²;
each C is a 2′-O-methyl (2′-O-Me) ribonucleoside;
each C′, independently, is a 2′-O-Me ribonucleoside or a 2′-fluoro (2′-F) ribonucleoside;
each D is a 2′-F ribonucleoside;
each P¹ is a phosphorothioate internucleoside linkage;
each P² is a phosphodiester internucleoside linkage;
j is an integer from 1 to 7 (e.g., 1, 2, 3, 4, 5, 6, or 7); and
k is an integer from 1 to 7 (e.g., 1, 2, 3, 4, 5, 6, or 7).

In some embodiments, the antisense strand has a structure represented by Formula A1, wherein Formula A1 is, in the 5′-to-3′ direction:

A-S-B-S-A-O-B-O-B-O-B-O-A-O-B-O-A-O-B-O-A-O-B-O-A-O-B-O-A-O-B-S-A-S-A-S-A-S-B-S-A Formula A1;
wherein A represents a 2′-O-Me ribonucleoside, B represents a 2′-F ribonucleoside, O represents a phosphodiester internucleoside linkage, and S represents a phosphorothioate internucleoside linkage.

In some embodiments, the antisense strand has a structure represented by Formula II, wherein Formula II is, in the 5′-to-3′ direction:

A-B-(A′)ⱼ-C-P²-D-P¹-(C-P¹)k-C′ Formula II;
wherein A is represented by the formula C-P¹-D-P¹;
each A′ is represented by the formula C-P²-D-P²;
B is represented by the formula C-P²-D-P²-D-P²-D-P²;
each C is a 2′-O-methyl (2′-O-Me) ribonucleoside;
each C′, independently, is a 2′-O-Me ribonucleoside or a 2′-fluoro (2′-F) ribonucleoside;
each D is a 2′-F ribonucleoside;
each P¹ is a phosphorothioate internucleoside linkage;
each P² is a phosphodiester internucleoside linkage;
j is an integer from 1 to 7 (e.g., 1, 2, 3, 4, 5, 6, or 7); and
k is an integer from 1 to 7 (e.g., 1, 2, 3, 4, 5, 6, or 7).

In some embodiments, antisense strand has a structure represented by Formula A2, wherein Formula A2 is, in the 5′-to-3′ direction:

A-S-B-S-A-O-B-O-B-O-B-O-A-O-B-O-A-O-B-O-A-O-B-O-A-O-B-O-A-O-B-S-A-S-A-S-A-S-A-S-A Formula A2;
wherein A represents a 2′-O-Me ribonucleoside, B represents a 2′-F ribonucleoside, O represents a phosphodiester internucleoside linkage, and S represents a phosphorothioate internucleoside linkage.

In some embodiments, the sense strand has a structure represented by Formula III, wherein Formula III is, in the 5′-to-3′ direction:

E-(A′)ₘ-F Formula III;
wherein E is represented by the formula (C-P¹)₂;
F is represented by the formula (C-P²)₃-D-P¹-C-P¹-C, (C-P²)₃-D-P²-C-P²-C, (C-P²)₃-D-P¹-C-P¹-D, or (C-P²)₃-D-P²-C-P²-D;
A′, C, D, P¹, and P² are as defined in Formula II; and
m is an integer from 1 to 7 (e.g., 1, 2, 3, 4, 5, 6, or 7).

In some embodiments, the sense strand has a structure represented by Formula S1, wherein Formula S1 is, in the 5′-to-3′ direction:

A-S-A-S-A-O-B-O-A-O-B-O-A-O-B-O-A-O-B-O-A-O-A-O-A-O-B-S-A-S-A Formula S1;
wherein A represents a 2′-O-Me ribonucleoside, B represents a 2′-F ribonucleoside, O represents a phosphodiester internucleoside linkage, and S represents a phosphorothioate internucleoside linkage.

In some embodiments, the sense strand has a structure represented by Formula S2, wherein Formula S2 is, in the 5′-to-3′ direction:

A-S-A-S-A-O-B-O-A-O-B-O-A-O-B-O-A-O-B-O-A-O-A-O-A-O-B-O-A-O-A Formula S2;
wherein A represents a 2′-O-Me ribonucleoside, B represents a 2′-F ribonucleoside, O represents a phosphodiester internucleoside linkage, and S represents a phosphorothioate internucleoside linkage.

In some embodiments, the sense strand has a structure represented by Formula S3, wherein Formula S3 is, in the 5′-to-3′ direction:

A-S-A-S-A-O-B-O-A-O-B-O-A-O-B-O-A-O-B-O-A-O-A-O-A-O-B-S-A-S-B Formula S3;
wherein A represents a 2′-O-Me ribonucleoside, B represents a 2′-F ribonucleoside, O represents a phosphodiester internucleoside linkage, and S represents a phosphorothioate internucleoside linkage.

In some embodiments, the sense strand has a structure represented by Formula S4, wherein Formula S4 is, in the 5′-to-3′ direction:

A-S-A-S-A-O-B-O-A-O-B-O-A-O-B-O-A-O-B-O-A-O-A-O-A-O-B-O-A-O-B Formula S4;
wherein A represents a 2′-O-Me ribonucleoside, B represents a 2′-F ribonucleoside, O represents a phosphodiester internucleoside linkage, and S represents a phosphorothioate internucleoside linkage.

In some embodiments, the antisense strand has a structure represented by Formula IV, wherein Formula IV is, in the 5′-to-3′ direction:

A-(A′)ⱼ-C-P²-B-(C-P¹)ₖ-C′ Formula IV;
wherein A is represented by the formula C-P¹-D-P¹;
each A′ is represented by the formula C-P²-D-P²;
B is represented by the formula D-P¹-C-P¹-D-P¹;
each C is a 2′-O-Me ribonucleoside;
each C′, independently, is a 2′-O-Me ribonucleoside or a 2′-F ribonucleoside;
each D is a 2′-F ribonucleoside;
each P¹ is a phosphorothioate internucleoside linkage;
each P² is a phosphodiester internucleoside linkage;
j is an integer from 1 to 7 (e.g., 1, 2, 3, 4, 5, 6, or 7); and
k is an integer from 1 to 7 (e.g., 1, 2, 3, 4, 5, 6, or 7).

In some embodiments, the antisense strand has a structure represented by Formula A3, wherein Formula A3 is, in the 5′-to-3′ direction:

A-S-B-S-A-O-B-O-A-O-B-O-A-O-B-O-A-O-B-O-A-O-B-O-A-O-B-O-A-O-B-S-A-S-B-S-A-S-A-S-A Formula A3;
wherein A represents a 2′-O-Me ribonucleoside, B represents a 2′-F ribonucleoside, O represents a phosphodiester internucleoside linkage, and S represents a phosphorothioate internucleoside linkage.

In some embodiments, the sense strand has a structure represented by Formula V, wherein Formula V is, in the 5′-to-3′ direction:

E-(A′)ₘ-C-P²-F Formula V;
wherein E is represented by the formula (C-P¹)₂;
F is represented by the formula D-P¹-C-P¹-C, D-P²-C-P²-C, D-P¹-C-P¹-D, or D-P²-C-P²-D;
A′, C, D, P¹ and P² are as defined in Formula IV; and
m is an integer from 1 to 7 (e.g., 1, 2, 3, 4, 5, 6, or 7).

In some embodiments, the sense strand has a structure represented by Formula S5, wherein Formula S5 is, in the 5′-to-3′ direction:

A-S-A-S-A-O-B-O-A-O-B-O-A-O-B-O-A-O-B-O-A-O-B-O-A-O-B-S-A-S-A Formula S5;
wherein A represents a 2′-O-Me ribonucleoside, B represents a 2′-F ribonucleoside, O represents a phosphodiester internucleoside linkage, and S represents a phosphorothioate internucleoside linkage.

In some embodiments, the sense strand has a structure represented by Formula S6, wherein Formula S6 is, in the 5′-to-3′ direction:

A-S-A-S-A-O-B-O-A-O-B-O-A-O-B-O-A-O-B-O-A-O-B-O-A-O-B-O-A-O-A Formula S6;
wherein A represents a 2′-O-Me ribonucleoside, B represents a 2′-F ribonucleoside, O represents a phosphodiester internucleoside linkage, and S represents a phosphorothioate internucleoside linkage.

In some embodiments, the sense strand has a structure represented by Formula S7, wherein Formula S7 is, in the 5′-to-3′ direction:

A-S-A-S-A-O-B-O-A-O-B-O-A-O-B-O-A-O-B-O-A-O-B-O-A-O-B-S-A-S-B Formula S7;
wherein A represents a 2′-O-Me ribonucleoside, B represents a 2′-F ribonucleoside, O represents a phosphodiester internucleoside linkage, and S represents a phosphorothioate internucleoside linkage.

In some embodiments, the sense strand has a structure represented by Formula S8, wherein Formula S8 is, in the 5′-to-3′ direction:

A-S-A-S-A-O-B-O-A-O-B-O-A-O-B-O-A-O-B-O-A-O-B-O-A-O-B-O-A-O-B Formula S8;
wherein A represents a 2′-O-Me ribonucleoside, B represents a 2′-F ribonucleoside, O represents a phosphodiester internucleoside linkage, and S represents a phosphorothioate internucleoside linkage.

In some embodiments, the antisense strand has a structure represented by Formula VI, wherein Formula VI is, in the 5′-to-3′ direction:

A-Bⱼ-E-Bₖ-E-F-Gₗ-D-P¹-C′ Formula VI;
wherein A is represented by the formula C-P¹-D-P¹;
each B is represented by the formula C-P²;
each C is a 2′-O-Me ribonucleoside;
each C′, independently, is a 2′-O-Me ribonucleoside or a 2′-F ribonucleoside;
each D is a 2′-F ribonucleoside;
each E is represented by the formula D-P²-C-P²;
F is represented by the formula D-P¹-C-P¹;
each G is represented by the formula C-P¹;
each P¹ is a phosphorothioate internucleoside linkage;
each P² is a phosphodiester internucleoside linkage;
j is an integer from 1 to 7 (e.g., 1, 2, 3, 4, 5, 6, or 7);
k is an integer from 1 to 7 (e.g., 1, 2, 3, 4, 5, 6, or 7); and
l is an integer from 1 to 7 (e.g., 1, 2, 3, 4, 5, 6, or 7).

In some embodiments, the antisense strand has a structure represented by Formula A4, wherein Formula A4 is, in the 5′-to-3′ direction:

A-S-B-S-A-O-A-O-A-O-B-O-A-O-A-O-A-O-A-O-A-O-A-O-A-O-B-O-A-O-B-S-A-S-A-S-A-S-B-S-A Formula A4;
wherein A represents a 2′-O-Me ribonucleoside, B represents a 2′-F ribonucleoside, O represents a phosphodiester internucleoside linkage, and S represents a phosphorothioate internucleoside linkage.

In some embodiments, the sense strand has a structure represented by Formula VII, wherein Formula VII is, in the 5′-to-3′ direction:

H-Bₘ-Iₙ-A′-Bₒ-H-C Formula VII;
wherein A′ is represented by the formula C-P²-D-P²;
each H is represented by the formula (C-P¹)₂;
each I is represented by the formula (D-P²);
B, C, D, P¹ and P² are as defined in Formula VI;
m is an integer from 1 to 7 (e.g., 1, 2, 3, 4, 5, 6, or 7);
n is an integer from 1 to 7 (e.g., 1, 2, 3, 4, 5, 6, or 7); and
is an integer from 1 to 7 (e.g., 1, 2, 3, 4, 5, 6, or 7).

In some embodiments, the sense strand has a structure represented by Formula S9, wherein Formula S9 is, in the 5′-to-3′ direction:

A-S-A-S-A-O-A-O-A-O-B-O-B-O-B-O-A-O-B-O-A-O-A-O-A-O-A-S-A-S-A Formula S9;
wherein A represents a 2′-O-Me ribonucleoside, B represents a 2′-F ribonucleoside, O represents a phosphodiester internucleoside linkage, and S represents a phosphorothioate internucleoside linkage.

In some embodiments, the antisense strand also has a 5′ phosphorus stabilizing moiety at the 5′ end of the antisense strand.

In some embodiments, the sense strand also has a 5′ phosphorus stabilizing moiety at the 5′ end of the sense strand.

In some embodiments, each 5′ phosphorus stabilizing moiety is, independently, represented by any one of Formulas IX, XX, XI, XII, XIII, XIV, XV, or XVI:

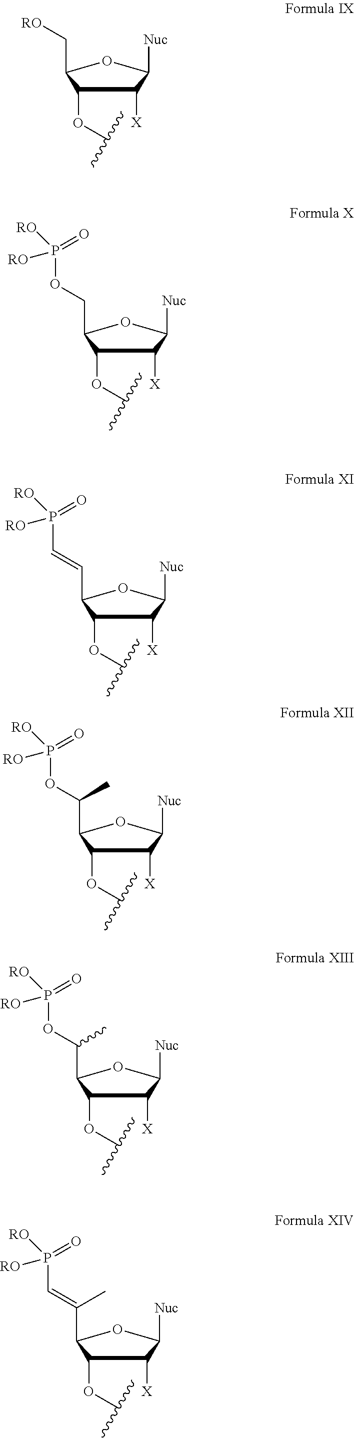

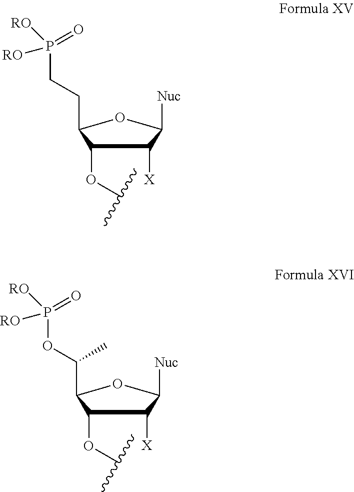

wherein Nuc represents a nucleobase, optionally wherein the nucleobase is selected from the group consisting of adenine, uracil, guanine, thymine, and cytosine, and R represents an optionally substituted alkyl, optionally substituted alkenyl, optionally substituted alkynyl, phenyl, benzyl, a cation (e.g., a monovalent cation), or hydrogen.

In some embodiments, the nucleobase is an adenine, uracil, guanine, thymine, or cytosine.

In some embodiments, the 5′ phosphorus stabilizing moiety is (E)-vinylphosphonate represented by Formula XI.

In some embodiments, the siRNA molecule also has a hydrophobic moiety at the 5′ or the 3′ end of the siRNA molecule.

In some embodiments, the hydrophobic moiety is selected from a group consisting of cholesterol, vitamin D, or tocopherol.

In some embodiments, the siRNA molecule is a branched siRNA molecule.

In some embodiments, the branched siRNA molecule is di-branched, tri-branched, or tetra-branched.

In some embodiments, the siRNA molecule is di-branched, optionally wherein the di-branched siRNA molecule is represented by any one of Formulas XVII, XVIII, or XIX:

RNA-L-RNA Formula XVII;

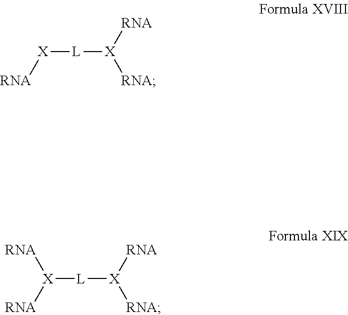

wherein each RNA is, independently, an siRNA molecule, L is a linker, and each X, independently, represents a branch point moiety.

In some embodiments, the di-branched siRNA molecule is represented by Formula XVII. In some embodiments, the di-branched siRNA molecule is represented by Formula XVIII. In some embodiments, the di-branched siRNA molecule is represented by Formula XIX.

In some embodiments, the siRNA molecule is tri-branched, optionally wherein the tri-branched siRNA molecule is represented by any one of Formulas XX, XXI, XXII, or XXIII:

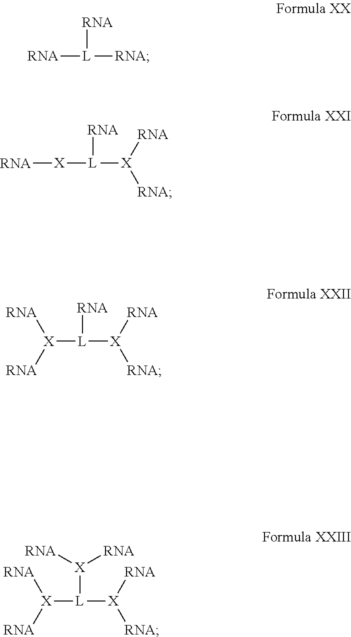

wherein each RNA is, independently, an siRNA molecule, L is a linker, and each X, independently, represents a branch point moiety.

In some embodiments, the tri-branched siRNA molecule is represented by Formula XX. In some embodiments, the tri-branched siRNA molecule is represented by Formula XXI. In some embodiments, the tri-branched siRNA molecule is represented by Formula XXII. In some embodiments, the tri-branched siRNA molecule is represented by Formula XXIII.

In some embodiments, the siRNA molecule is tetra-branched, optionally wherein the tetra-branched siRNA molecule is represented by any one of Formulas XXIV, XXV, XXVI, XXVII, or XXVIII:

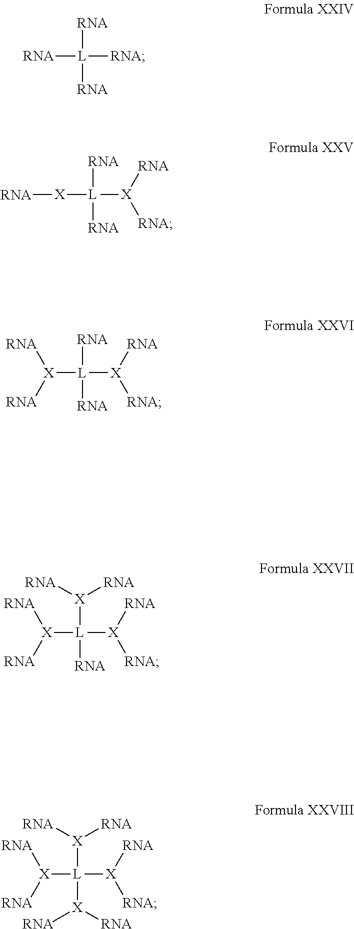

wherein each RNA is, independently, an siRNA molecule, L is a linker, and each X, independently, represents a branch point moiety.

In some embodiments, the tetra-branched siRNA molecule is represented by Formula XXIV. In some embodiments, the tetra-branched siRNA molecule is represented by Formula XXV. In some embodiments, the tetra-branched siRNA molecule is represented by Formula XXVI. In some embodiments, the tetra-branched siRNA molecule is represented by Formula XXVII. In some embodiments, the tetra-branched siRNA molecule is represented by Formula XXVIII.

In some embodiments of the branched siRNA, the linker is selected from a group consisting of one or more contiguous subunits of an ethylene glycol (e.g., polyethylene glycol (PEG), such as, e.g., triethylene glycol (TrEG) or tetraethylene glycol (TEG)), alkyl, carbohydrate, block copolymer, peptide, RNA, and DNA.

In some embodiments, the linker is an ethylene glycol oligomer. In some embodiments, the linker is an alkyl oligomer. In some embodiments, the linker is a carbohydrate oligomer. In some embodiments, the linker is a block copolymer. In some embodiments, the linker is a peptide oligomer. In some embodiments, the linker is an RNA oligomer. In some embodiments, the linker is a DNA oligomer.

In some embodiments, the ethylene glycol oligomer is a PEG. In some embodiments, the PEG is a TrEG. In some embodiments, the PEG is a TEG.

In some embodiments, the oligomer or copolymer contains 2 to 20 contiguous subunits (e.g., 2, 3, 4, 5, 6, 7, 8, 9, 10, 11, 12, 13, 14, 15, 16, 17, 18, 19, or 20 contiguous subunits).

In some embodiments, the linker attaches one or more (e.g., 1, 2, 3, 4, or more) siRNA molecules by way of a covalent bond-forming moiety.

In some embodiments, the covalent bond-forming moiety is selected from the group consisting of an alkyl, ester, amide, carbamate, phosphonate, phosphate, phosphorothioate, phosphoroamidate, triazole, urea, and formacetal.

In some embodiments, the linker includes a structure of Formula L1:

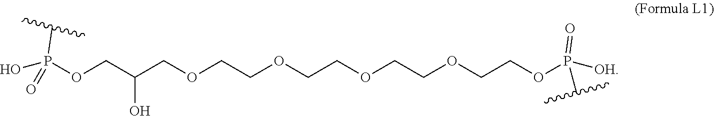 [SMILES: *P(=O)(O)OCCOCCOCCOCCOCC(O)COP(*)(=O)O]

In some embodiments, the linker includes a structure of Formula L2:

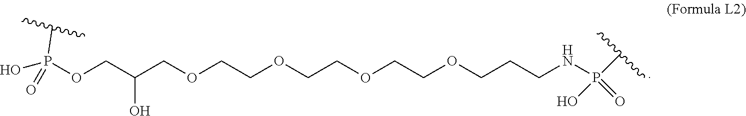 [SMILES: *P(=O)(O)NCCCOCCOCCOCCOCC(O)COP(*)(=O)O]

In some embodiments, the linker includes a structure of Formula L3:

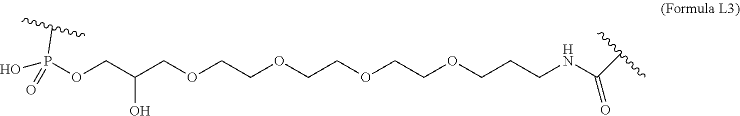 [SMILES: *C(=O)NCCCOCCOCCOCCOCC(O)COP(*)(=O)O]

In some embodiments, the linker includes a structure of Formula L4:

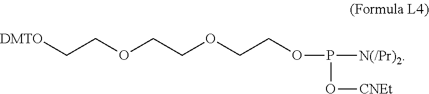

In some embodiments, the linker includes a structure of Formula L5:

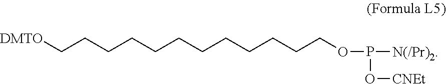

In some embodiments, the linker includes a structure of Formula L6:

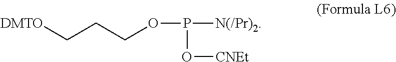

In some embodiments, the linker includes a structure of Formula L7:

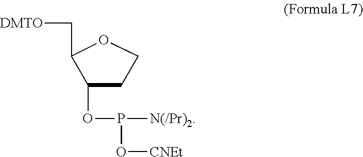

In some embodiments, the linker includes a structure of Formula L8:

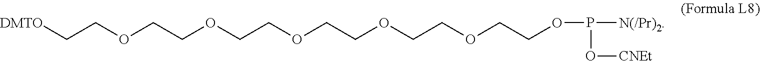

In some embodiments, the linker includes a structure of Formula L9:

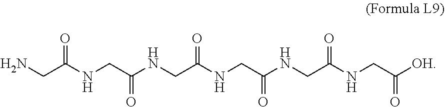 [SMILES: NCC(=O)NCC(=O)NCC(=O)NCC(=O)NCC(=O)NCC(=O)O]

In some embodiments of any of the siRNA molecules described herein, 50% or more of the ribonucleotides in the antisense strand are 2′-O-Me ribonucleotides (e.g., 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% of the ribonucleotides in the antisense strand may be 2′-O-Me ribonucleotides).

In some embodiments, 60% or more of the ribonucleotides in the antisense strand are 2′-O-Me ribonucleotides (e.g., 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% of the ribonucleotides in the antisense strand may be 2′-O-Me ribonucleotides).

In some embodiments, 70% or more of the ribonucleotides in the antisense strand are 2′-O-Me ribonucleotides (e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% of the ribonucleotides in the antisense strand may be 2′-O-Me ribonucleotides).

In some embodiments, 80% or more of the ribonucleotides in the antisense strand are 2′-O-Me ribonucleotides (e.g., 80%, 81%, 82%, 83%, 84%, 85%, 86%, 87%, 88%, 89%, 90%, 91%, 92%, 93%, 94%, 95%, 96%, 97%, 98%, 99%, or 100% of the ribonucleotides in the antisense strand may be 2′-O-Me ribonucleotides).

In some embodiments, 90% or more of the ribonucleotides in the antisense strand are 2′-O-Me ribonucleotides (e.g., 90%, 91%, 92%, 93%, 94%, 95%, 96%, 97%, 98%, 99%, or 100% of the ribonucleotides in the antisense strand may be 2′-O-Me ribonucleotides).

In some embodiments, 10% or less of the internucleoside linkages are phosphodiester linkages or phosphorothioate linkages. In some embodiments, at least 10%, 20%, 30%, 40%, 50%, 60%, 70%, 80%, or 90% of the internucleoside linkages are phosphodiester linkages or phosphorothioate linkages. In some embodiments, 100% of the internucleoside linkages are phosphodiester linkages or phosphorothioate linkages.

In some embodiments, 9 internucleoside linkages are phosphodiester linkages or phosphorothioate linkages.

In some embodiments, the length of the antisense strand is between 10 and 30 nucleotides (e.g., 10 nucleotides, 11 nucleotides, 12 nucleotides, 13 nucleotides, 14 nucleotides, 15 nucleotides, 16 nucleotides, 17 nucleotides, 18 nucleotides, 19 nucleotides, 20 nucleotides, 21 nucleotides, 22 nucleotides, 23 nucleotides, 24 nucleotides, 25 nucleotides, 26 nucleotides, 27 nucleotides, 28 nucleotides, 29 nucleotides, or 30 nucleotides), 15 and 25 nucleotides (e.g., 15 nucleotides, 16 nucleotides, 17 nucleotides, 18 nucleotides, 19 nucleotides, 20 nucleotides, 21 nucleotides, 22 nucleotides, 23 nucleotides, 24 nucleotides, or 25 nucleotides), or 18 and 23 nucleotides (e.g., 18 nucleotides, 19 nucleotides, 20 nucleotides, 21 nucleotides, 22 nucleotides, or 23 nucleotides). In some embodiments, the length of the antisense strand is 20 nucleotides. In some embodiments, the length of the antisense strand is 21 nucleotides. In some embodiments, the length of the antisense strand is 22 nucleotides. In some embodiments, the length of the antisense strand is 23 nucleotides. In some embodiments, the length of the antisense strand is 24 nucleotides. In some embodiments, the length of the antisense strand is 25 nucleotides. In some embodiments, the length of the antisense strand is 26 nucleotides. In some embodiments, the length of the antisense strand is 27 nucleotides. In some embodiments, the length of the antisense strand is 28 nucleotides. In some embodiments, the length of the antisense strand is 29 nucleotides. In some embodiments, the length of the antisense strand is 30 nucleotides.

In some embodiments, the siRNA molecules of the branched compound are joined to one another by way of a linker (e.g., an ethylene glycol oligomer, such as tetraethylene glycol). In some embodiments, the siRNA molecules of the branched compound are joined to one another by way of a linker between the sense strand of one siRNA molecule and the sense strand of the other siRNA molecule. In some embodiments, the siRNA molecules are joined by way of linkers between the antisense strand of one siRNA molecule and the antisense strand of the other siRNA molecule. In some embodiments, the siRNA molecules of the branched compound are joined to one another by way of a linker between the sense strand of one siRNA molecule and the antisense strand of the other siRNA molecule.

In some embodiments, the length of the sense strand is between 12 and 30 nucleotides (e.g., 12 nucleotides, 13 nucleotides, 14 nucleotides, 15 nucleotides, 16 nucleotides, 17 nucleotides, 18 nucleotides, 19 nucleotides, 20 nucleotides, 21 nucleotides, 22 nucleotides, 23 nucleotides, 24 nucleotides, 25 nucleotides, 26 nucleotides, 27 nucleotides, 28 nucleotides, 29 nucleotides, or 30 nucleotides), or 14 and 18 nucleotides (e.g., 14 nucleotides, 15 nucleotides, 16 nucleotides, 17 nucleotides, or 18 nucleotides). In some embodiments, the length of the sense strand is 15 nucleotides. In some embodiments, the length of the sense strand is 16 nucleotides. In some embodiments, the length of the sense strand is 17 nucleotides. In some embodiments, the length of the sense strand is 18 nucleotides. In some embodiments, the length of the sense strand is 19 nucleotides. In some embodiments, the length of the sense strand is 20 nucleotides. In some embodiments, the length of the sense strand is 21 nucleotides. In some embodiments, the length of the sense strand is 22 nucleotides. In some embodiments, the length of the sense strand is 23 nucleotides. In some embodiments, the length of the sense strand is 24 nucleotides. In some embodiments, the length of the sense strand is 25 nucleotides. In some embodiments, the length of the sense strand is 26 nucleotides. In some embodiments, the length of the sense strand is 27 nucleotides. In some embodiments, the length of the sense strand is 28 nucleotides. In some embodiments, the length of the sense strand is 29 nucleotides. In some embodiments, the length of the sense strand is 30 nucleotides.

In some embodiments, four internucleoside linkages are phosphorothioate linkages.

In some embodiments of the siRNA molecules described herein, the antisense strand is 18 nucleotides in length and the sense strand is 14 nucleotides in length. In some embodiments, the antisense strand is 18 nucleotides in length and the sense strand is 15 nucleotides in length. In some embodiments, the antisense strand is 18 nucleotides in length and the sense strand is 16 nucleotides in length. In some embodiments, the antisense strand is 18 nucleotides in length and the sense strand is 17 nucleotides in length. In some embodiments, the antisense strand is 18 nucleotides in length and the sense strand is 18 nucleotides in length. In some embodiments, the antisense strand is 19 nucleotides in length and the sense strand is 14 nucleotides in length. In some embodiments, the antisense strand is 19 nucleotides in length and the sense strand is 15 nucleotides in length. In some embodiments, the antisense strand is 19 nucleotides in length and the sense strand is 16 nucleotides in length. In some embodiments, the antisense strand is 19 nucleotides in length and the sense strand is 17 nucleotides in length. In some embodiments, the antisense strand is 19 nucleotides in length and the sense strand is 18 nucleotides in length. In some embodiments, the antisense strand is 19 nucleotides in length and the sense strand is 19 nucleotides in length. In some embodiments, the antisense strand is 20 nucleotides in length and the sense strand is 14 nucleotides in length. In some embodiments, the antisense strand is 20 nucleotides in length and the sense strand is 15 nucleotides in length. In some embodiments, the antisense strand is 20 nucleotides in length and the sense strand is 16 nucleotides in length. In some embodiments, the antisense strand is 20 nucleotides in length and the sense strand is 17 nucleotides in length. In some embodiments, the antisense strand is 20 nucleotides in length and the sense strand is 18 nucleotides in length. In some embodiments, the antisense strand is 20 nucleotides in length and the sense strand is 19 nucleotides in length. In some embodiments, the antisense strand is 20 nucleotides in length and the sense strand is 20 nucleotides in length. In some embodiments, the antisense strand is 21 nucleotides in length and the sense strand is 14 nucleotides in length. In some embodiments, the antisense strand is 21 nucleotides in length and the sense strand is 15 nucleotides in length. In some embodiments, the antisense strand is 21 nucleotides in length and the sense strand is 16 nucleotides in length. In some embodiments, the antisense strand is 21 nucleotides in length and the sense strand is 17 nucleotides in length. In some embodiments, the antisense strand is 21 nucleotides in length and the sense strand is 18 nucleotides in length. In some embodiments, the antisense strand is 21 nucleotides in length and the sense strand is 19 nucleotides in length. In some embodiments, the antisense strand is 21 nucleotides in length and the sense strand is 20 nucleotides in length. In some embodiments, the antisense strand is 21 nucleotides in length and the sense strand is 21 nucleotides in length. In some embodiments, the antisense strand is 22 nucleotides in length and the sense strand is 14 nucleotides in length. In some embodiments, the antisense strand is 22 nucleotides in length and the sense strand is 15 nucleotides in length. In some embodiments, the antisense strand is 22 nucleotides in length and the sense strand is 16 nucleotides in length. In some embodiments, the antisense strand is 22 nucleotides in length and the sense strand is 17 nucleotides in length. In some embodiments, the antisense strand is 22 nucleotides in length and the sense strand is 18 nucleotides in length. In some embodiments, the antisense strand is 22 nucleotides in length and the sense strand is 19 nucleotides in length. In some embodiments, the antisense strand is 22 nucleotides in length and the sense strand is 20 nucleotides in length. In some embodiments, the antisense strand is 22 nucleotides in length and the sense strand is 21 nucleotides in length. In some embodiments, the antisense strand is 22 nucleotides in length and the sense strand is 22 nucleotides in length. In some embodiments, the antisense strand is 23 nucleotides in length and the sense strand is 14 nucleotides in length. In some embodiments, the antisense strand is 23 nucleotides in length and the sense strand is 15 nucleotides in length. In some embodiments, the antisense strand is 23 nucleotides in length and the sense strand is 16 nucleotides in length. In some embodiments, the antisense strand is 23 nucleotides in length and the sense strand is 17 nucleotides in length. In some embodiments, the antisense strand is 23 nucleotides in length and the sense strand is 18 nucleotides in length. In some embodiments, the antisense strand is 23 nucleotides in length and the sense strand is 19 nucleotides in length. In some embodiments, the antisense strand is 23 nucleotides in length and the sense strand is 20 nucleotides in length. In some embodiments, the antisense strand is 23 nucleotides in length and the sense strand is 21 nucleotides in length. In some embodiments, the antisense strand is 23 nucleotides in length and the sense strand is 22 nucleotides in length. In some embodiments, the antisense strand is 23 nucleotides in length and the sense strand is 23 nucleotides in length. In some embodiments, the antisense strand is 24 nucleotides in length and the sense strand is 14 nucleotides in length. In some embodiments, the antisense strand is 24 nucleotides in length and the sense strand is 15 nucleotides in length. In some embodiments, the antisense strand is 24 nucleotides in length and the sense strand is 16 nucleotides in length. In some embodiments, the antisense strand is 24 nucleotides in length and the sense strand is 17 nucleotides in length. In some embodiments, the antisense strand is 24 nucleotides in length and the sense strand is 18 nucleotides in length. In some embodiments, the antisense strand is 24 nucleotides in length and the sense strand is 19 nucleotides in length. In some embodiments, the antisense strand is 24 nucleotides in length and the sense strand is 20 nucleotides in length. In some embodiments, the antisense strand is 24 nucleotides in length and the sense strand is 21 nucleotides in length. In some embodiments, the antisense strand is 24 nucleotides in length and the sense strand is 22 nucleotides in length. In some embodiments, the antisense strand is 24 nucleotides in length and the sense strand is 23 nucleotides in length. In some embodiments, the antisense strand is 24 nucleotides in length and the sense strand is 24 nucleotides in length. In some embodiments, the antisense strand is 25 nucleotides in length and the sense strand is 14 nucleotides in length. In some embodiments, the antisense strand is 25 nucleotides in length and the sense strand is 15 nucleotides in length. In some embodiments, the antisense strand is 25 nucleotides in length and the sense strand is 16 nucleotides in length. In some embodiments, the antisense strand is 25 nucleotides in length and the sense strand is 17 nucleotides in length. In some embodiments, the antisense strand is 25 nucleotides in length and the sense strand is 18 nucleotides in length. In some embodiments, the antisense strand is 25 nucleotides in length and the sense strand is 19 nucleotides in length. In some embodiments, the antisense strand is 25 nucleotides in length and the sense strand is 20 nucleotides in length. In some embodiments, the antisense strand is 25 nucleotides in length and the sense strand is 21 nucleotides in length. In some embodiments, the antisense strand is 25 nucleotides in length and the sense strand is 22 nucleotides in length. In some embodiments, the antisense strand is 25 nucleotides in length and the sense strand is 23 nucleotides in length. In some embodiments, the antisense strand is 25 nucleotides in length and the sense strand is 24 nucleotides in length. In some embodiments, the antisense strand is 25 nucleotides in length and the sense strand is 25 nucleotides in length. In some embodiments, the antisense strand is 26 nucleotides in length and the sense strand is 14 nucleotides in length. In some embodiments, the antisense strand is 26 nucleotides in length and the sense strand is 15 nucleotides in length. In some embodiments, the antisense strand is 26 nucleotides in length and the sense strand is 16 nucleotides in length. In some embodiments, the antisense strand is 26 nucleotides in length and the sense strand is 17 nucleotides in length. In some embodiments, the antisense strand is 26 nucleotides in length and the sense strand is 18 nucleotides in length. In some embodiments, the antisense strand is 26 nucleotides in length and the sense strand is 19 nucleotides in length. In some embodiments, the antisense strand is 26 nucleotides in length and the sense strand is 20 nucleotides in length. In some embodiments, the antisense strand is 26 nucleotides in length and the sense strand is 21 nucleotides in length. In some embodiments, the antisense strand is 26 nucleotides in length and the sense strand is 22 nucleotides in length. In some embodiments, the antisense strand is 26 nucleotides in length and the sense strand is 23 nucleotides in length. In some embodiments, the antisense strand is 26 nucleotides in length and the sense strand is 24 nucleotides in length. In some embodiments, the antisense strand is 26 nucleotides in length and the sense strand is 25 nucleotides in length. In some embodiments, the antisense strand is 26 nucleotides in length and the sense strand is 26 nucleotides in length. In some embodiments, the antisense strand is 27 nucleotides in length and the sense strand is 14 nucleotides in length. In some embodiments, the antisense strand is 27 nucleotides in length and the sense strand is 15 nucleotides in length. In some embodiments, the antisense strand is 27 nucleotides in length and the sense strand is 16 nucleotides in length. In some embodiments, the antisense strand is 27 nucleotides in length and the sense strand is 17 nucleotides in length. In some embodiments, the antisense strand is 27 nucleotides in length and the sense strand is 18 nucleotides in length. In some embodiments, the antisense strand is 27 nucleotides in length and the sense strand is 19 nucleotides in length. In some embodiments, the antisense strand is 27 nucleotides in length and the sense strand is 20 nucleotides in length. In some embodiments, the antisense strand is 27 nucleotides in length and the sense strand is 21 nucleotides in length. In some embodiments, the antisense strand is 27 nucleotides in length and the sense strand is 22 nucleotides in length. In some embodiments, the antisense strand is 27 nucleotides in length and the sense strand is 23 nucleotides in length. In some embodiments, the antisense strand is 27 nucleotides in length and the sense strand is 24 nucleotides in length. In some embodiments, the antisense strand is 27 nucleotides in length and the sense strand is 25 nucleotides in length. In some embodiments, the antisense strand is 27 nucleotides in length and the sense strand is 26 nucleotides in length. In some embodiments, the antisense strand is 27 nucleotides in length and the sense strand is 27 nucleotides in length. In some embodiments, the antisense strand is 28 nucleotides in length and the sense strand is 14 nucleotides in length. In some embodiments, the antisense strand is 28 nucleotides in length and the sense strand is 15 nucleotides in length. In some embodiments, the antisense strand is 28 nucleotides in length and the sense strand is 16 nucleotides in length. In some embodiments, the antisense strand is 28 nucleotides in length and the sense strand is 17 nucleotides in length. In some embodiments, the antisense strand is 28 nucleotides in length and the sense strand is 18 nucleotides in length. In some embodiments, the antisense strand is 28 nucleotides in length and the sense strand is 19 nucleotides in length. In some embodiments, the antisense strand is 28 nucleotides in length and the sense strand is 20 nucleotides in length. In some embodiments, the antisense strand is 28 nucleotides in length and the sense strand is 21 nucleotides in length. In some embodiments, the antisense strand is 28 nucleotides in length and the sense strand is 22 nucleotides in length. In some embodiments, the antisense strand is 28 nucleotides in length and the sense strand is 23 nucleotides in length. In some embodiments, the antisense strand is 28 nucleotides in length and the sense strand is 24 nucleotides in length. In some embodiments, the antisense strand is 28 nucleotides in length and the sense strand is 25 nucleotides in length. In some embodiments, the antisense strand is 28 nucleotides in length and the sense strand is 26 nucleotides in length. In some embodiments, the antisense strand is 28 nucleotides in length and the sense strand is 27 nucleotides in length. In some embodiments, the antisense strand is 28 nucleotides in length and the sense strand is 28 nucleotides in length. In some embodiments, the antisense strand is 29 nucleotides in length and the sense strand is 14 nucleotides in length. In some embodiments, the antisense strand is 29 nucleotides in length and the sense strand is 15 nucleotides in length. In some embodiments, the antisense strand is 29 nucleotides in length and the sense strand is 16 nucleotides in length. In some embodiments, the antisense strand is 29 nucleotides in length and the sense strand is 17 nucleotides in length. In some embodiments, the antisense strand is 29 nucleotides in length and the sense strand is 18 nucleotides in length. In some embodiments, the antisense strand is 29 nucleotides in length and the sense strand is 19 nucleotides in length. In some embodiments, the antisense strand is 29 nucleotides in length and the sense strand is 20 nucleotides in length. In some embodiments, the antisense strand is 29 nucleotides in length and the sense strand is 21 nucleotides in length. In some embodiments, the antisense strand is 29 nucleotides in length and the sense strand is 22 nucleotides in length. In some embodiments, the antisense strand is 29 nucleotides in length and the sense strand is 23 nucleotides in length. In some embodiments, the antisense strand is 29 nucleotides in length and the sense strand is 24 nucleotides in length. In some embodiments, the antisense strand is 29 nucleotides in length and the sense strand is 25 nucleotides in length. In some embodiments, the antisense strand is 29 nucleotides in length and the sense strand is 26 nucleotides in length. In some embodiments, the antisense strand is 29 nucleotides in length and the sense strand is 27 nucleotides in length. In some embodiments, the antisense strand is 29 nucleotides in length and the sense strand is 28 nucleotides in length. In some embodiments, the antisense strand is 29 nucleotides in length and the sense strand is 29 nucleotides in length. In some embodiments, the antisense strand is 30 nucleotides in length and the sense strand is 14 nucleotides in length. In some embodiments, the antisense strand is 30 nucleotides in length and the sense strand is 15 nucleotides in length. In some embodiments, the antisense strand is 30 nucleotides in length and the sense strand is 16 nucleotides in length. In some embodiments, the antisense strand is 30 nucleotides in length and the sense strand is 17 nucleotides in length. In some embodiments, the antisense strand is 30 nucleotides in length and the sense strand is 18 nucleotides in length. In some embodiments, the antisense strand is 30 nucleotides in length and the sense strand is 19 nucleotides in length. In some embodiments, the antisense strand is 30 nucleotides in length and the sense strand is 20 nucleotides in length. In some embodiments, the antisense strand is 30 nucleotides in length and the sense strand is 21 nucleotides in length. In some embodiments, the antisense strand is 30 nucleotides in length and the sense strand is 22 nucleotides in length. In some embodiments, the antisense strand is 30 nucleotides in length and the sense strand is 23 nucleotides in length. In some embodiments, the antisense strand is 30 nucleotides in length and the sense strand is 24 nucleotides in length. In some embodiments, the antisense strand is 30 nucleotides in length and the sense strand is 25 nucleotides in length. In some embodiments, the antisense strand is 30 nucleotides in length and the sense strand is 26 nucleotides in length. In some embodiments, the antisense strand is 30 nucleotides in length and the sense strand is 27 nucleotides in length. In some embodiments, the antisense strand is 30 nucleotides in length and the sense strand is 28 nucleotides in length. In some embodiments, the antisense strand is 30 nucleotides in length and the sense strand is 29 nucleotides in length. In some embodiments, the antisense strand is 30 nucleotides in length and the sense strand is 30 nucleotides in length.

In a further aspect, the disclosure provides a pharmaceutical composition containing an siRNA molecule of any of the preceding aspects or embodiments of the disclosure, and a pharmaceutically acceptable excipient, carrier, or diluent.

In a further aspect, the disclosure provides a method of delivering an siRNA molecule to the CNS or neurons of a subject experiencing pain or diagnosed as having pain or a pain disorder by administering a therapeutically effective amount of the siRNA molecule or a pharmaceutical composition of any of the preceding aspects or embodiments of the disclosure to the subject.

In a further aspect, the disclosure provides a method of treating pain or a pain disorder in a subject in need thereof by administering a therapeutically effective amount of an siRNA molecule or a pharmaceutical composition of any of the preceding aspects or embodiments of the disclosure to the CNS or neurons of the subject.

In some embodiments, the pain is neuropathic pain.

In some embodiments, the pain is nociceptive pain.

In some embodiments, the pain is post-operative pain. In some embodiments, the pain is persistent pain. In some embodiments, the pain is inflammatory pain.

In some embodiments, the pain disorder is Gerhardt disease, Mitchell disease, or Weir-Mitchell disease. In some embodiments, the subject has been diagnosed with erythromelalgia.

In another aspect, the disclosure provides a method of reducing SCN9A expression in a subject in need thereof by administering a therapeutically effective amount of an siRNA or pharmaceutical composition of any of the preceding aspects or embodiments of the disclosure to the CNS or neurons of the subject.

In some embodiments, the subject exhibits selective reduction in SCN9A expression compared to reduction in expression of one or more other voltage-gated sodium ion channel genes upon administration of an siRNA molecule or pharmaceutical composition of any of the preceding aspects or embodiments of the disclosure.

In some embodiments, the siRNA molecule or the pharmaceutical composition is administered to the subject by way of intrathecal injection or other delivery into the central nervous system.

In some embodiments, the subject is a human.

In another aspect, the disclosure provides a kit having an siRNA molecule or pharmaceutical composition of any of the preceding aspects or embodiments of the disclosure, and a package insert that instructs a user of the kit to perform the method of any of the preceding aspects or embodiments of the disclosure.

### BRIEF DESCRIPTION OF THE FIGURE

FIG. 1 is a graph showing the IC50 determination of two exemplary siRNA molecules of the disclosure having (1) an antisense strand of SEQ ID NO: 688 and a sense strand of SEQ ID NO: 880, having an IC50 of 0.0334 nM, and (2) an siRNA molecule having an antisense strand of SEQ ID NO: 586 and a sense strand of SEQ ID NO: 778, having an IC50 of 0.0166 nM.

### DEFINITIONS

Unless otherwise defined herein, scientific, and technical terms used herein have the meanings that are commonly understood by those of ordinary skill in the art. In the event of any latent ambiguity, definitions provided herein take precedent over any dictionary or extrinsic definition. Unless otherwise required by context, singular terms shall include pluralities and plural terms shall include the singular. The use of “or” means “and/or” unless stated otherwise. The use of the term “including,” as well as other forms, such as “includes” and “included,” is not limiting.

As used herein, the term “nucleic acids” refers to RNA or DNA molecules consisting of a chain of ribonucleotides or deoxyribonucleotides, respectively.

As used herein, the term “therapeutic nucleic acid” refers to a nucleic acid molecule (e.g., ribonucleic acid) that has partial or complete complementarity to, and interacts with, a disease-associated target mRNA and mediates silencing of expression of the mRNA.

As used herein, the term “carrier nucleic acid” refers to a nucleic acid molecule (e.g., ribonucleic acid) that has sequence complementarity with, and hybridizes with, a therapeutic nucleic acid. As used herein, the term “3′ end” refers to the end of the nucleic acid that contains an unmodified hydroxyl group at the 3′ carbon of the ribose ring.

As used herein, the term “nucleoside” refers to a molecule made up of a heterocyclic base and its sugar.

As used herein, the term “nucleotide” refers to a nucleoside having a phosphate group on its 3′ or 5′ sugar hydroxyl group.

In the context of this disclosure, the term “oligonucleotide” refers to an oligomer or polymer of ribonucleic acid (RNA) or deoxyribonucleic acid (DNA) or mimetics thereof. This term includes oligonucleotides composed of naturally-occurring nucleobases, sugars and covalent internucleoside (backbone) linkages as well as oligonucleotides having non-naturally-occurring (e.g., modified) portions that function similarly. Such modified or substituted oligonucleotides are often preferred over native forms because of desirable properties such as, for example, enhanced cellular uptake, enhanced affinity for nucleic acid target and increased stability in the presence of nucleases.

As used herein, the term “siRNA” refers to small interfering RNA duplexes that induce the RNA interference (RNAi) pathway. siRNA molecules may vary in length (generally, between 10 and 30 base pairs) and may contain varying degrees of complementarity to their target mRNA. The term “siRNA” includes duplexes of two separate strands, as well as single strands that optionally form hairpin structures including a duplex region.

As used herein, the term “antisense strand” refers to the strand of the siRNA duplex that contains some degree of complementarity to the target gene.

As used herein, the term “sense strand” refers to the strand of the siRNA duplex that contains complementarity to the antisense strand.

The term “interfering RNA molecule” refers to an RNA molecule, such as a small interfering RNA (siRNA), microRNA (miRNA), short hairpin RNA (shRNA), or an antisense oligonucleotide (ASO) that suppresses the endogenous function of a target RNA transcript.

As used herein, the terms “express” and “expression” refer to one or more of the following events: (1) production of an RNA template from a DNA sequence (e.g., by transcription); (2) processing of an RNA transcript (e.g., by splicing, editing, 5′ cap formation, and/or 3′ end processing); and (3) translation of an RNA into a polypeptide or protein. In the context of a gene that encodes a protein product, the terms “gene expression” and the like are used interchangeably with the terms “protein expression” and the like. Expression of a gene or protein of interest in a patient can manifest, for example, by detecting: an increase in the quantity or concentration of mRNA encoding corresponding protein (as assessed, e.g., using RNA detection procedures described herein or known in the art, such as quantitative polymerase chain reaction (qPCR) and RNA seq techniques), an increase in the quantity or concentration of the corresponding protein (as assessed, e.g., using protein detection methods described herein or known in the art, such as enzyme-linked immunosorbent assays (ELISA), among others), and/or an increase in the activity of the corresponding protein (e.g., in the case of an enzyme, as assessed using an enzymatic activity assay described herein or known in the art) in a sample obtained from the patient. As used herein, a cell is considered to “express” a gene or protein of interest if one or more, or all, of the above events can be detected in the cell or in a medium in which the cell resides. For example, a gene or protein of interest is considered to be “expressed” by a cell or population of cells if one can detect (i) production of a corresponding RNA transcript, such as an mRNA template, by the cell or population of cells (e.g., using RNA detection procedures described herein); (ii) processing of the RNA transcript (e.g., splicing, editing, 5′ cap formation, and/or 3′ end processing, such as using RNA detection procedures described herein); (iii) translation of the RNA template into a protein product (e.g., using protein detection procedures described herein); and/or (iv) post-translational modification of the protein product (e.g., using protein detection procedures described herein).

As used herein, the terms “target,” “targeting,” and “targeted,” in the context of the design of an siRNA, refers to generating an antisense strand so as to anneal the antisense strand to a region within the mRNA transcript of interest in a manner that results in a reduction in translation of the mRNA into the protein product.

As used herein, the terms “chemically modified nucleotide,” “nucleotide analog,” “altered nucleotide,” and “modified nucleotide” refer to a non-standard nucleotide, including non-naturally occurring ribonucleotides or deoxyribonucleotides. Exemplary nucleotide analogs are modified at any position so as to alter certain chemical properties of the nucleotide yet retain the ability of the nucleotide analog to perform its intended function.

As used herein, the term “metabolically stabilized” refers to RNA molecules that contain ribonucleotides that have been chemically modified in order to decrease the rate of metabolism of an RNA molecule that is administered to a subject. Exemplary modifications include 2′-hydroxy to 2′-O-methoxy or 2′-fluoro, and phosphodiester to phosphorothioate.

As used herein, the term “phosphorothioate” refers to a phosphate group of a nucleotide that is modified by substituting one or more of the oxygens of the phosphate group with sulfur.

As used herein, the terms “internucleoside” and “internucleotide” refer to the bonds between nucleosides and nucleotides, respectively.

As used herein, the term “antagomirs” refers to nucleic acids that can function as inhibitors of miRNA activity.

As used herein, the term “gapmers” refers to chimeric antisense nucleic acids that contain a central block of deoxynucleotide monomers sufficiently long to induce RNase H cleavage. The deoxynucleotide block is flanked by ribonucleotide monomers or ribonucleotide monomers containing modifications.

As used herein, the term “mixmers” refers to nucleic acids that contain a mix of locked nucleic acids (LNAs) and DNA.

As used herein, the term “guide RNAs” refers to nucleic acids that have sequence complementarity to a specific sequence in the genome immediately or 1 base pair upstream of the protospacer adjacent motif (PAM) sequence as used in CRISPR/Cas9 gene editing systems. Alternatively, “guide RNAs” may refer to nucleic acids that have sequence complementarity (e.g., are antisense) to a specific messenger RNA (mRNA) sequence. In this context, a guide RNA may also have sequence complementarity to a “passenger RNA” sequence of equal or shorter length, which is identical or substantially identical to the sequence of mRNA to which the guide RNA hybridizes.

As used herein, the term “branched siRNA” refers to a compound containing two or more double-stranded siRNA molecules covalently bound to one another. Branched siRNA molecules may be “di-branched,” also referred to herein as “di-siRNA,” wherein the siRNA molecule includes 2 siRNA molecules covalently bound to one another, e.g., by way of a linker. Branched siRNA molecules may be “tri-branched,” also referred to herein as “tri-siRNA,” wherein the siRNA molecule includes 3 siRNA molecules covalently bound to one another, e.g., by way of a linker. Branched siRNA molecules may be “tetra-branched,” also referred to herein as “tetra-siRNA,” wherein the siRNA molecule includes 4 siRNA molecules covalently bound to one another, e.g., by way of a linker.

As used herein, the term “branch point moiety” refers to a chemical moiety of a branched siRNA structure of the disclosure that may be covalently linked to a 5′ end or a 3′ end of an antisense strand or a sense strand of an siRNA molecule and which may support the attachment of additional single- or double-stranded siRNA molecules. Non-limiting examples of branch point moieties suitable for use in conjunction with the disclosed methods and compositions include, e.g., phosphoroamidite, tosylated solketal, 1,3-diaminopropanol, pentaerythritol, and any one of the branch point moieties described in U.S. Pat. No. 10,478,503.

The term “phosphate moiety” as used herein, refers to a terminal phosphate group that includes phosphates as well as modified phosphates. The phosphate moiety may be located at either terminus but is preferred at the 5′-terminal nucleoside. In one aspect, the terminal phosphate is unmodified having the formula —O—P(═O)(OH)OH. In another aspect, the terminal phosphate is modified such that one or more of the O and OH groups are replaced with H, O, S, N(R′) or alkyl where R′ is H, an amino protecting group or unsubstituted or substituted alkyl. In some embodiments, the 5′ and or 3′ terminal group may include from 1 to 3 phosphate moieties that are each, independently, unmodified (di- or tri-phosphates) or modified.

As used herein, the term “5′ phosphorus stabilizing moiety” refers to a terminal phosphate group that includes phosphates as well as modified phosphates (e.g., phosphorothioates, phosphodiesters, phosphonates). The phosphate moiety may be located at either terminus but is preferred at the 5′-terminal nucleoside. In one aspect, the terminal phosphate is unmodified having the formula —O—P(═O)(OH)OH. In another aspect, the terminal phosphate is modified such that one or more of the O and OH groups are replaced with H, O, S, N(R′), or alkyl where R′ is H, an amino protecting group, or unsubstituted or substituted alkyl. In some embodiments, the 5′ and or 3′ terminal group may include from 1 to 3 phosphate moieties that are each, independently, unmodified (di- or tri-phosphates) or modified.

The phosphate group of the nucleotide may also be modified, e.g., by substituting one or more of the oxygens of the phosphate group with sulfur (e.g., phosphorothioates), or by making other substitutions which allow the nucleotide to perform its intended function such as described in, for example, Eckstein, Antisense Nucleic Acid Drug Dev. 10:117-21, 2000; Rusckowski et al., Antisense Nucleic Acid Drug Dev. 10:333-45, 2000; Stein, Antisense Nucleic Acid Drug Dev. 11:317-25, 2001; Vorobjev et al., Antisense Nucleic Acid Drug Dev. 11:77-85, 2001; and U.S. Pat. No. 5,684,143.

As used herein, the term “complementary” refers to two nucleotides that form canonical Watson-Crick base pairs. For the avoidance of doubt, Watson-Crick base pairs in the context of the present disclosure include adenine-thymine, adenine-uracil, and cytosine-guanine base pairs. A proper Watson-Crick base pair is referred to in this context as a “match,” while each unpaired nucleotide, and each incorrectly paired nucleotide, is referred to as a “mismatch.” Alignment for purposes of determining percent nucleic acid sequence complementarity can be achieved in various ways that are within the capabilities of one of skill in the art, for example, using publicly available computer software such as BLAST, BLAST-2, or Megalign software.

“Percent (%) sequence complementarity” with respect to a reference polynucleotide sequence is defined as the percentage of nucleic acids in a candidate sequence that are complementary to the nucleic acids in the reference polynucleotide sequence, after aligning the sequences and introducing gaps, if necessary, to achieve the maximum percent sequence complementarity. A given nucleotide is considered to be “complementary” to a reference nucleotide as described herein if the two nucleotides form canonical Watson-Crick base pairs. For the avoidance of doubt, Watson-Crick base pairs in the context of the present disclosure include adenine-thymine, adenine-uracil, and cytosine-guanine base pairs. A proper Watson-Crick base pair is referred to in this context as a “match,” while each unpaired nucleotide, and each incorrectly paired nucleotide, is referred to as a “mismatch.” Alignment for purposes of determining percent nucleic acid sequence complementarity can be achieved in various ways that are within the capabilities of one of skill in the art, for example, using publicly available computer software such as BLAST, BLAST-2, or Megalign software. Those skilled in the art can determine appropriate parameters for aligning sequences, including any algorithms needed to achieve maximal complementarity over the full length of the sequences being compared. As an illustration, the percent sequence complementarity of a given nucleic acid sequence, A, to a given nucleic acid sequence, B, (which can alternatively be phrased as a given nucleic acid sequence, A that has a certain percent complementarity to a given nucleic acid sequence, B) is calculated as follows:

100 multiplied by (the fraction X/Y)
where X is the number of complementary base pairs in an alignment (e.g., as executed by computer software, such as BLAST) in that program's alignment of A and B, and where Y is the total number of nucleic acids in B. It will be appreciated that where the length of nucleic acid sequence A is not equal to the length of nucleic acid sequence B, the percent sequence complementarity of A to B will not equal the percent sequence complementarity of B to A. As used herein, a query nucleic acid sequence is considered to be “completely complementary” to a reference nucleic acid sequence if the query nucleic acid sequence has 100% sequence complementarity to the reference nucleic acid sequence.

“Percent (%) sequence identity” with respect to a reference polynucleotide or polypeptide sequence is defined as the percentage of nucleic acids or amino acids in a candidate sequence that are identical to the nucleic acids or amino acids in the reference polynucleotide or polypeptide sequence, after aligning the sequences and introducing gaps, if necessary, to achieve the maximum percent sequence identity. Alignment for purposes of determining percent nucleic acid or amino acid sequence identity can be achieved in various ways that are within the capabilities of one of skill in the art, for example, using publicly available computer software such as BLAST, BLAST-2, or Megalign software. Those skilled in the art can determine appropriate parameters for aligning sequences, including any algorithms needed to achieve maximal alignment over the full length of the sequences being compared. For example, percent sequence identity values may be generated using the sequence comparison computer program BLAST. As an illustration, the percent sequence identity of a given nucleic acid or amino acid sequence, A, to, with, or against a given nucleic acid or amino acid sequence, B, (which can alternatively be phrased as a given nucleic acid or amino acid sequence, A that has a certain percent sequence identity to, with, or against a given nucleic acid or amino acid sequence, B) is calculated as follows:

100 multiplied by (the fraction X/Y)
where X is the number of nucleotides or amino acids scored as identical matches by a sequence alignment program (e.g., BLAST) in that program's alignment of A and B, and where Y is the total number of nucleic acids in B. It will be appreciated that where the length of nucleic acid or amino acid sequence A is not equal to the length of nucleic acid or amino acid sequence B, the percent sequence identity of A to B will not equal the percent sequence identity of B to A.

The term “complementarity sufficient to hybridize,” as used herein, refers to a nucleic acid sequence or a portion thereof that need not be fully complementary (e.g., 100% complementary) to a target region or a nucleic acid sequence or a portion thereof that has one or more nucleotide mismatches relative to the target region but that is still capable of hybridizing to the target region under specified conditions. For example, the nucleic acid may be, e.g., 95% complementary, 90%, complementary, 85% complementary, 80% complementary, 75% complementary, 70% complementary, 65% complementary, 60% complementary, 55% complementary, 50% complementary, or less, but still form sufficient base pairs with the target so as to hybridize across its length.

“Hybridization” or “annealing” of nucleic acids is achieved when one or more nucleoside residues within a polynucleotide base pairs with one or more complementary nucleosides to form a stable duplex. The base pairing is typically driven by hydrogen bonding events. Hybridization includes Watson-Crick base pairs formed from natural and/or modified nucleobases. The hybridization can also include non-Watson-Crick base pairs, such as wobble base pairs (guanosine-uracil, hypoxanthine-uracil, hypoxanthine-adenine, and hypoxanthine-cytosine) and Hoogsteen base pairs. Nucleic acids need not be 100% complementary to undergo hybridization. For example, one nucleic acid may be, e.g., 95% complementary, 90%, complementary, 85% complementary, 80% complementary, 75% complementary, 70% complementary, 65% complementary, 60% complementary, 55% complementary, 50% complementary, or less, relative to another nucleic acid, but the two nucleic acids may still form sufficient base pairs with one another so as to hybridize.

The “stable duplex” formed upon the annealing/hybridization of one nucleic acid to another is a duplex structure that is not denatured by a stringent wash. Exemplary stringent wash conditions are known in the art and include temperatures of about 5° C. less than the melting temperature of an individual strand of the duplex and low concentrations of monovalent salts, such as monovalent salt concentrations (e.g., NaCl concentrations) of less than 0.2 M (e.g., 0.2 M, 0.19 M, 0.18 M, 0.17 M, 0.16 M, 0.15 M, 0.14 M, 0.13 M, 0.12 M, 0.11 M, 0.1 M, 0.09 M, 0.08 M, 0.07 M, 0.06 M, 0.05 M, 0.04 M, 0.03 M, 0.02 M, 0.01 M, or less).

The term “gene silencing” refers to the suppression of gene expression, e.g., endogenous gene expression of SCN9A, which may be mediated through processes that affect transcription and/or through processes that affect post-transcriptional mechanisms. In some embodiments, gene silencing occurs when an RNAi molecule initiates the inhibition or degradation of the mRNA transcribed from a gene of interest in a sequence-specific manner by way of RNA interference, thereby preventing translation of the gene's product.

The phrase “overactive disease driver gene,” as used herein, refers to a gene having increased activity and/or expression that contributes to or causes a disease state in a subject (e.g., a human). The disease state may be caused or exacerbated by the overactive disease driver gene directly or by way of an intermediate gene(s).

As used herein, the term “ethylene glycol chain” refers to a carbon chain with the formula ((CH₂OH)₂).

As used herein, “alkyl” refers to a saturated hydrocarbon group. Alkyl groups may be acyclic or cyclic and contain only C and H when unsubstituted. When an alkyl residue having a specific number of carbons is named, all geometric isomers having that number of carbons are intended to be encompassed and described; thus, for example, “butyl” is meant to include n-butyl, sec-butyl, and iso-butyl. Examples of alkyl include ethyl, propyl, butyl, pentyl, hexyl, heptyl, octyl, cyclopropyl, cyclobutyl, cyclopentyl, cyclohexyl, cycloheptyl, cyclooctyl, and the like. In some embodiments, alkyl may be substituted. Suitable substituents that may be introduced into an alkyl group include, for example, hydroxy, alkoxy, amino, alkylamino, and halo, among others.

As used herein, “alkenyl” refers to an acyclic or cyclic unsaturated hydrocarbon group having at least one site of olefinic unsaturation (i.e., having at least one moiety of the formula C═C). Alkenyl groups contain only C and H when unsubstituted. When an alkenyl residue having a specific number of carbons is named, all geometric isomers having that number of carbons are intended to be encompassed and described; thus, for example, “butenyl” is meant to include n-butenyl, sec-butenyl, and iso-butenyl. Examples of alkenyl include —CH═CH₂, —CH₂—CH═CH₂, and —CH₂—CH═CH—CH═CH₂. In some embodiments, alkenyl may be substituted. Suitable substituents that may be introduced into an alkenyl group include, for example, hydroxy, alkoxy, amino, alkylamino, and halo, among others.

As used herein, “alkynyl” refers to an acyclic or cyclic unsaturated hydrocarbon group having at least one site of acetylenic unsaturation (i.e., having at least one moiety of the formula C≡C). Alkynyl groups contain only C and H when unsubstituted. When an alkynyl residue having a specific number of carbons is named, all geometric isomers having that number of carbons are intended to be encompassed and described; thus, for example, “pentynyl” is meant to include n-pentynyl, sec-pentynyl, iso-pentynyl, and tert-pentynyl. Examples of alkynyl include —C≡CH and —C≡C—CH₃. In some embodiments, alkynyl may be substituted. Suitable substituents that may be introduced into an alkynyl group include, for example, hydroxy, alkoxy, amino, alkylamino, and halo, among others.

As used herein the term “phenyl” denotes a monocyclic arene in which one hydrogen atom from a carbon atom of the ring has been removed. A phenyl group may be unsubstituted or substituted with one or more suitable substituents, wherein the substituent replaces an H of the phenyl group.

As used herein, the term “benzyl” refers to monovalent radical obtained when a hydrogen atom attached to the methyl group of toluene is removed. A benzyl generally has the formula of phenyl-CH₂—. A benzyl group may be unsubstituted or substituted with one or more suitable substituents. For example, the substituent may replace an H of the phenyl component and/or an H of the methylene (—CH₂—) component.

As used herein, the term “amide” refers to an alkyl, alkenyl, alkynyl, or aromatic group that is attached to an amino-carbonyl functional group.

As used herein, the term “triazole” refers to heterocyclic compounds with the formula (C₂H₃N₃), having a five-membered ring of two carbons and three nitrogens, the positions of which can change resulting in multiple isomers.

As used herein, the term “terminal group” refers to the group at which a carbon chain or nucleic acid ends.

As used herein, an “amino acid” refers to a molecule containing amine and carboxyl functional groups and a side chain specific to the amino acid.

In some embodiments the amino acid is chosen from the group of proteinogenic amino acids. In some embodiments, the amino acid is an L-amino acid or a D-amino acid. In some embodiments, the amino acid is a synthetic amino acid (e.g., a beta-amino acid).

As used herein, the term “lipophilic amino acid” refers to an amino acid including a hydrophobic moiety (e.g., an alkyl chain or an aromatic ring).

As used herein, the term “target of delivery” refers to the organ or part of the body to which it is desired to deliver the branched oligonucleotide compositions.

As used herein, the term “between X and Y” is inclusive of the values of X and Y. For example, “between X and Y” refers to the range of values between the value of X and the value of Y, as well as the value of X and the value of Y.

As used herein, the terms “subject” and “patient” are used interchangeably and refer to an organism, such as a mammal (e.g., a human) that receives treatment for acute or chronic pain and/or contains a gain-of-function SCN9A variant gene. Examples of subjects and patients may also include those diagnosed with a pain disorder, such as Gerhardt disease, Mitchell disease, Weir-Mitchell disease, and/or exhibit symptoms of erythromelalgia.

As use herein, the term “pain” includes any and all forms of chronic and acute pain, including neuropathic pain and nociceptive pain, among others recited herein.

As used herein, the term “SCN9A” refers to the gene encoding the Nav1.7 voltage-gated sodium ion channel protein, including any native SCN9A gene from any source. The term encompasses “full-length,” unprocessed SCN9A as well as any form of SCN9A that results from processing in the cell. The term also encompasses naturally occurring variants of SCN9A, e.g., splice variants or allelic variants. The nucleic acid sequence of an exemplary SCN9A gene is shown in European Nucleotide Archive (ENA) Accession No. DQ857292.1. The amino acid sequence of an exemplary protein encoded by a SCN9A gene is shown in UNIPROT™ Accession No. Q15858.

As used herein, the terms “treat,” “treated,” and “treating” mean both therapeutic treatment and prophylactic or preventative measures wherein the object is to prevent, ameliorate, or slow down (lessen) an undesired physiological condition, disorder, or disease, or obtain beneficial or desired clinical results. Beneficial or desired clinical results include, but are not limited to, a reduction in a patient's reliance on analgesics; alleviation of symptoms; diminishment of the extent of a condition, disorder, or disease; stabilized (i.e., not worsening) state of condition, disorder, or disease; delay in onset or slowing of condition, disorder, or disease progression; amelioration of the condition, disorder, or disease state or remission (whether partial or total), whether detectable or undetectable; an amelioration of at least one measurable physical parameter, not necessarily discernible by the patient; or enhancement or improvement of condition, disorder, or disease. Treatment includes eliciting a clinically significant response without excessive levels of side effects. Treatment also includes prolonging survival as compared to expected survival if not receiving treatment.

As used herein, the terms “benefit” and “response” are used interchangeably in the context of a subject undergoing therapy for the treatment of, for example, acute pain, chronic pain, nociceptive pain, neuropathic pain, post-operative pain, inflammatory pain, erythromelalgia, primary erythromelalgia, secondary erythromelalgia, a pain disorder, Gerhardt disease, Mitchell disease, or Weir-Mitchell disease. For example, clinical benefits in the context of a subject administered an siRNA molecule or siRNA composition of the disclosure include, without limitation, a reduction of acute pain, chronic pain, reliance on analgesics, symptoms of erythromelalgia, wild type SCN9A transcripts, mutant SCN9A transcripts, variant SCN9A transcripts, splice isoforms of SCN9A transcripts, and/or overexpressed SCN9A transcripts thereof (relative to a healthy subject).

### DETAILED DESCRIPTION

The present disclosure provides compositions of small interfering RNA (siRNA) molecules with sequence homology to a sodium voltage-gated channel alpha subunit 9 (SCN9A) gene and methods for administering said siRNA molecules to the central nervous system of a subject. Furthermore, the siRNA molecules described herein may be composed as branched siRNA structures, such as di-branched, tri-branched, and tetra-branched siRNA structures and may further include specific patterns of chemical modifications (e.g., 2′ ribose modifications or internucleoside linkage modifications) to improve resistance against nuclease enzymes, toxicity profile, and physicochemical properties (e.g., thermostability). Small interfering RNA molecules are short, double-stranded RNA molecules. They are capable of mediating RNA interference (RNAi) by degrading mRNA with a complementary nucleotide sequence, thus preventing the translation of the target gene.

The siRNA molecules of the disclosure may exhibit, for example, robust gene-specific suppression of SCN9A, relative to other genes in the SCN family (e.g., SCN1A, SCN2A, SCN3A, SCN4A, SCN5A, SCN8A, SCN10A, and SCN11A). The siRNA sequences of the disclosure also avoid gain-of-function variants in SCN9A that cause spontaneous pain (primary erythromelalgia), thereby preserving the efficacy of siRNAs to produce analgesia in this genetically-defined population.

The siRNA molecules of the disclosure may feature an antisense strand having a nucleic acid sequence that is complementary to a region of a SCN9A mRNA transcript having the nucleic acid sequence of any one of SEQ ID NOs: 385-576 and 961-1152. The degree of complementarity of the antisense strand to the region of the SCN9A mRNA transcript may be sufficient for the antisense strand to anneal over the full length of the region of the SCN9A mRNA transcript. For example, the antisense strand may have a nucleic acid sequence that is at least 60% complementary (e.g., 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% complementary) to the region of the SCN9A mRNA transcript.

In some embodiments, the siRNA molecules of the disclosure feature an antisense strand having the nucleic acid sequence of any one of SEQ ID NOs: 1-192 and 577-768, or a nucleic acid sequence that is at least 60% identical thereto. For example, the siRNA molecules of the disclosure may feature an antisense strand having a nucleic acid sequence that is at least 60% identical (e.g., 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identical) to the nucleic acid sequence of any one of SEQ ID NOs: 1-192 and 577-768.

In some embodiments, the siRNA molecules of the disclosure feature a sense strand having the nucleic acid sequence of any one of SEQ ID NOs: 193-384 and 769-960, or a nucleic acid sequence that is at least 60% identical thereto. For example, the siRNA molecules of the disclosure may feature a sense strand having a nucleic acid sequence that is at least 60% identical (e.g., 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identical) to the nucleic acid sequence of any one of SEQ ID NOs: 193-384 and 769-960.

Exemplary siRNA molecules of the disclosure are those shown in Table 1, below. Table 1 summarizes the antisense strands, sense strands, and corresponding regions of a SCN9A mRNA transcript that are targeted by each antisense strand.

TABLE 1
Nucleotide sequences for gene-specific SCN9A-targeting siRNA

| | | | | Targeting | SCN9A |
|---|---|---|---|---|---|
| Antisense | | Sense | | Region | mRNA |
| SEQ ID | Antisense | SEQ | | SEQ | targeted |
| NO: | Strand | ID NO: | Sense Strand | ID NO: | sequence |
| 1 | UGUUCAAU | 193 | UUGCCCUC | 385 | GUCUCUUG |
| | GAGGGCAA | | AUUGAACA | | CCCUCAUU |
| | GAGAC | | | | GAACA |
| 2 | UUGUUCAAU | 194 | UGCCCUCA | 386 | UCUCUUGC |
| | GAGGGCAA | | UUGAACAA | | CCUCAUUGA |
| | GAGA | | | | ACAA |
| 3 | UCCUUUGU | 195 | AGUAUUGAA | 387 | UUCAUAGUA |
| | UCAAUACUA | | CAAAGGA | | UUGAACAAA |
| | UGAA | | | | GGG |
| 4 | UCCCUUUG | 196 | GUAUUGAAC | 388 | UCAUAGUAU |
| | UUCAAUACU | | AAAGGGA | | UGAACAAAG |
| | AUGA | | | | GGA |
| 5 | UUCCCUUU | 197 | UAUUGAACA | 389 | CAUAGUAUU |
| | GUUCAAUAC | | AAGGGAA | | GAACAAAGG |
| | UAUG | | | | GAA |
| 6 | UUUCCCUU | 198 | AUUGAACAA | 390 | AUAGUAUUG |
| | UGUUCAAUA | | AGGGAAA | | AACAAAGGG |
| | CUAU | | | | AAA |
| 7 | UUUUCCCU | 199 | UUGAACAAA | 391 | UAGUAUUGA |
| | UUGUUCAAU | | GGGAAAA | | ACAAAGGGA |
| | ACUA | | | | AAA |
| 8 | UUUGAAACG | 200 | AAUCUUCCG | 392 | AAAACAAUC |
| | GAAGAUUG | | UUUCAAA | | UUCCGUUU |
| | UUUU | | | | CAAU |
| 9 | UUUCUUAGA | 201 | UCAGUCCU | 393 | UCCUUUCA |
| | GGACUGAAA | | CUAAGAAA | | GUCCUCUAA |
| | GGA | | | | GAAG |
| 10 | UCUUCUUA | 202 | CAGUCCUC | 394 | CCUUUCAG |
| | GAGGACUG | | UAAGAAGA | | UCCUCUAAG |
| | AAAGG | | | | AAGA |
| 11 | UUUCUUCU | 203 | GUCCUCUAA | 395 | UUUCAGUC |
| | UAGAGGAC | | GAAGAAA | | CUCUAAGAA |
| | UGAAA | | | | GAAU |
| 12 | UAUUCUUCU | 204 | UCCUCUAAG | 396 | UUCAGUCC |
| | UAGAGGAC | | AAGAAUA | | UCUAAGAAG |
| | UGAA | | | | AAUA |
| 13 | UAUAUUCUU | 205 | CUCUAAGAA | 397 | CAGUCCUC |
| | CUUAGAGG | | GAAUAUA | | UAAGAAGAA |
| | ACUG | | | | UAUC |
| 14 | UGAUAUUCU | 206 | UCUAAGAAG | 398 | AGUCCUCUA |
| | UCUUAGAG | | AAUAUCA | | AGAAGAAUA |
| | GACU | | | | UCU |
| 15 | UAGAUAUUC | 207 | CUAAGAAGA | 399 | GUCCUCUAA |
| | UUCUUAGA | | AUAUCUA | | GAAGAAUAU |
| | GGAC | | | | CUA |
| 16 | UUAGAUAUU | 208 | UAAGAAGAA | 400 | UCCUCUAAG |
| | CUUCUUAGA | | UAUCUAA | | AAGAAUAUC |
| | GGA | | | | UAU |
| 17 | UAUAGAUAU | 209 | AAGAAGAAU | 401 | CCUCUAAGA |
| | UCUUCUUA | | AUCUAUA | | AGAAUAUCU |
| | GAGG | | | | AUU |
| 18 | UUGCUGAA | 210 | ACUCCUUAU | 402 | AGUACACUC |
| | UAAGGAGU | | UCAGCAA | | CUUAUUCAG |
| | GUACU | | | | CAU |
| 19 | UAUGCUGAA | 211 | CUCCUUAUU | 403 | GUACACUCC |
| | UAAGGAGU | | CAGCAUA | | UUAUUCAGC |
| | GUAC | | | | AUG |
| 20 | UCAUGCUG | 212 | UCCUUAUUC | 404 | UACACUCCU |
| | AAUAAGGAG | | AGCAUGA | | UAUUCAGCA |
| | UGUA | | | | UGC |
| 21 | UGCAUGCU | 213 | CCUUAUUCA | 405 | ACACUCCUU |
| | GAAUAAGGA | | GCAUGCA | | AUUCAGCAU |
| | GUGU | | | | GCU |
| 22 | UAGCAUGC | 214 | CUUAUUCAG | 406 | CACUCCUUA |
| | UGAAUAAGG | | CAUGCUA | | UUCAGCAU |
| | AGUG | | | | GCUC |
| 23 | UCAGUAAAA | 215 | AGUACACUU | 407 | UGUCGAGU |
| | GUGUACUC | | UUACUGA | | ACACUUUUA |
| | GACA | | | | CUGG |
| 24 | UCCAGUAAA | 216 | GUACACUUU | 408 | GUCGAGUA |
| | AGUGUACU | | UACUGGA | | CACUUUUAC |
| | CGAC | | | | UGGA |
| 25 | UAAGCCUCU | 217 | CCUUGCAA | 409 | AAAAUCCUU |
| | UGCAAGGA | | GAGGCUUA | | GCAAGAGG |
| | UUUU | | | | CUUC |
| 26 | UCAAAUUCU | 218 | AUUUAACAG | 410 | UGCGUAUU |
| | GUUAAAUAC | | AAUUUGA | | UAACAGAAU |
| | GCA | | | | UUGU |
| 27 | UACAAAUUC | 219 | UUUAACAGA | 411 | GCGUAUUU |
| | UGUUAAAUA | | AUUUGUA | | AACAGAAUU |
| | CGC | | | | UGUA |
| 28 | UGGAUUACA | 220 | CUAUUUCU | 412 | GAAAACUAU |
| | GAAAUAGUU | | GUAAUCCA | | UUCUGUAAU |
| | UUC | | | | CCC |
| 29 | UGGGAUUA | 221 | UAUUUCUG | 413 | AAAACUAUU |
| | CAGAAAUAG | | UAAUCCCA | | UCUGUAAUC |
| | UUUU | | | | CCA |
| 30 | UUUAGUGC | 222 | GUGUGUUU | 414 | UCUGAGUG |
| | AAACACACU | | GCACUAAA | | UGUUUGCA |
| | CAGA | | | | CUAAU |
| 31 | UAUUAGUG | 223 | UGUGUUUG | 415 | CUGAGUGU |
| | CAAACACAC | | CACUAAUA | | GUUUGCAC |
| | UCAG | | | | UAAUU |
| 32 | UCAAUUAGU | 224 | UGUUUGCA | 416 | GAGUGUGU |
| | GCAAACACA | | CUAAUUGA | | UUGCACUAA |
| | CUC | | | | UUGG |
| 33 | UCCAAUUAG | 225 | GUUUGCAC | 417 | AGUGUGUU |
| | UGCAAACAC | | UAAUUGGA | | UGCACUAAU |
| | ACU | | | | UGGA |
| 34 | UUAGUCCAA | 226 | GCACUAAUU | 418 | UGUUUGCA |
| | UUAGUGCAA | | GGACUAA | | CUAAUUGGA |
| | ACA | | | | CUAC |
| 35 | UGUAGUCC | 227 | CACUAAUUG | 419 | GUUUGCAC |
| | AAUUAGUGC | | GACUACA | | UAAUUGGAC |
| | AAAC | | | | UACA |
| 36 | UUGUAGUC | 228 | ACUAAUUGG | 420 | UUUGCACUA |
| | CAAUUAGUG | | ACUACAA | | AUUGGACUA |
| | CAAA | | | | CAG |
| 37 | UCUGUAGU | 229 | CUAAUUGGA | 421 | UUGCACUAA |
| | CCAAUUAGU | | CUACAGA | | UUGGACUA |
| | GCAA | | | | CAGC |
| 38 | UGCUGUAG | 230 | UAAUUGGAC | 422 | UGCACUAAU |
| | UCCAAUUAG | | UACAGCA | | UGGACUACA |
| | UGCA | | | | GCU |
| 39 | UCAGCUGU | 231 | AUUGGACUA | 423 | CACUAAUUG |
| | AGUCCAAUU | | CAGCUGA | | GACUACAGC |
| | AGUG | | | | UGU |
| 40 | UGAGCAUC | 232 | GAUCCAAAG | 424 | GGAAGGAU |
| | UUUGGAUC | | AUGCUCA | | CCAAAGAUG |
| | CUUCC | | | | CUCU |
| 41 | UAGAGCAUC | 233 | AUCCAAAGA | 425 | GAAGGAUC |
| | UUUGGAUC | | UGCUCUA | | CAAAGAUGC |
| | CUUC | | | | UCUC |
| 42 | UCUGAAUCU | 234 | UCAGCACAG | 426 | UGGUUUCA |
| | GUGCUGAA | | AUUCAGA | | GCACAGAUU |
| | ACCA | | | | CAGG |
| 43 | UCCUGAAUC | 235 | CAGCACAGA | 427 | GGUUUCAG |
| | UGUGCUGA | | UUCAGGA | | CACAGAUUC |
| | AACC | | | | AGGU |
| 44 | UGCUCGUG | 236 | UAUGGCUA | 428 | CUGAUUAU |
| | UAGCCAUAA | | CACGAGCA | | GGCUACAC |
| | UCAG | | | | GAGCU |
| 45 | UUCUGUUU | 237 | AAGAAGCUA | 429 | CAUUGAAGA |
| | AGCUUCUU | | AACAGAA | | AGCUAAACA |
| | CAAUG | | | | GAA |
| 46 | UUUCUGUU | 238 | AGAAGCUAA | 430 | AUUGAAGAA |
| | UAGCUUCU | | ACAGAAA | | GCUAAACAG |
| | UCAAU | | | | AAA |
| 47 | UUUUCUGU | 239 | GAAGCUAAA | 431 | UUGAAGAAG |
| | UUAGCUUC | | CAGAAAA | | CUAAACAGA |
| | UUCAA | | | | AAG |
| 48 | UCUUUCUG | 240 | AAGCUAAAC | 432 | UGAAGAAGC |
| | UUUAGCUU | | AGAAAGA | | UAAACAGAA |
| | CUUCA | | | | AGA |
| 49 | UCACGAAUG | 241 | CACUCAGCA | 433 | GUCACCACU |
| | CUGAGUGG | | UUCGUGA | | CAGCAUUC |
| | UGAC | | | | GUGG |
| 50 | UCCACGAAU | 242 | ACUCAGCAU | 434 | UCACCACUC |
| | GCUGAGUG | | UCGUGGA | | AGCAUUCG |
| | GUGA | | | | UGGC |
| 51 | UUGAUGUU | 243 | GCAGCAGU | 435 | GCGACGCA |
| | ACUGCUGC | | AACAUCAA | | GCAGUAACA |
| | GUCGC | | | | UCAG |
| 52 | UGAGCAUG | 244 | UCAGCCCU | 436 | GACGCUCA |
| | AGGGCUGA | | CAUGCUCA | | GCCCUCAU |
| | GCGUC | | | | GCUCC |
| 53 | UGGAGCAU | 245 | CAGCCCUCA | 437 | ACGCUCAG |
| | GAGGGCUG | | UGCUCCA | | CCCUCAUG |
| | AGCGU | | | | CUCCC |
| 54 | UGGGAGCA | 246 | AGCCCUCAU | 438 | CGCUCAGC |
| | UGAGGGCU | | GCUCCCA | | CCUCAUGC |
| | GAGCG | | | | UCCCC |
| 55 | UCAAGUUCU | 247 | CUGUGGAA | 439 | AAACACUGU |
| | UCCACAGU | | GAACUUGA | | GGAAGAACU |
| | GUUU | | | | UGA |
| 56 | UUCAAGUUC | 248 | UGUGGAAG | 440 | AACACUGUG |
| | UUCCACAGU | | AACUUGAA | | GAAGAACUU |
| | GUU | | | | GAA |
| 57 | UCAAAUCUG | 249 | GGUGGUAC | 441 | ACCUUGGU |
| | UACCACCAA | | AGAUUUGA | | GGUACAGA |
| | GGU | | | | UUUGC |
| 58 | UGCAAAUCU | 250 | GUGGUACA | 442 | CCUUGGUG |
| | GUACCACCA | | GAUUUGCA | | GUACAGAUU |
| | AGG | | | | UGCA |
| 59 | UGAGAGCAA | 251 | UCUGGAAU | 443 | CUUGAUCU |
| | UUCCAGAUC | | UGCUCUCA | | GGAAUUGC |
| | AAG | | | | UCUCC |
| 60 | UGGAGAGC | 252 | CUGGAAUU | 444 | UUGAUCUG |
| | AAUUCCAGA | | GCUCUCCA | | GAAUUGCU |
| | UCAA | | | | CUCCA |
| 61 | UAUGGAGA | 253 | GGAAUUGC | 445 | GAUCUGGA |
| | GCAAUUCCA | | UCUCCAUA | | AUUGCUCU |
| | GAUC | | | | CCAUA |
| 62 | UUAUGGAG | 254 | GAAUUGCU | 446 | AUCUGGAAU |
| | AGCAAUUCC | | CUCCAUAA | | UGCUCUCC |
| | AGAU | | | | AUAU |
| 63 | UAUAUGGA | 255 | AAUUGCUCU | 447 | UCUGGAAU |
| | GAGCAAUUC | | CCAUAUA | | UGCUCUCC |
| | CAGA | | | | AUAUU |
| 64 | UAAUAUGGA | 256 | AUUGCUCU | 448 | CUGGAAUU |
| | GAGCAAUUC | | CCAUAUUA | | GCUCUCCA |
| | CAG | | | | UAUUG |
| 65 | UCAAUAUGG | 257 | UUGCUCUC | 449 | UGGAAUUG |
| | AGAGCAAUU | | CAUAUUGA | | CUCUCCAUA |
| | CCA | | | | UUGG |
| 66 | UCCAAUAUG | 258 | UGCUCUCC | 450 | GGAAUUGC |
| | GAGAGCAAU | | AUAUUGGA | | UCUCCAUAU |
| | UCC | | | | UGGA |
| 67 | UAUCCAAUA | 259 | CUCUCCAUA | 451 | AAUUGCUCU |
| | UGGAGAGC | | UUGGAUA | | CCAUAUUG |
| | AAUU | | | | GAUA |
| 68 | UUAUCCAAU | 260 | UCUCCAUAU | 452 | AUUGCUCU |
| | AUGGAGAG | | UGGAUAA | | CCAUAUUG |
| | CAAU | | | | GAUAA |
| 69 | UUUAUCCAA | 261 | CUCCAUAUU | 453 | UUGCUCUC |
| | UAUGGAGA | | GGAUAAA | | CAUAUUGGA |
| | GCAA | | | | UAAA |
| 70 | UUUUAUCCA | 262 | UCCAUAUUG | 454 | UGCUCUCC |
| | AUAUGGAGA | | GAUAAAA | | AUAUUGGAU |
| | GCA | | | | AAAA |
| 71 | UAUUUUAUC | 263 | CAUAUUGGA | 455 | CUCUCCAUA |
| | CAAUAUGGA | | UAAAAUA | | UUGGAUAAA |
| | GAG | | | | AUU |
| 72 | UGAAUUUUA | 264 | UAUUGGAUA | 456 | CUCCAUAUU |
| | UCCAAUAUG | | AAAUUCA | | GGAUAAAAU |
| | GAG | | | | UCA |
| 73 | UAGAUCUAC | 265 | UCCUUUUG | 457 | AUGGAUCC |
| | AAAAGGAUC | | UAGAUCUA | | UUUUGUAG |
| | CAU | | | | AUCUU |
| 74 | UAAGAUCUA | 266 | CCUUUUGU | 458 | UGGAUCCU |
| | CAAAAGGAU | | AGAUCUUA | | UUUGUAGA |
| | CCA | | | | UCUUG |
| 75 | UCAAGAUCU | 267 | CUUUUGUA | 459 | GGAUCCUU |
| | ACAAAAGGA | | GAUCUUGA | | UUGUAGAU |
| | UCC | | | | CUUGC |
| 76 | UGCAAGAUC | 268 | UUUUGUAG | 460 | GAUCCUUU |
| | UACAAAAGG | | AUCUUGCA | | UGUAGAUC |
| | AUC | | | | UUGCA |
| 77 | UUGCAAGAU | 269 | UUUGUAGA | 461 | AUCCUUUU |
| | CUACAAAAG | | UCUUGCAA | | GUAGAUCU |
| | GAU | | | | UGCAA |
| 78 | UUUGCAAGA | 270 | UUGUAGAU | 462 | UCCUUUUG |
| | UCUACAAAA | | CUUGCAAA | | UAGAUCUU |
| | GGA | | | | GCAAU |
| 79 | UAUUGCAAG | 271 | UGUAGAUC | 463 | CCUUUUGU |
| | AUCUACAAA | | UUGCAAUA | | AGAUCUUG |
| | AGG | | | | CAAUU |
| 80 | UAAUUGCAA | 272 | GUAGAUCU | 464 | CUUUUGUA |
| | GAUCUACAA | | UGCAAUUA | | GAUCUUGC |
| | AAG | | | | AAUUA |
| 81 | UGUAAUUG | 273 | AGAUCUUG | 465 | UUUGUAGA |
| | CAAGAUCUA | | CAAUUACA | | UCUUGCAAU |
| | CAAA | | | | UACC |
| 82 | UGGUAAUU | 274 | GAUCUUGC | 466 | UUGUAGAU |
| | GCAAGAUCU | | AAUUACCA | | CUUGCAAUU |
| | ACAA | | | | ACCA |
| 83 | UUGGUAAU | 275 | AUCUUGCAA | 467 | UGUAGAUC |
| | UGCAAGAUC | | UUACCAA | | UUGCAAUUA |
| | UACA | | | | CCAU |
| 84 | UAUGGUAAU | 276 | UCUUGCAAU | 468 | GUAGAUCU |
| | UGCAAGAUC | | UACCAUA | | UGCAAUUAC |
| | UAC | | | | CAUU |
| 85 | UAAUGGUAA | 277 | CUUGCAAUU | 469 | UAGAUCUU |
| | UUGCAAGAU | | ACCAUUA | | GCAAUUACC |
| | CUA | | | | AUUU |
| 86 | UAAAUGGUA | 278 | UUGCAAUUA | 470 | AGAUCUUG |
| | AUUGCAAGA | | CCAUUUA | | CAAUUACCA |
| | UCU | | | | UUUG |
| 87 | UCAAAUGGU | 279 | UGCAAUUAC | 471 | GAUCUUGC |
| | AAUUGCAAG | | CAUUUGA | | AAUUACCAU |
| | AUC | | | | UUGC |
| 88 | UUAUGCAAA | 280 | AUUACCAUU | 472 | UUGCAAUUA |
| | UGGUAAUU | | UGCAUAA | | CCAUUUGCA |
| | GCAA | | | | UAG |
| 89 | UCUAUGCAA | 281 | UUACCAUUU | 473 | UGCAAUUAC |
| | AUGGUAAUU | | GCAUAGA | | CAUUUGCAU |
| | GCA | | | | AGU |
| 90 | UAACUAUGC | 282 | ACCAUUUGC | 474 | CAAUUACCA |
| | AAAUGGUAA | | AUAGUUA | | UUUGCAUA |
| | UUG | | | | GUUU |
| 91 | UGUUUAAAA | 283 | UGCAUAGU | 475 | CCAUUUGCA |
| | CUAUGCAAA | | UUUAAACA | | UAGUUUUAA |
| | UGG | | | | ACA |
| 92 | UCUUGGAAA | 284 | AUGAGUAUU | 476 | UCCAUAUGA |
| | UACUCAUAU | | UCCAAGA | | GUAUUUCCA |
| | GGA | | | | AGU |
| 93 | UACUUGGAA | 285 | UGAGUAUU | 477 | CCAUAUGAG |
| | AUACUCAUA | | UCCAAGUA | | UAUUUCCAA |
| | UGG | | | | GUA |
| 94 | UUACUUGG | 286 | GAGUAUUU | 478 | CAUAUGAGU |
| | AAAUACUCA | | CCAAGUAA | | AUUUCCAAG |
| | UAUG | | | | UAG |
| 95 | UCUACUUG | 287 | AGUAUUUCC | 479 | AUAUGAGUA |
| | GAAAUACUC | | AAGUAGA | | UUUCCAAGU |
| | AUAU | | | | AGG |
| 96 | UCCUACUU | 288 | GUAUUUCCA | 480 | UAUGAGUAU |
| | GGAAAUACU | | AGUAGGA | | UUCCAAGUA |
| | CAUA | | | | GGC |
| 97 | UCCUUCCAC | 289 | AGCAGAUG | 481 | UUUCUAGCA |
| | AUCUGCUA | | UGGAAGGA | | GAUGUGGA |
| | GAAA | | | | AGGA |
| 98 | UUCCUUCCA | 290 | GCAGAUGU | 482 | UUCUAGCA |
| | CAUCUGCUA | | GGAAGGAA | | GAUGUGGA |
| | GAA | | | | AGGAU |
| 99 | UCUUGAAGA | 291 | CUCCGAGU | 483 | GACUGCUC |
| | CUCGGAGC | | CUUCAAGA | | CGAGUCUU |
| | AGUC | | | | CAAGU |
| 100 | UACUUGAAG | 292 | UCCGAGUC | 484 | ACUGCUCC |
| | ACUCGGAG | | UUCAAGUA | | GAGUCUUC |
| | CAGU | | | | AAGUU |
| 101 | UAACUUGAA | 293 | CCGAGUCU | 485 | CUGCUCCG |
| | GACUCGGA | | UCAAGUUA | | AGUCUUCAA |
| | GCAG | | | | GUUG |
| 102 | UCAACUUGA | 294 | CGAGUCUU | 486 | UGCUCCGA |
| | AGACUCGG | | CAAGUUGA | | GUCUUCAA |
| | AGCA | | | | GUUGG |
| 103 | UUAAACAAU | 295 | GUGCCUUA | 487 | GCUAUGUG |
| | AAGGCACAU | | UUGUUUAA | | CCUUAUUG |
| | AGC | | | | UUUAC |
| 104 | UGUAAACAA | 296 | UGCCUUAU | 488 | CUAUGUGC |
| | UAAGGCACA | | UGUUUACA | | CUUAUUGU |
| | UAG | | | | UUACA |
| 105 | UAUGUAAAC | 297 | CCUUAUUG | 489 | AUGUGCCU |
| | AAUAAGGCA | | UUUACAUA | | UAUUGUUUA |
| | CAU | | | | CAUG |
| 106 | UCAUGUAAA | 298 | CUUAUUGU | 490 | UGUGCCUU |
| | CAAUAAGGC | | UUACAUGA | | AUUGUUUAC |
| | ACA | | | | AUGA |
| 107 | UUCAUGUAA | 299 | UUAUUGUU | 491 | GUGCCUUA |
| | ACAAUAAGG | | UACAUGAA | | UUGUUUACA |
| | CAC | | | | UGAU |
| 108 | UAUCAUGUA | 300 | UAUUGUUUA | 492 | UGCCUUAU |
| | AACAAUAAG | | CAUGAUA | | UGUUUACAU |
| | GCA | | | | GAUG |
| 109 | UCAUCAUGU | 301 | AUUGUUUAC | 493 | GCCUUAUU |
| | AAACAAUAA | | AUGAUGA | | GUUUACAU |
| | GGC | | | | GAUGG |
| 110 | UCCAUCAUG | 302 | UUGUUUACA | 494 | CCUUAUUG |
| | UAAACAAUA | | UGAUGGA | | UUUACAUGA |
| | AGG | | | | UGGU |
| 111 | UACCAUCAU | 303 | UGUUUACAU | 495 | CUUAUUGU |
| | GUAAACAAU | | GAUGGUA | | UUACAUGAU |
| | AAG | | | | GGUC |
| 112 | UGACCAUCA | 304 | GUUUACAU | 496 | UUAUUGUU |
| | UGUAAACAA | | GAUGGUCA | | UACAUGAUG |
| | UAA | | | | GUCA |
| 113 | UUGACCAUC | 305 | UUUACAUGA | 497 | UAUUGUUUA |
| | AUGUAAACA | | UGGUCAA | | CAUGAUGG |
| | AUA | | | | UCAU |
| 114 | UUUGCUGU | 306 | ACAAUCUUA | 498 | UUCAGACAA |
| | AAGAUUGUC | | CAGCAAA | | UCUUACAGC |
| | UGAA | | | | AAU |
| 115 | UAUUGCUG | 307 | CAAUCUUAC | 499 | UCAGACAAU |
| | UAAGAUUGU | | AGCAAUA | | CUUACAGCA |
| | CUGA | | | | AUU |
| 116 | UAAUUGCU | 308 | AAUCUUACA | 500 | CAGACAAUC |
| | GUAAGAUU | | GCAAUUA | | UUACAGCAA |
| | GUCUG | | | | UUG |
| 117 | UCAAUUGCU | 309 | AUCUUACAG | 501 | AGACAAUCU |
| | GUAAGAUU | | CAAUUGA | | UACAGCAAU |
| | GUCU | | | | UGA |
| 118 | UUCAAUUGC | 310 | UCUUACAGC | 502 | GACAAUCUU |
| | UGUAAGAUU | | AAUUGAA | | ACAGCAAUU |
| | GUC | | | | GAA |
| 119 | UGGAGGUU | 311 | AUGCAAACA | 503 | CCCUGAUG |
| | GUUUGCAU | | ACCUCCA | | CAAACAACC |
| | CAGGG | | | | UCCA |
| 120 | UCUGGAGG | 312 | GCAAACAAC | 504 | CUGAUGCAA |
| | UUGUUUGC | | CUCCAGA | | ACAACCUCC |
| | AUCAG | | | | AGA |
| 121 | UUCUGGAG | 313 | CAAACAACC | 505 | UGAUGCAAA |
| | GUUGUUUG | | UCCAGAA | | CAACCUCCA |
| | CAUCA | | | | GAU |
| 122 | UAUCUGGA | 314 | AAACAACCU | 506 | GAUGCAAAC |
| | GGUUGUUU | | CCAGAUA | | AACCUCCAG |
| | GCAUC | | | | AUU |
| 123 | UUCACUGU | 315 | CCAGCCUCA | 507 | CAAUCCCAG |
| | GAGGCUGG | | CAGUGAA | | CCUCACAGU |
| | GAUUG | | | | GAC |
| 124 | UGUCACUG | 316 | CAGCCUCAC | 508 | AAUCCCAGC |
| | UGAGGCUG | | AGUGACA | | CUCACAGU |
| | GGAUU | | | | GACA |
| 125 | UUGAAGCU | 317 | GGUUUGAA | 509 | CAGUUGGU |
| | UUCAAACCA | | AGCUUCAA | | UUGAAAGCU |
| | ACUG | | | | UCAU |
| 126 | UAUGAAGCU | 318 | GUUUGAAA | 510 | AGUUGGUU |
| | UUCAAACCA | | GCUUCAUA | | UGAAAGCUU |
| | ACU | | | | CAUU |
| 127 | UAGGAUAAU | 319 | CAUUAAGAU | 511 | AAGACCAUU |
| | CUUAAUGG | | UAUCCUA | | AAGAUUAUC |
| | UCUU | | | | CUG |
| 128 | UCAGGAUAA | 320 | AUUAAGAUU | 512 | AGACCAUUA |
| | UCUUAAUG | | AUCCUGA | | AGAUUAUCC |
| | GUCU | | | | UGG |
| 129 | UCCAGGAUA | 321 | UUAAGAUUA | 513 | GACCAUUAA |
| | AUCUUAAUG | | UCCUGGA | | GAUUAUCCU |
| | GUC | | | | GGA |
| 130 | UUCCAGGA | 322 | UAAGAUUAU | 514 | ACCAUUAAG |
| | UAAUCUUAA | | CCUGGAA | | AUUAUCCUG |
| | UGGU | | | | GAG |
| 131 | UCUCCAGG | 323 | AAGAUUAUC | 515 | CCAUUAAGA |
| | AUAAUCUUA | | CUGGAGA | | UUAUCCUG |
| | AUGG | | | | GAGU |
| 132 | UACUCCAG | 324 | AGAUUAUCC | 516 | CAUUAAGAU |
| | GAUAAUCUU | | UGGAGUA | | UAUCCUGG |
| | AAUG | | | | AGUA |
| 133 | UUACUCCAG | 325 | GAUUAUCCU | 517 | AUUAAGAUU |
| | GAUAAUCUU | | GGAGUAA | | AUCCUGGA |
| | AAU | | | | GUAU |
| 134 | UAUACUCCA | 326 | AUUAUCCUG | 518 | UUAAGAUUA |
| | GGAUAAUCU | | GAGUAUA | | UCCUGGAG |
| | UAA | | | | UAUG |
| 135 | UGCAUACUC | 327 | UAUCCUGG | 519 | AAGAUUAUC |
| | CAGGAUAAU | | AGUAUGCA | | CUGGAGUA |
| | CUU | | | | UGCA |
| 136 | UUUCCAGAA | 328 | AUCUUCAUU | 520 | CUUACAUCU |
| | UGAAGAUG | | CUGGAAA | | UCAUUCUG |
| | UAAG | | | | GAAA |
| 137 | UAGUAGCCA | 329 | ACACUCUUG | 521 | GGCAAACAC |
| | AGAGUGUU | | GCUACUA | | UCUUGGCU |
| | UGCC | | | | ACUC |
| 138 | UGAGUAGC | 330 | CACUCUUG | 522 | GCAAACACU |
| | CAAGAGUG | | GCUACUCA | | CUUGGCUA |
| | UUUGC | | | | CUCA |
| 139 | UUGAGUAG | 331 | ACUCUUGG | 523 | CAAACACUC |
| | CCAAGAGU | | CUACUCAA | | UUGGCUAC |
| | GUUUG | | | | UCAG |
| 140 | UCUGAGUA | 332 | CUCUUGGC | 524 | AAACACUCU |
| | GCCAAGAG | | UACUCAGA | | UGGCUACU |
| | UGUUU | | | | CAGA |
| 141 | UUUCCUUCA | 333 | CUAGAUUU | 525 | CUUAUCUAG |
| | AAUCUAGAU | | GAAGGAAA | | AUUUGAAG |
| | AAG | | | | GAAU |
| 142 | UCAUUCCUU | 334 | AGAUUUGAA | 526 | UAUCUAGAU |
| | CAAAUCUAG | | GGAAUGA | | UUGAAGGAA |
| | AUA | | | | UGA |
| 143 | UUCAUUCCU | 335 | GAUUUGAA | 527 | AUCUAGAUU |
| | UCAAAUCUA | | GGAAUGAA | | UGAAGGAAU |
| | GAU | | | | GAG |
| 144 | UUUGCUCC | 336 | CACUCAUAG | 528 | GAAUGCACU |
| | UAUGAGUG | | GAGCAAA | | CAUAGGAG |
| | CAUUC | | | | CAAU |
| 145 | UAUUGCUC | 337 | ACUCAUAGG | 529 | AAUGCACUC |
| | CUAUGAGU | | AGCAAUA | | AUAGGAGCA |
| | GCAUU | | | | AUU |
| 146 | UCUCAUAGA | 338 | GGCAAGUU | 530 | UUGCUGGC |
| | ACUUGCCA | | CUAUGAGA | | AAGUUCUAU |
| | GCAA | | | | GAGU |
| 147 | UACUCAUAG | 339 | GCAAGUUC | 531 | UGCUGGCA |
| | AACUUGCCA | | UAUGAGUA | | AGUUCUAU |
| | GCA | | | | GAGUG |
| 148 | UCACUCAUA | 340 | CAAGUUCUA | 532 | GCUGGCAA |
| | GAACUUGC | | UGAGUGA | | GUUCUAUG |
| | CAGC | | | | AGUGU |
| 149 | UACACUCAU | 341 | AAGUUCUAU | 533 | CUGGCAAG |
| | AGAACUUGC | | GAGUGUA | | UUCUAUGA |
| | CAG | | | | GUGUA |
| 150 | UUUUCCAUC | 342 | AAUGUGCG | 534 | GUCAAAAUG |
| | GCACAUUUU | | AUGGAAAA | | UGCGAUGG |
| | GAC | | | | AAAA |
| 151 | UUUGCAACU | 343 | UGCUUCAA | 535 | AUCUCUGC |
| | UGAAGCAGA | | GUUGCAAA | | UUCAAGUU |
| | GAU | | | | GCAAC |
| 152 | UGUUGCAA | 344 | GCUUCAAG | 536 | UCUCUGCU |
| | CUUGAAGCA | | UUGCAACA | | UCAAGUUG |
| | GAGA | | | | CAACU |
| 153 | UCUGCUGC | 345 | UUAUGUAU | 537 | GAUUAUUAU |
| | AUACAUAAU | | GCAGCAGA | | GUAUGCAG |
| | AAUC | | | | CAGU |
| 154 | UACUGCUG | 346 | UAUGUAUG | 538 | AUUAUUAUG |
| | CAUACAUAA | | CAGCAGUA | | UAUGCAGCA |
| | UAAU | | | | GUG |
| 155 | UGUUGGUU | 347 | AUAAUUUCA | 539 | CAUAGAUAA |
| | GAAAUUAUC | | ACCAACA | | UUUCAACCA |
| | UAUG | | | | ACA |
| 156 | UCUGUUGG | 348 | AAUUUCAAC | 540 | UAGAUAAUU |
| | UUGAAAUUA | | CAACAGA | | UCAACCAAC |
| | UCUA | | | | AGA |
| 157 | UUCUGUUG | 349 | AUUUCAACC | 541 | AGAUAAUUU |
| | GUUGAAAUU | | AACAGAA | | CAACCAACA |
| | AUCU | | | | GAA |
| 158 | UUUCUGUU | 350 | UUUCAACCA | 542 | GAUAAUUUC |
| | GGUUGAAA | | ACAGAAA | | AACCAACAG |
| | UUAUC | | | | AAA |
| 159 | UCUUGACC | 351 | AGCUUGGA | 543 | AAAGAAGCU |
| | UCCAAGCUU | | GGUCAAGA | | UGGAGGUC |
| | CUUU | | | | AAGA |
| 160 | UUCUUGAC | 352 | GCUUGGAG | 544 | AAGAAGCUU |
| | CUCCAAGCU | | GUCAAGAA | | GGAGGUCA |
| | UCUU | | | | AGAC |
| 161 | UCAAAUAUA | 353 | AAGGAUGUA | 545 | AAUCCAAGG |
| | CAUCCUUG | | UAUUUGA | | AUGUAUAUU |
| | GAUU | | | | UGA |
| 162 | UGUCAAAUA | 354 | GGAUGUAU | 546 | UCCAAGGAU |
| | UACAUCCUU | | AUUUGACA | | GUAUAUUU |
| | GGA | | | | GACC |
| 163 | UUGGUUAC | 355 | UCAACAUGG | 547 | CUGUCUCAA |
| | CAUGUUGA | | UAACCAA | | CAUGGUAAC |
| | GACAG | | | | CAU |
| 164 | UAUGGUUA | 356 | CAACAUGGU | 548 | UGUCUCAAC |
| | CCAUGUUG | | AACCAUA | | AUGGUAACC |
| | AGACA | | | | AUG |
| 165 | UUUCUACCA | 357 | ACCAUGAUG | 549 | UGGUAACCA |
| | UCAUGGUU | | GUAGAAA | | UGAUGGUA |
| | ACCA | | | | GAAA |
| 166 | UUUUCUACC | 358 | CCAUGAUG | 550 | GGUAACCAU |
| | AUCAUGGU | | GUAGAAAA | | GAUGGUAG |
| | UACC | | | | AAAA |
| 167 | UCAUGUCAU | 359 | GGAAUUAAU | 551 | AAGAUGGAA |
| | UAAUUCCAU | | GACAUGA | | UUAAUGACA |
| | CUU | | | | UGU |
| 168 | UACAUGUCA | 360 | GAAUUAAUG | 552 | AGAUGGAAU |
| | UUAAUUCCA | | ACAUGUA | | UAAUGACAU |
| | UCU | | | | GUU |
| 169 | UAACAUGUC | 361 | AAUUAAUGA | 553 | GAUGGAAU |
| | AUUAAUUCC | | CAUGUUA | | UAAUGACAU |
| | AUC | | | | GUUC |
| 170 | UGAACAUGU | 362 | AUUAAUGAC | 554 | AUGGAAUUA |
| | CAUUAAUUC | | AUGUUCA | | AUGACAUGU |
| | CAU | | | | UCA |
| 171 | UUGAACAUG | 363 | UUAAUGACA | 555 | UGGAAUUAA |
| | UCAUUAAUU | | UGUUCAA | | UGACAUGU |
| | CCA | | | | UCAA |
| 172 | UUUGAACAU | 364 | UAAUGACAU | 556 | GGAAUUAAU |
| | GUCAUUAAU | | GUUCAAA | | GACAUGUU |
| | UCC | | | | CAAU |
| 173 | UACGAAGAG | 365 | AUGGAUUC | 557 | GGGAGAUG |
| | AAUCCAUCU | | UCUUCGUA | | GAUUCUCU |
| | CCC | | | | UCGUU |
| 174 | UAACGAAGA | 366 | UGGAUUCU | 558 | GGAGAUGG |
| | GAAUCCAUC | | CUUCGUUA | | AUUCUCUUC |
| | UCC | | | | GUUC |
| 175 | UGAACGAAG | 367 | GGAUUCUC | 559 | GAGAUGGA |
| | AGAAUCCAU | | UUCGUUCA | | UUCUCUUC |
| | CUC | | | | GUUCA |
| 176 | UUGAACGAA | 368 | GAUUCUCU | 560 | AGAUGGAU |
| | GAGAAUCCA | | UCGUUCAA | | UCUCUUCG |
| | UCU | | | | UUCAC |
| 177 | UGUGAACG | 369 | AUUCUCUUC | 561 | GAUGGAUU |
| | AAGAGAAUC | | GUUCACA | | CUCUUCGU |
| | CAUC | | | | UCACA |
| 178 | UUGUGAAC | 370 | UUCUCUUC | 562 | AUGGAUUC |
| | GAAGAGAAU | | GUUCACAA | | UCUUCGUU |
| | CCAU | | | | CACAG |
| 179 | UCCAUCUG | 371 | UUCGUUCA | 563 | UUCUCUUC |
| | UGAACGAAG | | CAGAUGGA | | GUUCACAGA |
| | AGAA | | | | UGGA |
| 180 | UUCCAUCU | 372 | UCGUUCACA | 564 | UCUCUUCG |
| | GUGAACGAA | | GAUGGAA | | UUCACAGAU |
| | GAGA | | | | GGAA |
| 181 | UUUCCAUCU | 373 | CGUUCACA | 565 | CUCUUCGU |
| | GUGAACGAA | | GAUGGAAA | | UCACAGAUG |
| | GAG | | | | GAAG |
| 182 | UCUUCCAUC | 374 | GUUCACAGA | 566 | UCUUCGUU |
| | UGUGAACG | | UGGAAGA | | CACAGAUG |
| | AAGA | | | | GAAGA |
| 183 | UUCUUCCAU | 375 | UUCACAGAU | 567 | CUUCGUUC |
| | CUGUGAAC | | GGAAGAA | | ACAGAUGGA |
| | GAAG | | | | AGAA |
| 184 | UUUUCUUC | 376 | CACAGAUG | 568 | UCGUUCACA |
| | CAUCUGUG | | GAAGAAAA | | GAUGGAAG |
| | AACGA | | | | AAAG |
| 185 | UCUUUCUU | 377 | ACAGAUGGA | 569 | CGUUCACA |
| | CCAUCUGU | | AGAAAGA | | GAUGGAAG |
| | GAACG | | | | AAAGG |
| 186 | UCCUUUCU | 378 | CAGAUGGAA | 570 | GUUCACAGA |
| | UCCAUCUG | | GAAAGGA | | UGGAAGAAA |
| | UGAAC | | | | GGU |
| 187 | UACCUUUCU | 379 | AGAUGGAA | 571 | UUCACAGAU |
| | UCCAUCUG | | GAAAGGUA | | GGAAGAAAG |
| | UGAA | | | | GUU |
| 188 | UAACCUUUC | 380 | GAUGGAAG | 572 | UCACAGAUG |
| | UUCCAUCU | | AAAGGUUA | | GAAGAAAGG |
| | GUGA | | | | UUC |
| 189 | UGAACCUUU | 381 | AUGGAAGAA | 573 | CACAGAUG |
| | CUUCCAUCU | | AGGUUCA | | GAAGAAAGG |
| | GUG | | | | UUCA |
| 190 | UAUGAACCU | 382 | GGAAGAAAG | 574 | CAGAUGGAA |
| | UUCUUCCAU | | GUUCAUA | | GAAAGGUU |
| | CUG | | | | CAUG |
| 191 | UACAUGAAC | 383 | AAGAAAGGU | 575 | GAUGGAAG |
| | CUUUCUUC | | UCAUGUA | | AAAGGUUCA |
| | CAUC | | | | UGUC |
| 192 | UGUGAUGG | 384 | CUAUGAACC | 576 | GUGUCCUA |
| | GUUCAUAG | | CAUCACA | | UGAACCCAU |
| | GACAC | | | | CACA |
| 577 | UUCUUUUCA | 769 | UACAGGAU | 961 | AAAUAUACA |
| | UCCUGUAUA | | GAAAAGAA | | GGAUGAAAA |
| | UUU | | | | GAU |
| 578 | UUUGUUCAA | 770 | GCCCUCAU | 962 | CUCUUGCC |
| | UGAGGGCA | | UGAACAAA | | CUCAUUGAA |
| | AGAG | | | | CAAC |
| 579 | UGUUGUUC | 771 | CCCUCAUU | 963 | UCUUGCCC |
| | AAUGAGGG | | GAACAACA | | UCAUUGAAC |
| | CAAGA | | | | AACG |
| 580 | UCGUUGUU | 772 | CCUCAUUGA | 964 | CUUGCCCU |
| | CAAUGAGG | | ACAACGA | | CAUUGAACA |
| | GCAAG | | | | ACGC |
| 581 | UUGCGUUG | 773 | UCAUUGAAC | 965 | UGCCCUCA |
| | UUCAAUGAG | | AACGCAA | | UUGAACAAC |
| | GGCA | | | | GCAU |
| 582 | UUUUGUUC | 774 | AUAGUAUUG | 966 | CUUUCAUAG |
| | AAUACUAUG | | AACAAAA | | UAUUGAACA |
| | AAAG | | | | AAG |
| 583 | UCUUUGUU | 775 | UAGUAUUGA | 967 | UUUCAUAGU |
| | CAAUACUAU | | ACAAAGA | | AUUGAACAA |
| | GAAA | | | | AGG |
| 584 | UGUUUUCC | 776 | GAACAAAGG | 968 | GUAUUGAAC |
| | CUUUGUUC | | GAAAACA | | AAAGGGAAA |
| | AAUAC | | | | ACA |
| 585 | UUGUUUUC | 777 | AACAAAGGG | 969 | UAUUGAACA |
| | CCUUUGUU | | AAAACAA | | AAGGGAAAA |
| | CAAUA | | | | CAA |
| 586 | UAACGGAAG | 778 | AAACAAUCU | 970 | AGGGAAAAC |
| | AUUGUUUU | | UCCGUUA | | AAUCUUCCG |
| | CCCU | | | | UUU |
| 587 | UAAACGGAA | 779 | AACAAUCUU | 971 | GGGAAAACA |
| | GAUUGUUU | | CCGUUUA | | AUCUUCCG |
| | UCCC | | | | UUUC |
| 588 | UGAAACGGA | 780 | ACAAUCUUC | 972 | GGAAAACAA |
| | AGAUUGUU | | CGUUUCA | | UCUUCCGU |
| | UUCC | | | | UUCA |
| 589 | UUGAAACG | 781 | CAAUCUUCC | 973 | GAAAACAAU |
| | GAAGAUUG | | GUUUCAA | | CUUCCGUU |
| | UUUUC | | | | UCAA |
| 590 | UAUUGAAAC | 782 | AUCUUCCG | 974 | AAACAAUCU |
| | GGAAGAUU | | UUUCAAUA | | UCCGUUUC |
| | GUUU | | | | AAUG |
| 591 | UCAUUGAAA | 783 | UCUUCCGU | 975 | AACAAUCUU |
| | CGGAAGAU | | UUCAAUGA | | CCGUUUCAA |
| | UGUU | | | | UGC |
| 592 | UGCAUUGAA | 784 | CUUCCGUU | 976 | ACAAUCUUC |
| | ACGGAAGAU | | UCAAUGCA | | CGUUUCAAU |
| | UGU | | | | GCC |
| 593 | UGGCAUUG | 785 | UUCCGUUU | 977 | CAAUCUUCC |
| | AAACGGAAG | | CAAUGCCA | | GUUUCAAU |
| | AUUG | | | | GCCA |
| 594 | UUAGAGGA | 786 | CCUUUCAG | 978 | UUUCUCCU |
| | CUGAAAGGA | | UCCUCUAA | | UUCAGUCC |
| | GAAA | | | | UCUAA |
| 595 | UUUAGAGG | 787 | CUUUCAGU | 979 | UUCUCCUU |
| | ACUGAAAGG | | CCUCUAAA | | UCAGUCCU |
| | AGAA | | | | CUAAG |
| 596 | UCUUAGAG | 788 | UUUCAGUC | 980 | UCUCCUUU |
| | GACUGAAAG | | CUCUAAGA | | CAGUCCUC |
| | GAGA | | | | UAAGA |
| 597 | UUCUUAGA | 789 | UUCAGUCC | 981 | CUCCUUUCA |
| | GGACUGAAA | | UCUAAGAA | | GUCCUCUAA |
| | GGAG | | | | GAA |
| 598 | UUCUUCUUA | 790 | AGUCCUCUA | 982 | CUUUCAGU |
| | GAGGACUG | | AGAAGAA | | CCUCUAAGA |
| | AAAG | | | | AGAA |
| 599 | UUAUUCUUC | 791 | CCUCUAAGA | 983 | UCAGUCCU |
| | UUAGAGGA | | AGAAUAA | | CUAAGAAGA |
| | CUGA | | | | AUAU |
| 600 | UAAUAGAUA | 792 | AGAAGAAUA | 984 | CUCUAAGAA |
| | UUCUUCUUA | | UCUAUUA | | GAAUAUCUA |
| | GAG | | | | UUA |
| 601 | UUAAUAGAU | 793 | GAAGAAUAU | 985 | UCUAAGAAG |
| | AUUCUUCUU | | CUAUUAA | | AAUAUCUAU |
| | AGA | | | | UAA |
| 602 | UUUAAUAGA | 794 | AAGAAUAUC | 986 | CUAAGAAGA |
| | UAUUCUUCU | | UAUUAAA | | AUAUCUAUU |
| | UAG | | | | AAG |
| 603 | UCUUAAUAG | 795 | AGAAUAUCU | 987 | UAAGAAGAA |
| | AUAUUCUUC | | AUUAAGA | | UAUCUAUUA |
| | UUA | | | | AGA |
| 604 | UUCUUAAUA | 796 | GAAUAUCUA | 988 | AAGAAGAAU |
| | GAUAUUCUU | | UUAAGAA | | AUCUAUUAA |
| | CUU | | | | GAU |
| 605 | UAUCUUAAU | 797 | AAUAUCUAU | 989 | AGAAGAAUA |
| | AGAUAUUCU | | UAAGAUA | | UCUAUUAAG |
| | UCU | | | | AUU |
| 606 | UAAUCUUAA | 798 | AUAUCUAUU | 990 | GAAGAAUAU |
| | UAGAUAUUC | | AAGAUUA | | CUAUUAAGA |
| | UUC | | | | UUU |
| 607 | UCUAAAAUC | 799 | CUAUUAAGA | 991 | AAUAUCUAU |
| | UUAAUAGAU | | UUUUAGA | | UAAGAUUUU |
| | AUU | | | | AGU |
| 608 | UUAUGCAG | 800 | CUGACAAAC | 992 | CUAUUCUGA |
| | UUUGUCAG | | UGCAUAA | | CAAACUGCA |
| | AAUAG | | | | UAU |
| 609 | UAUAUGCAG | 801 | UGACAAACU | 993 | UAUUCUGAC |
| | UUUGUCAG | | GCAUAUA | | AAACUGCAU |
| | AAUA | | | | AUU |
| 610 | UAAAAGUGU | 802 | GUCGAGUA | 994 | AAAAUGUCG |
| | ACUCGACAU | | CACUUUUA | | AGUACACUU |
| | UUU | | | | UUA |
| 611 | UUAAAAGUG | 803 | UCGAGUACA | 995 | AAAUGUCGA |
| | UACUCGACA | | CUUUUAA | | GUACACUUU |
| | UUU | | | | UAC |
| 612 | UGUAAAAGU | 804 | CGAGUACAC | 996 | AAUGUCGA |
| | GUACUCGA | | UUUUACA | | GUACACUUU |
| | CAUU | | | | UACU |
| 613 | UAGUAAAAG | 805 | GAGUACACU | 997 | AUGUCGAG |
| | UGUACUCG | | UUUACUA | | UACACUUUU |
| | ACAU | | | | ACUG |
| 614 | UUCCAGUAA | 806 | UACACUUUU | 998 | UCGAGUACA |
| | AAGUGUACU | | ACUGGAA | | CUUUUACU |
| | CGA | | | | GGAA |
| 615 | UUUCCAGUA | 807 | ACACUUUUA | 999 | CGAGUACAC |
| | AAAGUGUAC | | CUGGAAA | | UUUUACUG |
| | UCG | | | | GAAU |
| 616 | UAUUCCAGU | 808 | CACUUUUAC | 1000 | GAGUACACU |
| | AAAAGUGUA | | UGGAAUA | | UUUACUGG |
| | CUC | | | | AAUA |
| 617 | UUAUUCCAG | 809 | ACUUUUACU | 1001 | AGUACACUU |
| | UAAAAGUGU | | GGAAUAA | | UUACUGGAA |
| | ACU | | | | UAU |
| 618 | UAUUCUGU | 810 | CGUAUUUAA | 1002 | UUUUGCGU |
| | UAAAUACGC | | CAGAAUA | | AUUUAACAG |
| | AAAA | | | | AAUU |
| 619 | UAAUUCUGU | 811 | GUAUUUAAC | 1003 | UUUGCGUA |
| | UAAAUACGC | | AGAAUUA | | UUUAACAGA |
| | AAA | | | | AUUU |
| 620 | UAAAUUCUG | 812 | UAUUUAACA | 1004 | UUGCGUAU |
| | UUAAAUACG | | GAAUUUA | | UUAACAGAA |
| | CAA | | | | UUUG |
| 621 | UUACAAAUU | 813 | UUAACAGAA | 1005 | CGUAUUUAA |
| | CUGUUAAAU | | UUUGUAA | | CAGAAUUUG |
| | ACG | | | | UAA |
| 622 | UUUACAAAU | 814 | UAACAGAAU | 1006 | GUAUUUAAC |
| | UCUGUUAAA | | UUGUAAA | | AGAAUUUGU |
| | UAC | | | | AAA |
| 623 | UGAUUACAG | 815 | ACUAUUUCU | 1007 | UGAAAACUA |
| | AAAUAGUUU | | GUAAUCA | | UUUCUGUAA |
| | UCA | | | | UCC |
| 624 | UCUGGGAU | 816 | UUUCUGUAA | 1008 | AACUAUUUC |
| | UACAGAAAU | | UCCCAGA | | UGUAAUCCC |
| | AGUU | | | | AGG |
| 625 | UUCCAAUUA | 817 | UUUGCACUA | 1009 | GUGUGUUU |
| | GUGCAAACA | | AUUGGAA | | GCACUAAUU |
| | CAC | | | | GGAC |
| 626 | UGUCCAAUU | 818 | UUGCACUAA | 1010 | UGUGUUUG |
| | AGUGCAAAC | | UUGGACA | | CACUAAUUG |
| | ACA | | | | GACU |
| 627 | UAGUCCAAU | 819 | UGCACUAAU | 1011 | GUGUUUGC |
| | UAGUGCAAA | | UGGACUA | | ACUAAUUGG |
| | CAC | | | | ACUA |
| 628 | UACAGCUG | 820 | UUGGACUA | 1012 | ACUAAUUGG |
| | UAGUCCAAU | | CAGCUGUA | | ACUACAGCU |
| | UAGU | | | | GUU |
| 629 | UCUAAUGUU | 821 | AUAAUGAAA | 1013 | UGAAAAUAA |
| | UCAUUAUUU | | CAUUAGA | | UGAAACAUU |
| | UCA | | | | AGA |
| 630 | UCUUUCUAA | 822 | UGAAACAUU | 1014 | AAUAAUGAA |
| | UGUUUCAU | | AGAAAGA | | ACAUUAGAA |
| | UAUU | | | | AGC |
| 631 | UUGCUUUC | 823 | AAACAUUAG | 1015 | UAAUGAAAC |
| | UAAUGUUUC | | AAAGCAA | | AUUAGAAAG |
| | AUUA | | | | CAU |
| 632 | UAUGCUUU | 824 | AACAUUAGA | 1016 | AAUGAAACA |
| | CUAAUGUUU | | AAGCAUA | | UUAGAAAGC |
| | CAUU | | | | AUA |
| 633 | UAAUCUGU | 825 | GUUUCAGC | 1017 | UUGUGGUU |
| | GCUGAAACC | | ACAGAUUA | | UCAGCACAG |
| | ACAA | | | | AUUC |
| 634 | UGUGUAGC | 826 | UGAUUAUG | 1018 | AACCCUGAU |
| | CAUAAUCAG | | GCUACACA | | UAUGGCUA |
| | GGUU | | | | CACG |
| 635 | UCGUGUAG | 827 | GAUUAUGG | 1019 | ACCCUGAUU |
| | CCAUAAUCA | | CUACACGA | | AUGGCUACA |
| | GGGU | | | | CGA |
| 636 | UUCGUGUA | 828 | AUUAUGGC | 1020 | CCCUGAUUA |
| | GCCAUAAUC | | UACACGAA | | UGGCUACA |
| | AGGG | | | | CGAG |
| 637 | UCUCGUGU | 829 | UUAUGGCU | 1021 | CCUGAUUAU |
| | AGCCAUAAU | | ACACGAGA | | GGCUACAC |
| | CAGG | | | | GAGC |
| 638 | UAGUGUCAA | 830 | ACGAGCUU | 1022 | GCUACACGA |
| | AGCUCGUG | | UGACACUA | | GCUUUGAC |
| | UAGC | | | | ACUU |
| 639 | UAAGUGUCA | 831 | CGAGCUUU | 1023 | CUACACGAG |
| | AAGCUCGU | | GACACUUA | | CUUUGACAC |
| | GUAG | | | | UUU |
| 640 | UAAAGUGUC | 832 | GAGCUUUG | 1024 | UACACGAGC |
| | AAAGCUCGU | | ACACUUUA | | UUUGACACU |
| | GUA | | | | UUC |
| 641 | UGAAAGUG | 833 | AGCUUUGA | 1025 | ACACGAGCU |
| | UCAAAGCUC | | CACUUUCA | | UUGACACUU |
| | GUGU | | | | UCA |
| 642 | UGACUUGU | 834 | GCAGCAGAA | 1026 | GCGAAGCA |
| | UCUGCUGC | | CAAGUCA | | GCAGAACAA |
| | UUCGC | | | | GUCU |
| 643 | UAGACUUG | 835 | CAGCAGAAC | 1027 | CGAAGCAG |
| | UUCUGCUG | | AAGUCUA | | CAGAACAAG |
| | CUUCG | | | | UCUU |
| 644 | UAAGUUCUU | 836 | ACUGUGGA | 1028 | CAAACACUG |
| | CCACAGUG | | AGAACUUA | | UGGAAGAAC |
| | UUUG | | | | UUG |
| 645 | UUCCAAUAU | 837 | GCUCUCCA | 1029 | GAAUUGCU |
| | GGAGAGCA | | UAUUGGAA | | CUCCAUAUU |
| | AUUC | | | | GGAU |
| 646 | UAAUUUUAU | 838 | AUAUUGGAU | 1030 | UCUCCAUAU |
| | CCAAUAUGG | | AAAAUUA | | UGGAUAAAA |
| | AGA | | | | UUC |
| 647 | UUGAAUUUU | 839 | AUUGGAUAA | 1031 | UCCAUAUUG |
| | AUCCAAUAU | | AAUUCAA | | GAUAAAAUU |
| | GGA | | | | CAA |
| 648 | UUUGAAUUU | 840 | UUGGAUAAA | 1032 | CCAUAUUG |
| | UAUCCAAUA | | AUUCAAA | | GAUAAAAUU |
| | UGG | | | | CAAA |
| 649 | UUUUGAAUU | 841 | UGGAUAAAA | 1033 | CAUAUUGGA |
| | UUAUCCAAU | | UUCAAAA | | UAAAAUUCA |
| | AUG | | | | AAA |
| 650 | UAAUAAAAU | 842 | UGUAUCUAU | 1034 | AAAAGUGUA |
| | AGAUACACU | | UUUAUUA | | UCUAUUUUA |
| | UUU | | | | UUG |
| 651 | UUUACAAUA | 843 | UCUAUUUUA | 1035 | GUGUAUCU |
| | AAAUAGAUA | | UUGUAAA | | AUUUUAUUG |
| | CAC | | | | UAAU |
| 652 | UAUUACAAU | 844 | CUAUUUUAU | 1036 | UGUAUCUAU |
| | AAAAUAGAU | | UGUAAUA | | UUUAUUGUA |
| | ACA | | | | AUG |
| 653 | UCCAUUACA | 845 | AUUUUAUUG | 1037 | UAUCUAUUU |
| | AUAAAAUAG | | UAAUGGA | | UAUUGUAAU |
| | AUA | | | | GGA |
| 654 | UUCCAUUAC | 846 | UUUUAUUG | 1038 | AUCUAUUUU |
| | AAUAAAAUA | | UAAUGGAA | | AUUGUAAUG |
| | GAU | | | | GAU |
| 655 | UAUCCAUUA | 847 | UUUAUUGUA | 1039 | UCUAUUUUA |
| | CAAUAAAAU | | AUGGAUA | | UUGUAAUG |
| | AGA | | | | GAUC |
| 656 | UGAUCCAUU | 848 | UUAUUGUAA | 1040 | CUAUUUUAU |
| | ACAAUAAAA | | UGGAUCA | | UGUAAUGG |
| | UAG | | | | AUCC |
| 657 | UGGAUCCA | 849 | UAUUGUAAU | 1041 | UAUUUUAUU |
| | UUACAAUAA | | GGAUCCA | | GUAAUGGA |
| | AAUA | | | | UCCU |
| 658 | UAGGAUCCA | 850 | AUUGUAAUG | 1042 | AUUUUAUUG |
| | UUACAAUAA | | GAUCCUA | | UAAUGGAUC |
| | AAU | | | | CUU |
| 659 | UAAGGAUCC | 851 | UUGUAAUG | 1043 | UUUUAUUG |
| | AUUACAAUA | | GAUCCUUA | | UAAUGGAUC |
| | AAA | | | | CUUU |
| 660 | UAAAGGAUC | 852 | UGUAAUGG | 1044 | UUUAUUGUA |
| | CAUUACAAU | | AUCCUUUA | | AUGGAUCC |
| | AAA | | | | UUUU |
| 661 | UAAAAGGAU | 853 | GUAAUGGA | 1045 | UUAUUGUAA |
| | CCAUUACAA | | UCCUUUUA | | UGGAUCCU |
| | UAA | | | | UUUG |
| 662 | UCAAAAGGA | 854 | UAAUGGAUC | 1046 | UAUUGUAAU |
| | UCCAUUACA | | CUUUUGA | | GGAUCCUU |
| | AUA | | | | UUGU |
| 663 | UACAAAAGG | 855 | AAUGGAUCC | 1047 | AUUGUAAUG |
| | AUCCAUUAC | | UUUUGUA | | GAUCCUUU |
| | AAU | | | | UGUA |
| 664 | UUACAAAAG | 856 | AUGGAUCC | 1048 | UUGUAAUG |
| | GAUCCAUUA | | UUUUGUAA | | GAUCCUUU |
| | CAA | | | | UGUAG |
| 665 | UUAAUUGCA | 857 | UAGAUCUU | 1049 | UUUUGUAG |
| | AGAUCUACA | | GCAAUUAA | | AUCUUGCAA |
| | AAA | | | | UUAC |
| 666 | UAAACUAUG | 858 | CCAUUUGCA | 1050 | AAUUACCAU |
| | CAAAUGGUA | | UAGUUUA | | UUGCAUAG |
| | AUU | | | | UUUU |
| 667 | UAAAACUAU | 859 | CAUUUGCAU | 1051 | AUUACCAUU |
| | GCAAAUGG | | AGUUUUA | | UGCAUAGU |
| | UAAU | | | | UUUA |
| 668 | UUAAAACUA | 860 | AUUUGCAUA | 1052 | UUACCAUUU |
| | UGCAAAUG | | GUUUUAA | | GCAUAGUU |
| | GUAA | | | | UUAA |
| 669 | UUUAAAACU | 861 | UUUGCAUA | 1053 | UACCAUUUG |
| | AUGCAAAUG | | GUUUUAAA | | CAUAGUUUU |
| | GUA | | | | AAA |
| 670 | UUUUAAAAC | 862 | UUGCAUAG | 1054 | ACCAUUUGC |
| | UAUGCAAAU | | UUUUAAAA | | AUAGUUUUA |
| | GGU | | | | AAC |
| 671 | UUGUUUAAA | 863 | GCAUAGUU | 1055 | CAUUUGCAU |
| | ACUAUGCAA | | UUAAACAA | | AGUUUUAAA |
| | AUG | | | | CAC |
| 672 | UGUGUUUA | 864 | CAUAGUUUU | 1056 | AUUUGCAUA |
| | AAACUAUGC | | AAACACA | | GUUUUAAAC |
| | AAAU | | | | ACA |
| 673 | UGGAAAUAC | 865 | CAUAUGAGU | 1057 | GGAUCCAUA |
| | UCAUAUGGA | | AUUUCCA | | UGAGUAUU |
| | UCC | | | | UCCA |
| 674 | UUGGAAAUA | 866 | AUAUGAGUA | 1058 | GAUCCAUAU |
| | CUCAUAUG | | UUUCCAA | | GAGUAUUU |
| | GAUC | | | | CCAA |
| 675 | UUUGGAAAU | 867 | UAUGAGUAU | 1059 | AUCCAUAUG |
| | ACUCAUAUG | | UUCCAAA | | AGUAUUUCC |
| | GAU | | | | AAG |
| 676 | UGCCUACU | 868 | UAUUUCCAA | 1060 | AUGAGUAUU |
| | UGGAAAUAC | | GUAGGCA | | UCCAAGUAG |
| | UCAU | | | | GCU |
| 677 | UACAAUAAG | 869 | UAUGUGCC | 1061 | CAAGCUAUG |
| | GCACAUAGC | | UUAUUGUA | | UGCCUUAU |
| | UUG | | | | UGUU |
| 678 | UAACAAUAA | 870 | AUGUGCCU | 1062 | AAGCUAUGU |
| | GGCACAUA | | UAUUGUUA | | GCCUUAUU |
| | GCUU | | | | GUUU |
| 679 | UGUCUUCU | 871 | GCAAUUGAA | 1063 | UUACAGCAA |
| | UCAAUUGCU | | GAAGACA | | UUGAAGAAG |
| | GUAA | | | | ACC |
| 680 | UACUGUGA | 872 | UCCCAGCC | 1064 | CACAAUCCC |
| | GGCUGGGA | | UCACAGUA | | AGCCUCACA |
| | UUGUG | | | | GUG |
| 681 | UAGGGUUA | 873 | ACAGUUGAU | 1065 | GCAGCACA |
| | UCAACUGU | | AACCCUA | | GUUGAUAAC |
| | GCUGC | | | | CCUU |
| 682 | UUGUUAACU | 874 | GCUGCCAA | 1066 | CUCAUGCU |
| | UGGCAGCA | | GUUAACAA | | GCCAAGUUA |
| | UGAG | | | | ACAU |
| 683 | UAUGUUAAC | 875 | CUGCCAAG | 1067 | UCAUGCUG |
| | UUGGCAGC | | UUAACAUA | | CCAAGUUAA |
| | AUGA | | | | CAUA |
| 684 | UUCUAUGU | 876 | CCAAGUUAA | 1068 | UGCUGCCA |
| | UAACUUGG | | CAUAGAA | | AGUUAACAU |
| | CAGCA | | | | AGAG |
| 685 | UAGCUUUCA | 877 | GUUGGUUU | 1069 | ACACAGUUG |
| | AACCAACUG | | GAAAGCUA | | GUUUGAAA |
| | UGU | | | | GCUU |
| 686 | UAAGCUUUC | 878 | UUGGUUUG | 1070 | CACAGUUG |
| | AAACCAACU | | AAAGCUUA | | GUUUGAAA |
| | GUG | | | | GCUUC |
| 687 | UGAAGCUU | 879 | UGGUUUGA | 1071 | ACAGUUGG |
| | UCAAACCAA | | AAGCUUCA | | UUUGAAAGC |
| | CUGU | | | | UUCA |
| 688 | UAAUGAAGC | 880 | UUUGAAAGC | 1072 | GUUGGUUU |
| | UUUCAAACC | | UUCAUUA | | GAAAGCUUC |
| | AAC | | | | AUUG |
| 689 | UCAAUGAAG | 881 | UUGAAAGCU | 1073 | UUGGUUUG |
| | CUUUCAAAC | | UCAUUGA | | AAAGCUUCA |
| | CAA | | | | UUGU |
| 690 | UACAAUGAA | 882 | UGAAAGCUU | 1074 | UGGUUUGA |
| | GCUUUCAAA | | CAUUGUA | | AAGCUUCAU |
| | CCA | | | | UGUC |
| 691 | UGACAAUGA | 883 | GAAAGCUUC | 1075 | GGUUUGAA |
| | AGCUUUCAA | | AUUGUCA | | AGCUUCAUU |
| | ACC | | | | GUCC |
| 692 | UGAUAAUCU | 884 | ACCAUUAAG | 1076 | AAAAGACCA |
| | UAAUGGUC | | AUUAUCA | | UUAAGAUUA |
| | UUUU | | | | UCC |
| 693 | UCACAUUCA | 885 | UCCAUCAUG | 1077 | UUCCUUCCA |
| | UGAUGGAA | | AAUGUGA | | UCAUGAAUG |
| | GGAA | | | | UGC |
| 694 | UUACACUCA | 886 | AGUUCUAU | 1078 | UGGCAAGU |
| | UAGAACUUG | | GAGUGUAA | | UCUAUGAG |
| | CCA | | | | UGUAU |
| 695 | UAUACACUC | 887 | GUUCUAUG | 1079 | GGCAAGUU |
| | AUAGAACUU | | AGUGUAUA | | CUAUGAGU |
| | GCC | | | | GUAUU |
| 696 | UAAUACACU | 888 | UUCUAUGA | 1080 | GCAAGUUC |
| | CAUAGAACU | | GUGUAUUA | | UAUGAGUG |
| | UGC | | | | UAUUA |
| 697 | UCAUCGCAC | 889 | UCAAAAUGU | 1081 | GUUAGUCAA |
| | AUUUUGACU | | GCGAUGA | | AAUGUGCG |
| | AAC | | | | AUGG |
| 698 | UCCAUCGCA | 890 | CAAAAUGUG | 1082 | UUAGUCAAA |
| | CAUUUUGAC | | CGAUGGA | | AUGUGCGA |
| | UAA | | | | UGGA |
| 699 | UUCCAUCG | 891 | AAAAUGUGC | 1083 | UAGUCAAAA |
| | CACAUUUUG | | GAUGGAA | | UGUGCGAU |
| | ACUA | | | | GGAA |
| 700 | UUUCCAUC | 892 | AAAUGUGC | 1084 | AGUCAAAAU |
| | GCACAUUUU | | GAUGGAAA | | GUGCGAUG |
| | GACU | | | | GAAA |
| 701 | UCAACUUGA | 893 | CUCUGCUU | 1085 | CCUAUCUCU |
| | AGCAGAGAU | | CAAGUUGA | | GCUUCAAG |
| | AGG | | | | UUGC |
| 702 | UGCAACUU | 894 | UCUGCUUC | 1086 | CUAUCUCU |
| | GAAGCAGA | | AAGUUGCA | | GCUUCAAG |
| | GAUAG | | | | UUGCA |
| 703 | UUGCAACUU | 895 | CUGCUUCAA | 1087 | UAUCUCUG |
| | GAAGCAGA | | GUUGCAA | | CUUCAAGUU |
| | GAUA | | | | GCAA |
| 704 | UAAGUUGCA | 896 | UUCAAGUU | 1088 | UCUGCUUC |
| | ACUUGAAGC | | GCAACUUA | | AAGUUGCAA |
| | AGA | | | | CUUU |
| 705 | UAAAGUUGC | 897 | UCAAGUUG | 1089 | CUGCUUCAA |
| | AACUUGAAG | | CAACUUUA | | GUUGCAAC |
| | CAG | | | | UUUU |
| 706 | UAAAAGUUG | 898 | CAAGUUGCA | 1090 | UGCUUCAA |
| | CAACUUGAA | | ACUUUUA | | GUUGCAAC |
| | GCA | | | | UUUUA |
| 707 | UCAAAGUGA | 899 | UCAUUCUUC | 1091 | UUGGGUCA |
| | AGAAUGACC | | ACUUUGA | | UUCUUCACU |
| | CAA | | | | UUGA |
| 708 | UUCAAAGUG | 900 | CAUUCUUCA | 1092 | UGGGUCAU |
| | AAGAAUGAC | | CUUUGAA | | UCUUCACUU |
| | CCA | | | | UGAA |
| 709 | UUUCAAAGU | 901 | AUUCUUCAC | 1093 | GGGUCAUU |
| | GAAGAAUGA | | UUUGAAA | | CUUCACUUU |
| | CCC | | | | GAAC |
| 710 | UGUUCAAAG | 902 | UUCUUCACU | 1094 | GGUCAUUC |
| | UGAAGAAUG | | UUGAACA | | UUCACUUU |
| | ACC | | | | GAACU |
| 711 | UCAAGUUCA | 903 | UUCACUUU | 1095 | CAUUCUUCA |
| | AAGUGAAGA | | GAACUUGA | | CUUUGAACU |
| | AUG | | | | UGU |
| 712 | UACAAGUUC | 904 | UCACUUUGA | 1096 | AUUCUUCAC |
| | AAAGUGAAG | | ACUUGUA | | UUUGAACUU |
| | AAU | | | | GUU |
| 713 | UAAUGAACA | 905 | UUGAACUU | 1097 | UCACUUUGA |
| | AGUUCAAAG | | GUUCAUUA | | ACUUGUUCA |
| | UGA | | | | UUG |
| 714 | UCCAAUGAA | 906 | GAACUUGU | 1098 | ACUUUGAAC |
| | CAAGUUCAA | | UCAUUGGA | | UUGUUCAU |
| | AGU | | | | UGGU |
| 715 | UACACCAAU | 907 | CUUGUUCA | 1099 | UUGAACUU |
| | GAACAAGUU | | UUGGUGUA | | GUUCAUUG |
| | CAA | | | | GUGUC |
| 716 | UUUGGUUG | 908 | GAUAAUUUC | 1100 | UCAUAGAUA |
| | AAAUUAUCU | | AACCAAA | | AUUUCAACC |
| | AUGA | | | | AAC |
| 717 | UUUUCUGU | 909 | UUCAACCAA | 1101 | AUAAUUUCA |
| | UGGUUGAA | | CAGAAAA | | ACCAACAGA |
| | AUUAU | | | | AAA |
| 718 | UAAUAUACA | 910 | CCAAGGAU | 1102 | AAAAUCCAA |
| | UCCUUGGA | | GUAUAUUA | | GGAUGUAU |
| | UUUU | | | | AUUU |
| 719 | UAAAUAUAC | 911 | CAAGGAUG | 1103 | AAAUCCAAG |
| | AUCCUUGG | | UAUAUUUA | | GAUGUAUAU |
| | AUUU | | | | UUG |
| 720 | UUCAAAUAU | 912 | AGGAUGUA | 1104 | AUCCAAGGA |
| | ACAUCCUUG | | UAUUUGAA | | UGUAUAUUU |
| | GAU | | | | GAC |
| 721 | UGGUCAAAU | 913 | GAUGUAUAU | 1105 | CCAAGGAU |
| | AUACAUCCU | | UUGACCA | | GUAUAUUU |
| | UGG | | | | GACCU |
| 722 | UAGGUCAAA | 914 | AUGUAUAUU | 1106 | CAAGGAUG |
| | UAUACAUCC | | UGACCUA | | UAUAUUUGA |
| | UUG | | | | CCUA |
| 723 | UUAGGUCAA | 915 | UGUAUAUUU | 1107 | AAGGAUGUA |
| | AUAUACAUC | | GACCUAA | | UAUUUGACC |
| | CUU | | | | UAG |
| 724 | UCUAGGUC | 916 | GUAUAUUU | 1108 | AGGAUGUA |
| | AAAUAUACA | | GACCUAGA | | UAUUUGACC |
| | UCCU | | | | UAGU |
| 725 | UAGAUAAGA | 917 | UCAUGGUU | 1109 | UAGUAUCAU |
| | ACCAUGAUA | | CUUAUCUA | | GGUUCUUA |
| | CUA | | | | UCUG |
| 726 | UUCAUGGU | 918 | ACAUGGUAA | 1110 | UCUCAACAU |
| | UACCAUGUU | | CCAUGAA | | GGUAACCAU |
| | GAGA | | | | GAU |
| 727 | UAUCAUGG | 919 | CAUGGUAAC | 1111 | CUCAACAUG |
| | UUACCAUGU | | CAUGAUA | | GUAACCAUG |
| | UGAG | | | | AUG |
| 728 | UCAUCAUG | 920 | AUGGUAACC | 1112 | UCAACAUGG |
| | GUUACCAU | | AUGAUGA | | UAACCAUGA |
| | GUUGA | | | | UGG |
| 729 | UCCAUCAUG | 921 | UGGUAACCA | 1113 | CAACAUGGU |
| | GUUACCAU | | UGAUGGA | | AACCAUGAU |
| | GUUG | | | | GGU |
| 730 | UUACCAUCA | 922 | GUAACCAUG | 1114 | ACAUGGUAA |
| | UGGUUACC | | AUGGUAA | | CCAUGAUG |
| | AUGU | | | | GUAG |
| 731 | UCUACCAUC | 923 | UAACCAUGA | 1115 | CAUGGUAAC |
| | AUGGUUAC | | UGGUAGA | | CAUGAUGG |
| | CAUG | | | | UAGA |
| 732 | UUGUCAUUA | 924 | AUGGAAUUA | 1116 | GGAAGAUG |
| | AUUCCAUCU | | AUGACAA | | GAAUUAAUG |
| | UCC | | | | ACAU |
| 733 | UAUGUCAUU | 925 | UGGAAUUAA | 1117 | GAAGAUGG |
| | AAUUCCAUC | | UGACAUA | | AAUUAAUGA |
| | UUC | | | | CAUG |
| 734 | UAUUGAACA | 926 | AAUGACAUG | 1118 | GAAUUAAUG |
| | UGUCAUUAA | | UUCAAUA | | ACAUGUUCA |
| | UUC | | | | AUU |
| 735 | UAAAUUGAA | 927 | UGACAUGU | 1119 | AUUAAUGAC |
| | CAUGUCAUU | | UCAAUUUA | | AUGUUCAAU |
| | AAU | | | | UUU |
| 736 | UAAAAUUGA | 928 | GACAUGUU | 1120 | UUAAUGACA |
| | ACAUGUCAU | | CAAUUUUA | | UGUUCAAUU |
| | UAA | | | | UUG |
| 737 | UCUGUGAA | 929 | UCUCUUCG | 1121 | UGGAUUCU |
| | CGAAGAGAA | | UUCACAGA | | CUUCGUUC |
| | UCCA | | | | ACAGA |
| 738 | UUCUGUGA | 930 | CUCUUCGU | 1122 | GGAUUCUC |
| | ACGAAGAGA | | UCACAGAA | | UUCGUUCA |
| | AUCC | | | | CAGAU |
| 739 | UAUCUGUG | 931 | UCUUCGUU | 1123 | GAUUCUCU |
| | AACGAAGAG | | CACAGAUA | | UCGUUCACA |
| | AAUC | | | | GAUG |
| 740 | UCAUCUGU | 932 | CUUCGUUC | 1124 | AUUCUCUUC |
| | GAACGAAGA | | ACAGAUGA | | GUUCACAGA |
| | GAAU | | | | UGG |
| 741 | UCAUGAACC | 933 | GAAGAAAGG | 1125 | AGAUGGAA |
| | UUUCUUCCA | | UUCAUGA | | GAAAGGUU |
| | UCU | | | | CAUGU |
| 742 | UGACAUGAA | 934 | AGAAAGGUU | 1126 | AUGGAAGAA |
| | CCUUUCUU | | CAUGUCA | | AGGUUCAU |
| | CCAU | | | | GUCU |
| 743 | UAGACAUGA | 935 | GAAAGGUU | 1127 | UGGAAGAAA |
| | ACCUUUCUU | | CAUGUCUA | | GGUUCAUG |
| | CCA | | | | UCUG |
| 744 | UCUUUUAU | 936 | GUAUAUACA | 1128 | AUCAAGUAU |
| | GUAUAUACU | | UAAAAGA | | AUACAUAAA |
| | UGAU | | | | AGA |
| 745 | UUCUUUUAU | 937 | UAUAUACAU | 1129 | UCAAGUAUA |
| | GUAUAUACU | | AAAAGAA | | UACAUAAAA |
| | UGA | | | | GAU |
| 746 | UAUCUUUUA | 938 | AUAUACAUA | 1130 | CAAGUAUAU |
| | UGUAUAUAC | | AAAGAUA | | ACAUAAAAG |
| | UUG | | | | AUG |
| 747 | UCAUCUUUU | 939 | UAUACAUAA | 1131 | AAGUAUAUA |
| | AUGUAUAUA | | AAGAUGA | | CAUAAAAGA |
| | CUU | | | | UGG |
| 748 | UCCAUCUUU | 940 | AUACAUAAA | 1132 | AGUAUAUAC |
| | UAUGUAUAU | | AGAUGGA | | AUAAAAGAU |
| | ACU | | | | GGA |
| 749 | UUCCAUCUU | 941 | UACAUAAAA | 1133 | GUAUAUACA |
| | UUAUGUAUA | | GAUGGAA | | UAAAAGAUG |
| | UAC | | | | GAG |
| 750 | UCUCCAUCU | 942 | ACAUAAAAG | 1134 | UAUAUACAU |
| | UUUAUGUAU | | AUGGAGA | | AAAAGAUGG |
| | AUA | | | | AGA |
| 751 | UGUCUCCA | 943 | AUAAAAGAU | 1135 | UAUACAUAA |
| | UCUUUUAU | | GGAGACA | | AAGAUGGA |
| | GUAUA | | | | GACA |
| 752 | UCUGGACU | 944 | AGAACUCAA | 1136 | UAAUGAGAA |
| | UGAGUUCU | | GUCCAGA | | CUCAAGUCC |
| | CAUUA | | | | AGA |
| 753 | UUCUGGAC | 945 | GAACUCAAG | 1137 | AAUGAGAAC |
| | UUGAGUUC | | UCCAGAA | | UCAAGUCCA |
| | UCAUU | | | | GAA |
| 754 | UUUCUGGA | 946 | AACUCAAGU | 1138 | AUGAGAACU |
| | CUUGAGUU | | CCAGAAA | | CAAGUCCAG |
| | CUCAU | | | | AAA |
| 755 | UGUGGUUA | 947 | UCAUAGAAU | 1139 | UUUGUUCA |
| | UUCUAUGAA | | AACCACA | | UAGAAUAAC |
| | CAAA | | | | CACA |
| 756 | UAGGAGUG | 948 | ACAUAUUAC | 1140 | UAAGUACAU |
| | UAAUAUGUA | | ACUCCUA | | AUUACACUC |
| | CUUA | | | | CUC |
| 757 | UUUAUUAUA | 949 | ACAUUUAUA | 1141 | AAAUUACAU |
| | UAAAUGUAA | | UAAUAAA | | UUAUAUAAU |
| | UUU | | | | AAA |
| 758 | UCUUAAUUG | 950 | AUACAUUCA | 1142 | UUUAAAUAC |
| | AAUGUAUUU | | AUUAAGA | | AUUCAAUUA |
| | AAA | | | | AGA |
| 759 | UUUAAUAGA | 951 | GUAAUUUUC | 1143 | CGUGUGUA |
| | AAAUUACAC | | UAUUAAA | | AUUUUCUAU |
| | ACG | | | | UAAU |
| 760 | UUUAAUAUG | 952 | GCACUGUC | 1144 | AGAAGGCAC |
| | ACAGUGCC | | AUAUUAAA | | UGUCAUAUU |
| | UUCU | | | | AAU |
| 761 | UAAACUAGG | 953 | AUAGUUACC | 1145 | UUUUGAUA |
| | UAACUAUCA | | UAGUUUA | | GUUACCUA |
| | AAA | | | | GUUUG |
| 762 | UGAAAUAGC | 954 | CUAAAUAGC | 1146 | CAGUUCUAA |
| | UAUUUAGAA | | UAUUUCA | | AUAGCUAUU |
| | CUG | | | | UCA |
| 763 | UUGUAAAGA | 955 | AUAGGAUUC | 1147 | UUUACAUAG |
| | AUCCUAUGU | | UUUACAA | | GAUUCUUUA |
| | AAA | | | | CAA |
| 764 | UAACAUUUU | 956 | AAUGCUCAA | 1148 | CUAUGAAUG |
| | GAGCAUUCA | | AAUGUUA | | CUCAAAAUG |
| | UAG | | | | UUU |
| 765 | UAAACAUUU | 957 | AUGCUCAAA | 1149 | UAUGAAUGC |
| | UGAGCAUU | | AUGUUUA | | UCAAAAUGU |
| | CAUA | | | | UUG |
| 766 | UCAAACAUU | 958 | UGCUCAAAA | 1150 | AUGAAUGCU |
| | UUGAGCAU | | UGUUUGA | | CAAAAUGUU |
| | UCAU | | | | UGA |
| 767 | UCAAGUAUA | 959 | UUGUAGUU | 1151 | UUAUAUUGU |
| | ACUACAAUA | | AUACUUGA | | AGUUAUACU |
| | UAA | | | | UGA |
| 768 | UUCAAGUAU | 960 | UGUAGUUA | 1152 | UAUAUUGUA |
| | AACUACAAU | | UACUUGAA | | GUUAUACUU |
| | AUA | | | | GAG |

siRNA Structure

The siRNA molecules of the disclosure may be in the form of a single-stranded (ss) or double-stranded (ds) oligonucleotide structure. In some embodiments, the siRNA molecules may be di-branched, tri-branched, or tetra-branched molecules. Furthermore, the siRNA molecules of the disclosure may contain one or more phosphodiester internucleoside linkages and/or an analog thereof, such as a phosphorothioate internucleoside linkage. The siRNA molecules of the disclosure may further contain chemically modified nucleosides having 2′ sugar modifications.

The simplest siRNAs consist of a ribonucleic acid, including a ss- or ds-structure, formed by a first strand (i.e., antisense strand), and in the case of a ds-siRNA, a second strand (i.e., sense strand). The first strand includes a stretch of contiguous nucleotides that is at least partially complementary to a target nucleic acid. The second strand also includes a stretch of contiguous nucleotides where the second stretch is at least partially identical to a target nucleic acid. The first strand and said second strand may be hybridized to each other to form a double-stranded structure. The hybridization typically occurs by Watson Crick base pairing.

Depending on the sequence of the first and second strand, the hybridization or base pairing is not necessarily complete or perfect, which means that the first and second strand are not 100% base-paired due to mismatches. One or more mismatches may also be present within the duplex without necessarily impacting the siRNA RNAi activity.

The first strand contains a stretch of contiguous nucleotides which is essentially complementary to a target nucleic acid. Typically, the target nucleic acid sequence is, in accordance with the mode of action of interfering ribonucleic acids, a ss-RNA, preferably an mRNA. Such hybridization occurs most likely through Watson Crick base pairing but is not necessarily limited thereto. The extent to which the first strand has a complementary stretch of contiguous nucleotides to a target nucleic acid sequence may be between 80% and 100%, e.g., 80%, 85%, 90%, 95%, or 100% complementary.

The siRNA molecules described herein may employ modifications to the nucleobase, phosphate backbone, ribose core, 5′- and 3′-ends, and branching, wherein multiple strands of siRNA may be covalently linked.

### Lengths of Small Interfering RNA Molecules

It is within the scope of the disclosure that any length, known and previously unknown in the art, may be employed for the current invention. As described herein, potential lengths for an antisense strand of the siRNA molecules of the present disclosure is between 10 and 30 nucleotides (e.g., 10 nucleotides, 11 nucleotides, 12 nucleotides, 13 nucleotides, 14 nucleotides, 15 nucleotides, 16 nucleotides, 17 nucleotides, 18 nucleotides, 19 nucleotides, 20 nucleotides, 21 nucleotides, 22 nucleotides, 23 nucleotides, 24 nucleotides, 25 nucleotides, 26 nucleotides, 27 nucleotides, 28 nucleotides, 29 nucleotides, or 30 nucleotides), 15 and 25 nucleotides (e.g., 15 nucleotides, 16 nucleotides, 17 nucleotides, 18 nucleotides, 19 nucleotides, 20 nucleotides, 21 nucleotides, 22 nucleotides, 23 nucleotides, 24 nucleotides, or 25 nucleotides), or 18 and 23 nucleotides (e.g., 18 nucleotides, 19 nucleotides, 20 nucleotides, 21 nucleotides, 22 nucleotides, or 23 nucleotides). In some embodiments, the antisense strand is 20 nucleotides. In some embodiments, the antisense strand is 21 nucleotides. In some embodiments, the antisense strand is 22 nucleotides. In some embodiments, the antisense strand is 23 nucleotides. In some embodiments, the antisense strand is 24 nucleotides. In some embodiments, the antisense strand is 25 nucleotides. In some embodiments, the antisense strand is 26 nucleotides. In some embodiments, the antisense strand is 27 nucleotides. In some embodiments, the antisense strand is 28 nucleotides. In some embodiments, the antisense strand is 29 nucleotides. In some embodiments, the antisense strand is 30 nucleotides.

In some embodiments, the sense strand of the siRNA molecules of the present disclosure is between 12 and 30 nucleotides (e.g., 12 nucleotides, 13 nucleotides, 14 nucleotides, 15 nucleotides, 16 nucleotides, 17 nucleotides, 18 nucleotides, 19 nucleotides, 20 nucleotides, 21 nucleotides, 22 nucleotides, 23 nucleotides, 24 nucleotides, 25 nucleotides, 26 nucleotides, 27 nucleotides, 28 nucleotides, 29 nucleotides, or 30 nucleotides), or 14 and 23 nucleotides (e.g., 14 nucleotides, 15 nucleotides, 16 nucleotides, 17 nucleotides, 18 nucleotides, 19 nucleotides, 20 nucleotides, 21 nucleotides, 22 nucleotides, or 23 nucleotides). In some embodiments, the sense strand is 15 nucleotides. In some embodiments, the sense strand is 16 nucleotides. In some embodiments, the sense strand is 17 nucleotides. In some embodiments, the sense strand is 18 nucleotides. In some embodiments, the sense strand is 19 nucleotides. In some embodiments, the sense strand is 20 nucleotides. In some embodiments, the sense strand is 21 nucleotides. In some embodiments, the sense strand is 22 nucleotides. In some embodiments, the sense strand is 23 nucleotides. In some embodiments, the sense strand is 24 nucleotides. In some embodiments, the sense strand is 25 nucleotides. In some embodiments, the sense strand is 26 nucleotides. In some embodiments, the sense strand is 27 nucleotides. In some embodiments, the sense strand is 28 nucleotides. In some embodiments, the sense strand is 29 nucleotides. In some embodiments, the sense strand is 30 nucleotides.

### 2′ Sugar Modifications

The present disclosure may include ss- and ds-siRNA molecule compositions including at least one (e.g., at least 1, 2, 3, 4, 5, 6, 7, 8, 9, 10, 11, or more) nucleosides having 2′ sugar modifications. Possible 2′-modifications include all possible orientations of OH; F; O-, S-, or N-alkyl; O-, S-, or N-alkenyl; O-, S- or N-alkynyl; or O-alkyl-O-alkyl, wherein the alkyl, alkenyl and alkynyl may be substituted or unsubstituted C1 to C10 alkyl or C2 to C10 alkenyl and alkynyl. In some embodiments, the modification includes a 2′-O-methyl (2′-O-Me) modification. Other potential sugar substituent groups include: C1 to C10 lower alkyl, substituted lower alkyl, alkenyl, alkynyl, alkaryl, aralkyl, O-alkaryl or O-aralkyl, SH, SCH₃, OCN, Cl, Br, CN, CF₃, OCF₃, SOCH₃, SO₂CH₃, ONO₂, NO₂, N₃, NH₂, heterocycloalkyl, heterocycloalkaryl, aminoalkylamino, polyalkylamino, substituted silyl, a group for improving the pharmacokinetic properties of an oligonucleotide, or a group for improving the pharmacodynamic properties of an oligonucleotide, and other substituents having similar properties. In some embodiments, the modification includes 2′-methoxyethoxy (2′-O—CH₂CH₂OCH₃, also known as 2′-O-(2-methoxyethyl) or 2′-MOE). In some embodiments, the modification includes 2′-dimethylaminooxyethoxy, i.e., a O(CH₂)₂ON(CH₃)₂ group, also known as 2′-DMAOE, and 2′-dimethylaminoethoxyethoxy (also known in the art as 2′-O-dimethylamino-ethoxy-ethyl or 2′-DMAEOE), i.e., 2′-O—CH₂OCH₂N(CH₃)₂. Other potential sugar substituent groups include, e.g., aminopropoxy (—OCH₂CH₂CH₂NH₂), allyl (—CH₂—CH═CH₂), —O-allyl (—O—CH₂—CH═CH₂) and fluoro (F). 2′-sugar substituent groups may be in the arabino (up) position or ribo (down) position. In some embodiments, the 2′-arabino modification is 2′-F. Similar modifications may also be made at other positions on the siRNA molecule, particularly the 3′ position of the sugar on the 3′ terminal nucleoside or in 2′-5′ linked oligonucleotides and the 5′ position of 5′ terminal nucleotide. Oligonucleotides may also have sugar mimetics such as cyclobutyl moieties in place of the pentofuranosyl sugar.

### Nucleobase Modifications

The siRNA molecules of the disclosure may also include nucleosides or other surrogate or mimetic monomeric subunits that include a nucleobase (often referred to in the art simply as “base” or “heterocyclic base moiety”). The nucleobase is another moiety that has been extensively modified or substituted and such modified and or substituted nucleobases are amenable to the present disclosure. As used herein, “unmodified” or “natural” nucleobases include the purine bases adenine (A) and guanine (G), and the pyrimidine bases thymine (T), cytosine (C) and uracil (U). Modified nucleobases also referred herein as heterocyclic base moieties include other synthetic and natural nucleobases such as 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl (—C═C—CH3) uracil and cytosine and other alkynyl derivatives of pyrimidine bases, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 2-F-adenine, 2-amino-adenine, 8-azaguanine and 8-azaadenine, 7-deazaguanine and 7-deazaadenine and 3-deazaguanine and 3-deazaadenine. Nucleobases may also include those in which the purine or pyrimidine base is replaced with other heterocycles, for example 7-deaza-adenine, 7-deazaguanosine, 2-aminopyridine and 2-pyridone. Further nucleobases include those disclosed in U.S. Pat. No. 3,687,808, those disclosed in Kroschwitz, J. I., ed. The Concise Encyclopedia of Polymer Science and Engineering, New York, John Wiley & Sons, 1990, pp. 858-859; those disclosed by Englisch et al., Angewandte Chemie, International Edition 30:613, 1991; and those disclosed by Sanghvi, Y. S., Chapter 16, Antisense Research and Applications, CRC Press, Gait, M. J. ed., 1993, pp. 289-302. The siRNA molecules of the present disclosure may also include polycyclic heterocyclic compounds in place of one or more heterocyclic base moieties. A number of tricyclic heterocyclic compounds have been previously reported. These compounds are routinely used in antisense applications to increase the binding properties of the modified strand to a target strand.

Representative cytosine analogs that make three hydrogen bonds with a guanosine in a second strand include 1,3-diazaphenoxazine-2-one (Kurchavov et al., Nucleosides and Nucleotides, 16:1837-46, 1997), 1,3-diazaphenothiazine-2-one (Lin et al. Am. Chem. Soc., 117:3873-4, 1995), and 6,7,8,9-tetrafluoro-1,3-diazaphenoxazine-2-one (Wang et al., Tetrahedron Lett., 39:8385-8, 1998). Incorporated into oligonucleotides, these base modifications were shown to hybridize with complementary guanine and the latter was also shown to hybridize with adenine and to enhance helical thermal stability by extended stacking interactions (also see U.S. Ser. No. 10/155,920 and U.S. Ser. No. 10/013,295, both of which are herein incorporated by reference in their entirety). Further helix-stabilizing properties have been observed when a cytosine analog/substitute has an aminoethoxy moiety attached to the rigid 1,3-diazaphenoxazine-2-one scaffold (Lin et al., Am. Chem. Soc., 120:8531-2, 1998).

### Internucleoside Linkage Modifications

Another variable in the design of the present disclosure is the internucleoside linkage making up the phosphate backbone of the siRNA molecule. Although the natural RNA phosphate backbone may be employed here, derivatives thereof may be used which enhance desirable characteristics of the siRNA molecule. Although not limiting, of particular importance in the present disclosure is protecting parts, or the whole, of the siRNA molecule from hydrolysis. One example of a modification that decreases the rate of hydrolysis is phosphorothioates. Any portion or the whole of the backbone may contain phosphate substitutions (e.g., phosphorothioates). For instance, the internucleoside linkages may be between 0 and 100% phosphorothioate, e.g., between 0 and 100%, 10 and 100%, 20 and 100%, 30 and 100%, 40 and 100%, 50 and 100%, 60 and 100%, 70 and 100%, 80 and 100%, 90 and 100%, 0 and 90%, 0 and 80%, 0 and 70%, 0 and 60%, 0 and 50%, 0 and 40%, 0 and 30%, 0 and 20%, 0 and 10%, 10 and 90%, 20 and 80%, 30 and 70%, 40 and 60%, 10 and 40%, 20 and 50%, 30 and 60%, 40 and 70%, 50 and 80%, or 60 and 90% phosphorothioate linkages. Similarly, the internucleoside linkages may be between 0 and 100% phosphodiester linkages, e.g., between 0 and 100%, 10 and 100%, 20 and 100%, 30 and 100%, 40 and 100%, 50 and 100%, 60 and 100% 70 and 100%, 80 and 100%, 90 and 100%, 0 and 90%, 0 and 80%, 0 and 70%, 0 and 60%, 0 and 50%, 0 and 40%, 0 and 30%, 0 and 20%, 0 and 10%, 10 and 90%, 20 and 80%, 30 and 70%, 40 and 60%, 10 and 40%, 20 and 50%, 30 and 60%, 40 and 70%, 50 and 80%, or 60 and 90% phosphodiester linkages.

Specific examples of some potential siRNA molecules useful in this invention include oligonucleotides containing modified e.g., non-naturally occurring internucleoside linkages. As defined in this specification, oligonucleotides having modified internucleoside linkages include internucleoside linkages that retain a phosphorus atom and internucleoside linkages that do not have a phosphorus atom. For the purposes of this specification, and as sometimes referenced in the art, modified oligonucleotides that do not have a phosphorus atom in their internucleoside backbone can also be considered to be oligonucleosides. A preferred phosphorus containing modified internucleoside linkage is the phosphorothioate internucleoside linkage. In some embodiments, the modified oligonucleotide backbones containing a phosphorus atom therein include, for example, phosphorothioates, phosphorodithioates, phosphotriesters, aminoalkylphosphotriesters, methyl and other alkyl phosphonates including 3-alkylene phosphonates, 5′-alkylene phosphonates, phosphinates, phosphoramidates including 3-amino phosphoramidate and aminoalkylphosphoramidates, thionophosphoramidates, thionoalkylphosphonates, thionoalkylphosphotriesters, selenophosphates, and boranophosphates having normal 3-5′ linkages, 2′-5′ linked analogs of these, and those having inverted polarity wherein one or more internucleotide linkages is a 3′ to 3′, 5′ to 5′ or 2′ to 2′ linkage. Exemplary U.S. patents describing the preparation of phosphorus-containing linkages include but are not limited to, U.S. Pat. Nos. 3,687,808; 4,469,863; 4,476,301; 5,023,243; 5,177,195; 5,188,897; 5,264,423; 5,276,019; 5,278,302; 5,286,717; 5,321,131; 5,399,676; 5,405,939; 5,453,496; 5,455,233; 5,466,677; 5,476,925; 5,519,126; 5,536,821; 5,541,316; 5,550,111; 5,563,253; 5,571,799; 5,587,361; 5,625,050; 6,028,188; 6,124,445; 6,160,109; 6,169,170; 6,172,209; 6,239,265; 6,277,603; 6,326,199; 6,346,614; 6,444,423; 6,531,590; 6,534,639; 6,608,035; 6,683,167; 6,858,715; 6,867,294; 6,878,805; 7,015,315; 7,041,816; 7,273,933; 7,321,029; and U.S. Pat. RE39464, the entire contents of each of which are hereby incorporated herein by reference.

In some embodiments, the modified oligonucleotide backbones that do not include a phosphorus atom therein have backbones that are formed by short chain alkyl or cycloalkyl internucleoside linkages, mixed heteroatom and alkyl or cycloalkyl internucleoside linkages, or one or more short chain heteroatomic or heterocyclic internucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; riboacetyl backbones; alkene containing backbones; sulfamate backbones; methyleneimino and methylenehydrazino backbones; sulfonate and sulfonamide backbones; amide backbones; and others having mixed N, O, S and CH₂ component parts. Non-limiting examples of U.S. patents that teach the preparation of non-phosphorus backbones include, but are not limited to, U.S. Pat. Nos. 5,034,506; 5,166,315; 5,185,444; 5,214,134; 5,216,141; 5,235,033; 5,64,562; 5,264,564; 5,405,938; 5,434,257; 5,466,677; 5,470,967; 5,489,677; 5,541,307; 5,561,225; 5,596,086; 5,602,240; 5,608,046; 5,610,289; 5,618,704; 5,623,070; 5,663,312; 5,633,360; 5,677,437; and 5,677,439, the entire contents of each of which are hereby incorporated herein by reference.

Patterns of Modifications of siRNA Molecules

The following section provides a set of exemplary scaffolds into which the siRNA molecules of the disclosure may be incorporated.

In some embodiments of the disclosure, the siRNA may contain an antisense strand including a region represented by Formula I, wherein Formula I is, in the 5′-to-3′ direction:

A-B-(A′)ⱼ-C-P²-D-P¹-(C′-P¹)ₖ-C′ Formula I;
wherein A is represented by the formula C-P¹-D-P¹; each A′ is represented by the formula C-P²-D-P²; B is represented by the formula C-P²-D-P²-D-P²-D-P²; each C is a 2′-O-methyl (2′-O-Me) ribonucleoside; each C′, independently, is a 2′-O-Me ribonucleoside or a 2′-fluoro (2′-F) ribonucleoside; each D is a 2′-F ribonucleoside; each P¹ is a phosphorothioate internucleoside linkage; each P² is a phosphodiester internucleoside linkage; j is an integer from 1 to 7 (e.g., 1, 2, 3, 4, 5, 6, or 7); and k is an integer from 1 to 7 (e.g., 1, 2, 3, 4, 5, 6, or 7). In some embodiments, j is 4. In some embodiments, k is 4. In some embodiments, j is 4 and k is 4. The antisense is complementary (e.g., fully or partially complementary) to a target nucleic acid sequence.

In some embodiments, the antisense strand includes a structure represented by Formula A1, wherein Formula A1 is, in the 5′-to-3′ direction:

A-S-B-S-A-O-B-O-B-O-B-O-A-O-B-O-A-O-B-O-A-O-B-O-A-O-B-O-A-O-B-S-A-S-A-S-A-S-B-S-A Formula A1;
wherein A represents a 2′-O-Me ribonucleoside, B represents a 2′-F ribonucleoside, O represents a phosphodiester internucleoside linkage, and S represents a phosphorothioate internucleoside linkage.

In some embodiments of the disclosure, the siRNA may contain an antisense strand including a region represented by Formula II, wherein Formula II is, in the 5′-to-3′ direction:

A-B-(A′)ⱼ-C-P²-D-P¹-(C-P¹)ₖ-C′ Formula II;
wherein A is represented by the formula C-P¹-D-P¹; each A′ is represented by the formula C-P²-D-P²; B is represented by the formula C-P²-D-P²-D-P²-D-P²; each C is a 2′-O-methyl (2′-O-Me) ribonucleoside; each C′, independently, is a 2′-O-Me ribonucleoside or a 2′-fluoro (2′-F) ribonucleoside; each D is a 2′-F ribonucleoside; each P¹ is a phosphorothioate internucleoside linkage; each P² is a phosphodiester internucleoside linkage; j is an integer from 1 to 7 (e.g., 1, 2, 3, 4, 5, 6, or 7); and k is an integer from 1 to 7 (e.g., 1, 2, 3, 4, 5, 6, or 7). In some embodiments, j is 4. In some embodiments, k is 4. In some embodiments, j is 4 and k is 4. The antisense is complementary (e.g., fully or partially complementary) to a target nucleic acid sequence.

In some embodiments of the disclosure, the antisense strand includes a structure represented by Formula A2, wherein Formula A2 is, in the 5′-to-3′ direction:

A-S-B-S-A-O-B-O-B-O-B-O-A-O-B-O-A-O-B-O-A-O-B-O-A-O-B-O-A-O-B-S-A-S-A-S-A-S-A-S-A Formula A2;
wherein A represents a 2′-O-Me ribonucleoside, B represents a 2′-F ribonucleoside, O represents a phosphodiester internucleoside linkage, and S represents a phosphorothioate internucleoside linkage.

In some embodiments of the disclosure, the sense strand includes a structure represented by Formula III, wherein Formula III is, in the 5′-to-3′ direction:

E-(A′)ₘ-F Formula III;
wherein E is represented by the formula (C-P¹)₂; F is represented by the formula (C-P²)₃-D-P¹-C-P¹-C, (C-P²)₃-D-P²-C-P²-C, (C-P²)₃-D-P¹-C-P¹-D, or (C-P²)₃-D-P²-C-P²-D; A′, C, D, P¹, and P² are as defined in Formula I; and m is an integer from 1 to 7 (e.g., 1, 2, 3, 4, 5, 6, or 7). In some embodiments, m is 4. The sense strand is complementary (e.g., fully or partially complementary) to the antisense strand.

In some embodiments of the disclosure, the sense strand includes a structure represented by Formula S1, wherein Formula S1 is, in the 5′-to-3′ direction:

A-S-A-S-A-O-B-O-A-O-B-O-A-O-B-O-A-O-B-O-A-O-A-O-A-O-B-S-A-S-A Formula S1;
wherein A represents a 2′-O-Me ribonucleoside, B represents a 2′-F ribonucleoside, O represents a phosphodiester internucleoside linkage, and S represents a phosphorothioate internucleoside linkage.

In some embodiments of the disclosure, the sense strand includes a structure represented by Formula S2, wherein Formula S2 is, in the 5′-to-3′ direction:

A-S-A-S-A-O-B-O-A-O-B-O-A-O-B-O-A-O-B-O-A-O-A-O-A-O-B-O-A-O-A Formula S2;
wherein A represents a 2′-O-Me ribonucleoside, B represents a 2′-F ribonucleoside, O represents a phosphodiester internucleoside linkage, and S represents a phosphorothioate internucleoside linkage.

In some embodiments of the disclosure, the sense strand includes a structure represented by Formula S3, wherein Formula S3 is, in the 5′-to-3′ direction:

A-S-A-S-A-O-B-O-A-O-B-O-A-O-B-O-A-O-B-O-A-O-A-O-A-O-B-S-A-S-B Formula S3;
wherein A represents a 2′-O-Me ribonucleoside, B represents a 2′-F ribonucleoside, O represents a phosphodiester internucleoside linkage, and S represents a phosphorothioate internucleoside linkage.

In some embodiments of the disclosure, the sense strand includes a structure represented by Formula S4, wherein Formula S4 is, in the 5′-to-3′ direction:

A-S-A-S-A-O-B-O-A-O-B-O-A-O-B-O-A-O-B-O-A-O-A-O-A-O-B-O-A-O-B Formula S4;
wherein A represents a 2′-O-Me ribonucleoside, B represents a 2′-F ribonucleoside, O represents a phosphodiester internucleoside linkage, and S represents a phosphorothioate internucleoside linkage.

In some embodiments of the disclosure, the siRNA may contain an antisense strand including a region represented by Formula IV, wherein Formula IV is, in the 5′-to-3′ direction:

A-(A′)ⱼ-C-P²-B-(C-P¹)ₖ-C′ Formula IV;
wherein A is represented by the formula C-P¹-D-P¹; each A′ is represented by the formula C-P²-D-P²; B is represented by the formula D-P¹-C-P¹-D-P¹; each C is a 2′-O-Me ribonucleoside; each C′, independently, is a 2′-O-Me ribonucleoside or a 2′-F ribonucleoside; each D is a 2′-F ribonucleoside; each P¹ is a phosphorothioate internucleoside linkage; each P² is a phosphodiester internucleoside linkage; j is an integer from 1 to 7 (e.g., 1, 2, 3, 4, 5, 6, or 7); and k is an integer from 1 to 7 (e.g., 1, 2, 3, 4, 5, 6, or 7). In some embodiments, j is 6. In some embodiments, k is 4. In some embodiments, j is 6 and k is 4. The antisense strand is complementary (e.g., fully or partially complementary) to a target nucleic acid.

In some embodiments of the disclosure, the antisense strand includes a structure represented by Formula A3, wherein Formula A3 is, in the 5′-to-3′ direction:

A-S-B-S-A-O-B-O-A-O-B-O-A-O-B-O-A-O-B-O-A-O-B-O-A-O-B-O-A-O-B-S-A-S-B-S-A-S-A-S-A Formula A3;
wherein A represents a 2′-O-Me ribonucleoside, B represents a 2′-F ribonucleoside, O represents a phosphodiester internucleoside linkage, and S represents a phosphorothioate internucleoside linkage.

In some embodiments of the disclosure, the siRNA of the disclosure may have a sense strand represented by Formula V, wherein Formula V is, in the 5′-to-3′ direction:

E-(A′)ₘ-C-P²-F Formula V;
wherein E is represented by the formula (C-P¹)₂; F is represented by the formula D-P¹-C-P¹-C, D-P²-C-P²-C, D-P¹-C-P¹-D, or D-P²-C-P²-D; A′, C, D, P¹, and P² are as defined in Formula IV; and m is an integer from 1 to 7 (e.g., 1, 2, 3, 4, 5, 6, or 7). In some embodiments, m is 5. The sense strand is complementary (e.g., fully or partially complementary) to the antisense strand.

In some embodiments of the disclosure, the sense strand includes a structure represented by Formula S5, wherein Formula S5 is, in the 5′-to-3′ direction:

A-S-A-S-A-O-B-O-A-O-B-O-A-O-B-O-A-O-B-O-A-O-B-O-A-O-B-S-A-S-A Formula S5;
wherein A represents a 2′-O-Me ribonucleoside, B represents a 2′-F ribonucleoside, O represents a phosphodiester internucleoside linkage, and S represents a phosphorothioate internucleoside linkage.

In some embodiments of the disclosure, the sense strand includes a structure represented by Formula S6, wherein Formula S6 is, in the 5′-to-3′ direction:

A-S-A-S-A-O-B-O-A-O-B-O-A-O-B-O-A-O-B-O-A-O-B-O-A-O-B-O-A-O-A Formula S6;
wherein A represents a 2′-O-Me ribonucleoside, B represents a 2′-F ribonucleoside, O represents a phosphodiester internucleoside linkage, and S represents a phosphorothioate internucleoside linkage.

In some embodiments of the disclosure, the sense strand includes a structure represented by Formula S7, wherein Formula S7 is, in the 5′-to-3′ direction:

A-S-A-S-A-O-B-O-A-O-B-O-A-O-B-O-A-O-B-O-A-O-B-O-A-O-B-S-A-S-B Formula S7;
wherein A represents a 2′-O-Me ribonucleoside, B represents a 2′-F ribonucleoside, O represents a phosphodiester internucleoside linkage, and S represents a phosphorothioate internucleoside linkage.

In some embodiments of the disclosure, the sense strand includes a structure represented by Formula S8, wherein Formula S8 is, in the 5′-to-3′ direction:

A-S-A-S-A-O-B-O-A-O-B-O-A-O-B-O-A-O-B-O-A-O-B-O-A-O-B-O-A-O-B Formula S8;
wherein A represents a 2′-O-Me ribonucleoside, B represents a 2′-F ribonucleoside, O represents a phosphodiester internucleoside linkage, and S represents a phosphorothioate internucleoside linkage.

In some embodiments of the disclosure, the siRNA may contain an antisense strand including a region represented by Formula VI, wherein Formula VI is, in the 5′-to-3′ direction:

A-Bⱼ-E-Bₖ-E-F-Gₗ-D-P¹-C′ Formula VI;
wherein A is represented by the formula C-P¹-D-P¹; each B is represented by the formula C-P²; each C is a 2′-O-Me ribonucleoside; each C′, independently, is a 2′-O-Me ribonucleoside or a 2′-F ribonucleoside; each D is a 2′-F ribonucleoside; each E is represented by the formula D-P²-C-P²; F is represented by the formula D-P¹-C-P¹; each G is represented by the formula C-P¹; each P¹ is a phosphorothioate internucleoside linkage; each P² is a phosphodiester internucleoside linkage; j is an integer from 1 to 7 (e.g., 1, 2, 3, 4, 5, 6, or 7); k is an integer from 1 to 7 (e.g., 1, 2, 3, 4, 5, 6, or 7); and l is an integer from 1 to 7 (e.g., 1, 2, 3, 4, 5, 6, or 7). In some embodiments, j is 3. In some embodiments, k is 6. In some embodiments, l is 2. In some embodiments, j is 3, k is 6, and l is 2. The antisense strand is complementary (e.g., fully or partially complementary) to a target nucleic acid.

In some embodiments of the disclosure, the antisense strand includes a structure represented by Formula A4, wherein Formula A4 is, in the 5′-to-3′ direction:

A-S-B-S-A-O-A-O-A-O-B-O-A-O-A-O-A-O-A-O-A-O-A-O-A-O-B-O-A-O-B-S-A-S-A-S-A-S-B-S-A Formula A4;
wherein A represents a 2′-O-Me ribonucleoside, B represents a 2′-F ribonucleoside, O represents a phosphodiester internucleoside linkage, and S represents a phosphorothioate internucleoside linkage.

In some embodiments of the disclosure, the siRNA may contain a sense strand including a region represented by Formula VII, wherein Formula VII is, in the 5′-to-3′ direction:

H-Bₘ-Iₙ-A′-Bₒ-H-C Formula VII;
wherein A′ is represented by the formula C-P²-D-P²; each H is represented by the formula (C-P¹)₂; each I is represented by the formula (D-P²); B, C, D, P¹, and P² are as defined in Formula VI; m is an integer from 1 to 7 (e.g., 1, 2, 3, 4, 5, 6, or 7); n is an integer from 1 to 7 (e.g., 1, 2, 3, 4, 5, 6, or 7); and o is an integer from 1 to 7 (e.g., 1, 2, 3, 4, 5, 6, or 7). In some embodiments, m is 3. In some embodiments, n is 3. In some embodiments, o is 3. In some embodiments, m is 3, n is 3, and o is 3. The sense strand is complementary (e.g., fully or partially complementary) to the antisense strand.

In some embodiments of the disclosure, the sense strand includes a structure represented by Formula S9, wherein Formula S9 is, in the 5′-to-3′ direction:

A-S-A-S-A-O-A-O-A-O-B-O-B-O-B-O-A-O-B-O-A-O-A-O-A-O-A-S-A-S-A Formula S9;
wherein A represents a 2′-O-Me ribonucleoside, B represents a 2′-F ribonucleoside, O represents a phosphodiester internucleoside linkage, and S represents a phosphorothioate internucleoside linkage.

In some embodiments of the disclosure, the siRNA may contain an antisense strand including a region that is represented by Formula VIII:

Z-((A-P-)ₙ(B-P-)ₘ)_{q}; Formula VIII
wherein Z is a 5′ phosphorus stabilizing moiety; each A is a 2′-O-methyl (2′-O-Me) ribonucleoside; each B is a 2′-fluoro-ribonucleoside; each P is, independently, an internucleoside linkage selected from a phosphodiester linkage and a phosphorothioate linkage; n is an integer from 1 to 5 (e.g., 1, 2, 3, 4, or 5); m is an integer from 1 to 5 (e.g., 1, 2, 3, 4, or 5); and q is an integer between 1 and 30 (1, 2, 3, 4, 5, 6, 7, 8, 9, 10, 11, 12, 13, 14, 15, 16, 17, 18, 19, 20, 21, 22, 23, 24, 25, 26, 27, 28, 29, or 30).
   Methods of siRNA Synthesis

The siRNA molecules of the disclosure can be synthesized by standard methods known in the art as further discussed below, e.g., by use of an automated DNA synthesizer, such as are commercially available from, for example, Biosearch, Applied Biosystems, Inc.

The siRNA agent can be prepared using solution-phase or solid-phase organic synthesis or both. Organic synthesis offers the advantage that the oligonucleotide including unnatural or modified nucleotides can be easily prepared. siRNA molecules of the disclosure can be prepared using solution-phase or solid-phase organic synthesis or both.

Further, it is contemplated that for any siRNA agent disclosed herein, further optimization could be achieved by systematically either adding or removing linked nucleosides to generate longer or shorter sequences. Further still, such optimized sequences can be adjusted by, e.g., the introduction of modified nucleosides, and/or modified internucleoside linkages as described herein or as known in the art, including alternative nucleosides, alternative sugar moieties, and/or alternative internucleoside linkages as known in the art and/or discussed herein to further optimize the molecule (e.g., increasing serum stability or circulating half-life, increasing thermal stability, enhancing transmembrane delivery, and/or targeting to a particular location or cell type).

### 5′ Phosphorus Stabilizing Moieties

To further protect the siRNA molecules of this disclosure from degradation, a 5′-phosphorus stabilizing moiety may be employed. A 5′-phosphorus stabilizing moiety replaces the 5′-phosphate to prevent hydrolysis of the phosphate. Hydrolysis of the 5′-phosphate prevents binding to RISC, a necessary step in gene silencing. Any replacement for phosphate that does not impede binding to RISC is contemplated in this disclosure. In some embodiments, the replacement for the 5′-phosphate is also stable to in vivo hydrolysis. Each strand of a siRNA molecule may independently and optionally employ any suitable 5′-phosphorus stabilizing moiety.

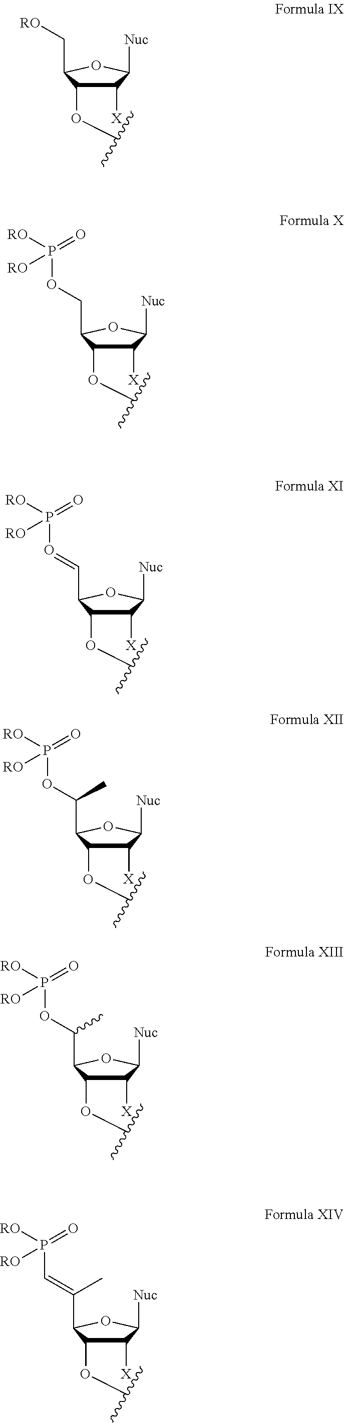

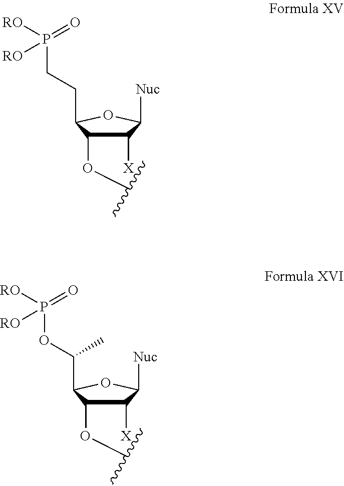

Some exemplary endcaps are demonstrated in Formulas IX-XVI. Nuc in Formulas IX-XVI represents a nucleobase or nucleobase derivative or replacement as described herein. X in formula IX-XVI represents a 2′-modification as described herein. Some embodiments employ hydroxy as in Formula IX, phosphate as in Formula X, vinylphosphonates as in Formula XI and XIV, 5′-methyl-substitued phosphates as in Formula XII, XIII, and XVI, methylenephosphonates as in Formula XV, or vinyl 5′-vinylphsophonate as a 5′-phosphorus stabilizing moiety as demonstrated in Formula XI.

### Hydrophobic Moieties

The present disclosure further provides siRNA molecules having one or more hydrophobic moieties attached thereto. The hydrophobic moiety may be covalently attached to the 5′ end or the 3′ end of the siRNA molecules of the disclosure. Non-limiting examples of hydrophobic moieties suitable for use with the siRNA molecules of the disclosure may include cholesterol, vitamin D, tocopherol, phosphatidylcholine (PC), docosahexaenoic acid, docosanoic acid, PC-docosanoic acid, eicosapentaenoic acid, lithocholic acid or any combination of the aforementioned hydrophobic moieties with PC.

siRNA Branching

The siRNA molecules of the disclosure may be branched. For example, the siRNA molecules of the disclosure may have one of several branching patterns, as described herein.

According to the present disclosure, the siRNA molecules disclosed herein may be branched siRNA molecules. The siRNA molecule may not be branched, or may be di-branched, tri-branched, or tetra-branched, connected through a linker. Each main branch may be further branched to allow for 2, 3, 4, 5, 6, 7, or 8 separate RNA single- or double-strands. The branch points on the linker may stem from the same atom, or separate atoms along the linker. Some exemplary embodiments are listed in Table 2.

TABLE 2
Branched siRNA structures

| Di-branched | Tri-branched | Tetra-branched |
|---|---|---|
| RNA—L—RNA Formula XVII | 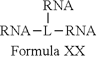 | 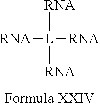 |
| 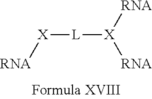 | 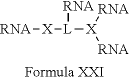 | 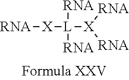 |
| 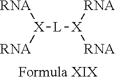 | 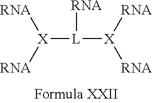 | 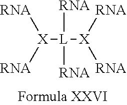 |
| | 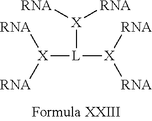 | 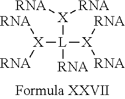 |
| | | 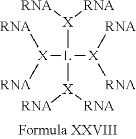 |

In some embodiments, the siRNA molecule is a branched siRNA molecule. In some embodiments, the branched siRNA molecule is di-branched, tri-branched, or tetra-branched. In some embodiments, the di-branched siRNA molecule is represented by any one of Formulas XVII-XIX, wherein each RNA, independently, is an siRNA molecule, L is a linker, and each X, independently, represents a branch point moiety (e.g., phosphoroamidite, tosylated solketal, 1,3-diaminopropanol, pentaerythritol, or any one of the branch point moieties described in U.S. Pat. No. 10,478,503).

In some embodiments, the tri-branched siRNA molecule represented by any one of Formulas XX-XXIII, wherein each RNA, independently, is an siRNA molecule, L is a linker, and each X, independently, represents a branch point moiety.

In some embodiments, the tetra-branched siRNA molecule represented by any one of Formulas XXIV-XXVIII, wherein each RNA, independently, is an siRNA molecule, L is a linker, and each X, independently, represents a branch point moiety.

### Linkers

Multiple strands of siRNA described herein may be covalently attached by way of a linker. The effect of this branching improves, inter alia, cell permeability allowing better access into cells (e.g., neurons or glial cells) in the CNS. Any linking moiety may be employed which is not incompatible with the siRNAs of the present invention. Linkers include ethylene glycol chains of 2 to 10 subunits (e.g., 2, 3, 4, 5, 6, 7, 8, 9, or 10 subunits), alkyl chains, carbohydrate chains, block copolymers, peptides, RNA, DNA, and others. In some embodiments, any carbon or oxygen atom of the linker is optionally replaced with a nitrogen atom, bears a hydroxyl substituent, or bears an oxo substituent. In some embodiments, the linker is a poly-ethylene glycol (PEG) linker. The PEG linkers suitable for use with the disclosed compositions and methods include linear or non-linear PEG linkers. Examples of non-linear PEG linkers include branched PEGs, linear forked PEGs, or branched forked PEGs.

PEG linkers of various weights may be used with the disclosed compositions and methods. For example, the PEG linker may have a weight that is between 5 and 500 Daltons. In some embodiments, a PEG linker having a weight that is between 500 and 1,000 Dalton may be used. In some embodiments, a PEG linker having a weight that is between 1,000 and 10,000 Dalton may be used. In some embodiments, a PEG linker having a weight that is between 200 and 20,000 Dalton may be used. In some embodiments, the linker is covalently attached to a sense strand of the siRNA. In some embodiments, the linker is covalently attached to an antisense strand of the siRNA. In some embodiments, the PEG linker is a triethylene glycol (TrEG) linker. In some embodiments, the PEG linker is a tetraethylene linker (TEG).

In some embodiments, the linker is an alkyl chain linker. In some embodiments, the linker is a peptide linker. In some embodiments, the linker is an RNA linker. In some embodiments, the linker is a DNA linker.

Linkers may covalently link 2, 3, 4, or 5 unique siRNA strands. The linker may covalently bind to any part of the siRNA oligomer. In some embodiments, the linker attaches to the 3′ end of nucleosides of each siRNA strand. In some embodiments, the linker attaches to the 5′ end of nucleosides of each siRNA strand. In some embodiments, the linker attaches to a nucleoside of an siRNA strand (e.g., sense or antisense strand) by way of a covalent bond-forming moiety. In some embodiments, the covalent-bond-forming moiety is selected from the group consisting of an alkyl, ester, amide, carbonate, carbamate, triazole, urea, formacetal, phosphonate, phosphate, and phosphate derivative (e.g., phosphorothioate, phosphoramidate, etc.).

In some embodiments, the linker has a structure of Formula L1:

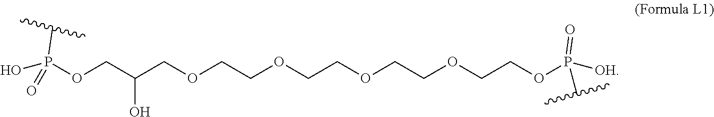 [SMILES: *P(=O)(O)OCCOCCOCCOCCOCC(O)COP(*)(=O)O]

In some embodiments, the linker has a structure of Formula L2:

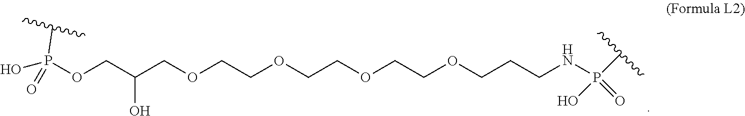 [SMILES: *P(=O)(O)NCCCOCCOCCOCCOCC(O)COP(*)(=O)O]

In some embodiments, the linker has a structure of Formula L3:

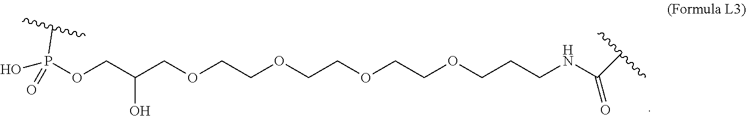 [SMILES: *C(=O)NCCCOCCOCCOCCOCC(O)COP(*)(=O)O]

In some embodiments, the linker has a structure of Formula L4:

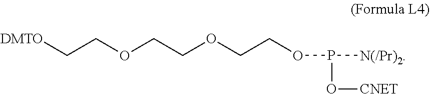

In some embodiments, the linker has a structure of Formula L5:

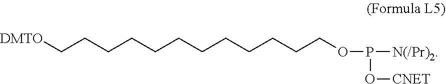

In some embodiments, the linker has a structure of Formula L6:

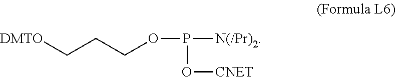

In some embodiments, the linker has a structure of Formula L7:

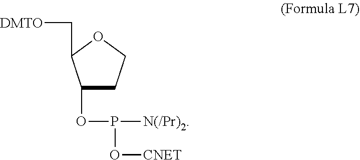

In some embodiments, the linker has a structure of Formula L8:

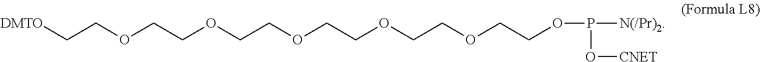

In some embodiments, the linker has a structure of Formula L9:

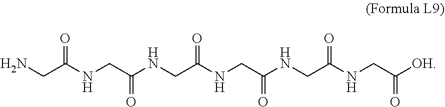 [SMILES: NCC(=O)NCC(=O)NCC(=O)NCC(=O)NCC(=O)NCC(=O)O]

In some embodiments, the selection of a linker for use with one or more of the branched siRNA molecules disclosed herein may be based on the hydrophobicity of the linker, such that, e.g., desirable hydrophobicity is achieved for the one or more branched siRNA molecules of the disclosure. For example, a linker containing an alkyl chain may be used to increase the hydrophobicity of the branched siRNA molecule as compared to a branched siRNA molecule having a less hydrophobic linker or a hydrophilic linker.

The siRNA agents disclosed herein may be synthesized and/or modified by methods well established in the art, such as those described in Beaucage, S. L. et al. (edrs.), Current Protocols in Nucleic Acid Chemistry, John Wiley & Sons, Inc., New York, N.Y., 2000, which is hereby incorporated herein by reference.

### Methods of Treatment

The SCN9A-targeting siRNA molecules of the disclosure may be delivered to a subject, for example, as an analgesic effective against multiple forms of acute or chronic pain (e.g., nociceptive pain or neuropathic pain). Furthermore, the siRNA molecules of the disclosure may also be delivered to a subject having a gain-of-function variant of the SCN9A gene (e.g., primary erythromelalgia) for which siRNA-mediated gene silencing of the SCN9A variant gene reduces the expression level of SCN9A transcript, thereby reducing the level of pain experienced by the subject and/or mitigating symptoms of a pain disorder, such as Gerhardt disease, Mitchell disease, or Weir-Mitchell disease.

The disclosure provides methods of treating a subject by way of SCN9A gene silencing with one or more of the small interfering RNA (siRNA) molecules described herein. The gene silencing may be performed in a subject to silence wild type SCN9A transcripts, mutant SCN9A transcripts, splice isoforms of SCN9A transcripts, and/or overexpressed SCN9A transcripts thereof, relative to a healthy subject. The method may include delivering to the CNS or neurons of the subject (e.g., a human) the siRNA molecules of the disclosure or a pharmaceutical composition containing the same by any appropriate route of administration (e.g., intrathecal injection, direct injection into a specific nerve or ganglion (ganglia), such as trigenminal or dorsal root ganglia, or by intra-cisterna magna injection by catheterization). The active compound can be administered in any suitable dose. The actual dosage amount of a composition of the present disclosure administered to a patient can be determined by physical and physiological factors such as body weight, severity of condition, previous or concurrent therapeutic interventions, idiopathy of the patient and on the route of administration. Depending upon the dosage and the route of administration, the number of administrations of a preferred dosage and/or an effective amount may vary according to the response of the subject. The practitioner responsible for administration will, in any event, determine the concentration of active ingredient(s) in a composition and appropriate dose(s) for the individual subject. Administration may occur any suitable number of times per day, and for as long as necessary. Subjects may be adult or pediatric humans, with or without comorbid diseases.

### Selection of Subjects

Subjects that may be treated with the small interfering RNA (siRNA) molecules disclosed herein are subjects in need of treatment for chronic, persistent, or acute symptoms of pain. Such symptoms of pain may be neuropathic or nociceptive in nature. Additionally, subjects in need of treatment of pain may be characterized as having spontaneous pain (e.g., primary erythromelalgia or secondary erythromelalgia) or may be diagnosed with a pain disorder (e.g., Gerhardt disease, Mitchell disease, or Weir-Mitchell disease). Subjects that may be treated with the siRNA molecules disclosed herein may include, for example, humans, monkeys, rats, mice, pigs, and other mammals containing at least one orthologous copy of the SCN9A gene. Subjects may be adult or pediatric humans, with or without comorbid diseases.

### Pharmaceutical Compositions

The siRNA molecules in the present disclosure may be formulated into a pharmaceutical composition for administration to a subject in a biologically compatible form suitable for administration in vivo. Accordingly, the present disclosure provides a pharmaceutical composition containing a siRNA molecule of the disclosure in admixture with a suitable diluent, carrier, or excipient. The siRNA molecules may be administered, for example, directly into the CNS or affected tissues or neurons of the subject (e.g., by way of intracerebroventricular injection, intrastriatal injection, intrathecal injection, intra-cisterna magna injection by catheterization, intraparenchymal injection, direct injection into a specific nerve or ganglion (ganglia) (e.g., trigenminal or dorsal root ganglia), intravenous injection, subcutaneous injection, or intramuscular injection).

Conventional procedures and ingredients for the selection and preparation of suitable formulations are described, for example, in Remington, J. P. The Science and Practice of Pharmacy, Easton, PA. Mack Publishers, 2012, 22^{nd} ed. and in The United States Pharmacopeial Convention, The National Formulary, United States Pharmacopeial, 2015, USP 38 NF 33).

Under ordinary conditions of storage and use, a pharmaceutical composition may contain a preservative, e.g., to prevent the growth of microorganisms. Pharmaceutical compositions may include sterile aqueous solutions, dispersions, or powders, e.g., for the extemporaneous preparation of sterile solutions or dispersions. In all cases the form may be sterilized using techniques known in the art and may be fluidized to the extent that may be easily administered to a subject in need of treatment.

A pharmaceutical composition may be administered to a subject, e.g., a human subject, alone or in combination with pharmaceutically acceptable carriers, as noted herein, the proportion of which may be determined by the solubility and/or chemical nature of the compound, chosen route of administration, and standard pharmaceutical practice.

### Dosing Regimens

A physician having ordinary skill in the art can readily determine an effective amount of the siRNA molecule for administration to a mammalian subject (e.g., a human) in need thereof. For example, a physician could start prescribing doses of one the siRNA molecules of the disclosure at levels lower than that required to achieve the desired therapeutic effect and gradually increase the dosage until the desired effect is achieved. Alternatively, a physician may begin a treatment regimen by administering one of the siRNA molecules of the disclosure at a high dose and subsequently administer progressively lower doses until reaching a minimal dosage at which a therapeutic effect is achieved (e.g., a reduction in expression of a target gene sequence). In general, a suitable daily dose of one of the siRNA molecules of the disclosure will be an amount of the siRNA molecule which is the lowest dose effective to produce a therapeutic effect. The ss- or ds-siRNA molecules of the disclosure may be administered by injection, e.g., intrathecally, intracerebroventricularly, by intra-cisterna magna injection by catheterization, intraparenchymally, by direct injection into a specific nerve or ganglion (ganglia) (e.g., trigeminal or dorsal root ganglia), intravenously, subcutaneously, or intramuscularly. A daily dose of a therapeutic composition of the siRNA molecules of the disclosure may be administered as a single dose or as two, three, four, five, six or more doses administered separately at appropriate intervals throughout the day, week, month, or year, optionally, in unit dosage forms. While it is possible for the siRNA molecules of the disclosure to be administered alone, it may also be administered as a pharmaceutical formulation in combination with excipients, carriers, and optionally, additional therapeutic agents.

### Routes of Administration

The method of the disclosure contemplates any route of administration tolerated by the therapeutic composition. Some embodiments of the method include injection intrathecally or by intra-cisterna magna injection by catheterization. Some embodiments of the method include direct injection into a specific nerve or ganglion (ganglia) (e.g., trigeminal or dorsal root ganglia).

Intrathecal injection is the direct injection into the spinal column or subarachnoid space. By injecting directly into the CSF of the spinal column the siRNA molecules of the disclosure have direct access to cells (e.g., neurons and glial cells) in the spinal column and a route to access the cells in the brain by bypassing the blood brain barrier, or a route to access cell bodies of those neurons that are outside the blood brain barrier.

Intracerebroventricular (ICV) injection is a method to directly inject into the CSF of the cerebral ventricles. Similar to intrathecal injection, ICV is a method of injection which bypasses the blood brain barrier. Using ICV allows the advantage of access to the cells of the brain and spinal column without the danger of the therapeutic being degraded in the blood.

Intrastriatal injection is the direct injection into the striatum, or corpus striatum. The striatum is an area in the subcortical basal ganglia in the brain. Injecting into the striatum bypasses the blood brain barrier and the pharmacokinetic challenges of injection into the blood stream and allows for direct access to the cells of the brain.

Intraparenchymal administration is the direct injection into the parenchyma (e.g., the brain parenchyma). Injection into the brain parenchyma allows for injection directly into brain regions affected by a disease or disorder while bypassing the blood brain barrier.

Intra-cisterna magna injection by catheterization is the direct injection into the cisterna magna. The cisterna magna is the area of the brain located between the cerebellum and the dorsal surface of the medulla oblongata. Injecting into the cisterna magna results in more direct delivery to the cells of the cerebellum, brainstem, and spinal cord.

In some embodiments of the methods described herein, the therapeutic composition may be delivered to the subject by way of systemic administration, e.g., intravenously, intramuscularly, or subcutaneously.

Intravenous (IV) injection is a method to directly inject into the bloodstream of a subject. The IV administration may be in the form of a bolus dose or by way of continuous infusion, or any other method tolerated by the therapeutic composition.

Intramuscular (IM) injection is injection into a muscle of a subject, such as the deltoid muscle or gluteal muscle. IM may allow for rapid absorption of the therapeutic composition.

Subcutaneous injection is injection into subcutaneous tissue. Absorption of compositions delivered subcutaneously may be slower than IV or IM injection, which may be beneficial for compositions requiring continuous absorption.

### EXAMPLES

The following examples are put forth so as to provide those of ordinary skill in the art with a description of how the compositions and methods described herein may be used, made, and evaluated, and are intended to be purely exemplary of the disclosure and are not intended to limit the scope of what the inventors regard as their disclosure.

### Example 1. Knockdown of SCN9A with siRNA Molecules of the Disclosure

SCN9A-targeting siRNA molecules of the disclosure were screened for activity. G402 cells were cultured in the presence of an siRNA molecule of the disclosure at either 2 μM or 0.5 μM concentration. After 72 hours, cells were lysed, and mRNA levels of SCN9A and a housekeeping gene (GAPDH) were assessed via reverse transcription-quantitative polymerase chain reaction (RT-qPCR), using standard reagents and Applied Biosystems TaqMan Assays. Results are presented in Table 3, below, as the percent residual SCN9A mRNA relative to untreated control cells in the same assay, corrected for any changes in expression due to housekeeping gene (% UNT SCN9A mRNA). STDEV=standard deviation, ND=not determined.

TABLE 3
SCN9A Knockdown with siRNA molecules of the disclosure

| | | | % UNT | | % UNT | |
|---|---|---|---|---|---|---|
| | Sense | Targeting | SCN9A | | SCN9A | |
| Antisense | SEQ | Region | mRNA | | mRNA | |
| SEQ ID | ID | SEQ ID | expression | | expression | |
| NO: | NO: | NO: | at 2 uM | STDEV | at 0.5 uM | STDEV |
| 1 | 193 | 385 | 97.75 | 4.60 | 78.02 | 10.08 |
| 2 | 194 | 386 | 95.30 | 5.36 | 72.92 | 8.20 |
| 3 | 195 | 387 | 72.19 | 0.50 | 73.81 | 9.75 |
| 4 | 196 | 388 | 64.58 | 6.07 | 64.63 | 3.74 |
| 5 | 197 | 389 | 70.36 | 1.62 | 63.42 | 6.54 |
| 6 | 198 | 390 | 49.09 | 4.41 | 49.93 | 1.53 |
| 7 | 199 | 391 | 63.58 | 0.95 | 61.83 | 1.64 |
| 8 | 200 | 392 | 77.79 | 6.72 | 59.82 | 2.11 |
| 9 | 201 | 393 | 68.79 | 7.38 | 53.20 | 1.45 |
| 10 | 202 | 394 | 67.28 | 4.62 | 58.97 | 1.25 |
| 11 | 203 | 395 | 73.45 | 7.45 | 74.97 | 3.80 |
| 12 | 204 | 396 | 98.13 | 6.89 | 77.09 | 1.98 |
| 13 | 205 | 397 | 62.29 | 5.38 | 71.24 | 10.14 |
| 14 | 206 | 398 | 80.10 | 3.88 | 71.22 | 2.23 |
| 15 | 207 | 399 | 55.55 | 5.00 | 47.45 | 1.85 |
| 16 | 208 | 400 | 62.43 | 11.29 | 54.07 | 2.72 |
| 17 | 209 | 401 | 67.18 | 0.51 | 47.44 | 0.19 |
| 18 | 210 | 402 | 122.71 | 20.27 | 90.03 | 5.76 |
| 19 | 211 | 403 | 121.62 | 20.39 | 80.27 | 3.51 |
| 20 | 212 | 404 | 91.57 | 11.67 | 82.61 | 0.49 |
| 21 | 213 | 405 | 76.30 | 5.63 | 74.56 | 3.83 |
| 22 | 214 | 406 | 78.28 | 3.63 | 82.45 | 4.97 |
| 23 | 215 | 407 | 54.27 | 2.51 | 63.68 | 3.61 |
| 24 | 216 | 408 | 56.02 | 2.79 | 58.23 | 1.28 |
| 25 | 217 | 409 | 81.89 | 4.02 | 83.16 | 3.13 |
| 26 | 218 | 410 | 59.70 | 7.24 | 65.56 | 3.68 |
| 27 | 219 | 411 | 38.42 | 1.35 | ND | ND |
| 28 | 220 | 412 | 44.02 | 0.60 | ND | ND |
| 29 | 221 | 413 | 43.56 | 4.35 | ND | ND |
| 30 | 222 | 414 | 47.98 | 7.04 | ND | ND |
| 31 | 223 | 415 | 55.36 | 10.21 | ND | ND |
| 32 | 224 | 416 | 49.58 | 0.76 | ND | ND |
| 33 | 225 | 417 | 61.08 | 5.04 | ND | ND |
| 34 | 226 | 418 | 83.07 | ND | ND | ND |
| 35 | 227 | 419 | 66.59 | ND | ND | ND |
| 36 | 228 | 420 | 63.84 | 11.90 | ND | ND |
| 37 | 229 | 421 | 75.49 | ND | ND | ND |
| 38 | 230 | 422 | 50.87 | 0.48 | ND | ND |
| 39 | 231 | 423 | 43.55 | 1.32 | ND | ND |
| 40 | 232 | 424 | 66.43 | 5.84 | ND | ND |
| 41 | 233 | 425 | 47.88 | 4.76 | ND | ND |
| 42 | 234 | 426 | 72.32 | 4.70 | ND | ND |
| 43 | 235 | 427 | 71.45 | 0.76 | ND | ND |
| 44 | 236 | 428 | 55.65 | 2.60 | ND | ND |
| 45 | 237 | 429 | 65.68 | 1.20 | ND | ND |
| 46 | 238 | 430 | 62.46 | 4.95 | ND | ND |
| 47 | 239 | 431 | 38.70 | 1.70 | ND | ND |
| 48 | 240 | 432 | 49.94 | 0.62 | ND | ND |
| 49 | 241 | 433 | 60.63 | 5.37 | ND | ND |
| 50 | 242 | 434 | 69.32 | 3.81 | ND | ND |
| 51 | 243 | 435 | 65.15 | 5.70 | ND | ND |
| 52 | 244 | 436 | 52.22 | 3.63 | ND | ND |
| 53 | 245 | 437 | 72.33 | 3.17 | 80.69 | 0.78 |
| 54 | 246 | 438 | 82.98 | 22.17 | 82.58 | 2.12 |
| 55 | 247 | 439 | 60.28 | 13.26 | 65.74 | 4.62 |
| 56 | 248 | 440 | 66.24 | 15.36 | 70.83 | 0.85 |
| 57 | 249 | 441 | 65.77 | 11.67 | 81.48 | 0.35 |
| 58 | 250 | 442 | 83.93 | 4.71 | 103.29 | 10.63 |
| 59 | 251 | 443 | 65.17 | 4.85 | 90.97 | 3.31 |
| 60 | 252 | 444 | 69.48 | 7.42 | 77.44 | 13.14 |
| 61 | 253 | 445 | 112.83 | 12.66 | 84.17 | 1.09 |
| 62 | 254 | 446 | 90.53 | 5.61 | 76.38 | 2.32 |
| 63 | 255 | 447 | 75.85 | 8.91 | 69.48 | 3.15 |
| 64 | 256 | 448 | 90.38 | 22.85 | 74.11 | 4.55 |
| 65 | 257 | 449 | 79.42 | 8.11 | 86.33 | 4.37 |
| 66 | 258 | 450 | 94.93 | 9.49 | 91.71 | 2.22 |
| 67 | 259 | 451 | 102.34 | 13.30 | 83.65 | 0.04 |
| 68 | 260 | 452 | 74.62 | 13.07 | 92.60 | 1.25 |
| 69 | 261 | 453 | 88.11 | 11.67 | 89.12 | ND |
| 70 | 262 | 454 | 93.23 | 3.72 | 79.22 | 1.37 |
| 71 | 263 | 455 | 84.10 | 8.42 | 73.71 | 0.88 |
| 72 | 264 | 456 | 66.32 | 5.08 | 60.34 | 5.05 |
| 73 | 265 | 457 | 50.76 | ND | 53.12 | 0.72 |
| 74 | 266 | 458 | 47.81 | ND | 47.64 | 2.33 |
| 75 | 267 | 459 | 92.44 | ND | 78.42 | 3.07 |
| 76 | 268 | 460 | 95.37 | 4.44 | 72.88 | 1.41 |
| 77 | 269 | 461 | 107.93 | 0.28 | 78.90 | 2.15 |
| 78 | 270 | 462 | 97.77 | 20.85 | 73.13 | 3.50 |
| 79 | 271 | 463 | 85.28 | 6.27 | 47.18 | 0.92 |
| 80 | 272 | 464 | 87.02 | 4.72 | 53.62 | 1.44 |
| 81 | 273 | 465 | 66.14 | 3.41 | 47.10 | 3.28 |
| 82 | 274 | 466 | 71.90 | 10.13 | 49.18 | 2.51 |
| 83 | 275 | 467 | 92.18 | 2.95 | 60.76 | 1.71 |
| 84 | 276 | 468 | 45.59 | 1.28 | 39.73 | 1.75 |
| 85 | 277 | 469 | 113.79 | 7.71 | 95.14 | 0.22 |
| 86 | 278 | 470 | 114.44 | 15.18 | 89.92 | 6.57 |
| 87 | 279 | 471 | 97.89 | 6.79 | 79.19 | 3.99 |
| 88 | 280 | 472 | 66.65 | 4.44 | 62.64 | 2.69 |
| 89 | 281 | 473 | 129.28 | 29.89 | 58.68 | 0.05 |
| 90 | 282 | 474 | 94.19 | 21.08 | 48.91 | 0.98 |
| 91 | 283 | 475 | 112.42 | 19.71 | 57.64 | 1.92 |
| 92 | 284 | 476 | 116.73 | 8.20 | 57.56 | 0.40 |
| 93 | 285 | 477 | 85.67 | 2.64 | 47.13 | 1.08 |
| 94 | 286 | 478 | 115.46 | 12.08 | 65.76 | 3.67 |
| 95 | 287 | 479 | 143.28 | 23.78 | 84.64 | 7.76 |
| 96 | 288 | 480 | 103.23 | 0.49 | 44.29 | ND |
| 97 | 289 | 481 | 117.10 | 11.48 | 75.20 | 1.17 |
| 98 | 290 | 482 | 86.54 | 18.94 | 66.58 | 1.22 |
| 99 | 291 | 483 | 176.69 | 11.84 | 65.61 | 0.21 |
| 100 | 292 | 484 | 171.74 | 8.26 | 67.17 | 3.73 |
| 101 | 293 | 485 | 133.89 | 2.24 | 68.27 | 1.82 |
| 102 | 294 | 486 | 139.48 | 20.02 | 68.38 | 2.96 |
| 103 | 295 | 487 | 113.23 | 9.20 | 66.23 | 1.61 |
| 104 | 296 | 488 | 123.02 | 11.23 | 77.37 | 1.26 |
| 105 | 297 | 489 | 64.53 | 8.01 | 71.64 | 2.45 |
| 106 | 298 | 490 | 70.10 | 11.61 | 74.34 | 4.09 |
| 107 | 299 | 491 | 73.77 | 3.02 | 74.08 | 1.64 |
| 108 | 300 | 492 | 75.30 | 13.09 | 71.57 | 1.05 |
| 109 | 301 | 493 | 71.15 | 3.46 | 77.65 | 1.98 |
| 110 | 302 | 494 | 88.07 | 20.37 | 77.67 | 4.19 |
| 111 | 303 | 495 | 83.52 | 23.63 | 72.19 | 1.57 |
| 112 | 304 | 496 | 86.43 | 11.92 | 72.12 | 1.91 |
| 113 | 305 | 497 | 87.97 | 9.82 | 77.85 | 1.50 |
| 114 | 306 | 498 | 80.76 | 17.45 | 75.02 | 7.43 |
| 115 | 307 | 499 | 74.04 | 14.48 | 91.67 | 11.04 |
| 116 | 308 | 500 | 107.62 | 32.98 | 97.13 | 10.03 |
| 117 | 309 | 501 | 102.20 | 22.34 | 100.43 | 16.37 |
| 118 | 310 | 502 | 113.14 | 39.17 | 86.36 | 5.01 |
| 119 | 311 | 503 | 133.82 | 56.67 | 85.60 | 5.27 |
| 120 | 312 | 504 | 159.34 | 70.28 | 87.67 | 6.83 |
| 121 | 313 | 505 | 100.33 | 37.26 | 80.38 | 1.20 |
| 122 | 314 | 506 | 72.18 | ND | 90.58 | 0.79 |
| 123 | 315 | 507 | 68.49 | ND | 89.58 | 0.64 |
| 124 | 316 | 508 | 158.43 | 85.10 | 87.89 | 1.22 |
| 125 | 317 | 509 | 78.04 | 15.74 | 107.01 | 19.24 |
| 126 | 318 | 510 | 64.63 | 13.42 | 78.75 | 20.57 |
| 127 | 319 | 511 | 76.63 | 9.96 | 72.40 | 6.91 |
| 128 | 320 | 512 | 99.49 | 0.95 | 84.58 | 10.46 |
| 129 | 321 | 513 | 80.03 | 3.47 | 71.94 | 0.33 |
| 130 | 322 | 514 | 77.92 | 9.38 | 92.70 | 6.31 |
| 131 | 323 | 515 | 88.26 | 4.83 | 90.89 | 22.17 |
| 132 | 324 | 516 | 56.05 | 11.39 | 65.27 | 1.42 |
| 133 | 325 | 517 | 72.67 | 16.79 | 76.33 | 1.31 |
| 134 | 326 | 518 | 69.70 | 11.12 | 72.00 | 1.95 |
| 135 | 327 | 519 | 60.83 | 5.43 | 75.66 | 6.54 |
| 136 | 328 | 520 | 61.32 | 7.42 | 83.96 | 11.17 |
| 137 | 329 | 521 | 54.88 | 7.97 | 73.16 | 4.78 |
| 138 | 330 | 522 | 54.95 | 15.55 | 79.24 | 5.04 |
| 139 | 331 | 523 | 48.12 | 6.61 | 75.85 | 12.07 |
| 140 | 332 | 524 | 65.27 | 18.62 | 76.84 | 4.96 |
| 141 | 333 | 525 | 140.03 | 27.31 | 108.67 | 19.63 |
| 142 | 334 | 526 | 127.28 | 17.88 | 93.16 | 5.81 |
| 143 | 335 | 527 | 102.44 | 3.95 | 95.25 | 9.83 |
| 144 | 336 | 528 | 90.33 | 1.13 | 88.77 | 5.05 |
| 145 | 337 | 529 | 74.49 | 0.80 | 91.46 | 6.88 |
| 146 | 338 | 530 | 74.33 | 2.47 | 90.54 | 4.69 |
| 147 | 339 | 531 | 57.39 | 1.95 | 83.08 | 1.08 |
| 148 | 340 | 532 | 59.82 | 2.79 | 85.74 | 0.63 |
| 149 | 341 | 533 | 56.71 | 4.84 | 93.14 | 0.27 |
| 150 | 342 | 534 | 45.93 | 1.20 | 70.91 | 1.80 |
| 151 | 343 | 535 | 95.78 | 6.79 | 75.01 | 2.62 |
| 152 | 344 | 536 | 94.45 | 3.93 | 77.96 | 2.11 |
| 153 | 345 | 537 | 81.80 | 2.94 | 76.72 | 4.09 |
| 154 | 346 | 538 | 82.65 | 3.64 | 89.10 | 7.48 |
| 155 | 347 | 539 | 74.74 | 10.87 | 87.82 | 5.29 |
| 156 | 348 | 540 | 84.39 | 0.35 | 92.81 | 11.19 |
| 157 | 349 | 541 | 124.73 | 16.18 | 68.61 | 3.46 |
| 158 | 350 | 542 | 116.59 | 8.60 | 63.38 | 4.79 |
| 159 | 351 | 543 | 162.66 | 38.43 | 74.34 | 4.98 |
| 160 | 352 | 544 | 129.01 | 14.45 | 71.74 | 5.11 |
| 161 | 353 | 545 | 115.11 | 17.75 | 73.97 | 5.44 |
| 162 | 354 | 546 | 81.32 | 13.59 | 69.88 | 2.43 |
| 163 | 355 | 547 | 105.04 | 12.07 | 79.94 | 5.11 |
| 164 | 356 | 548 | 116.78 | 11.57 | 81.16 | 12.58 |
| 165 | 357 | 549 | 97.71 | 28.43 | 72.36 | 8.12 |
| 166 | 358 | 550 | 92.75 | 33.63 | 67.75 | 11.80 |
| 167 | 359 | 551 | 156.27 | 13.66 | 66.67 | 0.02 |
| 168 | 360 | 552 | 102.33 | ND | 53.48 | 4.74 |
| 169 | 361 | 553 | 76.16 | 4.45 | 45.54 | 1.31 |
| 170 | 362 | 554 | 83.17 | 11.09 | 45.85 | 2.70 |
| 171 | 363 | 555 | 79.65 | 19.67 | 54.93 | 6.13 |
| 172 | 364 | 556 | 89.79 | 29.47 | 60.12 | 6.57 |
| 173 | 365 | 557 | 96.00 | 7.81 | 67.77 | 2.81 |
| 174 | 366 | 558 | 75.92 | 8.03 | 57.73 | 1.32 |
| 175 | 367 | 559 | 64.69 | 0.56 | 57.88 | 1.54 |
| 176 | 368 | 560 | 64.83 | 2.72 | 66.19 | 6.35 |
| 177 | 369 | 561 | 164.71 | 3.31 | 57.47 | 2.05 |
| 178 | 370 | 562 | 104.77 | ND | 63.78 | 0.07 |
| 179 | 371 | 563 | 141.99 | 17.38 | 79.39 | 1.38 |
| 180 | 372 | 564 | 112.24 | 20.83 | 63.32 | 0.76 |
| 181 | 373 | 565 | 169.81 | 42.18 | 86.72 | 0.61 |
| 182 | 374 | 566 | 168.14 | ND | 74.26 | 0.65 |
| 183 | 375 | 567 | 177.67 | 24.06 | 64.57 | 3.12 |
| 184 | 376 | 568 | 144.64 | 19.10 | 57.26 | 1.66 |
| 185 | 377 | 569 | 94.39 | 15.46 | 52.90 | 2.03 |
| 186 | 378 | 570 | 126.24 | 8.32 | 70.27 | 2.50 |
| 187 | 379 | 571 | 118.08 | 7.58 | 58.69 | 6.29 |
| 188 | 380 | 572 | 91.30 | 21.91 | 53.46 | 1.62 |
| 189 | 381 | 573 | 73.79 | 6.42 | 62.02 | 3.80 |
| 190 | 382 | 574 | 62.78 | 8.96 | 53.87 | 2.70 |
| 191 | 383 | 575 | 94.62 | 6.56 | 61.55 | 0.98 |
| 192 | 384 | 576 | 86.90 | 4.16 | 76.45 | 0.17 |
| 577 | 769 | 961 | 92.46 | 3.86 | 84.03 | 0.51 |
| 578 | 770 | 962 | 105.19 | 0.27 | 95.47 | 2.52 |
| 579 | 771 | 963 | 105.75 | 2.05 | 91.36 | 0.43 |
| 580 | 772 | 964 | 89.39 | 6.99 | 82.97 | 2.03 |
| 581 | 773 | 965 | 86.10 | 4.31 | 78.54 | 4.86 |
| 582 | 774 | 966 | 90.10 | 9.74 | 75.13 | 0.02 |
| 583 | 775 | 967 | 105.49 | 7.70 | 84.84 | 2.07 |
| 584 | 776 | 968 | 71.49 | 0.02 | 68.46 | 0.67 |
| 585 | 777 | 969 | 94.69 | 2.34 | 83.47 | 1.96 |
| 586 | 778 | 970 | 51.90 | 1.27 | 49.09 | 0.89 |
| 587 | 779 | 971 | 67.35 | ND | 67.86 | 3.80 |
| 588 | 780 | 972 | 72.86 | ND | 75.25 | 10.55 |
| 589 | 781 | 973 | 65.82 | ND | 62.84 | 5.76 |
| 590 | 782 | 974 | 64.47 | ND | 59.67 | 7.18 |
| 591 | 783 | 975 | 73.47 | ND | 73.00 | 4.56 |
| 592 | 784 | 976 | 85.24 | ND | 77.89 | 9.56 |
| 593 | 785 | 977 | 72.70 | ND | 70.63 | 7.59 |
| 594 | 786 | 978 | 79.46 | ND | 87.21 | 1.68 |
| 595 | 787 | 979 | 79.36 | ND | 73.70 | 10.85 |
| 596 | 788 | 980 | 77.07 | ND | 67.62 | 2.40 |
| 597 | 789 | 981 | 110.12 | 2.28 | 99.46 | 2.21 |
| 598 | 790 | 982 | 93.05 | 2.87 | 88.72 | 0.04 |
| 599 | 791 | 983 | 87.34 | 2.23 | 90.55 | 4.50 |
| 600 | 792 | 984 | 79.18 | 3.35 | 79.73 | 2.65 |
| 601 | 793 | 985 | 68.06 | 0.37 | 75.22 | 1.26 |
| 602 | 794 | 986 | 69.15 | 1.12 | 73.42 | 6.50 |
| 603 | 795 | 987 | 53.41 | 1.31 | 56.03 | 1.13 |
| 604 | 796 | 988 | 56.66 | 2.51 | 61.28 | 3.59 |
| 605 | 797 | 989 | 58.58 | 0.04 | 61.53 | 0.47 |
| 606 | 798 | 990 | 63.45 | 0.92 | 68.47 | 1.59 |
| 607 | 799 | 991 | 78.69 | 1.94 | 83.13 | 1.32 |
| 608 | 800 | 992 | 90.63 | 1.15 | 90.29 | 2.97 |
| 609 | 801 | 993 | 66.76 | 3.08 | 72.37 | 0.09 |
| 610 | 802 | 994 | 88.88 | 2.27 | 86.28 | 0.35 |
| 611 | 803 | 995 | 73.02 | 3.98 | 77.79 | 0.03 |
| 612 | 804 | 996 | 75.95 | 0.58 | 76.05 | 2.13 |
| 613 | 805 | 997 | 71.56 | 0.69 | 84.87 | 2.85 |
| 614 | 806 | 998 | 71.05 | 3.46 | 79.09 | 2.17 |
| 615 | 807 | 999 | 60.62 | 3.21 | 63.79 | 4.57 |
| 616 | 808 | 1000 | 60.75 | 2.67 | 64.72 | 5.60 |
| 617 | 809 | 1001 | 71.05 | 5.18 | 64.70 | 8.00 |
| 618 | 810 | 1002 | 84.09 | ND | 71.16 | 5.03 |
| 619 | 811 | 1003 | 71.30 | 3.88 | 64.39 | 6.51 |
| 620 | 812 | 1004 | 76.67 | 2.79 | 71.60 | 5.32 |
| 621 | 813 | 1005 | 68.82 | 6.12 | 63.58 | 6.13 |
| 622 | 814 | 1006 | 77.82 | 3.12 | 71.47 | 4.97 |
| 623 | 815 | 1007 | 87.13 | 1.83 | 98.91 | 0.07 |
| 624 | 816 | 1008 | 87.22 | 1.89 | 88.45 | 3.72 |
| 625 | 817 | 1009 | 64.99 | 4.22 | 73.08 | 4.06 |
| 626 | 818 | 1010 | 79.77 | 4.04 | 91.95 | 0.48 |
| 627 | 819 | 1011 | 85.95 | 4.34 | 101.60 | 2.29 |
| 628 | 820 | 1012 | 89.00 | 1.49 | 97.59 | 0.80 |
| 629 | 821 | 1013 | 99.94 | 3.47 | 93.27 | 3.94 |
| 630 | 822 | 1014 | 80.09 | 0.87 | 89.56 | 2.49 |
| 631 | 823 | 1015 | 98.01 | 4.12 | 84.39 | 4.80 |
| 632 | 824 | 1016 | 84.89 | 4.28 | 86.46 | 3.15 |
| 633 | 825 | 1017 | 105.65 | 6.82 | 95.39 | 1.27 |
| 634 | 826 | 1018 | 99.48 | 3.64 | 92.33 | 0.34 |
| 635 | 827 | 1019 | 126.39 | 1.80 | 94.03 | 0.23 |
| 636 | 828 | 1020 | 123.75 | 4.93 | 85.65 | 11.77 |
| 637 | 829 | 1021 | 76.65 | 2.09 | 73.60 | 6.19 |
| 638 | 830 | 1022 | 61.01 | 0.90 | 105.53 | ND |
| 639 | 831 | 1023 | 69.40 | 3.19 | 85.20 | 1.17 |
| 640 | 832 | 1024 | 93.89 | 0.50 | 97.24 | 1.61 |
| 641 | 833 | 1025 | 104.96 | 6.66 | 96.06 | 1.24 |
| 642 | 834 | 1026 | 94.76 | 3.43 | 99.36 | 7.73 |
| 643 | 835 | 1027 | 87.00 | 4.17 | 93.87 | 0.41 |
| 644 | 836 | 1028 | 94.30 | 5.23 | 95.28 | 6.77 |
| 645 | 837 | 1029 | 87.79 | 1.07 | 99.08 | 3.70 |
| 646 | 838 | 1030 | 124.33 | 28.51 | 89.23 | 6.39 |
| 647 | 839 | 1031 | 107.46 | 24.49 | 76.77 | 3.97 |
| 648 | 840 | 1032 | 127.66 | 32.41 | 83.70 | 9.45 |
| 649 | 841 | 1033 | 100.95 | 1.99 | 108.87 | 11.52 |
| 650 | 842 | 1034 | 77.74 | 4.09 | 85.23 | 6.90 |
| 651 | 843 | 1035 | 69.63 | 1.25 | 80.95 | 2.44 |
| 652 | 844 | 1036 | 80.12 | 0.52 | 88.88 | 0.34 |
| 653 | 845 | 1037 | 84.68 | 5.47 | 90.49 | 3.01 |
| 654 | 846 | 1038 | 81.17 | 2.54 | 91.83 | 9.67 |
| 655 | 847 | 1039 | 118.45 | 3.66 | 90.02 | 2.94 |
| 656 | 848 | 1040 | 126.80 | 5.13 | 90.48 | 3.85 |
| 657 | 849 | 1041 | 148.19 | 2.45 | 91.46 | 3.42 |
| 658 | 850 | 1042 | 144.20 | 6.84 | 80.26 | 0.81 |
| 659 | 851 | 1043 | 142.70 | 6.65 | 79.29 | 3.46 |
| 660 | 852 | 1044 | 116.40 | 7.99 | 81.51 | 3.38 |
| 661 | 853 | 1045 | 100.66 | 5.89 | 81.85 | 2.41 |
| 662 | 854 | 1046 | 112.13 | 3.29 | 80.45 | 1.32 |
| 663 | 855 | 1047 | 110.57 | 6.63 | 64.25 | 2.31 |
| 664 | 856 | 1048 | 71.66 | 1.10 | 79.78 | 5.37 |
| 665 | 857 | 1049 | 112.61 | 0.78 | 89.03 | 0.07 |
| 666 | 858 | 1050 | 128.70 | 6.17 | 75.78 | 0.10 |
| 667 | 859 | 1051 | 101.14 | 0.01 | 82.02 | 1.96 |
| 668 | 860 | 1052 | 101.47 | 2.55 | 82.91 | 1.03 |
| 669 | 861 | 1053 | 104.09 | 3.51 | 68.45 | 1.67 |
| 670 | 862 | 1054 | 79.31 | 7.17 | 86.54 | 1.28 |
| 671 | 863 | 1055 | 116.84 | 5.13 | 80.55 | 3.46 |
| 672 | 864 | 1056 | 107.43 | 3.22 | 83.11 | 2.65 |
| 673 | 865 | 1057 | 85.84 | 4.36 | 80.71 | 0.64 |
| 674 | 866 | 1058 | 116.38 | 8.51 | 80.54 | 0.13 |
| 675 | 867 | 1059 | 96.36 | 0.08 | 94.82 | 4.36 |
| 676 | 868 | 1060 | 86.00 | 2.53 | 101.63 | 7.24 |
| 677 | 869 | 1061 | 115.82 | 2.13 | 92.81 | 2.22 |
| 678 | 870 | 1062 | 129.61 | 2.23 | 88.71 | 2.07 |
| 679 | 871 | 1063 | 118.20 | 3.61 | 99.26 | 6.01 |
| 680 | 872 | 1064 | 98.36 | 0.45 | 103.85 | 7.75 |
| 681 | 873 | 1065 | 126.28 | ND | 88.21 | 1.32 |
| 682 | 874 | 1066 | 84.03 | 5.83 | 89.72 | 2.58 |
| 683 | 875 | 1067 | 128.37 | ND | 93.00 | 0.72 |
| 684 | 876 | 1068 | 81.50 | 7.21 | 90.83 | 3.53 |
| 685 | 877 | 1069 | 62.09 | 1.95 | 74.06 | 2.81 |
| 686 | 878 | 1070 | 62.23 | 6.72 | 77.04 | 0.24 |
| 687 | 879 | 1071 | 60.84 | 1.29 | 75.82 | 0.08 |
| 688 | 880 | 1072 | 58.74 | 0.86 | 73.38 | 0.89 |
| 689 | 881 | 1073 | 70.34 | 4.92 | 86.12 | 0.29 |
| 690 | 882 | 1074 | 68.16 | 0.70 | 76.82 | 0.04 |
| 691 | 883 | 1075 | 71.48 | ND | 73.10 | 2.04 |
| 692 | 884 | 1076 | 97.75 | ND | 86.30 | 5.82 |
| 693 | 885 | 1077 | 116.90 | ND | 97.90 | 11.95 |
| 694 | 886 | 1078 | 81.04 | ND | 82.40 | 2.96 |
| 695 | 887 | 1079 | 76.17 | ND | 79.68 | 6.47 |
| 696 | 888 | 1080 | 86.54 | ND | 85.94 | 0.41 |
| 697 | 889 | 1081 | 72.90 | ND | 73.39 | 0.75 |
| 698 | 890 | 1082 | 79.80 | ND | 79.23 | 3.94 |
| 699 | 891 | 1083 | 80.35 | ND | 82.03 | 2.98 |
| 700 | 892 | 1084 | 86.43 | ND | 84.53 | 7.44 |
| 701 | 893 | 1085 | 85.38 | 0.92 | 85.15 | 3.69 |
| 702 | 894 | 1086 | 71.14 | 8.76 | 79.87 | 3.57 |
| 703 | 895 | 1087 | 91.42 | 4.11 | 96.30 | 0.84 |
| 704 | 896 | 1088 | 85.58 | 4.21 | 100.36 | 2.91 |
| 705 | 897 | 1089 | 72.46 | 2.18 | 83.78 | 3.74 |
| 706 | 898 | 1090 | 74.44 | 4.91 | 91.55 | 2.60 |
| 707 | 899 | 1091 | 70.26 | ND | 68.31 | 0.86 |
| 708 | 900 | 1092 | 64.67 | 2.72 | 74.73 | 2.37 |
| 709 | 901 | 1093 | 60.10 | 1.66 | 70.06 | 1.20 |
| 710 | 902 | 1094 | 77.68 | 2.71 | 79.86 | 1.70 |
| 711 | 903 | 1095 | 80.87 | 6.93 | 88.45 | 0.26 |
| 712 | 904 | 1096 | 71.77 | 2.82 | 77.15 | 2.49 |
| 713 | 905 | 1097 | 74.06 | 6.45 | 71.78 | ND |
| 714 | 906 | 1098 | 95.36 | 2.53 | 90.70 | 0.12 |
| 715 | 907 | 1099 | 69.22 | 3.55 | 73.53 | 1.78 |
| 716 | 908 | 1100 | 75.88 | 3.79 | 83.50 | 3.68 |
| 717 | 909 | 1101 | 88.08 | 3.45 | 88.50 | 1.56 |
| 718 | 910 | 1102 | 86.90 | 4.00 | 80.60 | 2.21 |
| 719 | 911 | 1103 | 75.91 | 1.03 | 78.74 | 0.67 |
| 720 | 912 | 1104 | 73.73 | 1.54 | 81.50 | 7.00 |
| 721 | 913 | 1105 | 82.27 | 0.19 | 84.35 | ND |
| 722 | 914 | 1106 | 89.62 | 2.49 | 85.72 | ND |
| 723 | 915 | 1107 | 86.04 | 1.61 | 89.23 | 5.61 |
| 724 | 916 | 1108 | 73.72 | 3.32 | 78.28 | 4.53 |
| 725 | 917 | 1109 | 68.99 | 1.56 | 74.19 | 4.12 |
| 726 | 918 | 1110 | 94.34 | 3.29 | 84.91 | 11.00 |
| 727 | 919 | 1111 | 103.47 | 2.56 | 104.13 | 1.91 |
| 728 | 920 | 1112 | 100.31 | 7.79 | 97.07 | 0.88 |
| 729 | 921 | 1113 | 97.91 | 5.02 | 100.13 | 2.09 |
| 730 | 922 | 1114 | 84.68 | 3.18 | 93.19 | 2.70 |
| 731 | 923 | 1115 | 81.38 | 7.98 | 95.51 | 3.67 |
| 732 | 924 | 1116 | 81.85 | 2.73 | 93.07 | 2.15 |
| 733 | 925 | 1117 | 84.52 | 2.37 | 84.81 | 4.04 |
| 734 | 926 | 1118 | 93.08 | 3.32 | 88.28 | 1.77 |
| 735 | 927 | 1119 | 95.30 | 0.64 | 81.68 | 0.81 |
| 736 | 928 | 1120 | 106.28 | 6.53 | 92.75 | 3.38 |
| 737 | 929 | 1121 | 90.21 | 6.73 | 86.80 | 0.68 |
| 738 | 930 | 1122 | 101.80 | 3.65 | 92.58 | 0.34 |
| 739 | 931 | 1123 | 93.95 | 6.69 | 82.79 | 0.30 |
| 740 | 932 | 1124 | 93.12 | 8.07 | 83.03 | 1.30 |
| 741 | 933 | 1125 | 67.53 | 3.34 | 69.37 | 2.83 |
| 742 | 934 | 1126 | 83.72 | 6.03 | 86.19 | 1.20 |
| 743 | 935 | 1127 | 58.26 | 0.61 | 66.88 | 0.04 |
| 744 | 936 | 1128 | 72.73 | 3.24 | 78.72 | 2.13 |
| 745 | 937 | 1129 | 80.74 | 1.94 | 82.42 | 0.76 |
| 746 | 938 | 1130 | 70.86 | 0.42 | 75.36 | 1.10 |
| 747 | 939 | 1131 | 78.97 | 7.40 | 76.39 | 0.62 |
| 748 | 940 | 1132 | 93.04 | 2.05 | 82.68 | 4.05 |
| 749 | 941 | 1133 | 90.99 | 3.24 | 86.48 | 0.83 |
| 750 | 942 | 1134 | 72.37 | 0.89 | 76.62 | 0.76 |
| 751 | 943 | 1135 | 81.25 | 0.50 | 88.27 | 1.91 |
| 752 | 944 | 1136 | 84.21 | 1.77 | 85.36 | 2.46 |
| 753 | 945 | 1137 | 91.49 | 8.94 | 95.76 | 5.34 |
| 754 | 946 | 1138 | 94.32 | 12.14 | 93.06 | 2.32 |
| 755 | 947 | 1139 | 91.12 | 6.78 | 85.98 | 2.61 |
| 756 | 948 | 1140 | 86.35 | 8.68 | 83.18 | 5.45 |
| 757 | 949 | 1141 | 91.59 | 6.48 | 87.31 | 3.57 |
| 758 | 950 | 1142 | 90.14 | 1.65 | 86.73 | 2.14 |
| 759 | 951 | 1143 | 68.86 | 0.07 | 67.06 | ND |
| 760 | 952 | 1144 | 91.97 | 3.07 | 80.84 | ND |
| 761 | 953 | 1145 | 88.44 | 3.85 | 80.32 | 1.04 |
| 762 | 954 | 1146 | 81.95 | 7.60 | 69.60 | 3.18 |
| 763 | 955 | 1147 | 86.70 | 5.34 | 82.01 | 2.86 |
| 764 | 956 | 1148 | 72.72 | 4.57 | 69.85 | 0.13 |
| 765 | 957 | 1149 | 77.28 | 2.85 | 70.08 | 1.55 |
| 766 | 958 | 1150 | 74.82 | 3.42 | 76.48 | 2.07 |
| 767 | 959 | 1151 | 83.21 | 0.43 | 77.47 | 1.13 |
| 768 | 960 | 1152 | 80.25 | 5.26 | 87.11 | 2.08 |

### Example 2. IC50 Potency Determination of siRNA Molecules of the Disclosure

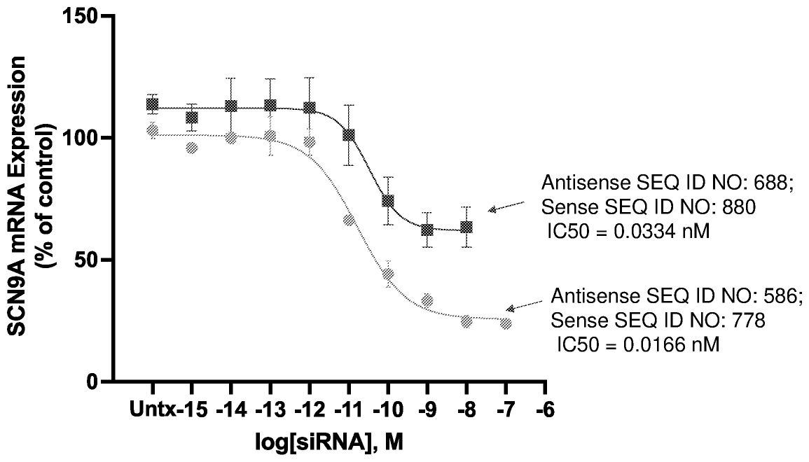

For analysis of compound potency, G402 cells were actively transfected with SCN9A-targeting siRNA at concentrations of 1 fM to 100 nM. Expression of SCN9A mRNA was assessed at 72 hours using RT-qPCR as described above in Example 1, and the IC50 of each compound was calculated. Two siRNA molecules were tested in this assay: (1) an siRNA molecule having an antisense strand of SEQ ID NO: 688 and a sense strand of SEQ ID NO: 880, having an IC50 of 0.0334 nM, and (2) an siRNA molecule having an antisense strand of SEQ ID NO: 586 and a sense strand of SEQ ID NO: 778, having an IC50 of 0.0166 nM. The IC50 curves are shown in FIG. 1.

### Example 3. Generating SCN9A-Targeting siRNA Molecules

The small interfering RNA (siRNA) molecules of the disclosure can be synthesized by standard methods known in the art as further discussed below, e.g., by use of an automated DNA synthesizer, such as are commercially available from, for example, Biosearch, Applied Biosystems, Inc.

The siRNA agent can be prepared using solution-phase or solid-phase organic synthesis or both. Organic synthesis offers the advantage that the oligonucleotide including unnatural or modified nucleotides can be easily prepared. Specific examples of siRNA molecules, with the nucleotide sequence of the sense and antisense strand, as well as the sodium voltage-gated channel alpha subunit 9 (SCN9A) mRNA target sequence, are shown above in Table 1. It is appreciated that one of skill in the art could anneal the antisense (AS) strand to the corresponding sense (S) strand to yield a ds-siRNA molecule. Alternatively, one of skill in the art could derive a ss-siRNA molecule using antisense strand only.

### Example 4. Optimizing SCN9A-Targeting siRNA Molecules

It is contemplated that for siRNA agent disclosed herein, modifications to the siRNA may further optimize the molecule's efficacy or biophysical properties (e.g., increasing serum stability or circulating half-life, increasing thermal stability, enhancing transmembrane delivery, and/or targeting to a particular location or cell type). Such optimization could be achieved by systematically either adding or removing linked nucleosides to generate longer or shorter sequences. Further siRNA optimization could include the incorporation of, for example, one or more alternative nucleosides, alternative 2′ sugar moieties, and/or alternative internucleoside linkages. Further still, such optimized siRNA molecules may include the introduction of hydrophobic and/or stabilizing moieties at the 5′ and/or 3′ ends.

siRNA Optimization with Alternative Nucleosides

Optimization of the siRNA molecules of the disclosure may include one or more of the following nucleoside modifications: 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl (—C═C—CH3) uracil and cytosine and other alkynyl derivatives of pyrimidine bases, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 2-F-adenine, 2-amino-adenine, 8-azaguanine and 8-azaadenine, 7-deazaguanine and 7-deazaadenine, and/or 3-deazaguanine and 3-deazaadenine. The siRNA molecules may also include nucleobases in which the purine or pyrimidine base is replaced with other heterocycles, for example 7-deaza-adenine, 7-deazaguanosine, 2-aminopyridine, and/or 2-pyridone. Further optimization of the siRNA molecules of the disclosure may include nucleobases disclosed in U.S. Pat. No. 3,687,808; Kroschwitz, J. I., ed. The Concise Encyclopedia of Polymer Science and Engineering, New York, John Wiley & Sons, 1990, pp. 858-859; Englisch et al., Angewandte Chemie, International Edition 30:613, 1991; and Sanghvi, Y. S., Chapter 16, Antisense Research and Applications, CRC Press, Gait, M. J. ed., 1993, pp. 289-302.

siRNA Optimization with Alternative Sugar Modifications

Optimization of the siRNA molecules of the disclosure may include one or more of the following 2′ sugar modifications: 2′-O-methyl (2′-O-Me), 2′-methoxyethoxy (2′-O—CH₂CH₂OCH₃, also known as 2′-O-(2-methoxyethyl) or 2′-MOE), 2′-dimethylaminooxyethoxy, i.e., a O(CH₂)₂ON(CH₃)₂ group, also known as 2′-DMAOE, and/or 2′-dimethylaminoethoxyethoxy (also known in the art as 2′-O-dimethylamino-ethoxy-ethyl or 2′-DMAEOE), i.e., 2′-O—CH₂OCH₂N(CH₃)₂. Other possible 2′-modifications that can optimize the siRNA molecules of the disclosure include all possible orientations of OH; F; O-, S-, or N-alkyl; O-, S-, or N-alkenyl; O-, S- or N-alkynyl; or O-alkyl-O-alkyl, wherein the alkyl, alkenyl and alkynyl may be substituted or unsubstituted C1 to C10 alkyl or C2 to C10 alkenyl and alkynyl. Other potential sugar substituent groups include, e.g., aminopropoxy (—OCH₂CH₂CH₂NH₂), allyl (—CH₂—CH═CH₂), —O-allyl (—O—CH₂—CH═CH₂) and fluoro (F). 2′-sugar substituent groups may be in the arabino (up) position or ribo (down) position. In some embodiments, the 2′-arabino modification is 2′-F. Similar modifications may also be made at other positions on the siRNA molecule, particularly the 3′ position of the sugar on the 3′ terminal nucleoside or in 2′-5′ linked oligonucleotides and the 5′ position of 5′ terminal nucleotide. Oligonucleotides may also have sugar mimetics such as cyclobutyl moieties in place of the pentofuranosyl sugar.

siRNA Optimization with Alternative Internucleoside Linkages

Optimization of the siRNA molecules of the disclosure may include one or more of the following internucleoside modifications: phosphorothioates, phosphorodithioates, phosphotriesters, aminoalkylphosphotriesters, methyl and other alkyl phosphonates including 3-alkylene phosphonates, 5′-alkylene phosphonates, phosphinates, phosphoramidates including 3-amino phosphoramidate and aminoalkylphosphoramidates, thionophosphoramidates, thionoalkylphosphonates, thionoalkylphosphotriesters, selenophosphates, and boranophosphates having normal 3-5′ linkages, 2′-5′ linked analogs of these, and those having inverted polarity wherein one or more internucleotide linkages is a 3′ to 3′, 5′ to 5′ or 2′ to 2′ linkage.

siRNA Optimization with Hydrophobic Moieties

Optimization of the siRNA molecules of the disclosure may include hydrophobic moieties covalently attached to the 5′ end or the 3′ end. Non-limiting examples of hydrophobic moieties suitable for use with the siRNA molecules of the disclosure may include cholesterol, vitamin D, tocopherol, phosphatidylcholine (PC), docohexaenoic acid, docosanoic acid, PC-docosanoic acid, eicosapentaenoic acid, lithocholic acid or any combination of the aforementioned hydrophobic moieties with PC.

siRNA Optimization with Stabilizing Moieties

Optimization of the siRNA molecules of the disclosure may include a 5′-phosphorous stabilizing moiety that protects the siRNA molecules from degradation. A 5′-phosphorus stabilizing moiety replaces the 5′-phosphate to prevent hydrolysis of the phosphate. Hydrolysis of the 5′-phosphate prevents binding to RISC, a necessary step in gene silencing. Any replacement for phosphate that does not impede binding to RISC is contemplated in this disclosure. In some embodiments, the replacement for the 5′-phosphate is also stable to in vivo hydrolysis. Each siRNA strand may independently and optionally employ any suitable 5′-phosphorus stabilizing moiety. Non-limiting examples of 5′ stabilizing moieties suitable for use with the siRNA molecules of the disclosure may include those demonstrated by Formulas IX-XVI above.

siRNA Optimization with Branched siRNA

Optimization of the siRNA molecules of the disclosure may include the incorporation of branching patterns, such as, for example, di-branched, tri-branched, or tetra-branched siRNAs connected by way of a linker. Each main branch may be further branched to allow for 2, 3, 4, 5, 6, 7, or 8 separate RNA single- or double-strands. The branch points on the linker may stem from the same atom, or separate atoms along the linker. Some exemplary embodiments are listed in Table 2, above.

The siRNA composition of the disclosure may be optimized to be in the form of: di-branched siRNA molecules, as represented by any one of Formulas XVII-XIX; tri-branched siRNA molecules, as represented by any one of Formulas XX-XXIII; and/or tetra-branched siRNA molecules, as represented by any one of Formulas XXIV-XXVIII, wherein each RNA, independently, is an siRNA molecule, L is a linker, and each X, independently, represents a branch point moiety (e.g., phosphoroamidite, tosylated solketal, 1,3-diaminopropanol, pentaerythritol, or any one of the branch point moieties described in U.S. Pat. No. 10,478,503).

### Example 5. Preparation and Administrating SCN9A-Targeting siRNA Molecules

The siRNA molecules in the present disclosure may be formulated into a pharmaceutical composition for administration to a subject in a biologically compatible form suitable for administration in vivo. For example, the siRNA molecules of the disclosure may be administered in a suitable diluent, carrier, or excipient, and may further contain a preservative, e.g., to prevent the growth of microorganisms. Conventional procedures and ingredients for the selection and preparation of suitable formulations are described, for example, in Remington, J. P. The Science and Practice of Pharmacy, Easton, PA. Mack Publishers, 2012, 22^{nd} ed. and in The United States Pharmacopeial Convention, The National Formulary, United States Pharmacopeial, 2015, USP 38 NF 33).

The method of the disclosure contemplates any route of administration to the subject's CNS or neurons that is tolerated by the siRNA compositions of the disclosure. Non-limiting examples of siRNA injections into the CNS or neurons include intrathecal injection, intra-cisterna magna injection by catheterization, or direct injection into a specific nerve or ganglion (ganglia) (e.g., trigeminal or dorsal root ganglia). A physician having ordinary skill in the art can readily determine an effective route of administration.

### Example 6. Methods for the Treatment of Pain Using SCN9A-Targeting siRNA Molecules

A subject in need of treatment for chronic, persistent, or acute symptoms of pain, including pain that is nociceptive or neuropathic in nature, is treated with a dosage of the siRNA molecule or siRNA composition of the disclosure, formulated as a salt, at frequency determined by a practitioner. A physician having ordinary skill in the art can readily determine an effective amount of the siRNA molecule for administration to a mammalian subject (e.g., a human) in need thereof. For example, a physician could start prescribing doses of one of the siRNA molecules of the disclosure at levels lower than that required to achieve the desired therapeutic effect and gradually increase the dosage until the desired effect is achieved. Alternatively, a physician may begin a treatment regimen by administering one of the siRNA molecules of the disclosure at a high dose and subsequently administer progressively lower doses until a therapeutic effect is achieved (e.g., a reduction in expression of SCN9A mRNA). In general, a suitable daily dose of one of one of the siRNA molecules of the disclosure will be an amount which is the lowest dose effective to produce a therapeutic effect. The ss- or ds-siRNA molecules of the disclosure may be administered by injection, e.g., intrathecally, directly into a specific nerve or ganglion (ganglia) (e.g., trigeminal or dorsal root ganglia), or by intra-cisterna magna injection via catheterization. A daily dose of a therapeutic composition of one of the siRNA molecules of the disclosure may be administered as a single dose or as two, three, four, five, six or more doses administered separately at appropriate intervals throughout the day, week, month, or year, optionally, in unit dosage forms. While it is possible for any of the siRNA molecules of the disclosure to be administered alone, it may also be administered as a pharmaceutical formulation in combination with excipients, carriers, and optionally, additional therapeutic agents. Dosage and frequency are determined based on the subject's height, weight, age, sex, and other disorders.

The siRNA molecule(s) of the disclosure is selected by the practitioner for compatibility with the subject. Single- or double-stranded siRNA molecules (e.g., non-branched siRNA, di-branched siRNA, tri-branched siRNA, tetra-branched siRNA) are available for selection. The siRNA molecule chosen has an antisense strand and may have a sense strand with a sequence and RNA modifications (e.g., natural and non-natural internucleoside linkages, modified sugars, 5′-phosphorus stabilizing moieties, hydrophobic moieties, and/or branching sructures) best suited to the patient.

The siRNA molecule is delivered by the route best suited the patient (e.g., intrathecally, intracerebroventricularly, intrastriatally, by direct injection into a specific nerve or ganglion (ganglia) such as trigeminal or dorsal root ganglia, or by intra-cisterna magna injection via catheterization) and condition at a rate tolerable to the patient until the subject has reached a maximum tolerated dose, or until the symptoms of pain are ameliorated satisfactorily.

### Example 7. Methods for the Treatment of Pain Associated with a Pain Disorder

The small interfering RNA (siRNA) molecules of the disclosure can be used for the treatment of pain disorders, such as those characterized as erythromelalgia (e.g., episodes of pain, redness, and swelling, typically at the extremities) and/or those induced by gain-of-function SCN9A gene variants. Non-limiting examples of clinical diagnoses suitable for treatment with the siRNA molecules of the disclosure include Gerhardt disease, Mitchell disease, or Weir-Mitchell disease.

A subject with a condition of erythromelalgia is treated with a dosage of the siRNA molecule or composition of the disclosure, formulated as a salt, at frequency determined by a practitioner. A physician having ordinary skill in the art can readily determine an effective amount of the siRNA molecule for administration to a mammalian subject (e.g., a human) in need thereof. For example, a physician could start prescribing doses of one of the siRNA molecules of the disclosure at levels lower than that required to achieve the desired therapeutic effect and gradually increase the dosage until the desired effect is achieved. Alternatively, a physician may begin a treatment regimen by administering one of the siRNA molecules of the disclosure at a high dose and subsequently administer progressively lower doses until a therapeutic effect is achieved (e.g., a reduction in expression of SCN9A mRNA). In general, a suitable daily dose of one of one of the siRNA molecules of the disclosure will be an amount which is the lowest dose effective to produce a therapeutic effect. The ss- or ds-interfering RNA molecules of the disclosure may be administered by injection, e.g., intrathecally, by direct injection into a specific nerve or ganglion (ganglia) (e.g., trigeminal or dorsal root ganglia) or by intra-cisterna magna injection via catheterization. A daily dose of a therapeutic composition of one of the siRNA molecules of the disclosure may be administered as a single dose or as two, three, four, five, six or more doses administered separately at appropriate intervals throughout the day, week, month, or year, optionally, in unit dosage forms. While it is possible for any of the siRNA molecules of the disclosure to be administered alone, it may also be administered as a pharmaceutical formulation in combination with excipients, carriers, and optionally, additional therapeutic agents. Dosage and frequency are determined based on the subject's height, weight, age, sex, and other disorders.

The siRNA molecule(s) of the disclosure is selected by the practitioner for compatibility with the subject. Single- or double-stranded siRNA molecules (e.g., non-branched siRNA, di-branched siRNA, tri-branched siRNA, tetra-branched siRNA) are available for selection. The siRNA molecule chosen has an antisense strand and may have a sense strand with a sequence and RNA modifications (e.g., natural and non-natural internucleoside linkages, modified sugars, 5′-phosphorus stabilizing moieties, hydrophobic moieties, and/or branching sructures) best suited to the patient.

The siRNA molecule is delivered by the route best suited the patient (e.g., intrathecally, by direct injection into a specific nerve or ganglion (ganglia), or by intra-cisterna magna injection via catheterization) and condition at a rate tolerable to the patient until the subject has reached a maximum tolerated dose, or until the symptoms of pain are ameliorated satisfactorily.

### Other Embodiments

All publications, patents, and patent applications mentioned in this specification are incorporated herein by reference to the same extent as if each independent publication or patent application was specifically and individually indicated to be incorporated by reference.

While the invention has been described in connection with specific embodiments thereof, it will be understood that it is capable of further modifications and this application is intended to cover any variations, uses, or adaptations of the invention following, in general, the principles of the invention and including such departures from the invention that come within known or customary practice within the art to which the invention pertains and may be applied to the essential features hereinbefore set forth, and follows in the scope of the claims.

Other embodiments are within the claims.

1. A small interfering RNA (siRNA) molecule comprising an antisense strand and sense strand having complementarity to the antisense strand, wherein the antisense strand has complementarity sufficient to hybridize to a region within a sodium voltage-gated channel alpha subunit 9 (SCN9A) mRNA transcript having the nucleic acid sequence of any one of SEQ ID NOs: 385-576 and 961-1152.

2. The siRNA molecule of claim 1, wherein the antisense strand has at least 70% complementarity to a region of 19, 20, 21, or more contiguous nucleobases within the SCN9A mRNA transcript having the nucleic acid sequence of any one of SEQ ID NOs: 385-576 and 961-1152, optionally wherein the antisense strand has at least 70% complementarity to the SCN9A mRNA transcript having the nucleic acid sequence of any one of SEQ ID NOs: 385-576 and 961-1152.

3. The siRNA molecule of claim 2, wherein the antisense strand has at least 75% complementarity to the region within the SCN9A mRNA transcript having the nucleic acid sequence of any one of SEQ ID NOs: 385-576 and 961-1152, optionally wherein the antisense strand has at least 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% complementarity to the region within the SCN9A mRNA transcript having the nucleic acid sequence of any one of SEQ ID Nos: 385-576 and 961-1152.

4. The siRNA molecule of any one of claims 1-3, wherein the antisense strand comprises at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, or 30 contiguous nucleotides that are fully complementary to a contiguous polynucleotide segment of equal length within the region of the SCN9A RNA transcript having the nucleic acid sequence of any one of SEQ ID NOs: 385-576 and 961-1152.

5. The siRNA molecule of claim 4, wherein the antisense strand comprises from 10 to 30 contiguous nucleotides that are fully complementary to a contiguous polynucleotide segment of equal length within the region of the SCN9A RNA transcript having the nucleic acid sequence of any one of SEQ ID NOs: 385-576 and 961-1152.

6. The siRNA molecule of claim 5, wherein the antisense strand comprises from 12 to 30 contiguous nucleotides that are fully complementary to a contiguous polynucleotide segment of equal length within the region of the SCN9A RNA transcript having the nucleic acid sequence of any one of SEQ ID NOs: 385-576 and 961-1152.

7. The siRNA molecule of claim 6, wherein the antisense strand comprises from 15 to 30 contiguous nucleotides that are fully complementary to a contiguous polynucleotide segment of equal length within the region of the SCN9A RNA transcript having the nucleic acid sequence of any one of SEQ ID NOs: 385-576 and 961-1152.

8. The siRNA molecule of claim 7, wherein the antisense strand comprises from 18 to 30 contiguous nucleotides that are fully complementary to a contiguous polynucleotide segment of equal length within the region of the SCN9A RNA transcript having the nucleic acid sequence of any one of SEQ ID NOs: 385-576 and 961-1152.

9. The siRNA molecule of claim 8, wherein the antisense strand comprises from 18 to 25 contiguous nucleotides that are fully complementary to a contiguous polynucleotide segment of equal length within the region of the SCN9A RNA transcript having the nucleic acid sequence of any one of SEQ ID Nos: 385-576 and 961-1152.

10. The siRNA molecule of claim 9, wherein the antisense strand comprises from 18 to 21 contiguous nucleotides that are fully complementary to a contiguous polynucleotide segment of equal length within the region of the SCN9A RNA transcript having the nucleic acid sequence of any one of SEQ ID NOs: 385-576 and 961-1152.

11. The siRNA molecule of claim 10, wherein the antisense strand comprises 21 contiguous nucleotides that are fully complementary to a contiguous polynucleotide segment of equal length within the region of the SCN9A RNA transcript having the nucleic acid sequence of any one of SEQ ID NOs: 385-576 and 961-1152.

12. The siRNA molecule of any one of claims 1-11, wherein the antisense strand comprises 9 or fewer nucleotide mismatches relative to the region of the SCN9A RNA transcript having the nucleic acid sequence of any one of SEQ ID NOs: 385-576 and 961-1152, optionally wherein the antisense strand comprises 8 or fewer, 7 or fewer, 6 or fewer, 5 or fewer, 4 or fewer, 3 or fewer, 2 or fewer, or only 1 mismatch relative to the region of the SCN9A RNA transcript having the nucleic acid sequence of any one of SEQ ID NOs: 385-576 and 961-1152.

13. The siRNA molecule of any one of claims 1-12, wherein the antisense strand has a nucleic acid sequence that is at least 85% identical to the nucleic acid sequence of any one of SEQ ID NOs: 1-192 and 577-768.

14. The siRNA molecule of claim 13, wherein the antisense strand has a nucleic acid sequence that is at least 90% identical to the nucleic acid sequence of any one of SEQ ID NOs: 1-192 and 577-768.

15. The siRNA molecule of claim 14, wherein the antisense strand has a nucleic acid sequence that is at least 95% identical to the nucleic acid sequence of SEQ ID NOs: 1-192 and 577-768, optionally wherein the antisense strand has a nucleic acid sequence that is at least 96%, 97%, 98%, or 99% identical to the nucleic acid sequence of any one of SEQ ID NOs: 1-192 and 577-768.

16. The siRNA molecule of claim 15, wherein the antisense strand has the nucleic acid sequence of any one of SEQ ID NOs: 1-192 and 577-768.

17. The siRNA molecule of any one of claims 1-16, wherein the sense strand has a nucleic acid sequence that is at least 85% identical to the nucleic acid sequence of any one of SEQ ID NOs: 193-384 and 769-960.

18. The siRNA molecule of claim 17, wherein the sense strand has a nucleic acid sequence that is at least 90% identical to the nucleic acid sequence of any one of SEQ ID NOs: 193-384 and 769-960.

19. The siRNA molecule of claim 18, wherein the sense strand has a nucleic acid sequence that is at least 95% identical to the nucleic acid sequence of SEQ ID NOs: 193-384 and 769-960, optionally wherein the sense strand has a nucleic acid sequence that is at least 96%, 97%, 98%, or 99% identical to the nucleic acid sequence of any one of SEQ ID NOs: 193-384 and 769-960.

20. The siRNA molecule of claim 19, wherein the sense strand has the nucleic acid sequence of any one of SEQ ID NOs: 193-384 and 769-960.

21. The siRNA molecule of any one of claims 1-20, wherein the antisense strand comprises a structure represented by Formula I, wherein Formula I is, in the 5′-to-3′ direction:
A-B-(A′)ⱼ-C-P²-D-P¹-(C′-P¹)ₖ-C′ Formula I;
wherein A is represented by the formula C-P¹-D-P¹;
each A′ is represented by the formula C-P²-D-P²;
B is represented by the formula C-P²-D-P²-D-P²-D-P²;
each C is a 2′-O-methyl (2′-O-Me) ribonucleoside;
each C′, independently, is a 2′-O-Me ribonucleoside or a 2′-fluoro (2′-F) ribonucleoside;
each D is a 2′-F ribonucleoside;
each P¹ is a phosphorothioate internucleoside linkage;
each P² is a phosphodiester internucleoside linkage;
j is an integer from 1 to 7; and
k is an integer from 1 to 7.

22. The siRNA molecule of claim 21, wherein the antisense strand comprises a structure represented by Formula A1, wherein Formula A1 is, in the 5′-to-3′ direction:
A-S-B-S-A-O-B-O-B-O-B-O-A-O-B-O-A-O-B-O-A-O-B-O-A-O-B-O-A-O-B-S-A-S-A-S-A-S-B-S-A Formula A1;
wherein A represents a 2′-O-Me ribonucleoside, B represents a 2′-F ribonucleoside, O represents a phosphodiester internucleoside linkage, and S represents a phosphorothioate internucleoside linkage.

23. The siRNA molecule of any one of claims 1-20, wherein the antisense strand comprises a structure represented by Formula II, wherein Formula II is, in the 5′-to-3′ direction:
A-B-(A′)ⱼ-C-P²-D-P¹-(C-P¹)ₖ-C′ Formula II;
wherein A is represented by the formula C-P¹-D-P¹;
each A′ is represented by the formula C-P²-D-P²;
B is represented by the formula C-P²-D-P²-D-P²-D-P²;
each C is a 2′-O-methyl (2′-O-Me) ribonucleoside;
each C′, independently, is a 2′-O-Me ribonucleoside or a 2′-fluoro (2′-F) ribonucleoside;
each D is a 2′-F ribonucleoside;
each P¹ is a phosphorothioate internucleoside linkage;
each P² is a phosphodiester internucleoside linkage;
j is an integer from 1 to 7; and
k is an integer from 1 to 7.

24. The siRNA molecule of claim 23, wherein the antisense strand comprises a structure represented by Formula A2, wherein Formula A2 is, in the 5′-to-3′ direction:
A-S-B-S-A-O-B-O-B-O-B-O-A-O-B-O-A-O-B-O-A-O-B-O-A-O-B-O-A-O-B-S-A-S-A-S-A-S-A-S-A Formula A2;
wherein A represents a 2′-O-Me ribonucleoside, B represents a 2′-F ribonucleoside, O represents a phosphodiester internucleoside linkage, and S represents a phosphorothioate internucleoside linkage.

25. The siRNA molecule of any one of claims 1-24, wherein the sense strand comprises a structure represented by Formula III, wherein Formula III is, in the 5′-to-3′ direction:
E-(A′)ₘ-F Formula III;
wherein E is represented by the formula (C-P¹)₂;
F is represented by the formula (C-P²)₃-D-P¹-C-P¹-C, (C-P²)₃-D-P²-C-P²-C, (C-P²)₃-D-P¹-C-P¹-D, or (C-P²)₃-D-P²-C-P²-D;
A′, C, D, P¹, and P² are as defined in Formula II; and
m is an integer from 1 to 7.

26. The siRNA molecule of claim 25, wherein the sense strand comprises a structure represented by Formula S1, wherein Formula S1 is, in the 5′-to-3′ direction:
A-S-A-S-A-O-B-O-A-O-B-O-A-O-B-O-A-O-B-O-A-O-A-O-A-O-B-S-A-S-A Formula S1;
wherein A represents a 2′-O-Me ribonucleoside, B represents a 2′-F ribonucleoside, O represents a phosphodiester internucleoside linkage, and S represents a phosphorothioate internucleoside linkage.

27. The siRNA molecule of claim 25, wherein the sense strand comprises a structure represented by Formula S2, wherein Formula S2 is, in the 5′-to-3′ direction:
A-S-A-S-A-O-B-O-A-O-B-O-A-O-B-O-A-O-B-O-A-O-A-O-A-O-B-O-A-O-A Formula S2;
wherein A represents a 2′-O-Me ribonucleoside, B represents a 2′-F ribonucleoside, O represents a phosphodiester internucleoside linkage, and S represents a phosphorothioate internucleoside linkage.

28. The siRNA molecule of claim 25, wherein the sense strand comprises a structure represented by Formula S3, wherein Formula S3 is, in the 5′-to-3′ direction:
A-S-A-S-A-O-B-O-A-O-B-O-A-O-B-O-A-O-B-O-A-O-A-O-A-O-B-S-A-S-B Formula S3;
wherein A represents a 2′-O-Me ribonucleoside, B represents a 2′-F ribonucleoside, O represents a phosphodiester internucleoside linkage, and S represents a phosphorothioate internucleoside linkage.

29. The siRNA molecule of claim 25, wherein the sense strand comprises a structure represented by Formula S4, wherein Formula S4 is, in the 5′-to-3′ direction:
A-S-A-S-A-O-B-O-A-O-B-O-A-O-B-O-A-O-B-O-A-O-A-O-A-O-B-O-A-O-B Formula S4;
wherein A represents a 2′-O-Me ribonucleoside, B represents a 2′-F ribonucleoside, O represents a phosphodiester internucleoside linkage, and S represents a phosphorothioate internucleoside linkage.

30. The siRNA molecule of any one of claims 1-20 and 25-29, wherein the antisense strand comprises a structure represented by Formula IV, wherein Formula IV is, in the 5′-to-3′ direction:
A-(A′)ⱼ-C-P²-B-(C-P¹)ₖ-C′ Formula IV;
wherein A is represented by the formula C-P¹-D-P¹;
each A′ is represented by the formula C-P²-D-P²;
B is represented by the formula D-P¹-C-P¹-D-P¹;
each C is a 2′-O-Me ribonucleoside;
each C′, independently, is a 2′-O-Me ribonucleoside or a 2′-F ribonucleoside;
each D is a 2′-F ribonucleoside;
each P¹ is a phosphorothioate internucleoside linkage;
each P² is a phosphodiester internucleoside linkage;
j is an integer from 1 to 7; and
k is an integer from 1 to 7.

31. The siRNA molecule of claim 30, wherein the antisense strand comprises a structure represented by Formula A3, wherein Formula A3 is, in the 5′-to-3′ direction:
A-S-B-S-A-O-B-O-A-O-B-O-A-O-B-O-A-O-B-O-A-O-B-O-A-O-B-O-A-O-B-S-A-S-B-S-A-S-A-S-A Formula A3;
wherein A represents a 2′-O-Me ribonucleoside, B represents a 2′-F ribonucleoside, O represents a phosphodiester internucleoside linkage, and S represents a phosphorothioate internucleoside linkage.

32. The siRNA molecule of any one of claims 1-24, 30, and 31, wherein the sense strand comprises a structure represented by Formula V, wherein Formula V is, in the 5′-to-3′ direction:
E-(A′)ₘ-C-P²-F Formula V;
wherein E is represented by the formula (C-P¹)₂;
F is represented by the formula D-P¹-C-P¹-C, D-P²-C-P²-C, D-P¹-C-P¹-D, or D-P²-C-P²-D;
A′, C, D, P¹ and P² are as defined in Formula IV; and
m is an integer from 1 to 7.

33. The siRNA molecule of claim 32, wherein the sense strand comprises a structure represented by Formula S5, wherein Formula S5 is, in the 5′-to-3′ direction:
A-S-A-S-A-O-B-O-A-O-B-O-A-O-B-O-A-O-B-O-A-O-B-O-A-O-B-S-A-S-A Formula S5;
wherein A represents a 2′-O-Me ribonucleoside, B represents a 2′-F ribonucleoside, O represents a phosphodiester internucleoside linkage, and S represents a phosphorothioate internucleoside linkage.

34. The siRNA molecule of claim 32, wherein the sense strand comprises a structure represented by Formula S6, wherein Formula S6 is, in the 5′-to-3′ direction:
A-S-A-S-A-O-B-O-A-O-B-O-A-O-B-O-A-O-B-O-A-O-B-O-A-O-B-O-A-O-A Formula S6;
wherein A represents a 2′-O-Me ribonucleoside, B represents a 2′-F ribonucleoside, O represents a phosphodiester internucleoside linkage, and S represents a phosphorothioate internucleoside linkage.

35. The siRNA molecule of claim 32, wherein the sense strand comprises a structure represented by Formula S7, wherein Formula S7 is, in the 5′-to-3′ direction:
A-S-A-S-A-O-B-O-A-O-B-O-A-O-B-O-A-O-B-O-A-O-B-O-A-O-B-S-A-S-B Formula S7;
wherein A represents a 2′-O-Me ribonucleoside, B represents a 2′-F ribonucleoside, O represents a phosphodiester internucleoside linkage, and S represents a phosphorothioate internucleoside linkage.

36. The siRNA molecule of claim 32, wherein the sense strand comprises a structure represented by Formula S8, wherein Formula S8 is, in the 5′-to-3′ direction:
A-S-A-S-A-O-B-O-A-O-B-O-A-O-B-O-A-O-B-O-A-O-B-O-A-O-B-O-A-O-B Formula S8;
wherein A represents a 2′-O-Me ribonucleoside, B represents a 2′-F ribonucleoside, O represents a phosphodiester internucleoside linkage, and S represents a phosphorothioate internucleoside linkage.

37. The siRNA molecule of any one of claims 1-20, 25-29, and 32-36, wherein the antisense strand comprises a structure represented by Formula VI, wherein Formula VI is, in the 5′-to-3′ direction:
A-Bⱼ-E-Bₖ-E-F-Gₗ-D-P¹-C′ Formula VI;
wherein A is represented by the formula C-P¹-D-P¹;
each B is represented by the formula C-P²;
each C is a 2′-O-Me ribonucleoside;
each C′, independently, is a 2′-O-Me ribonucleoside or a 2′-F ribonucleoside;
each D is a 2′-F ribonucleoside;
each E is represented by the formula D-P²-C-P²;
F is represented by the formula D-P¹-C-P¹;
each G is represented by the formula C-P¹;
each P¹ is a phosphorothioate internucleoside linkage;
each P² is a phosphodiester internucleoside linkage;
j is an integer from 1 to 7;
k is an integer from 1 to 7; and
l is an integer from 1 to 7.

38. The siRNA molecule of claim 37, wherein the antisense strand comprises a structure represented by Formula A4, wherein Formula A4 is, in the 5′-to-3′ direction:
A-S-B-S-A-O-A-O-A-O-B-O-A-O-A-O-A-O-A-O-A-O-A-O-A-O-B-O-A-O-B-S-A-S-A-S-A-S-B-S-A Formula A4;
wherein A represents a 2′-O-Me ribonucleoside, B represents a 2′-F ribonucleoside, O represents a phosphodiester internucleoside linkage, and S represents a phosphorothioate internucleoside linkage.

39. The siRNA molecule of any one of claims 1-24, 30, 31, 37, and 38, wherein the sense strand comprises a structure represented by Formula VII, wherein Formula VII is, in the 5′-to-3′ direction:
H-Bₘ-Iₙ-A′-Bₒ-H-C Formula VII;
wherein A′ is represented by the formula C-P²-D-P²;
each H is represented by the formula (C-P¹)₂;
each I is represented by the formula (D-P²);
B, C, D, P¹ and P² are as defined in Formula VI;
m is an integer from 1 to 7;
n is an integer from 1 to 7; and
o is an integer from 1 to 7.

40. The siRNA molecule of claim 39, wherein the sense strand comprises a structure represented by Formula S9, wherein Formula S9 is, in the 5′-to-3′ direction:
A-S-A-S-A-O-A-O-A-O-B-O-B-O-B-O-A-O-B-O-A-O-A-O-A-O-A-S-A-S-A Formula S9;
wherein A represents a 2′-O-Me ribonucleoside, B represents a 2′-F ribonucleoside, O represents a phosphodiester internucleoside linkage, and S represents a phosphorothioate internucleoside linkage.

41. The siRNA molecule of any one of claims 1-40, wherein the antisense strand further comprises a 5′ phosphorus stabilizing moiety at the 5′ end of the antisense strand.

42. The siRNA molecule of any one of claims 1-41, wherein the sense strand further comprises a 5′ phosphorus stabilizing moiety at the 5′ end of the sense strand.

43. The siRNA molecule of claim 41 or 42, wherein each 5′ phosphorus stabilizing moiety is, independently, represented by any one of Formulas IX-XVI: 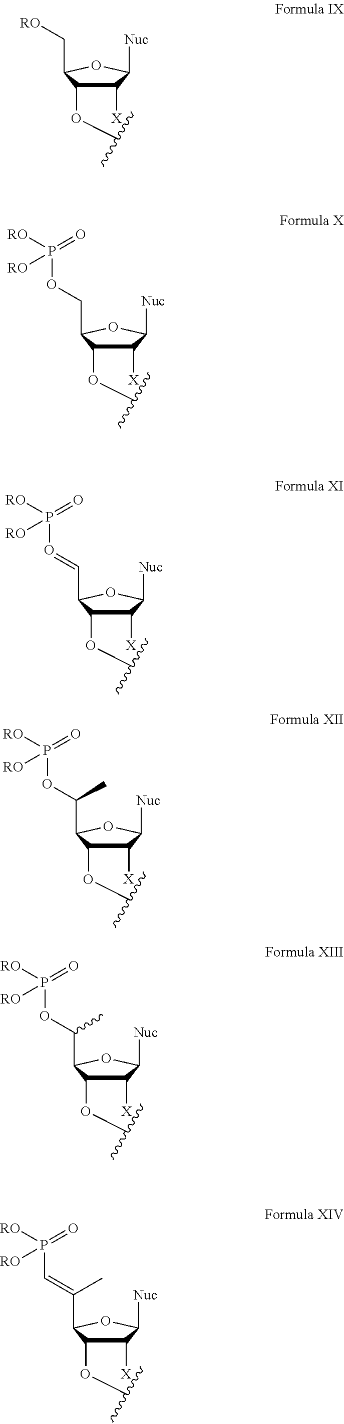 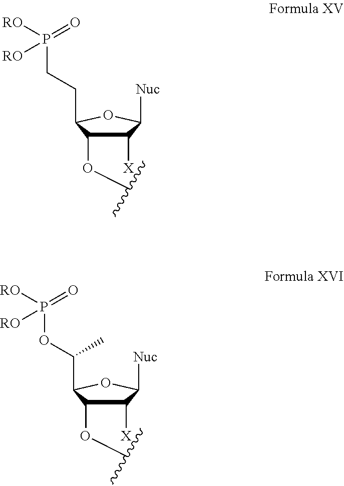
wherein Nuc represents a nucleobase selected from the group consisting of adenine, uracil, guanine, thymine, and cytosine, and R represents an optionally substituted alkyl, optionally substituted alkenyl, optionally substituted alkynyl, phenyl, benzyl, hydroxy, or hydrogen.

44. The siRNA molecule of claim 43, wherein the nucleobase is an adenine, uracil, guanine, thymine, or cytosine.

45. The siRNA molecule of any one of claims 41-44, wherein the 5′ phosphorus stabilizing moiety is (E)-vinylphosphonate represented by Formula XI.

46. The siRNA molecule of any one of claims 1-45, wherein the siRNA molecule further comprises a hydrophobic moiety at the 5′ or the 3′ end of the siRNA molecule.

47. The siRNA molecule of claim 46, wherein the hydrophobic moiety is selected from a group consisting of cholesterol, vitamin D, or tocopherol.

48. The siRNA molecule of any one of claims 1-47, wherein the length of the sense strand is between 10 and 30 nucleotides.

49. The siRNA molecule of claim 48, wherein the length of the sense strand is between 10 and 25 nucleotides.

50. The siRNA molecule of claim 49, wherein the length of the sense strand is between 12 and 25 nucleotides.

51. The siRNA molecule of claim 50, wherein the length of the sense strand is between 12 and 20 nucleotides.

52. The siRNA molecule of claim 51, wherein the length of the sense strand is between 12 and 19 nucleotides.

53. The siRNA molecule of claim 52, wherein the length of the sense strand is 15 nucleotides.

54. The siRNA molecule of claim 52, wherein the length of the sense strand is 16 nucleotides.

55. The siRNA molecule of claim 52, wherein the length of the sense strand is 18 nucleotides.

56. The siRNA molecule of any one of claims 1-55, wherein the length of the antisense strand is between 10 and 30 nucleotides.

57. The siRNA molecule of claim 56, wherein the length of the antisense strand is between 12 and 30 nucleotides.

58. The siRNA molecule of claim 57, wherein the length of the antisense strand is between 15 and 30 nucleotides.

59. The siRNA molecule of claim 58, wherein the length of the antisense strand is between 18 and 30 nucleotides.

60. The siRNA molecule of claim 59, wherein the length of the antisense strand is between 18 and 25 nucleotides.

61. The siRNA molecule of claim 60, wherein the length of the antisense strand is between 18 and 21 nucleotides.

62. The siRNA molecule of claim 61, wherein the length of the antisense strand is 18 nucleotides.

63. The siRNA molecule of claim 61, wherein the length of the antisense strand is 20 nucleotides.

64. The siRNA molecule of claim 61, wherein the length of the antisense strand is 21 nucleotides.

65. The siRNA molecule of any one of claims 1-64, wherein the siRNA molecule is a branched siRNA molecule.

66. The siRNA molecule of claim 65, wherein the branched siRNA molecule is di-branched, tri-branched, or tetra-branched.

67. The siRNA molecule of claim 66, wherein the siRNA molecule is a di-branched siRNA molecule, optionally wherein the di-branched siRNA molecule is represented by any one of Formulas XVII-XIX:
RNA-L-RNA Formula XVII; 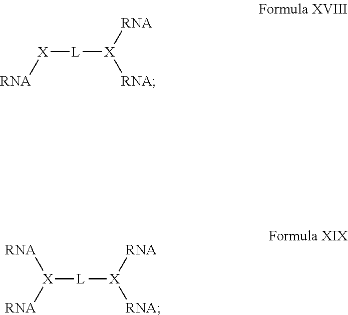
wherein each RNA is, independently, an siRNA molecule, L is a linker, and each X, independently, represents a branch point moiety.

68. The siRNA molecule of claim 66, wherein the siRNA molecule is a tri-branched siRNA molecule, optionally wherein the tri-branched siRNA molecule is represented by any one of Formulas XX-XXIII: 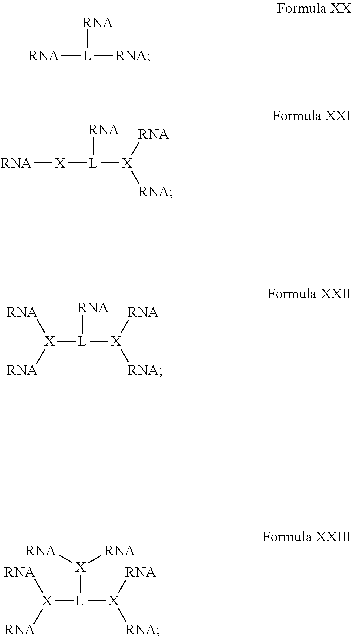
wherein each RNA is, independently, an siRNA molecule, L is a linker, and each X, independently, represents a branch point moiety.

69. The siRNA molecule of claim 66, wherein the siRNA molecule is a tetra-branched siRNA molecule, optionally wherein the tetra-branched siRNA molecule is represented by any one of Formulas XXIV-XXVIII: 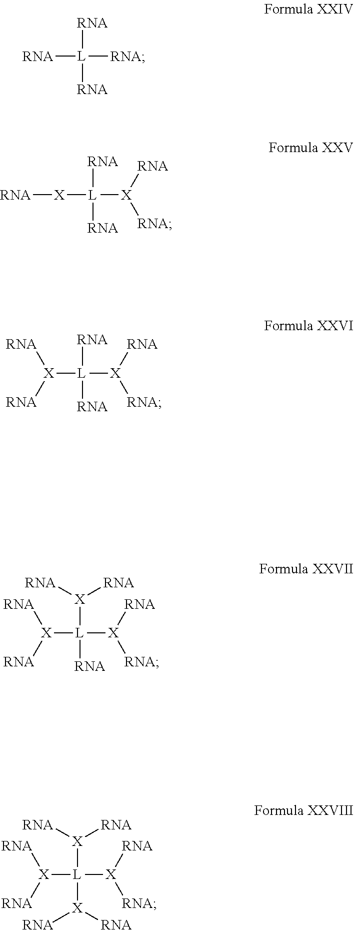
wherein each RNA is, independently, an siRNA molecule, L is a linker, and each X, independently, represents a branch point moiety.

70. The siRNA molecule of any one of claims 67-69, wherein the linker is selected from a group consisting of one or more contiguous subunits of an ethylene glycol, alkyl, carbohydrate, block copolymer, peptide, RNA, and DNA.

71. The siRNA molecule of claim 70, wherein the one or more contiguous subunits is 2 to 20 contiguous subunits.

72. A pharmaceutical composition comprising the siRNA molecule of any one of claims 1-71 and a pharmaceutically acceptable excipient, carrier, or diluent.

73. A method of delivering an siRNA molecule to the central nervous system (CNS) or neurons of a subject experiencing pain or diagnosed as having a pain disorder, the method comprising administering a therapeutically effective amount of the siRNA molecule of any one of claims 1-71 or the pharmaceutical composition of claim 72 to the subject.

74. A method of treating pain or a pain disorder in a subject in need thereof, the method comprising administering a therapeutically effective amount of the siRNA molecule of any one of claims 1-71 or the pharmaceutical composition of claim 72 to the subject.

75. The method of claim 73 or 74, wherein the pain is neuropathic pain.

76. The method of claim 73 or 74, wherein the pain is nociceptive pain.

77. The method of claim 73 or 74, wherein the pain is post-operative pain.

78. The method of claim 73 or 74, wherein the pain is persistent pain.

79. The method of claim 73 or 74, wherein the pain is inflammatory pain.

80. The method of claim 73 or 74, wherein the pain disorder is Gerhardt disease, Mitchell disease, or Weir-Mitchell disease.

81. The method of claim 73 or 74, wherein the subject has been diagnosed with erythromelalgia.

82. A method of reducing SCN9A expression in a subject in need thereof, the method comprising administering a therapeutically effective amount of the siRNA molecule of any one of claims 1-71 or the pharmaceutical composition of claim 72 to the CNS of the subject.

83. The method of claim 82, wherein, upon administration of the siRNA molecule or pharmaceutical composition to the subject, the subject exhibits selective reduction in SCN9A expression over expression of one or more other voltage-gated sodium ion channel genes.

84. The method of any one of claims 73-83 wherein the siRNA molecule or the pharmaceutical composition is administered to the subject by way of intrathecal injection or by direct injection into a specific nerve or ganglion.

85. The method of any one of claims 73-84, wherein the subject is a human.

86. A kit comprising the siRNA molecule of any one of claims 1-71, or the pharmaceutical composition of claim 72, and a package insert, wherein the package insert instructs a user of the kit to perform the method of any one of claims 73-85.


SEQUENCE LISTING (1152 sequences)

SEQ ID NO: 1 - RNA, 21 nt, synthetic construct
  source 1..21: mol_type = other RNA
tgttcaatgagggcaagagac

SEQ ID NO: 2 - RNA, 21 nt, synthetic construct
  source 1..21: mol_type = other RNA
ttgttcaatgagggcaagaga

SEQ ID NO: 3 - RNA, 21 nt, synthetic construct
  source 1..21: mol_type = other RNA
tcctttgttcaatactatgaa

SEQ ID NO: 4 - RNA, 21 nt, synthetic construct
  source 1..21: mol_type = other RNA
tccctttgttcaatactatga

SEQ ID NO: 5 - RNA, 21 nt, synthetic construct
  source 1..21: mol_type = other RNA
ttccctttgttcaatactatg

SEQ ID NO: 6 - RNA, 21 nt, synthetic construct
  source 1..21: mol_type = other RNA
tttccctttgttcaatactat

SEQ ID NO: 7 - RNA, 21 nt, synthetic construct
  source 1..21: mol_type = other RNA
ttttccctttgttcaatacta

SEQ ID NO: 8 - RNA, 21 nt, synthetic construct
  source 1..21: mol_type = other RNA
tttgaaacggaagattgtttt

SEQ ID NO: 9 - RNA, 21 nt, synthetic construct
  source 1..21: mol_type = other RNA
tttcttagaggactgaaagga

SEQ ID NO: 10 - RNA, 21 nt, synthetic construct
  source 1..21: mol_type = other RNA
tcttcttagaggactgaaagg

SEQ ID NO: 11 - RNA, 21 nt, synthetic construct
  source 1..21: mol_type = other RNA
tttcttcttagaggactgaaa

SEQ ID NO: 12 - RNA, 21 nt, synthetic construct
  source 1..21: mol_type = other RNA
tattcttcttagaggactgaa

SEQ ID NO: 13 - RNA, 21 nt, synthetic construct
  source 1..21: mol_type = other RNA
tatattcttcttagaggactg

SEQ ID NO: 14 - RNA, 21 nt, synthetic construct
  source 1..21: mol_type = other RNA
tgatattcttcttagaggact

SEQ ID NO: 15 - RNA, 21 nt, synthetic construct
  source 1..21: mol_type = other RNA
tagatattcttcttagaggac

SEQ ID NO: 16 - RNA, 21 nt, synthetic construct
  source 1..21: mol_type = other RNA
ttagatattcttcttagagga

SEQ ID NO: 17 - RNA, 21 nt, synthetic construct
  source 1..21: mol_type = other RNA
tatagatattcttcttagagg

SEQ ID NO: 18 - RNA, 21 nt, synthetic construct
  source 1..21: mol_type = other RNA
ttgctgaataaggagtgtact

SEQ ID NO: 19 - RNA, 21 nt, synthetic construct
  source 1..21: mol_type = other RNA
tatgctgaataaggagtgtac

SEQ ID NO: 20 - RNA, 21 nt, synthetic construct
  source 1..21: mol_type = other RNA
tcatgctgaataaggagtgta

SEQ ID NO: 21 - RNA, 21 nt, synthetic construct
  source 1..21: mol_type = other RNA
tgcatgctgaataaggagtgt

SEQ ID NO: 22 - RNA, 21 nt, synthetic construct
  source 1..21: mol_type = other RNA
tagcatgctgaataaggagtg

SEQ ID NO: 23 - RNA, 21 nt, synthetic construct
  source 1..21: mol_type = other RNA
tcagtaaaagtgtactcgaca

SEQ ID NO: 24 - RNA, 21 nt, synthetic construct
  source 1..21: mol_type = other RNA
tccagtaaaagtgtactcgac

SEQ ID NO: 25 - RNA, 21 nt, synthetic construct
  source 1..21: mol_type = other RNA
taagcctcttgcaaggatttt

SEQ ID NO: 26 - RNA, 21 nt, synthetic construct
  source 1..21: mol_type = other RNA
tcaaattctgttaaatacgca

SEQ ID NO: 27 - RNA, 21 nt, synthetic construct
  source 1..21: mol_type = other RNA
tacaaattctgttaaatacgc

SEQ ID NO: 28 - RNA, 21 nt, synthetic construct
  source 1..21: mol_type = other RNA
tggattacagaaatagttttc

SEQ ID NO: 29 - RNA, 21 nt, synthetic construct
  source 1..21: mol_type = other RNA
tgggattacagaaatagtttt

SEQ ID NO: 30 - RNA, 21 nt, synthetic construct
  source 1..21: mol_type = other RNA
tttagtgcaaacacactcaga

SEQ ID NO: 31 - RNA, 21 nt, synthetic construct
  source 1..21: mol_type = other RNA
tattagtgcaaacacactcag

SEQ ID NO: 32 - RNA, 21 nt, synthetic construct
  source 1..21: mol_type = other RNA
tcaattagtgcaaacacactc

SEQ ID NO: 33 - RNA, 21 nt, synthetic construct
  source 1..21: mol_type = other RNA
tccaattagtgcaaacacact

SEQ ID NO: 34 - RNA, 21 nt, synthetic construct
  source 1..21: mol_type = other RNA
ttagtccaattagtgcaaaca

SEQ ID NO: 35 - RNA, 21 nt, synthetic construct
  source 1..21: mol_type = other RNA
tgtagtccaattagtgcaaac

SEQ ID NO: 36 - RNA, 21 nt, synthetic construct
  source 1..21: mol_type = other RNA
ttgtagtccaattagtgcaaa

SEQ ID NO: 37 - RNA, 21 nt, synthetic construct
  source 1..21: mol_type = other RNA
tctgtagtccaattagtgcaa

SEQ ID NO: 38 - RNA, 21 nt, synthetic construct
  source 1..21: mol_type = other RNA
tgctgtagtccaattagtgca

SEQ ID NO: 39 - RNA, 21 nt, synthetic construct
  source 1..21: mol_type = other RNA
tcagctgtagtccaattagtg

SEQ ID NO: 40 - RNA, 21 nt, synthetic construct
  source 1..21: mol_type = other RNA
tgagcatctttggatccttcc

SEQ ID NO: 41 - RNA, 21 nt, synthetic construct
  source 1..21: mol_type = other RNA
tagagcatctttggatccttc

SEQ ID NO: 42 - RNA, 21 nt, synthetic construct
  source 1..21: mol_type = other RNA
tctgaatctgtgctgaaacca

SEQ ID NO: 43 - RNA, 21 nt, synthetic construct
  source 1..21: mol_type = other RNA
tcctgaatctgtgctgaaacc

SEQ ID NO: 44 - RNA, 21 nt, synthetic construct
  source 1..21: mol_type = other RNA
tgctcgtgtagccataatcag

SEQ ID NO: 45 - RNA, 21 nt, synthetic construct
  source 1..21: mol_type = other RNA
ttctgtttagcttcttcaatg

SEQ ID NO: 46 - RNA, 21 nt, synthetic construct
  source 1..21: mol_type = other RNA
tttctgtttagcttcttcaat

SEQ ID NO: 47 - RNA, 21 nt, synthetic construct
  source 1..21: mol_type = other RNA
ttttctgtttagcttcttcaa

SEQ ID NO: 48 - RNA, 21 nt, synthetic construct
  source 1..21: mol_type = other RNA
tctttctgtttagcttcttca

SEQ ID NO: 49 - RNA, 21 nt, synthetic construct
  source 1..21: mol_type = other RNA
tcacgaatgctgagtggtgac

SEQ ID NO: 50 - RNA, 21 nt, synthetic construct
  source 1..21: mol_type = other RNA
tccacgaatgctgagtggtga

SEQ ID NO: 51 - RNA, 21 nt, synthetic construct
  source 1..21: mol_type = other RNA
ttgatgttactgctgcgtcgc

SEQ ID NO: 52 - RNA, 21 nt, synthetic construct
  source 1..21: mol_type = other RNA
tgagcatgagggctgagcgtc

SEQ ID NO: 53 - RNA, 21 nt, synthetic construct
  source 1..21: mol_type = other RNA
tggagcatgagggctgagcgt

SEQ ID NO: 54 - RNA, 21 nt, synthetic construct
  source 1..21: mol_type = other RNA
tgggagcatgagggctgagcg

SEQ ID NO: 55 - RNA, 21 nt, synthetic construct
  source 1..21: mol_type = other RNA
tcaagttcttccacagtgttt

SEQ ID NO: 56 - RNA, 21 nt, synthetic construct
  source 1..21: mol_type = other RNA
ttcaagttcttccacagtgtt

SEQ ID NO: 57 - RNA, 21 nt, synthetic construct
  source 1..21: mol_type = other RNA
tcaaatctgtaccaccaaggt

SEQ ID NO: 58 - RNA, 21 nt, synthetic construct
  source 1..21: mol_type = other RNA
tgcaaatctgtaccaccaagg

SEQ ID NO: 59 - RNA, 21 nt, synthetic construct
  source 1..21: mol_type = other RNA
tgagagcaattccagatcaag

SEQ ID NO: 60 - RNA, 21 nt, synthetic construct
  source 1..21: mol_type = other RNA
tggagagcaattccagatcaa

SEQ ID NO: 61 - RNA, 21 nt, synthetic construct
  source 1..21: mol_type = other RNA
tatggagagcaattccagatc

SEQ ID NO: 62 - RNA, 21 nt, synthetic construct
  source 1..21: mol_type = other RNA
ttatggagagcaattccagat

SEQ ID NO: 63 - RNA, 21 nt, synthetic construct
  source 1..21: mol_type = other RNA
tatatggagagcaattccaga

SEQ ID NO: 64 - RNA, 21 nt, synthetic construct
  source 1..21: mol_type = other RNA
taatatggagagcaattccag

SEQ ID NO: 65 - RNA, 21 nt, synthetic construct
  source 1..21: mol_type = other RNA
tcaatatggagagcaattcca

SEQ ID NO: 66 - RNA, 21 nt, synthetic construct
  source 1..21: mol_type = other RNA
tccaatatggagagcaattcc

SEQ ID NO: 67 - RNA, 21 nt, synthetic construct
  source 1..21: mol_type = other RNA
tatccaatatggagagcaatt

SEQ ID NO: 68 - RNA, 21 nt, synthetic construct
  source 1..21: mol_type = other RNA
ttatccaatatggagagcaat

SEQ ID NO: 69 - RNA, 21 nt, synthetic construct
  source 1..21: mol_type = other RNA
tttatccaatatggagagcaa

SEQ ID NO: 70 - RNA, 21 nt, synthetic construct
  source 1..21: mol_type = other RNA
ttttatccaatatggagagca

SEQ ID NO: 71 - RNA, 21 nt, synthetic construct
  source 1..21: mol_type = other RNA
tattttatccaatatggagag

SEQ ID NO: 72 - RNA, 21 nt, synthetic construct
  source 1..21: mol_type = other RNA
tgaattttatccaatatggag

SEQ ID NO: 73 - RNA, 21 nt, synthetic construct
  source 1..21: mol_type = other RNA
tagatctacaaaaggatccat

SEQ ID NO: 74 - RNA, 21 nt, synthetic construct
  source 1..21: mol_type = other RNA
taagatctacaaaaggatcca

SEQ ID NO: 75 - RNA, 21 nt, synthetic construct
  source 1..21: mol_type = other RNA
tcaagatctacaaaaggatcc

SEQ ID NO: 76 - RNA, 21 nt, synthetic construct
  source 1..21: mol_type = other RNA
tgcaagatctacaaaaggatc

SEQ ID NO: 77 - RNA, 21 nt, synthetic construct
  source 1..21: mol_type = other RNA
ttgcaagatctacaaaaggat

SEQ ID NO: 78 - RNA, 21 nt, synthetic construct
  source 1..21: mol_type = other RNA
tttgcaagatctacaaaagga

SEQ ID NO: 79 - RNA, 21 nt, synthetic construct
  source 1..21: mol_type = other RNA
tattgcaagatctacaaaagg

SEQ ID NO: 80 - RNA, 21 nt, synthetic construct
  source 1..21: mol_type = other RNA
taattgcaagatctacaaaag

SEQ ID NO: 81 - RNA, 21 nt, synthetic construct
  source 1..21: mol_type = other RNA
tgtaattgcaagatctacaaa

SEQ ID NO: 82 - RNA, 21 nt, synthetic construct
  source 1..21: mol_type = other RNA
tggtaattgcaagatctacaa

SEQ ID NO: 83 - RNA, 21 nt, synthetic construct
  source 1..21: mol_type = other RNA
ttggtaattgcaagatctaca

SEQ ID NO: 84 - RNA, 21 nt, synthetic construct
  source 1..21: mol_type = other RNA
tatggtaattgcaagatctac

SEQ ID NO: 85 - RNA, 21 nt, synthetic construct
  source 1..21: mol_type = other RNA
taatggtaattgcaagatcta

SEQ ID NO: 86 - RNA, 21 nt, synthetic construct
  source 1..21: mol_type = other RNA
taaatggtaattgcaagatct

SEQ ID NO: 87 - RNA, 21 nt, synthetic construct
  source 1..21: mol_type = other RNA
tcaaatggtaattgcaagatc

SEQ ID NO: 88 - RNA, 21 nt, synthetic construct
  source 1..21: mol_type = other RNA
ttatgcaaatggtaattgcaa

SEQ ID NO: 89 - RNA, 21 nt, synthetic construct
  source 1..21: mol_type = other RNA
tctatgcaaatggtaattgca

SEQ ID NO: 90 - RNA, 21 nt, synthetic construct
  source 1..21: mol_type = other RNA
taactatgcaaatggtaattg

SEQ ID NO: 91 - RNA, 21 nt, synthetic construct
  source 1..21: mol_type = other RNA
tgtttaaaactatgcaaatgg

SEQ ID NO: 92 - RNA, 21 nt, synthetic construct
  source 1..21: mol_type = other RNA
tcttggaaatactcatatgga

SEQ ID NO: 93 - RNA, 21 nt, synthetic construct
  source 1..21: mol_type = other RNA
tacttggaaatactcatatgg

SEQ ID NO: 94 - RNA, 21 nt, synthetic construct
  source 1..21: mol_type = other RNA
ttacttggaaatactcatatg

SEQ ID NO: 95 - RNA, 21 nt, synthetic construct
  source 1..21: mol_type = other RNA
tctacttggaaatactcatat

SEQ ID NO: 96 - RNA, 21 nt, synthetic construct
  source 1..21: mol_type = other RNA
tcctacttggaaatactcata

SEQ ID NO: 97 - RNA, 21 nt, synthetic construct
  source 1..21: mol_type = other RNA
tccttccacatctgctagaaa

SEQ ID NO: 98 - RNA, 21 nt, synthetic construct
  source 1..21: mol_type = other RNA
ttccttccacatctgctagaa

SEQ ID NO: 99 - RNA, 21 nt, synthetic construct
  source 1..21: mol_type = other RNA
tcttgaagactcggagcagtc

SEQ ID NO: 100 - RNA, 21 nt, synthetic construct
  source 1..21: mol_type = other RNA
tacttgaagactcggagcagt

SEQ ID NO: 101 - RNA, 21 nt, synthetic construct
  source 1..21: mol_type = other RNA
taacttgaagactcggagcag

SEQ ID NO: 102 - RNA, 21 nt, synthetic construct
  source 1..21: mol_type = other RNA
tcaacttgaagactcggagca

SEQ ID NO: 103 - RNA, 21 nt, synthetic construct
  source 1..21: mol_type = other RNA
ttaaacaataaggcacatagc

SEQ ID NO: 104 - RNA, 21 nt, synthetic construct
  source 1..21: mol_type = other RNA
tgtaaacaataaggcacatag

SEQ ID NO: 105 - RNA, 21 nt, synthetic construct
  source 1..21: mol_type = other RNA
tatgtaaacaataaggcacat

SEQ ID NO: 106 - RNA, 21 nt, synthetic construct
  source 1..21: mol_type = other RNA
tcatgtaaacaataaggcaca

SEQ ID NO: 107 - RNA, 21 nt, synthetic construct
  source 1..21: mol_type = other RNA
ttcatgtaaacaataaggcac

SEQ ID NO: 108 - RNA, 21 nt, synthetic construct
  source 1..21: mol_type = other RNA
tatcatgtaaacaataaggca

SEQ ID NO: 109 - RNA, 21 nt, synthetic construct
  source 1..21: mol_type = other RNA
tcatcatgtaaacaataaggc

SEQ ID NO: 110 - RNA, 21 nt, synthetic construct
  source 1..21: mol_type = other RNA
tccatcatgtaaacaataagg

SEQ ID NO: 111 - RNA, 21 nt, synthetic construct
  source 1..21: mol_type = other RNA
taccatcatgtaaacaataag

SEQ ID NO: 112 - RNA, 21 nt, synthetic construct
  source 1..21: mol_type = other RNA
tgaccatcatgtaaacaataa

SEQ ID NO: 113 - RNA, 21 nt, synthetic construct
  source 1..21: mol_type = other RNA
ttgaccatcatgtaaacaata

SEQ ID NO: 114 - RNA, 21 nt, synthetic construct
  source 1..21: mol_type = other RNA
tttgctgtaagattgtctgaa

SEQ ID NO: 115 - RNA, 21 nt, synthetic construct
  source 1..21: mol_type = other RNA
tattgctgtaagattgtctga

SEQ ID NO: 116 - RNA, 21 nt, synthetic construct
  source 1..21: mol_type = other RNA
taattgctgtaagattgtctg

SEQ ID NO: 117 - RNA, 21 nt, synthetic construct
  source 1..21: mol_type = other RNA
tcaattgctgtaagattgtct

SEQ ID NO: 118 - RNA, 21 nt, synthetic construct
  source 1..21: mol_type = other RNA
ttcaattgctgtaagattgtc

SEQ ID NO: 119 - RNA, 21 nt, synthetic construct
  source 1..21: mol_type = other RNA
tggaggttgtttgcatcaggg

SEQ ID NO: 120 - RNA, 21 nt, synthetic construct
  source 1..21: mol_type = other RNA
tctggaggttgtttgcatcag

SEQ ID NO: 121 - RNA, 21 nt, synthetic construct
  source 1..21: mol_type = other RNA
ttctggaggttgtttgcatca

SEQ ID NO: 122 - RNA, 21 nt, synthetic construct
  source 1..21: mol_type = other RNA
tatctggaggttgtttgcatc

SEQ ID NO: 123 - RNA, 21 nt, synthetic construct
  source 1..21: mol_type = other RNA
ttcactgtgaggctgggattg

SEQ ID NO: 124 - RNA, 21 nt, synthetic construct
  source 1..21: mol_type = other RNA
tgtcactgtgaggctgggatt

SEQ ID NO: 125 - RNA, 21 nt, synthetic construct
  source 1..21: mol_type = other RNA
ttgaagctttcaaaccaactg

SEQ ID NO: 126 - RNA, 21 nt, synthetic construct
  source 1..21: mol_type = other RNA
tatgaagctttcaaaccaact

SEQ ID NO: 127 - RNA, 21 nt, synthetic construct
  source 1..21: mol_type = other RNA
taggataatcttaatggtctt

SEQ ID NO: 128 - RNA, 21 nt, synthetic construct
  source 1..21: mol_type = other RNA
tcaggataatcttaatggtct

SEQ ID NO: 129 - RNA, 21 nt, synthetic construct
  source 1..21: mol_type = other RNA
tccaggataatcttaatggtc

SEQ ID NO: 130 - RNA, 21 nt, synthetic construct
  source 1..21: mol_type = other RNA
ttccaggataatcttaatggt

SEQ ID NO: 131 - RNA, 21 nt, synthetic construct
  source 1..21: mol_type = other RNA
tctccaggataatcttaatgg

SEQ ID NO: 132 - RNA, 21 nt, synthetic construct
  source 1..21: mol_type = other RNA
tactccaggataatcttaatg

SEQ ID NO: 133 - RNA, 21 nt, synthetic construct
  source 1..21: mol_type = other RNA
ttactccaggataatcttaat

SEQ ID NO: 134 - RNA, 21 nt, synthetic construct
  source 1..21: mol_type = other RNA
tatactccaggataatcttaa

SEQ ID NO: 135 - RNA, 21 nt, synthetic construct
  source 1..21: mol_type = other RNA
tgcatactccaggataatctt

SEQ ID NO: 136 - RNA, 21 nt, synthetic construct
  source 1..21: mol_type = other RNA
tttccagaatgaagatgtaag

SEQ ID NO: 137 - RNA, 21 nt, synthetic construct
  source 1..21: mol_type = other RNA
tagtagccaagagtgtttgcc

SEQ ID NO: 138 - RNA, 21 nt, synthetic construct
  source 1..21: mol_type = other RNA
tgagtagccaagagtgtttgc

SEQ ID NO: 139 - RNA, 21 nt, synthetic construct
  source 1..21: mol_type = other RNA
ttgagtagccaagagtgtttg

SEQ ID NO: 140 - RNA, 21 nt, synthetic construct
  source 1..21: mol_type = other RNA
tctgagtagccaagagtgttt

SEQ ID NO: 141 - RNA, 21 nt, synthetic construct
  source 1..21: mol_type = other RNA
tttccttcaaatctagataag

SEQ ID NO: 142 - RNA, 21 nt, synthetic construct
  source 1..21: mol_type = other RNA
tcattccttcaaatctagata

SEQ ID NO: 143 - RNA, 21 nt, synthetic construct
  source 1..21: mol_type = other RNA
ttcattccttcaaatctagat

SEQ ID NO: 144 - RNA, 21 nt, synthetic construct
  source 1..21: mol_type = other RNA
tttgctcctatgagtgcattc

SEQ ID NO: 145 - RNA, 21 nt, synthetic construct
  source 1..21: mol_type = other RNA
tattgctcctatgagtgcatt

SEQ ID NO: 146 - RNA, 21 nt, synthetic construct
  source 1..21: mol_type = other RNA
tctcatagaacttgccagcaa

SEQ ID NO: 147 - RNA, 21 nt, synthetic construct
  source 1..21: mol_type = other RNA
tactcatagaacttgccagca

SEQ ID NO: 148 - RNA, 21 nt, synthetic construct
  source 1..21: mol_type = other RNA
tcactcatagaacttgccagc

SEQ ID NO: 149 - RNA, 21 nt, synthetic construct
  source 1..21: mol_type = other RNA
tacactcatagaacttgccag

SEQ ID NO: 150 - RNA, 21 nt, synthetic construct
  source 1..21: mol_type = other RNA
ttttccatcgcacattttgac

SEQ ID NO: 151 - RNA, 21 nt, synthetic construct
  source 1..21: mol_type = other RNA
tttgcaacttgaagcagagat

SEQ ID NO: 152 - RNA, 21 nt, synthetic construct
  source 1..21: mol_type = other RNA
tgttgcaacttgaagcagaga

SEQ ID NO: 153 - RNA, 21 nt, synthetic construct
  source 1..21: mol_type = other RNA
tctgctgcatacataataatc

SEQ ID NO: 154 - RNA, 21 nt, synthetic construct
  source 1..21: mol_type = other RNA
tactgctgcatacataataat

SEQ ID NO: 155 - RNA, 21 nt, synthetic construct
  source 1..21: mol_type = other RNA
tgttggttgaaattatctatg

SEQ ID NO: 156 - RNA, 21 nt, synthetic construct
  source 1..21: mol_type = other RNA
tctgttggttgaaattatcta

SEQ ID NO: 157 - RNA, 21 nt, synthetic construct
  source 1..21: mol_type = other RNA
ttctgttggttgaaattatct

SEQ ID NO: 158 - RNA, 21 nt, synthetic construct
  source 1..21: mol_type = other RNA
tttctgttggttgaaattatc

SEQ ID NO: 159 - RNA, 21 nt, synthetic construct
  source 1..21: mol_type = other RNA
tcttgacctccaagcttcttt

SEQ ID NO: 160 - RNA, 21 nt, synthetic construct
  source 1..21: mol_type = other RNA
ttcttgacctccaagcttctt

SEQ ID NO: 161 - RNA, 21 nt, synthetic construct
  source 1..21: mol_type = other RNA
tcaaatatacatccttggatt

SEQ ID NO: 162 - RNA, 21 nt, synthetic construct
  source 1..21: mol_type = other RNA
tgtcaaatatacatccttgga

SEQ ID NO: 163 - RNA, 21 nt, synthetic construct
  source 1..21: mol_type = other RNA
ttggttaccatgttgagacag

SEQ ID NO: 164 - RNA, 21 nt, synthetic construct
  source 1..21: mol_type = other RNA
tatggttaccatgttgagaca

SEQ ID NO: 165 - RNA, 21 nt, synthetic construct
  source 1..21: mol_type = other RNA
tttctaccatcatggttacca

SEQ ID NO: 166 - RNA, 21 nt, synthetic construct
  source 1..21: mol_type = other RNA
ttttctaccatcatggttacc

SEQ ID NO: 167 - RNA, 21 nt, synthetic construct
  source 1..21: mol_type = other RNA
tcatgtcattaattccatctt

SEQ ID NO: 168 - RNA, 21 nt, synthetic construct
  source 1..21: mol_type = other RNA
tacatgtcattaattccatct

SEQ ID NO: 169 - RNA, 21 nt, synthetic construct
  source 1..21: mol_type = other RNA
taacatgtcattaattccatc

SEQ ID NO: 170 - RNA, 21 nt, synthetic construct
  source 1..21: mol_type = other RNA
tgaacatgtcattaattccat

SEQ ID NO: 171 - RNA, 21 nt, synthetic construct
  source 1..21: mol_type = other RNA
ttgaacatgtcattaattcca

SEQ ID NO: 172 - RNA, 21 nt, synthetic construct
  source 1..21: mol_type = other RNA
tttgaacatgtcattaattcc

SEQ ID NO: 173 - RNA, 21 nt, synthetic construct
  source 1..21: mol_type = other RNA
tacgaagagaatccatctccc

SEQ ID NO: 174 - RNA, 21 nt, synthetic construct
  source 1..21: mol_type = other RNA
taacgaagagaatccatctcc

SEQ ID NO: 175 - RNA, 21 nt, synthetic construct
  source 1..21: mol_type = other RNA
tgaacgaagagaatccatctc

SEQ ID NO: 176 - RNA, 21 nt, synthetic construct
  source 1..21: mol_type = other RNA
ttgaacgaagagaatccatct

SEQ ID NO: 177 - RNA, 21 nt, synthetic construct
  source 1..21: mol_type = other RNA
tgtgaacgaagagaatccatc

SEQ ID NO: 178 - RNA, 21 nt, synthetic construct
  source 1..21: mol_type = other RNA
ttgtgaacgaagagaatccat

SEQ ID NO: 179 - RNA, 21 nt, synthetic construct
  source 1..21: mol_type = other RNA
tccatctgtgaacgaagagaa

SEQ ID NO: 180 - RNA, 21 nt, synthetic construct
  source 1..21: mol_type = other RNA
ttccatctgtgaacgaagaga

SEQ ID NO: 181 - RNA, 21 nt, synthetic construct
  source 1..21: mol_type = other RNA
tttccatctgtgaacgaagag

SEQ ID NO: 182 - RNA, 21 nt, synthetic construct
  source 1..21: mol_type = other RNA
tcttccatctgtgaacgaaga

SEQ ID NO: 183 - RNA, 21 nt, synthetic construct
  source 1..21: mol_type = other RNA
ttcttccatctgtgaacgaag

SEQ ID NO: 184 - RNA, 21 nt, synthetic construct
  source 1..21: mol_type = other RNA
ttttcttccatctgtgaacga

SEQ ID NO: 185 - RNA, 21 nt, synthetic construct
  source 1..21: mol_type = other RNA
tctttcttccatctgtgaacg

SEQ ID NO: 186 - RNA, 21 nt, synthetic construct
  source 1..21: mol_type = other RNA
tcctttcttccatctgtgaac

SEQ ID NO: 187 - RNA, 21 nt, synthetic construct
  source 1..21: mol_type = other RNA
tacctttcttccatctgtgaa

SEQ ID NO: 188 - RNA, 21 nt, synthetic construct
  source 1..21: mol_type = other RNA
taacctttcttccatctgtga

SEQ ID NO: 189 - RNA, 21 nt, synthetic construct
  source 1..21: mol_type = other RNA
tgaacctttcttccatctgtg

SEQ ID NO: 190 - RNA, 21 nt, synthetic construct
  source 1..21: mol_type = other RNA
tatgaacctttcttccatctg

SEQ ID NO: 191 - RNA, 21 nt, synthetic construct
  source 1..21: mol_type = other RNA
tacatgaacctttcttccatc

SEQ ID NO: 192 - RNA, 21 nt, synthetic construct
  source 1..21: mol_type = other RNA
tgtgatgggttcataggacac

SEQ ID NO: 193 - RNA, 16 nt, synthetic construct
  source 1..16: mol_type = other RNA
ttgccctcattgaaca

SEQ ID NO: 194 - RNA, 16 nt, synthetic construct
  source 1..16: mol_type = other RNA
tgccctcattgaacaa

SEQ ID NO: 195 - RNA, 16 nt, synthetic construct
  source 1..16: mol_type = other RNA
agtattgaacaaagga

SEQ ID NO: 196 - RNA, 16 nt, synthetic construct
  source 1..16: mol_type = other RNA
gtattgaacaaaggga

SEQ ID NO: 197 - RNA, 16 nt, synthetic construct
  source 1..16: mol_type = other RNA
tattgaacaaagggaa

SEQ ID NO: 198 - RNA, 16 nt, synthetic construct
  source 1..16: mol_type = other RNA
attgaacaaagggaaa

SEQ ID NO: 199 - RNA, 16 nt, synthetic construct
  source 1..16: mol_type = other RNA
ttgaacaaagggaaaa

SEQ ID NO: 200 - RNA, 16 nt, synthetic construct
  source 1..16: mol_type = other RNA
aatcttccgtttcaaa

SEQ ID NO: 201 - RNA, 16 nt, synthetic construct
  source 1..16: mol_type = other RNA
tcagtcctctaagaaa

SEQ ID NO: 202 - RNA, 16 nt, synthetic construct
  source 1..16: mol_type = other RNA
cagtcctctaagaaga

SEQ ID NO: 203 - RNA, 16 nt, synthetic construct
  source 1..16: mol_type = other RNA
gtcctctaagaagaaa

SEQ ID NO: 204 - RNA, 16 nt, synthetic construct
  source 1..16: mol_type = other RNA
tcctctaagaagaata

SEQ ID NO: 205 - RNA, 16 nt, synthetic construct
  source 1..16: mol_type = other RNA
ctctaagaagaatata

SEQ ID NO: 206 - RNA, 16 nt, synthetic construct
  source 1..16: mol_type = other RNA
tctaagaagaatatca

SEQ ID NO: 207 - RNA, 16 nt, synthetic construct
  source 1..16: mol_type = other RNA
ctaagaagaatatcta

SEQ ID NO: 208 - RNA, 16 nt, synthetic construct
  source 1..16: mol_type = other RNA
taagaagaatatctaa

SEQ ID NO: 209 - RNA, 16 nt, synthetic construct
  source 1..16: mol_type = other RNA
aagaagaatatctata

SEQ ID NO: 210 - RNA, 16 nt, synthetic construct
  source 1..16: mol_type = other RNA
actccttattcagcaa

SEQ ID NO: 211 - RNA, 16 nt, synthetic construct
  source 1..16: mol_type = other RNA
ctccttattcagcata

SEQ ID NO: 212 - RNA, 16 nt, synthetic construct
  source 1..16: mol_type = other RNA
tccttattcagcatga

SEQ ID NO: 213 - RNA, 16 nt, synthetic construct
  source 1..16: mol_type = other RNA
ccttattcagcatgca

SEQ ID NO: 214 - RNA, 16 nt, synthetic construct
  source 1..16: mol_type = other RNA
cttattcagcatgcta

SEQ ID NO: 215 - RNA, 16 nt, synthetic construct
  source 1..16: mol_type = other RNA
agtacacttttactga

SEQ ID NO: 216 - RNA, 16 nt, synthetic construct
  source 1..16: mol_type = other RNA
gtacacttttactgga

SEQ ID NO: 217 - RNA, 16 nt, synthetic construct
  source 1..16: mol_type = other RNA
ccttgcaagaggctta

SEQ ID NO: 218 - RNA, 16 nt, synthetic construct
  source 1..16: mol_type = other RNA
atttaacagaatttga

SEQ ID NO: 219 - RNA, 16 nt, synthetic construct
  source 1..16: mol_type = other RNA
tttaacagaatttgta

SEQ ID NO: 220 - RNA, 16 nt, synthetic construct
  source 1..16: mol_type = other RNA
ctatttctgtaatcca

SEQ ID NO: 221 - RNA, 16 nt, synthetic construct
  source 1..16: mol_type = other RNA
tatttctgtaatccca

SEQ ID NO: 222 - RNA, 16 nt, synthetic construct
  source 1..16: mol_type = other RNA
gtgtgtttgcactaaa

SEQ ID NO: 223 - RNA, 16 nt, synthetic construct
  source 1..16: mol_type = other RNA
tgtgtttgcactaata

SEQ ID NO: 224 - RNA, 16 nt, synthetic construct
  source 1..16: mol_type = other RNA
tgtttgcactaattga

SEQ ID NO: 225 - RNA, 16 nt, synthetic construct
  source 1..16: mol_type = other RNA
gtttgcactaattgga

SEQ ID NO: 226 - RNA, 16 nt, synthetic construct
  source 1..16: mol_type = other RNA
gcactaattggactaa

SEQ ID NO: 227 - RNA, 16 nt, synthetic construct
  source 1..16: mol_type = other RNA
cactaattggactaca

SEQ ID NO: 228 - RNA, 16 nt, synthetic construct
  source 1..16: mol_type = other RNA
actaattggactacaa

SEQ ID NO: 229 - RNA, 16 nt, synthetic construct
  source 1..16: mol_type = other RNA
ctaattggactacaga

SEQ ID NO: 230 - RNA, 16 nt, synthetic construct
  source 1..16: mol_type = other RNA
taattggactacagca

SEQ ID NO: 231 - RNA, 16 nt, synthetic construct
  source 1..16: mol_type = other RNA
attggactacagctga

SEQ ID NO: 232 - RNA, 16 nt, synthetic construct
  source 1..16: mol_type = other RNA
gatccaaagatgctca

SEQ ID NO: 233 - RNA, 16 nt, synthetic construct
  source 1..16: mol_type = other RNA
atccaaagatgctcta

SEQ ID NO: 234 - RNA, 16 nt, synthetic construct
  source 1..16: mol_type = other RNA
tcagcacagattcaga

SEQ ID NO: 235 - RNA, 16 nt, synthetic construct
  source 1..16: mol_type = other RNA
cagcacagattcagga

SEQ ID NO: 236 - RNA, 16 nt, synthetic construct
  source 1..16: mol_type = other RNA
tatggctacacgagca

SEQ ID NO: 237 - RNA, 16 nt, synthetic construct
  source 1..16: mol_type = other RNA
aagaagctaaacagaa

SEQ ID NO: 238 - RNA, 16 nt, synthetic construct
  source 1..16: mol_type = other RNA
agaagctaaacagaaa

SEQ ID NO: 239 - RNA, 16 nt, synthetic construct
  source 1..16: mol_type = other RNA
gaagctaaacagaaaa

SEQ ID NO: 240 - RNA, 16 nt, synthetic construct
  source 1..16: mol_type = other RNA
aagctaaacagaaaga

SEQ ID NO: 241 - RNA, 16 nt, synthetic construct
  source 1..16: mol_type = other RNA
cactcagcattcgtga

SEQ ID NO: 242 - RNA, 16 nt, synthetic construct
  source 1..16: mol_type = other RNA
actcagcattcgtgga

SEQ ID NO: 243 - RNA, 16 nt, synthetic construct
  source 1..16: mol_type = other RNA
gcagcagtaacatcaa

SEQ ID NO: 244 - RNA, 16 nt, synthetic construct
  source 1..16: mol_type = other RNA
tcagccctcatgctca

SEQ ID NO: 245 - RNA, 16 nt, synthetic construct
  source 1..16: mol_type = other RNA
cagccctcatgctcca

SEQ ID NO: 246 - RNA, 16 nt, synthetic construct
  source 1..16: mol_type = other RNA
agccctcatgctccca

SEQ ID NO: 247 - RNA, 16 nt, synthetic construct
  source 1..16: mol_type = other RNA
ctgtggaagaacttga

SEQ ID NO: 248 - RNA, 16 nt, synthetic construct
  source 1..16: mol_type = other RNA
tgtggaagaacttgaa

SEQ ID NO: 249 - RNA, 16 nt, synthetic construct
  source 1..16: mol_type = other RNA
ggtggtacagatttga

SEQ ID NO: 250 - RNA, 16 nt, synthetic construct
  source 1..16: mol_type = other RNA
gtggtacagatttgca

SEQ ID NO: 251 - RNA, 16 nt, synthetic construct
  source 1..16: mol_type = other RNA
tctggaattgctctca

SEQ ID NO: 252 - RNA, 16 nt, synthetic construct
  source 1..16: mol_type = other RNA
ctggaattgctctcca

SEQ ID NO: 253 - RNA, 16 nt, synthetic construct
  source 1..16: mol_type = other RNA
ggaattgctctccata

SEQ ID NO: 254 - RNA, 16 nt, synthetic construct
  source 1..16: mol_type = other RNA
gaattgctctccataa

SEQ ID NO: 255 - RNA, 16 nt, synthetic construct
  source 1..16: mol_type = other RNA
aattgctctccatata

SEQ ID NO: 256 - RNA, 16 nt, synthetic construct
  source 1..16: mol_type = other RNA
attgctctccatatta

SEQ ID NO: 257 - RNA, 16 nt, synthetic construct
  source 1..16: mol_type = other RNA
ttgctctccatattga

SEQ ID NO: 258 - RNA, 16 nt, synthetic construct
  source 1..16: mol_type = other RNA
tgctctccatattgga

SEQ ID NO: 259 - RNA, 16 nt, synthetic construct
  source 1..16: mol_type = other RNA
ctctccatattggata

SEQ ID NO: 260 - RNA, 16 nt, synthetic construct
  source 1..16: mol_type = other RNA
tctccatattggataa

SEQ ID NO: 261 - RNA, 16 nt, synthetic construct
  source 1..16: mol_type = other RNA
ctccatattggataaa

SEQ ID NO: 262 - RNA, 16 nt, synthetic construct
  source 1..16: mol_type = other RNA
tccatattggataaaa

SEQ ID NO: 263 - RNA, 16 nt, synthetic construct
  source 1..16: mol_type = other RNA
catattggataaaata

SEQ ID NO: 264 - RNA, 16 nt, synthetic construct
  source 1..16: mol_type = other RNA
tattggataaaattca

SEQ ID NO: 265 - RNA, 16 nt, synthetic construct
  source 1..16: mol_type = other RNA
tccttttgtagatcta

SEQ ID NO: 266 - RNA, 16 nt, synthetic construct
  source 1..16: mol_type = other RNA
ccttttgtagatctta

SEQ ID NO: 267 - RNA, 16 nt, synthetic construct
  source 1..16: mol_type = other RNA
cttttgtagatcttga

SEQ ID NO: 268 - RNA, 16 nt, synthetic construct
  source 1..16: mol_type = other RNA
ttttgtagatcttgca

SEQ ID NO: 269 - RNA, 16 nt, synthetic construct
  source 1..16: mol_type = other RNA
tttgtagatcttgcaa

SEQ ID NO: 270 - RNA, 16 nt, synthetic construct
  source 1..16: mol_type = other RNA
ttgtagatcttgcaaa

SEQ ID NO: 271 - RNA, 16 nt, synthetic construct
  source 1..16: mol_type = other RNA
tgtagatcttgcaata

SEQ ID NO: 272 - RNA, 16 nt, synthetic construct
  source 1..16: mol_type = other RNA
gtagatcttgcaatta

SEQ ID NO: 273 - RNA, 16 nt, synthetic construct
  source 1..16: mol_type = other RNA
agatcttgcaattaca

SEQ ID NO: 274 - RNA, 16 nt, synthetic construct
  source 1..16: mol_type = other RNA
gatcttgcaattacca

SEQ ID NO: 275 - RNA, 16 nt, synthetic construct
  source 1..16: mol_type = other RNA
atcttgcaattaccaa

SEQ ID NO: 276 - RNA, 16 nt, synthetic construct
  source 1..16: mol_type = other RNA
tcttgcaattaccata

SEQ ID NO: 277 - RNA, 16 nt, synthetic construct
  source 1..16: mol_type = other RNA
cttgcaattaccatta

SEQ ID NO: 278 - RNA, 16 nt, synthetic construct
  source 1..16: mol_type = other RNA
ttgcaattaccattta

SEQ ID NO: 279 - RNA, 16 nt, synthetic construct
  source 1..16: mol_type = other RNA
tgcaattaccatttga

SEQ ID NO: 280 - RNA, 16 nt, synthetic construct
  source 1..16: mol_type = other RNA
attaccatttgcataa

SEQ ID NO: 281 - RNA, 16 nt, synthetic construct
  source 1..16: mol_type = other RNA
ttaccatttgcataga

SEQ ID NO: 282 - RNA, 16 nt, synthetic construct
  source 1..16: mol_type = other RNA
accatttgcatagtta

SEQ ID NO: 283 - RNA, 16 nt, synthetic construct
  source 1..16: mol_type = other RNA
tgcatagttttaaaca

SEQ ID NO: 284 - RNA, 16 nt, synthetic construct
  source 1..16: mol_type = other RNA
atgagtatttccaaga

SEQ ID NO: 285 - RNA, 16 nt, synthetic construct
  source 1..16: mol_type = other RNA
tgagtatttccaagta

SEQ ID NO: 286 - RNA, 16 nt, synthetic construct
  source 1..16: mol_type = other RNA
gagtatttccaagtaa

SEQ ID NO: 287 - RNA, 16 nt, synthetic construct
  source 1..16: mol_type = other RNA
agtatttccaagtaga

SEQ ID NO: 288 - RNA, 16 nt, synthetic construct
  source 1..16: mol_type = other RNA
gtatttccaagtagga

SEQ ID NO: 289 - RNA, 16 nt, synthetic construct
  source 1..16: mol_type = other RNA
agcagatgtggaagga

SEQ ID NO: 290 - RNA, 16 nt, synthetic construct
  source 1..16: mol_type = other RNA
gcagatgtggaaggaa

SEQ ID NO: 291 - RNA, 16 nt, synthetic construct
  source 1..16: mol_type = other RNA
ctccgagtcttcaaga

SEQ ID NO: 292 - RNA, 16 nt, synthetic construct
  source 1..16: mol_type = other RNA
tccgagtcttcaagta

SEQ ID NO: 293 - RNA, 16 nt, synthetic construct
  source 1..16: mol_type = other RNA
ccgagtcttcaagtta

SEQ ID NO: 294 - RNA, 16 nt, synthetic construct
  source 1..16: mol_type = other RNA
cgagtcttcaagttga

SEQ ID NO: 295 - RNA, 16 nt, synthetic construct
  source 1..16: mol_type = other RNA
gtgccttattgtttaa

SEQ ID NO: 296 - RNA, 16 nt, synthetic construct
  source 1..16: mol_type = other RNA
tgccttattgtttaca

SEQ ID NO: 297 - RNA, 16 nt, synthetic construct
  source 1..16: mol_type = other RNA
ccttattgtttacata

SEQ ID NO: 298 - RNA, 16 nt, synthetic construct
  source 1..16: mol_type = other RNA
cttattgtttacatga

SEQ ID NO: 299 - RNA, 16 nt, synthetic construct
  source 1..16: mol_type = other RNA
ttattgtttacatgaa

SEQ ID NO: 300 - RNA, 16 nt, synthetic construct
  source 1..16: mol_type = other RNA
tattgtttacatgata

SEQ ID NO: 301 - RNA, 16 nt, synthetic construct
  source 1..16: mol_type = other RNA
attgtttacatgatga

SEQ ID NO: 302 - RNA, 16 nt, synthetic construct
  source 1..16: mol_type = other RNA
ttgtttacatgatgga

SEQ ID NO: 303 - RNA, 16 nt, synthetic construct
  source 1..16: mol_type = other RNA
tgtttacatgatggta

SEQ ID NO: 304 - RNA, 16 nt, synthetic construct
  source 1..16: mol_type = other RNA
gtttacatgatggtca

SEQ ID NO: 305 - RNA, 16 nt, synthetic construct
  source 1..16: mol_type = other RNA
tttacatgatggtcaa

SEQ ID NO: 306 - RNA, 16 nt, synthetic construct
  source 1..16: mol_type = other RNA
acaatcttacagcaaa

SEQ ID NO: 307 - RNA, 16 nt, synthetic construct
  source 1..16: mol_type = other RNA
caatcttacagcaata

SEQ ID NO: 308 - RNA, 16 nt, synthetic construct
  source 1..16: mol_type = other RNA
aatcttacagcaatta

SEQ ID NO: 309 - RNA, 16 nt, synthetic construct
  source 1..16: mol_type = other RNA
atcttacagcaattga

SEQ ID NO: 310 - RNA, 16 nt, synthetic construct
  source 1..16: mol_type = other RNA
tcttacagcaattgaa

SEQ ID NO: 311 - RNA, 16 nt, synthetic construct
  source 1..16: mol_type = other RNA
atgcaaacaacctcca

SEQ ID NO: 312 - RNA, 16 nt, synthetic construct
  source 1..16: mol_type = other RNA
gcaaacaacctccaga

SEQ ID NO: 313 - RNA, 16 nt, synthetic construct
  source 1..16: mol_type = other RNA
caaacaacctccagaa

SEQ ID NO: 314 - RNA, 16 nt, synthetic construct
  source 1..16: mol_type = other RNA
aaacaacctccagata

SEQ ID NO: 315 - RNA, 16 nt, synthetic construct
  source 1..16: mol_type = other RNA
ccagcctcacagtgaa

SEQ ID NO: 316 - RNA, 16 nt, synthetic construct
  source 1..16: mol_type = other RNA
cagcctcacagtgaca

SEQ ID NO: 317 - RNA, 16 nt, synthetic construct
  source 1..16: mol_type = other RNA
ggtttgaaagcttcaa

SEQ ID NO: 318 - RNA, 16 nt, synthetic construct
  source 1..16: mol_type = other RNA
gtttgaaagcttcata

SEQ ID NO: 319 - RNA, 16 nt, synthetic construct
  source 1..16: mol_type = other RNA
cattaagattatccta

SEQ ID NO: 320 - RNA, 16 nt, synthetic construct
  source 1..16: mol_type = other RNA
attaagattatcctga

SEQ ID NO: 321 - RNA, 16 nt, synthetic construct
  source 1..16: mol_type = other RNA
ttaagattatcctgga

SEQ ID NO: 322 - RNA, 16 nt, synthetic construct
  source 1..16: mol_type = other RNA
taagattatcctggaa

SEQ ID NO: 323 - RNA, 16 nt, synthetic construct
  source 1..16: mol_type = other RNA
aagattatcctggaga

SEQ ID NO: 324 - RNA, 16 nt, synthetic construct
  source 1..16: mol_type = other RNA
agattatcctggagta

SEQ ID NO: 325 - RNA, 16 nt, synthetic construct
  source 1..16: mol_type = other RNA
gattatcctggagtaa

SEQ ID NO: 326 - RNA, 16 nt, synthetic construct
  source 1..16: mol_type = other RNA
attatcctggagtata

SEQ ID NO: 327 - RNA, 16 nt, synthetic construct
  source 1..16: mol_type = other RNA
tatcctggagtatgca

SEQ ID NO: 328 - RNA, 16 nt, synthetic construct
  source 1..16: mol_type = other RNA
atcttcattctggaaa

SEQ ID NO: 329 - RNA, 16 nt, synthetic construct
  source 1..16: mol_type = other RNA
acactcttggctacta

SEQ ID NO: 330 - RNA, 16 nt, synthetic construct
  source 1..16: mol_type = other RNA
cactcttggctactca

SEQ ID NO: 331 - RNA, 16 nt, synthetic construct
  source 1..16: mol_type = other RNA
actcttggctactcaa

SEQ ID NO: 332 - RNA, 16 nt, synthetic construct
  source 1..16: mol_type = other RNA
ctcttggctactcaga

SEQ ID NO: 333 - RNA, 16 nt, synthetic construct
  source 1..16: mol_type = other RNA
ctagatttgaaggaaa

SEQ ID NO: 334 - RNA, 16 nt, synthetic construct
  source 1..16: mol_type = other RNA
agatttgaaggaatga

SEQ ID NO: 335 - RNA, 16 nt, synthetic construct
  source 1..16: mol_type = other RNA
gatttgaaggaatgaa

SEQ ID NO: 336 - RNA, 16 nt, synthetic construct
  source 1..16: mol_type = other RNA
cactcataggagcaaa

SEQ ID NO: 337 - RNA, 16 nt, synthetic construct
  source 1..16: mol_type = other RNA
actcataggagcaata

SEQ ID NO: 338 - RNA, 16 nt, synthetic construct
  source 1..16: mol_type = other RNA
ggcaagttctatgaga

SEQ ID NO: 339 - RNA, 16 nt, synthetic construct
  source 1..16: mol_type = other RNA
gcaagttctatgagta

SEQ ID NO: 340 - RNA, 16 nt, synthetic construct
  source 1..16: mol_type = other RNA
caagttctatgagtga

SEQ ID NO: 341 - RNA, 16 nt, synthetic construct
  source 1..16: mol_type = other RNA
aagttctatgagtgta

SEQ ID NO: 342 - RNA, 16 nt, synthetic construct
  source 1..16: mol_type = other RNA
aatgtgcgatggaaaa

SEQ ID NO: 343 - RNA, 16 nt, synthetic construct
  source 1..16: mol_type = other RNA
tgcttcaagttgcaaa

SEQ ID NO: 344 - RNA, 16 nt, synthetic construct
  source 1..16: mol_type = other RNA
gcttcaagttgcaaca

SEQ ID NO: 345 - RNA, 16 nt, synthetic construct
  source 1..16: mol_type = other RNA
ttatgtatgcagcaga

SEQ ID NO: 346 - RNA, 16 nt, synthetic construct
  source 1..16: mol_type = other RNA
tatgtatgcagcagta

SEQ ID NO: 347 - RNA, 16 nt, synthetic construct
  source 1..16: mol_type = other RNA
ataatttcaaccaaca

SEQ ID NO: 348 - RNA, 16 nt, synthetic construct
  source 1..16: mol_type = other RNA
aatttcaaccaacaga

SEQ ID NO: 349 - RNA, 16 nt, synthetic construct
  source 1..16: mol_type = other RNA
atttcaaccaacagaa

SEQ ID NO: 350 - RNA, 16 nt, synthetic construct
  source 1..16: mol_type = other RNA
tttcaaccaacagaaa

SEQ ID NO: 351 - RNA, 16 nt, synthetic construct
  source 1..16: mol_type = other RNA
agcttggaggtcaaga

SEQ ID NO: 352 - RNA, 16 nt, synthetic construct
  source 1..16: mol_type = other RNA
gcttggaggtcaagaa

SEQ ID NO: 353 - RNA, 16 nt, synthetic construct
  source 1..16: mol_type = other RNA
aaggatgtatatttga

SEQ ID NO: 354 - RNA, 16 nt, synthetic construct
  source 1..16: mol_type = other RNA
ggatgtatatttgaca

SEQ ID NO: 355 - RNA, 16 nt, synthetic construct
  source 1..16: mol_type = other RNA
tcaacatggtaaccaa

SEQ ID NO: 356 - RNA, 16 nt, synthetic construct
  source 1..16: mol_type = other RNA
caacatggtaaccata

SEQ ID NO: 357 - RNA, 16 nt, synthetic construct
  source 1..16: mol_type = other RNA
accatgatggtagaaa

SEQ ID NO: 358 - RNA, 16 nt, synthetic construct
  source 1..16: mol_type = other RNA
ccatgatggtagaaaa

SEQ ID NO: 359 - RNA, 16 nt, synthetic construct
  source 1..16: mol_type = other RNA
ggaattaatgacatga

SEQ ID NO: 360 - RNA, 16 nt, synthetic construct
  source 1..16: mol_type = other RNA
gaattaatgacatgta

SEQ ID NO: 361 - RNA, 16 nt, synthetic construct
  source 1..16: mol_type = other RNA
aattaatgacatgtta

SEQ ID NO: 362 - RNA, 16 nt, synthetic construct
  source 1..16: mol_type = other RNA
attaatgacatgttca

SEQ ID NO: 363 - RNA, 16 nt, synthetic construct
  source 1..16: mol_type = other RNA
ttaatgacatgttcaa

SEQ ID NO: 364 - RNA, 16 nt, synthetic construct
  source 1..16: mol_type = other RNA
taatgacatgttcaaa

SEQ ID NO: 365 - RNA, 16 nt, synthetic construct
  source 1..16: mol_type = other RNA
atggattctcttcgta

SEQ ID NO: 366 - RNA, 16 nt, synthetic construct
  source 1..16: mol_type = other RNA
tggattctcttcgtta

SEQ ID NO: 367 - RNA, 16 nt, synthetic construct
  source 1..16: mol_type = other RNA
ggattctcttcgttca

SEQ ID NO: 368 - RNA, 16 nt, synthetic construct
  source 1..16: mol_type = other RNA
gattctcttcgttcaa

SEQ ID NO: 369 - RNA, 16 nt, synthetic construct
  source 1..16: mol_type = other RNA
attctcttcgttcaca

SEQ ID NO: 370 - RNA, 16 nt, synthetic construct
  source 1..16: mol_type = other RNA
ttctcttcgttcacaa

SEQ ID NO: 371 - RNA, 16 nt, synthetic construct
  source 1..16: mol_type = other RNA
ttcgttcacagatgga

SEQ ID NO: 372 - RNA, 16 nt, synthetic construct
  source 1..16: mol_type = other RNA
tcgttcacagatggaa

SEQ ID NO: 373 - RNA, 16 nt, synthetic construct
  source 1..16: mol_type = other RNA
cgttcacagatggaaa

SEQ ID NO: 374 - RNA, 16 nt, synthetic construct
  source 1..16: mol_type = other RNA
gttcacagatggaaga

SEQ ID NO: 375 - RNA, 16 nt, synthetic construct
  source 1..16: mol_type = other RNA
ttcacagatggaagaa

SEQ ID NO: 376 - RNA, 16 nt, synthetic construct
  source 1..16: mol_type = other RNA
cacagatggaagaaaa

SEQ ID NO: 377 - RNA, 16 nt, synthetic construct
  source 1..16: mol_type = other RNA
acagatggaagaaaga

SEQ ID NO: 378 - RNA, 16 nt, synthetic construct
  source 1..16: mol_type = other RNA
cagatggaagaaagga

SEQ ID NO: 379 - RNA, 16 nt, synthetic construct
  source 1..16: mol_type = other RNA
agatggaagaaaggta

SEQ ID NO: 380 - RNA, 16 nt, synthetic construct
  source 1..16: mol_type = other RNA
gatggaagaaaggtta

SEQ ID NO: 381 - RNA, 16 nt, synthetic construct
  source 1..16: mol_type = other RNA
atggaagaaaggttca

SEQ ID NO: 382 - RNA, 16 nt, synthetic construct
  source 1..16: mol_type = other RNA
ggaagaaaggttcata

SEQ ID NO: 383 - RNA, 16 nt, synthetic construct
  source 1..16: mol_type = other RNA
aagaaaggttcatgta

SEQ ID NO: 384 - RNA, 16 nt, synthetic construct
  source 1..16: mol_type = other RNA
ctatgaacccatcaca

SEQ ID NO: 385 - RNA, 21 nt, Homo sapiens
  source 1..21: mol_type = mRNA
gtctcttgccctcattgaaca

SEQ ID NO: 386 - RNA, 21 nt, Homo sapiens
  source 1..21: mol_type = mRNA
tctcttgccctcattgaacaa

SEQ ID NO: 387 - RNA, 21 nt, Homo sapiens
  source 1..21: mol_type = mRNA
ttcatagtattgaacaaaggg

SEQ ID NO: 388 - RNA, 21 nt, Homo sapiens
  source 1..21: mol_type = mRNA
tcatagtattgaacaaaggga

SEQ ID NO: 389 - RNA, 21 nt, Homo sapiens
  source 1..21: mol_type = mRNA
catagtattgaacaaagggaa

SEQ ID NO: 390 - RNA, 21 nt, Homo sapiens
  source 1..21: mol_type = mRNA
atagtattgaacaaagggaaa

SEQ ID NO: 391 - RNA, 21 nt, Homo sapiens
  source 1..21: mol_type = mRNA
tagtattgaacaaagggaaaa

SEQ ID NO: 392 - RNA, 21 nt, Homo sapiens
  source 1..21: mol_type = mRNA
aaaacaatcttccgtttcaat

SEQ ID NO: 393 - RNA, 21 nt, Homo sapiens
  source 1..21: mol_type = mRNA
tcctttcagtcctctaagaag

SEQ ID NO: 394 - RNA, 21 nt, Homo sapiens
  source 1..21: mol_type = mRNA
cctttcagtcctctaagaaga

SEQ ID NO: 395 - RNA, 21 nt, Homo sapiens
  source 1..21: mol_type = mRNA
tttcagtcctctaagaagaat

SEQ ID NO: 396 - RNA, 21 nt, Homo sapiens
  source 1..21: mol_type = mRNA
ttcagtcctctaagaagaata

SEQ ID NO: 397 - RNA, 21 nt, Homo sapiens
  source 1..21: mol_type = mRNA
cagtcctctaagaagaatatc

SEQ ID NO: 398 - RNA, 21 nt, Homo sapiens
  source 1..21: mol_type = mRNA
agtcctctaagaagaatatct

SEQ ID NO: 399 - RNA, 21 nt, Homo sapiens
  source 1..21: mol_type = mRNA
gtcctctaagaagaatatcta

SEQ ID NO: 400 - RNA, 21 nt, Homo sapiens
  source 1..21: mol_type = mRNA
tcctctaagaagaatatctat

SEQ ID NO: 401 - RNA, 21 nt, Homo sapiens
  source 1..21: mol_type = mRNA
cctctaagaagaatatctatt

SEQ ID NO: 402 - RNA, 21 nt, Homo sapiens
  source 1..21: mol_type = mRNA
agtacactccttattcagcat

SEQ ID NO: 403 - RNA, 21 nt, Homo sapiens
  source 1..21: mol_type = mRNA
gtacactccttattcagcatg

SEQ ID NO: 404 - RNA, 21 nt, Homo sapiens
  source 1..21: mol_type = mRNA
tacactccttattcagcatgc

SEQ ID NO: 405 - RNA, 21 nt, Homo sapiens
  source 1..21: mol_type = mRNA
acactccttattcagcatgct

SEQ ID NO: 406 - RNA, 21 nt, Homo sapiens
  source 1..21: mol_type = mRNA
cactccttattcagcatgctc

SEQ ID NO: 407 - RNA, 21 nt, Homo sapiens
  source 1..21: mol_type = mRNA
tgtcgagtacacttttactgg

SEQ ID NO: 408 - RNA, 21 nt, Homo sapiens
  source 1..21: mol_type = mRNA
gtcgagtacacttttactgga

SEQ ID NO: 409 - RNA, 21 nt, Homo sapiens
  source 1..21: mol_type = mRNA
aaaatccttgcaagaggcttc

SEQ ID NO: 410 - RNA, 21 nt, Homo sapiens
  source 1..21: mol_type = mRNA
tgcgtatttaacagaatttgt

SEQ ID NO: 411 - RNA, 21 nt, Homo sapiens
  source 1..21: mol_type = mRNA
gcgtatttaacagaatttgta

SEQ ID NO: 412 - RNA, 21 nt, Homo sapiens
  source 1..21: mol_type = mRNA
gaaaactatttctgtaatccc

SEQ ID NO: 413 - RNA, 21 nt, Homo sapiens
  source 1..21: mol_type = mRNA
aaaactatttctgtaatccca

SEQ ID NO: 414 - RNA, 21 nt, Homo sapiens
  source 1..21: mol_type = mRNA
tctgagtgtgtttgcactaat

SEQ ID NO: 415 - RNA, 21 nt, Homo sapiens
  source 1..21: mol_type = mRNA
ctgagtgtgtttgcactaatt

SEQ ID NO: 416 - RNA, 21 nt, Homo sapiens
  source 1..21: mol_type = mRNA
gagtgtgtttgcactaattgg

SEQ ID NO: 417 - RNA, 21 nt, Homo sapiens
  source 1..21: mol_type = mRNA
agtgtgtttgcactaattgga

SEQ ID NO: 418 - RNA, 21 nt, Homo sapiens
  source 1..21: mol_type = mRNA
tgtttgcactaattggactac

SEQ ID NO: 419 - RNA, 21 nt, Homo sapiens
  source 1..21: mol_type = mRNA
gtttgcactaattggactaca

SEQ ID NO: 420 - RNA, 21 nt, Homo sapiens
  source 1..21: mol_type = mRNA
tttgcactaattggactacag

SEQ ID NO: 421 - RNA, 21 nt, Homo sapiens
  source 1..21: mol_type = mRNA
ttgcactaattggactacagc

SEQ ID NO: 422 - RNA, 21 nt, Homo sapiens
  source 1..21: mol_type = mRNA
tgcactaattggactacagct

SEQ ID NO: 423 - RNA, 21 nt, Homo sapiens
  source 1..21: mol_type = mRNA
cactaattggactacagctgt

SEQ ID NO: 424 - RNA, 21 nt, Homo sapiens
  source 1..21: mol_type = mRNA
ggaaggatccaaagatgctct

SEQ ID NO: 425 - RNA, 21 nt, Homo sapiens
  source 1..21: mol_type = mRNA
gaaggatccaaagatgctctc

SEQ ID NO: 426 - RNA, 21 nt, Homo sapiens
  source 1..21: mol_type = mRNA
tggtttcagcacagattcagg

SEQ ID NO: 427 - RNA, 21 nt, Homo sapiens
  source 1..21: mol_type = mRNA
ggtttcagcacagattcaggt

SEQ ID NO: 428 - RNA, 21 nt, Homo sapiens
  source 1..21: mol_type = mRNA
ctgattatggctacacgagct

SEQ ID NO: 429 - RNA, 21 nt, Homo sapiens
  source 1..21: mol_type = mRNA
cattgaagaagctaaacagaa

SEQ ID NO: 430 - RNA, 21 nt, Homo sapiens
  source 1..21: mol_type = mRNA
attgaagaagctaaacagaaa

SEQ ID NO: 431 - RNA, 21 nt, Homo sapiens
  source 1..21: mol_type = mRNA
ttgaagaagctaaacagaaag

SEQ ID NO: 432 - RNA, 21 nt, Homo sapiens
  source 1..21: mol_type = mRNA
tgaagaagctaaacagaaaga

SEQ ID NO: 433 - RNA, 21 nt, Homo sapiens
  source 1..21: mol_type = mRNA
gtcaccactcagcattcgtgg

SEQ ID NO: 434 - RNA, 21 nt, Homo sapiens
  source 1..21: mol_type = mRNA
tcaccactcagcattcgtggc

SEQ ID NO: 435 - RNA, 21 nt, Homo sapiens
  source 1..21: mol_type = mRNA
gcgacgcagcagtaacatcag

SEQ ID NO: 436 - RNA, 21 nt, Homo sapiens
  source 1..21: mol_type = mRNA
gacgctcagccctcatgctcc

SEQ ID NO: 437 - RNA, 21 nt, Homo sapiens
  source 1..21: mol_type = mRNA
acgctcagccctcatgctccc

SEQ ID NO: 438 - RNA, 21 nt, Homo sapiens
  source 1..21: mol_type = mRNA
cgctcagccctcatgctcccc

SEQ ID NO: 439 - RNA, 21 nt, Homo sapiens
  source 1..21: mol_type = mRNA
aaacactgtggaagaacttga

SEQ ID NO: 440 - RNA, 21 nt, Homo sapiens
  source 1..21: mol_type = mRNA
aacactgtggaagaacttgaa

SEQ ID NO: 441 - RNA, 21 nt, Homo sapiens
  source 1..21: mol_type = mRNA
accttggtggtacagatttgc

SEQ ID NO: 442 - RNA, 21 nt, Homo sapiens
  source 1..21: mol_type = mRNA
ccttggtggtacagatttgca

SEQ ID NO: 443 - RNA, 21 nt, Homo sapiens
  source 1..21: mol_type = mRNA
cttgatctggaattgctctcc

SEQ ID NO: 444 - RNA, 21 nt, Homo sapiens
  source 1..21: mol_type = mRNA
ttgatctggaattgctctcca

SEQ ID NO: 445 - RNA, 21 nt, Homo sapiens
  source 1..21: mol_type = mRNA
gatctggaattgctctccata

SEQ ID NO: 446 - RNA, 21 nt, Homo sapiens
  source 1..21: mol_type = mRNA
atctggaattgctctccatat

SEQ ID NO: 447 - RNA, 21 nt, Homo sapiens
  source 1..21: mol_type = mRNA
tctggaattgctctccatatt

SEQ ID NO: 448 - RNA, 21 nt, Homo sapiens
  source 1..21: mol_type = mRNA
ctggaattgctctccatattg

SEQ ID NO: 449 - RNA, 21 nt, Homo sapiens
  source 1..21: mol_type = mRNA
tggaattgctctccatattgg

SEQ ID NO: 450 - RNA, 21 nt, Homo sapiens
  source 1..21: mol_type = mRNA
ggaattgctctccatattgga

SEQ ID NO: 451 - RNA, 21 nt, Homo sapiens
  source 1..21: mol_type = mRNA
aattgctctccatattggata

SEQ ID NO: 452 - RNA, 21 nt, Homo sapiens
  source 1..21: mol_type = mRNA
attgctctccatattggataa

SEQ ID NO: 453 - RNA, 21 nt, Homo sapiens
  source 1..21: mol_type = mRNA
ttgctctccatattggataaa

SEQ ID NO: 454 - RNA, 21 nt, Homo sapiens
  source 1..21: mol_type = mRNA
tgctctccatattggataaaa

SEQ ID NO: 455 - RNA, 21 nt, Homo sapiens
  source 1..21: mol_type = mRNA
ctctccatattggataaaatt

SEQ ID NO: 456 - RNA, 21 nt, Homo sapiens
  source 1..21: mol_type = mRNA
ctccatattggataaaattca

SEQ ID NO: 457 - RNA, 21 nt, Homo sapiens
  source 1..21: mol_type = mRNA
atggatccttttgtagatctt

SEQ ID NO: 458 - RNA, 21 nt, Homo sapiens
  source 1..21: mol_type = mRNA
tggatccttttgtagatcttg

SEQ ID NO: 459 - RNA, 21 nt, Homo sapiens
  source 1..21: mol_type = mRNA
ggatccttttgtagatcttgc

SEQ ID NO: 460 - RNA, 21 nt, Homo sapiens
  source 1..21: mol_type = mRNA
gatccttttgtagatcttgca

SEQ ID NO: 461 - RNA, 21 nt, Homo sapiens
  source 1..21: mol_type = mRNA
atccttttgtagatcttgcaa

SEQ ID NO: 462 - RNA, 21 nt, Homo sapiens
  source 1..21: mol_type = mRNA
tccttttgtagatcttgcaat

SEQ ID NO: 463 - RNA, 21 nt, Homo sapiens
  source 1..21: mol_type = mRNA
ccttttgtagatcttgcaatt

SEQ ID NO: 464 - RNA, 21 nt, Homo sapiens
  source 1..21: mol_type = mRNA
cttttgtagatcttgcaatta

SEQ ID NO: 465 - RNA, 21 nt, Homo sapiens
  source 1..21: mol_type = mRNA
tttgtagatcttgcaattacc

SEQ ID NO: 466 - RNA, 21 nt, Homo sapiens
  source 1..21: mol_type = mRNA
ttgtagatcttgcaattacca

SEQ ID NO: 467 - RNA, 21 nt, Homo sapiens
  source 1..21: mol_type = mRNA
tgtagatcttgcaattaccat

SEQ ID NO: 468 - RNA, 21 nt, Homo sapiens
  source 1..21: mol_type = mRNA
gtagatcttgcaattaccatt

SEQ ID NO: 469 - RNA, 21 nt, Homo sapiens
  source 1..21: mol_type = mRNA
tagatcttgcaattaccattt

SEQ ID NO: 470 - RNA, 21 nt, Homo sapiens
  source 1..21: mol_type = mRNA
agatcttgcaattaccatttg

SEQ ID NO: 471 - RNA, 21 nt, Homo sapiens
  source 1..21: mol_type = mRNA
gatcttgcaattaccatttgc

SEQ ID NO: 472 - RNA, 21 nt, Homo sapiens
  source 1..21: mol_type = mRNA
ttgcaattaccatttgcatag

SEQ ID NO: 473 - RNA, 21 nt, Homo sapiens
  source 1..21: mol_type = mRNA
tgcaattaccatttgcatagt

SEQ ID NO: 474 - RNA, 21 nt, Homo sapiens
  source 1..21: mol_type = mRNA
caattaccatttgcatagttt

SEQ ID NO: 475 - RNA, 21 nt, Homo sapiens
  source 1..21: mol_type = mRNA
ccatttgcatagttttaaaca

SEQ ID NO: 476 - RNA, 21 nt, Homo sapiens
  source 1..21: mol_type = mRNA
tccatatgagtatttccaagt

SEQ ID NO: 477 - RNA, 21 nt, Homo sapiens
  source 1..21: mol_type = mRNA
ccatatgagtatttccaagta

SEQ ID NO: 478 - RNA, 21 nt, Homo sapiens
  source 1..21: mol_type = mRNA
catatgagtatttccaagtag

SEQ ID NO: 479 - RNA, 21 nt, Homo sapiens
  source 1..21: mol_type = mRNA
atatgagtatttccaagtagg

SEQ ID NO: 480 - RNA, 21 nt, Homo sapiens
  source 1..21: mol_type = mRNA
tatgagtatttccaagtaggc

SEQ ID NO: 481 - RNA, 21 nt, Homo sapiens
  source 1..21: mol_type = mRNA
tttctagcagatgtggaagga

SEQ ID NO: 482 - RNA, 21 nt, Homo sapiens
  source 1..21: mol_type = mRNA
ttctagcagatgtggaaggat

SEQ ID NO: 483 - RNA, 21 nt, Homo sapiens
  source 1..21: mol_type = mRNA
gactgctccgagtcttcaagt

SEQ ID NO: 484 - RNA, 21 nt, Homo sapiens
  source 1..21: mol_type = mRNA
actgctccgagtcttcaagtt

SEQ ID NO: 485 - RNA, 21 nt, Homo sapiens
  source 1..21: mol_type = mRNA
ctgctccgagtcttcaagttg

SEQ ID NO: 486 - RNA, 21 nt, Homo sapiens
  source 1..21: mol_type = mRNA
tgctccgagtcttcaagttgg

SEQ ID NO: 487 - RNA, 21 nt, Homo sapiens
  source 1..21: mol_type = mRNA
gctatgtgccttattgtttac

SEQ ID NO: 488 - RNA, 21 nt, Homo sapiens
  source 1..21: mol_type = mRNA
ctatgtgccttattgtttaca

SEQ ID NO: 489 - RNA, 21 nt, Homo sapiens
  source 1..21: mol_type = mRNA
atgtgccttattgtttacatg

SEQ ID NO: 490 - RNA, 21 nt, Homo sapiens
  source 1..21: mol_type = mRNA
tgtgccttattgtttacatga

SEQ ID NO: 491 - RNA, 21 nt, Homo sapiens
  source 1..21: mol_type = mRNA
gtgccttattgtttacatgat

SEQ ID NO: 492 - RNA, 21 nt, Homo sapiens
  source 1..21: mol_type = mRNA
tgccttattgtttacatgatg

SEQ ID NO: 493 - RNA, 21 nt, Homo sapiens
  source 1..21: mol_type = mRNA
gccttattgtttacatgatgg

SEQ ID NO: 494 - RNA, 21 nt, Homo sapiens
  source 1..21: mol_type = mRNA
ccttattgtttacatgatggt

SEQ ID NO: 495 - RNA, 21 nt, Homo sapiens
  source 1..21: mol_type = mRNA
cttattgtttacatgatggtc

SEQ ID NO: 496 - RNA, 21 nt, Homo sapiens
  source 1..21: mol_type = mRNA
ttattgtttacatgatggtca

SEQ ID NO: 497 - RNA, 21 nt, Homo sapiens
  source 1..21: mol_type = mRNA
tattgtttacatgatggtcat

SEQ ID NO: 498 - RNA, 21 nt, Homo sapiens
  source 1..21: mol_type = mRNA
ttcagacaatcttacagcaat

SEQ ID NO: 499 - RNA, 21 nt, Homo sapiens
  source 1..21: mol_type = mRNA
tcagacaatcttacagcaatt

SEQ ID NO: 500 - RNA, 21 nt, Homo sapiens
  source 1..21: mol_type = mRNA
cagacaatcttacagcaattg

SEQ ID NO: 501 - RNA, 21 nt, Homo sapiens
  source 1..21: mol_type = mRNA
agacaatcttacagcaattga

SEQ ID NO: 502 - RNA, 21 nt, Homo sapiens
  source 1..21: mol_type = mRNA
gacaatcttacagcaattgaa

SEQ ID NO: 503 - RNA, 21 nt, Homo sapiens
  source 1..21: mol_type = mRNA
ccctgatgcaaacaacctcca

SEQ ID NO: 504 - RNA, 21 nt, Homo sapiens
  source 1..21: mol_type = mRNA
ctgatgcaaacaacctccaga

SEQ ID NO: 505 - RNA, 21 nt, Homo sapiens
  source 1..21: mol_type = mRNA
tgatgcaaacaacctccagat

SEQ ID NO: 506 - RNA, 21 nt, Homo sapiens
  source 1..21: mol_type = mRNA
gatgcaaacaacctccagatt

SEQ ID NO: 507 - RNA, 21 nt, Homo sapiens
  source 1..21: mol_type = mRNA
caatcccagcctcacagtgac

SEQ ID NO: 508 - RNA, 21 nt, Homo sapiens
  source 1..21: mol_type = mRNA
aatcccagcctcacagtgaca

SEQ ID NO: 509 - RNA, 21 nt, Homo sapiens
  source 1..21: mol_type = mRNA
cagttggtttgaaagcttcat

SEQ ID NO: 510 - RNA, 21 nt, Homo sapiens
  source 1..21: mol_type = mRNA
agttggtttgaaagcttcatt

SEQ ID NO: 511 - RNA, 21 nt, Homo sapiens
  source 1..21: mol_type = mRNA
aagaccattaagattatcctg

SEQ ID NO: 512 - RNA, 21 nt, Homo sapiens
  source 1..21: mol_type = mRNA
agaccattaagattatcctgg

SEQ ID NO: 513 - RNA, 21 nt, Homo sapiens
  source 1..21: mol_type = mRNA
gaccattaagattatcctgga

SEQ ID NO: 514 - RNA, 21 nt, Homo sapiens
  source 1..21: mol_type = mRNA
accattaagattatcctggag

SEQ ID NO: 515 - RNA, 21 nt, Homo sapiens
  source 1..21: mol_type = mRNA
ccattaagattatcctggagt

SEQ ID NO: 516 - RNA, 21 nt, Homo sapiens
  source 1..21: mol_type = mRNA
cattaagattatcctggagta

SEQ ID NO: 517 - RNA, 21 nt, Homo sapiens
  source 1..21: mol_type = mRNA
attaagattatcctggagtat

SEQ ID NO: 518 - RNA, 21 nt, Homo sapiens
  source 1..21: mol_type = mRNA
ttaagattatcctggagtatg

SEQ ID NO: 519 - RNA, 21 nt, Homo sapiens
  source 1..21: mol_type = mRNA
aagattatcctggagtatgca

SEQ ID NO: 520 - RNA, 21 nt, Homo sapiens
  source 1..21: mol_type = mRNA
cttacatcttcattctggaaa

SEQ ID NO: 521 - RNA, 21 nt, Homo sapiens
  source 1..21: mol_type = mRNA
ggcaaacactcttggctactc

SEQ ID NO: 522 - RNA, 21 nt, Homo sapiens
  source 1..21: mol_type = mRNA
gcaaacactcttggctactca

SEQ ID NO: 523 - RNA, 21 nt, Homo sapiens
  source 1..21: mol_type = mRNA
caaacactcttggctactcag

SEQ ID NO: 524 - RNA, 21 nt, Homo sapiens
  source 1..21: mol_type = mRNA
aaacactcttggctactcaga

SEQ ID NO: 525 - RNA, 21 nt, Homo sapiens
  source 1..21: mol_type = mRNA
cttatctagatttgaaggaat

SEQ ID NO: 526 - RNA, 21 nt, Homo sapiens
  source 1..21: mol_type = mRNA
tatctagatttgaaggaatga

SEQ ID NO: 527 - RNA, 21 nt, Homo sapiens
  source 1..21: mol_type = mRNA
atctagatttgaaggaatgag

SEQ ID NO: 528 - RNA, 21 nt, Homo sapiens
  source 1..21: mol_type = mRNA
gaatgcactcataggagcaat

SEQ ID NO: 529 - RNA, 21 nt, Homo sapiens
  source 1..21: mol_type = mRNA
aatgcactcataggagcaatt

SEQ ID NO: 530 - RNA, 21 nt, Homo sapiens
  source 1..21: mol_type = mRNA
ttgctggcaagttctatgagt

SEQ ID NO: 531 - RNA, 21 nt, Homo sapiens
  source 1..21: mol_type = mRNA
tgctggcaagttctatgagtg

SEQ ID NO: 532 - RNA, 21 nt, Homo sapiens
  source 1..21: mol_type = mRNA
gctggcaagttctatgagtgt

SEQ ID NO: 533 - RNA, 21 nt, Homo sapiens
  source 1..21: mol_type = mRNA
ctggcaagttctatgagtgta

SEQ ID NO: 534 - RNA, 21 nt, Homo sapiens
  source 1..21: mol_type = mRNA
gtcaaaatgtgcgatggaaaa

SEQ ID NO: 535 - RNA, 21 nt, Homo sapiens
  source 1..21: mol_type = mRNA
atctctgcttcaagttgcaac

SEQ ID NO: 536 - RNA, 21 nt, Homo sapiens
  source 1..21: mol_type = mRNA
tctctgcttcaagttgcaact

SEQ ID NO: 537 - RNA, 21 nt, Homo sapiens
  source 1..21: mol_type = mRNA
gattattatgtatgcagcagt

SEQ ID NO: 538 - RNA, 21 nt, Homo sapiens
  source 1..21: mol_type = mRNA
attattatgtatgcagcagtg

SEQ ID NO: 539 - RNA, 21 nt, Homo sapiens
  source 1..21: mol_type = mRNA
catagataatttcaaccaaca

SEQ ID NO: 540 - RNA, 21 nt, Homo sapiens
  source 1..21: mol_type = mRNA
tagataatttcaaccaacaga

SEQ ID NO: 541 - RNA, 21 nt, Homo sapiens
  source 1..21: mol_type = mRNA
agataatttcaaccaacagaa

SEQ ID NO: 542 - RNA, 21 nt, Homo sapiens
  source 1..21: mol_type = mRNA
gataatttcaaccaacagaaa

SEQ ID NO: 543 - RNA, 21 nt, Homo sapiens
  source 1..21: mol_type = mRNA
aaagaagcttggaggtcaaga

SEQ ID NO: 544 - RNA, 21 nt, Homo sapiens
  source 1..21: mol_type = mRNA
aagaagcttggaggtcaagac

SEQ ID NO: 545 - RNA, 21 nt, Homo sapiens
  source 1..21: mol_type = mRNA
aatccaaggatgtatatttga

SEQ ID NO: 546 - RNA, 21 nt, Homo sapiens
  source 1..21: mol_type = mRNA
tccaaggatgtatatttgacc

SEQ ID NO: 547 - RNA, 21 nt, Homo sapiens
  source 1..21: mol_type = mRNA
ctgtctcaacatggtaaccat

SEQ ID NO: 548 - RNA, 21 nt, Homo sapiens
  source 1..21: mol_type = mRNA
tgtctcaacatggtaaccatg

SEQ ID NO: 549 - RNA, 21 nt, Homo sapiens
  source 1..21: mol_type = mRNA
tggtaaccatgatggtagaaa

SEQ ID NO: 550 - RNA, 21 nt, Homo sapiens
  source 1..21: mol_type = mRNA
ggtaaccatgatggtagaaaa

SEQ ID NO: 551 - RNA, 21 nt, Homo sapiens
  source 1..21: mol_type = mRNA
aagatggaattaatgacatgt

SEQ ID NO: 552 - RNA, 21 nt, Homo sapiens
  source 1..21: mol_type = mRNA
agatggaattaatgacatgtt

SEQ ID NO: 553 - RNA, 21 nt, Homo sapiens
  source 1..21: mol_type = mRNA
gatggaattaatgacatgttc

SEQ ID NO: 554 - RNA, 21 nt, Homo sapiens
  source 1..21: mol_type = mRNA
atggaattaatgacatgttca

SEQ ID NO: 555 - RNA, 21 nt, Homo sapiens
  source 1..21: mol_type = mRNA
tggaattaatgacatgttcaa

SEQ ID NO: 556 - RNA, 21 nt, Homo sapiens
  source 1..21: mol_type = mRNA
ggaattaatgacatgttcaat

SEQ ID NO: 557 - RNA, 21 nt, Homo sapiens
  source 1..21: mol_type = mRNA
gggagatggattctcttcgtt

SEQ ID NO: 558 - RNA, 21 nt, Homo sapiens
  source 1..21: mol_type = mRNA
ggagatggattctcttcgttc

SEQ ID NO: 559 - RNA, 21 nt, Homo sapiens
  source 1..21: mol_type = mRNA
gagatggattctcttcgttca

SEQ ID NO: 560 - RNA, 21 nt, Homo sapiens
  source 1..21: mol_type = mRNA
agatggattctcttcgttcac

SEQ ID NO: 561 - RNA, 21 nt, Homo sapiens
  source 1..21: mol_type = mRNA
gatggattctcttcgttcaca

SEQ ID NO: 562 - RNA, 21 nt, Homo sapiens
  source 1..21: mol_type = mRNA
atggattctcttcgttcacag

SEQ ID NO: 563 - RNA, 21 nt, Homo sapiens
  source 1..21: mol_type = mRNA
ttctcttcgttcacagatgga

SEQ ID NO: 564 - RNA, 21 nt, Homo sapiens
  source 1..21: mol_type = mRNA
tctcttcgttcacagatggaa

SEQ ID NO: 565 - RNA, 21 nt, Homo sapiens
  source 1..21: mol_type = mRNA
ctcttcgttcacagatggaag

SEQ ID NO: 566 - RNA, 21 nt, Homo sapiens
  source 1..21: mol_type = mRNA
tcttcgttcacagatggaaga

SEQ ID NO: 567 - RNA, 21 nt, Homo sapiens
  source 1..21: mol_type = mRNA
cttcgttcacagatggaagaa

SEQ ID NO: 568 - RNA, 21 nt, Homo sapiens
  source 1..21: mol_type = mRNA
tcgttcacagatggaagaaag

SEQ ID NO: 569 - RNA, 21 nt, Homo sapiens
  source 1..21: mol_type = mRNA
cgttcacagatggaagaaagg

SEQ ID NO: 570 - RNA, 21 nt, Homo sapiens
  source 1..21: mol_type = mRNA
gttcacagatggaagaaaggt

SEQ ID NO: 571 - RNA, 21 nt, Homo sapiens
  source 1..21: mol_type = mRNA
ttcacagatggaagaaaggtt

SEQ ID NO: 572 - RNA, 21 nt, Homo sapiens
  source 1..21: mol_type = mRNA
tcacagatggaagaaaggttc

SEQ ID NO: 573 - RNA, 21 nt, Homo sapiens
  source 1..21: mol_type = mRNA
cacagatggaagaaaggttca

SEQ ID NO: 574 - RNA, 21 nt, Homo sapiens
  source 1..21: mol_type = mRNA
cagatggaagaaaggttcatg

SEQ ID NO: 575 - RNA, 21 nt, Homo sapiens
  source 1..21: mol_type = mRNA
gatggaagaaaggttcatgtc

SEQ ID NO: 576 - RNA, 21 nt, Homo sapiens
  source 1..21: mol_type = mRNA
gtgtcctatgaacccatcaca

SEQ ID NO: 577 - RNA, 21 nt, synthetic construct
  source 1..21: mol_type = other RNA
ttcttttcatcctgtatattt

SEQ ID NO: 578 - RNA, 21 nt, synthetic construct
  source 1..21: mol_type = other RNA
tttgttcaatgagggcaagag

SEQ ID NO: 579 - RNA, 21 nt, synthetic construct
  source 1..21: mol_type = other RNA
tgttgttcaatgagggcaaga

SEQ ID NO: 580 - RNA, 21 nt, synthetic construct
  source 1..21: mol_type = other RNA
tcgttgttcaatgagggcaag

SEQ ID NO: 581 - RNA, 21 nt, synthetic construct
  source 1..21: mol_type = other RNA
ttgcgttgttcaatgagggca

SEQ ID NO: 582 - RNA, 21 nt, synthetic construct
  source 1..21: mol_type = other RNA
ttttgttcaatactatgaaag

SEQ ID NO: 583 - RNA, 21 nt, synthetic construct
  source 1..21: mol_type = other RNA
tctttgttcaatactatgaaa

SEQ ID NO: 584 - RNA, 21 nt, synthetic construct
  source 1..21: mol_type = other RNA
tgttttccctttgttcaatac

SEQ ID NO: 585 - RNA, 21 nt, synthetic construct
  source 1..21: mol_type = other RNA
ttgttttccctttgttcaata

SEQ ID NO: 586 - RNA, 21 nt, synthetic construct
  source 1..21: mol_type = other RNA
taacggaagattgttttccct

SEQ ID NO: 587 - RNA, 21 nt, synthetic construct
  source 1..21: mol_type = other RNA
taaacggaagattgttttccc

SEQ ID NO: 588 - RNA, 21 nt, synthetic construct
  source 1..21: mol_type = other RNA
tgaaacggaagattgttttcc

SEQ ID NO: 589 - RNA, 21 nt, synthetic construct
  source 1..21: mol_type = other RNA
ttgaaacggaagattgttttc

SEQ ID NO: 590 - RNA, 21 nt, synthetic construct
  source 1..21: mol_type = other RNA
tattgaaacggaagattgttt

SEQ ID NO: 591 - RNA, 21 nt, synthetic construct
  source 1..21: mol_type = other RNA
tcattgaaacggaagattgtt

SEQ ID NO: 592 - RNA, 21 nt, synthetic construct
  source 1..21: mol_type = other RNA
tgcattgaaacggaagattgt

SEQ ID NO: 593 - RNA, 21 nt, synthetic construct
  source 1..21: mol_type = other RNA
tggcattgaaacggaagattg

SEQ ID NO: 594 - RNA, 21 nt, synthetic construct
  source 1..21: mol_type = other RNA
ttagaggactgaaaggagaaa

SEQ ID NO: 595 - RNA, 21 nt, synthetic construct
  source 1..21: mol_type = other RNA
tttagaggactgaaaggagaa

SEQ ID NO: 596 - RNA, 21 nt, synthetic construct
  source 1..21: mol_type = other RNA
tcttagaggactgaaaggaga

SEQ ID NO: 597 - RNA, 21 nt, synthetic construct
  source 1..21: mol_type = other RNA
ttcttagaggactgaaaggag

SEQ ID NO: 598 - RNA, 21 nt, synthetic construct
  source 1..21: mol_type = other RNA
ttcttcttagaggactgaaag

SEQ ID NO: 599 - RNA, 21 nt, synthetic construct
  source 1..21: mol_type = other RNA
ttattcttcttagaggactga

SEQ ID NO: 600 - RNA, 21 nt, synthetic construct
  source 1..21: mol_type = other RNA
taatagatattcttcttagag

SEQ ID NO: 601 - RNA, 21 nt, synthetic construct
  source 1..21: mol_type = other RNA
ttaatagatattcttcttaga

SEQ ID NO: 602 - RNA, 21 nt, synthetic construct
  source 1..21: mol_type = other RNA
tttaatagatattcttcttag

SEQ ID NO: 603 - RNA, 21 nt, synthetic construct
  source 1..21: mol_type = other RNA
tcttaatagatattcttctta

SEQ ID NO: 604 - RNA, 21 nt, synthetic construct
  source 1..21: mol_type = other RNA
ttcttaatagatattcttctt

SEQ ID NO: 605 - RNA, 21 nt, synthetic construct
  source 1..21: mol_type = other RNA
tatcttaatagatattcttct

SEQ ID NO: 606 - RNA, 21 nt, synthetic construct
  source 1..21: mol_type = other RNA
taatcttaatagatattcttc

SEQ ID NO: 607 - RNA, 21 nt, synthetic construct
  source 1..21: mol_type = other RNA
tctaaaatcttaatagatatt

SEQ ID NO: 608 - RNA, 21 nt, synthetic construct
  source 1..21: mol_type = other RNA
ttatgcagtttgtcagaatag

SEQ ID NO: 609 - RNA, 21 nt, synthetic construct
  source 1..21: mol_type = other RNA
tatatgcagtttgtcagaata

SEQ ID NO: 610 - RNA, 21 nt, synthetic construct
  source 1..21: mol_type = other RNA
taaaagtgtactcgacatttt

SEQ ID NO: 611 - RNA, 21 nt, synthetic construct
  source 1..21: mol_type = other RNA
ttaaaagtgtactcgacattt

SEQ ID NO: 612 - RNA, 21 nt, synthetic construct
  source 1..21: mol_type = other RNA
tgtaaaagtgtactcgacatt

SEQ ID NO: 613 - RNA, 21 nt, synthetic construct
  source 1..21: mol_type = other RNA
tagtaaaagtgtactcgacat

SEQ ID NO: 614 - RNA, 21 nt, synthetic construct
  source 1..21: mol_type = other RNA
ttccagtaaaagtgtactcga

SEQ ID NO: 615 - RNA, 21 nt, synthetic construct
  source 1..21: mol_type = other RNA
tttccagtaaaagtgtactcg

SEQ ID NO: 616 - RNA, 21 nt, synthetic construct
  source 1..21: mol_type = other RNA
tattccagtaaaagtgtactc

SEQ ID NO: 617 - RNA, 21 nt, synthetic construct
  source 1..21: mol_type = other RNA
ttattccagtaaaagtgtact

SEQ ID NO: 618 - RNA, 21 nt, synthetic construct
  source 1..21: mol_type = other RNA
tattctgttaaatacgcaaaa

SEQ ID NO: 619 - RNA, 21 nt, synthetic construct
  source 1..21: mol_type = other RNA
taattctgttaaatacgcaaa

SEQ ID NO: 620 - RNA, 21 nt, synthetic construct
  source 1..21: mol_type = other RNA
taaattctgttaaatacgcaa

SEQ ID NO: 621 - RNA, 21 nt, synthetic construct
  source 1..21: mol_type = other RNA
ttacaaattctgttaaatacg

SEQ ID NO: 622 - RNA, 21 nt, synthetic construct
  source 1..21: mol_type = other RNA
tttacaaattctgttaaatac

SEQ ID NO: 623 - RNA, 21 nt, synthetic construct
  source 1..21: mol_type = other RNA
tgattacagaaatagttttca

SEQ ID NO: 624 - RNA, 21 nt, synthetic construct
  source 1..21: mol_type = other RNA
tctgggattacagaaatagtt

SEQ ID NO: 625 - RNA, 21 nt, synthetic construct
  source 1..21: mol_type = other RNA
ttccaattagtgcaaacacac

SEQ ID NO: 626 - RNA, 21 nt, synthetic construct
  source 1..21: mol_type = other RNA
tgtccaattagtgcaaacaca

SEQ ID NO: 627 - RNA, 21 nt, synthetic construct
  source 1..21: mol_type = other RNA
tagtccaattagtgcaaacac

SEQ ID NO: 628 - RNA, 21 nt, synthetic construct
  source 1..21: mol_type = other RNA
tacagctgtagtccaattagt

SEQ ID NO: 629 - RNA, 21 nt, synthetic construct
  source 1..21: mol_type = other RNA
tctaatgtttcattattttca

SEQ ID NO: 630 - RNA, 21 nt, synthetic construct
  source 1..21: mol_type = other RNA
tctttctaatgtttcattatt

SEQ ID NO: 631 - RNA, 21 nt, synthetic construct
  source 1..21: mol_type = other RNA
ttgctttctaatgtttcatta

SEQ ID NO: 632 - RNA, 21 nt, synthetic construct
  source 1..21: mol_type = other RNA
tatgctttctaatgtttcatt

SEQ ID NO: 633 - RNA, 21 nt, synthetic construct
  source 1..21: mol_type = other RNA
taatctgtgctgaaaccacaa

SEQ ID NO: 634 - RNA, 21 nt, synthetic construct
  source 1..21: mol_type = other RNA
tgtgtagccataatcagggtt

SEQ ID NO: 635 - RNA, 21 nt, synthetic construct
  source 1..21: mol_type = other RNA
tcgtgtagccataatcagggt

SEQ ID NO: 636 - RNA, 21 nt, synthetic construct
  source 1..21: mol_type = other RNA
ttcgtgtagccataatcaggg

SEQ ID NO: 637 - RNA, 21 nt, synthetic construct
  source 1..21: mol_type = other RNA
tctcgtgtagccataatcagg

SEQ ID NO: 638 - RNA, 21 nt, synthetic construct
  source 1..21: mol_type = other RNA
tagtgtcaaagctcgtgtagc

SEQ ID NO: 639 - RNA, 21 nt, synthetic construct
  source 1..21: mol_type = other RNA
taagtgtcaaagctcgtgtag

SEQ ID NO: 640 - RNA, 21 nt, synthetic construct
  source 1..21: mol_type = other RNA
taaagtgtcaaagctcgtgta

SEQ ID NO: 641 - RNA, 21 nt, synthetic construct
  source 1..21: mol_type = other RNA
tgaaagtgtcaaagctcgtgt

SEQ ID NO: 642 - RNA, 21 nt, synthetic construct
  source 1..21: mol_type = other RNA
tgacttgttctgctgcttcgc

SEQ ID NO: 643 - RNA, 21 nt, synthetic construct
  source 1..21: mol_type = other RNA
tagacttgttctgctgcttcg

SEQ ID NO: 644 - RNA, 21 nt, synthetic construct
  source 1..21: mol_type = other RNA
taagttcttccacagtgtttg

SEQ ID NO: 645 - RNA, 21 nt, synthetic construct
  source 1..21: mol_type = other RNA
ttccaatatggagagcaattc

SEQ ID NO: 646 - RNA, 21 nt, synthetic construct
  source 1..21: mol_type = other RNA
taattttatccaatatggaga

SEQ ID NO: 647 - RNA, 21 nt, synthetic construct
  source 1..21: mol_type = other RNA
ttgaattttatccaatatgga

SEQ ID NO: 648 - RNA, 21 nt, synthetic construct
  source 1..21: mol_type = other RNA
tttgaattttatccaatatgg

SEQ ID NO: 649 - RNA, 21 nt, synthetic construct
  source 1..21: mol_type = other RNA
ttttgaattttatccaatatg

SEQ ID NO: 650 - RNA, 21 nt, synthetic construct
  source 1..21: mol_type = other RNA
taataaaatagatacactttt

SEQ ID NO: 651 - RNA, 21 nt, synthetic construct
  source 1..21: mol_type = other RNA
tttacaataaaatagatacac

SEQ ID NO: 652 - RNA, 21 nt, synthetic construct
  source 1..21: mol_type = other RNA
tattacaataaaatagataca

SEQ ID NO: 653 - RNA, 21 nt, synthetic construct
  source 1..21: mol_type = other RNA
tccattacaataaaatagata

SEQ ID NO: 654 - RNA, 21 nt, synthetic construct
  source 1..21: mol_type = other RNA
ttccattacaataaaatagat

SEQ ID NO: 655 - RNA, 21 nt, synthetic construct
  source 1..21: mol_type = other RNA
tatccattacaataaaataga

SEQ ID NO: 656 - RNA, 21 nt, synthetic construct
  source 1..21: mol_type = other RNA
tgatccattacaataaaatag

SEQ ID NO: 657 - RNA, 21 nt, synthetic construct
  source 1..21: mol_type = other RNA
tggatccattacaataaaata

SEQ ID NO: 658 - RNA, 21 nt, synthetic construct
  source 1..21: mol_type = other RNA
taggatccattacaataaaat

SEQ ID NO: 659 - RNA, 21 nt, synthetic construct
  source 1..21: mol_type = other RNA
taaggatccattacaataaaa

SEQ ID NO: 660 - RNA, 21 nt, synthetic construct
  source 1..21: mol_type = other RNA
taaaggatccattacaataaa

SEQ ID NO: 661 - RNA, 21 nt, synthetic construct
  source 1..21: mol_type = other RNA
taaaaggatccattacaataa

SEQ ID NO: 662 - RNA, 21 nt, synthetic construct
  source 1..21: mol_type = other RNA
tcaaaaggatccattacaata

SEQ ID NO: 663 - RNA, 21 nt, synthetic construct
  source 1..21: mol_type = other RNA
tacaaaaggatccattacaat

SEQ ID NO: 664 - RNA, 21 nt, synthetic construct
  source 1..21: mol_type = other RNA
ttacaaaaggatccattacaa

SEQ ID NO: 665 - RNA, 21 nt, synthetic construct
  source 1..21: mol_type = other RNA
ttaattgcaagatctacaaaa

SEQ ID NO: 666 - RNA, 21 nt, synthetic construct
  source 1..21: mol_type = other RNA
taaactatgcaaatggtaatt

SEQ ID NO: 667 - RNA, 21 nt, synthetic construct
  source 1..21: mol_type = other RNA
taaaactatgcaaatggtaat

SEQ ID NO: 668 - RNA, 21 nt, synthetic construct
  source 1..21: mol_type = other RNA
ttaaaactatgcaaatggtaa

SEQ ID NO: 669 - RNA, 21 nt, synthetic construct
  source 1..21: mol_type = other RNA
tttaaaactatgcaaatggta

SEQ ID NO: 670 - RNA, 21 nt, synthetic construct
  source 1..21: mol_type = other RNA
ttttaaaactatgcaaatggt

SEQ ID NO: 671 - RNA, 21 nt, synthetic construct
  source 1..21: mol_type = other RNA
ttgtttaaaactatgcaaatg

SEQ ID NO: 672 - RNA, 21 nt, synthetic construct
  source 1..21: mol_type = other RNA
tgtgtttaaaactatgcaaat

SEQ ID NO: 673 - RNA, 21 nt, synthetic construct
  source 1..21: mol_type = other RNA
tggaaatactcatatggatcc

SEQ ID NO: 674 - RNA, 21 nt, synthetic construct
  source 1..21: mol_type = other RNA
ttggaaatactcatatggatc

SEQ ID NO: 675 - RNA, 21 nt, synthetic construct
  source 1..21: mol_type = other RNA
tttggaaatactcatatggat

SEQ ID NO: 676 - RNA, 21 nt, synthetic construct
  source 1..21: mol_type = other RNA
tgcctacttggaaatactcat

SEQ ID NO: 677 - RNA, 21 nt, synthetic construct
  source 1..21: mol_type = other RNA
tacaataaggcacatagcttg

SEQ ID NO: 678 - RNA, 21 nt, synthetic construct
  source 1..21: mol_type = other RNA
taacaataaggcacatagctt

SEQ ID NO: 679 - RNA, 21 nt, synthetic construct
  source 1..21: mol_type = other RNA
tgtcttcttcaattgctgtaa

SEQ ID NO: 680 - RNA, 21 nt, synthetic construct
  source 1..21: mol_type = other RNA
tactgtgaggctgggattgtg

SEQ ID NO: 681 - RNA, 21 nt, synthetic construct
  source 1..21: mol_type = other RNA
tagggttatcaactgtgctgc

SEQ ID NO: 682 - RNA, 21 nt, synthetic construct
  source 1..21: mol_type = other RNA
ttgttaacttggcagcatgag

SEQ ID NO: 683 - RNA, 21 nt, synthetic construct
  source 1..21: mol_type = other RNA
tatgttaacttggcagcatga

SEQ ID NO: 684 - RNA, 21 nt, synthetic construct
  source 1..21: mol_type = other RNA
ttctatgttaacttggcagca

SEQ ID NO: 685 - RNA, 21 nt, synthetic construct
  source 1..21: mol_type = other RNA
tagctttcaaaccaactgtgt

SEQ ID NO: 686 - RNA, 21 nt, synthetic construct
  source 1..21: mol_type = other RNA
taagctttcaaaccaactgtg

SEQ ID NO: 687 - RNA, 21 nt, synthetic construct
  source 1..21: mol_type = other RNA
tgaagctttcaaaccaactgt

SEQ ID NO: 688 - RNA, 21 nt, synthetic construct
  source 1..21: mol_type = other RNA
taatgaagctttcaaaccaac

SEQ ID NO: 689 - RNA, 21 nt, synthetic construct
  source 1..21: mol_type = other RNA
tcaatgaagctttcaaaccaa

SEQ ID NO: 690 - RNA, 21 nt, synthetic construct
  source 1..21: mol_type = other RNA
tacaatgaagctttcaaacca

SEQ ID NO: 691 - RNA, 21 nt, synthetic construct
  source 1..21: mol_type = other RNA
tgacaatgaagctttcaaacc

SEQ ID NO: 692 - RNA, 21 nt, synthetic construct
  source 1..21: mol_type = other RNA
tgataatcttaatggtctttt

SEQ ID NO: 693 - RNA, 21 nt, synthetic construct
  source 1..21: mol_type = other RNA
tcacattcatgatggaaggaa

SEQ ID NO: 694 - RNA, 21 nt, synthetic construct
  source 1..21: mol_type = other RNA
ttacactcatagaacttgcca

SEQ ID NO: 695 - RNA, 21 nt, synthetic construct
  source 1..21: mol_type = other RNA
tatacactcatagaacttgcc

SEQ ID NO: 696 - RNA, 21 nt, synthetic construct
  source 1..21: mol_type = other RNA
taatacactcatagaacttgc

SEQ ID NO: 697 - RNA, 21 nt, synthetic construct
  source 1..21: mol_type = other RNA
tcatcgcacattttgactaac

SEQ ID NO: 698 - RNA, 21 nt, synthetic construct
  source 1..21: mol_type = other RNA
tccatcgcacattttgactaa

SEQ ID NO: 699 - RNA, 21 nt, synthetic construct
  source 1..21: mol_type = other RNA
ttccatcgcacattttgacta

SEQ ID NO: 700 - RNA, 21 nt, synthetic construct
  source 1..21: mol_type = other RNA
tttccatcgcacattttgact

SEQ ID NO: 701 - RNA, 21 nt, synthetic construct
  source 1..21: mol_type = other RNA
tcaacttgaagcagagatagg

SEQ ID NO: 702 - RNA, 21 nt, synthetic construct
  source 1..21: mol_type = other RNA
tgcaacttgaagcagagatag

SEQ ID NO: 703 - RNA, 21 nt, synthetic construct
  source 1..21: mol_type = other RNA
ttgcaacttgaagcagagata

SEQ ID NO: 704 - RNA, 21 nt, synthetic construct
  source 1..21: mol_type = other RNA
taagttgcaacttgaagcaga

SEQ ID NO: 705 - RNA, 21 nt, synthetic construct
  source 1..21: mol_type = other RNA
taaagttgcaacttgaagcag

SEQ ID NO: 706 - RNA, 21 nt, synthetic construct
  source 1..21: mol_type = other RNA
taaaagttgcaacttgaagca

SEQ ID NO: 707 - RNA, 21 nt, synthetic construct
  source 1..21: mol_type = other RNA
tcaaagtgaagaatgacccaa

SEQ ID NO: 708 - RNA, 21 nt, synthetic construct
  source 1..21: mol_type = other RNA
ttcaaagtgaagaatgaccca

SEQ ID NO: 709 - RNA, 21 nt, synthetic construct
  source 1..21: mol_type = other RNA
tttcaaagtgaagaatgaccc

SEQ ID NO: 710 - RNA, 21 nt, synthetic construct
  source 1..21: mol_type = other RNA
tgttcaaagtgaagaatgacc

SEQ ID NO: 711 - RNA, 21 nt, synthetic construct
  source 1..21: mol_type = other RNA
tcaagttcaaagtgaagaatg

SEQ ID NO: 712 - RNA, 21 nt, synthetic construct
  source 1..21: mol_type = other RNA
tacaagttcaaagtgaagaat

SEQ ID NO: 713 - RNA, 21 nt, synthetic construct
  source 1..21: mol_type = other RNA
taatgaacaagttcaaagtga

SEQ ID NO: 714 - RNA, 21 nt, synthetic construct
  source 1..21: mol_type = other RNA
tccaatgaacaagttcaaagt

SEQ ID NO: 715 - RNA, 21 nt, synthetic construct
  source 1..21: mol_type = other RNA
tacaccaatgaacaagttcaa

SEQ ID NO: 716 - RNA, 21 nt, synthetic construct
  source 1..21: mol_type = other RNA
tttggttgaaattatctatga

SEQ ID NO: 717 - RNA, 21 nt, synthetic construct
  source 1..21: mol_type = other RNA
ttttctgttggttgaaattat

SEQ ID NO: 718 - RNA, 21 nt, synthetic construct
  source 1..21: mol_type = other RNA
taatatacatccttggatttt

SEQ ID NO: 719 - RNA, 21 nt, synthetic construct
  source 1..21: mol_type = other RNA
taaatatacatccttggattt

SEQ ID NO: 720 - RNA, 21 nt, synthetic construct
  source 1..21: mol_type = other RNA
ttcaaatatacatccttggat

SEQ ID NO: 721 - RNA, 21 nt, synthetic construct
  source 1..21: mol_type = other RNA
tggtcaaatatacatccttgg

SEQ ID NO: 722 - RNA, 21 nt, synthetic construct
  source 1..21: mol_type = other RNA
taggtcaaatatacatccttg

SEQ ID NO: 723 - RNA, 21 nt, synthetic construct
  source 1..21: mol_type = other RNA
ttaggtcaaatatacatcctt

SEQ ID NO: 724 - RNA, 21 nt, synthetic construct
  source 1..21: mol_type = other RNA
tctaggtcaaatatacatcct

SEQ ID NO: 725 - RNA, 21 nt, synthetic construct
  source 1..21: mol_type = other RNA
tagataagaaccatgatacta

SEQ ID NO: 726 - RNA, 21 nt, synthetic construct
  source 1..21: mol_type = other RNA
ttcatggttaccatgttgaga

SEQ ID NO: 727 - RNA, 21 nt, synthetic construct
  source 1..21: mol_type = other RNA
tatcatggttaccatgttgag

SEQ ID NO: 728 - RNA, 21 nt, synthetic construct
  source 1..21: mol_type = other RNA
tcatcatggttaccatgttga

SEQ ID NO: 729 - RNA, 21 nt, synthetic construct
  source 1..21: mol_type = other RNA
tccatcatggttaccatgttg

SEQ ID NO: 730 - RNA, 21 nt, synthetic construct
  source 1..21: mol_type = other RNA
ttaccatcatggttaccatgt

SEQ ID NO: 731 - RNA, 21 nt, synthetic construct
  source 1..21: mol_type = other RNA
tctaccatcatggttaccatg

SEQ ID NO: 732 - RNA, 21 nt, synthetic construct
  source 1..21: mol_type = other RNA
ttgtcattaattccatcttcc

SEQ ID NO: 733 - RNA, 21 nt, synthetic construct
  source 1..21: mol_type = other RNA
tatgtcattaattccatcttc

SEQ ID NO: 734 - RNA, 21 nt, synthetic construct
  source 1..21: mol_type = other RNA
tattgaacatgtcattaattc

SEQ ID NO: 735 - RNA, 21 nt, synthetic construct
  source 1..21: mol_type = other RNA
taaattgaacatgtcattaat

SEQ ID NO: 736 - RNA, 21 nt, synthetic construct
  source 1..21: mol_type = other RNA
taaaattgaacatgtcattaa

SEQ ID NO: 737 - RNA, 21 nt, synthetic construct
  source 1..21: mol_type = other RNA
tctgtgaacgaagagaatcca

SEQ ID NO: 738 - RNA, 21 nt, synthetic construct
  source 1..21: mol_type = other RNA
ttctgtgaacgaagagaatcc

SEQ ID NO: 739 - RNA, 21 nt, synthetic construct
  source 1..21: mol_type = other RNA
tatctgtgaacgaagagaatc

SEQ ID NO: 740 - RNA, 21 nt, synthetic construct
  source 1..21: mol_type = other RNA
tcatctgtgaacgaagagaat

SEQ ID NO: 741 - RNA, 21 nt, synthetic construct
  source 1..21: mol_type = other RNA
tcatgaacctttcttccatct

SEQ ID NO: 742 - RNA, 21 nt, synthetic construct
  source 1..21: mol_type = other RNA
tgacatgaacctttcttccat

SEQ ID NO: 743 - RNA, 21 nt, synthetic construct
  source 1..21: mol_type = other RNA
tagacatgaacctttcttcca

SEQ ID NO: 744 - RNA, 21 nt, synthetic construct
  source 1..21: mol_type = other RNA
tcttttatgtatatacttgat

SEQ ID NO: 745 - RNA, 21 nt, synthetic construct
  source 1..21: mol_type = other RNA
ttcttttatgtatatacttga

SEQ ID NO: 746 - RNA, 21 nt, synthetic construct
  source 1..21: mol_type = other RNA
tatcttttatgtatatacttg

SEQ ID NO: 747 - RNA, 21 nt, synthetic construct
  source 1..21: mol_type = other RNA
tcatcttttatgtatatactt

SEQ ID NO: 748 - RNA, 21 nt, synthetic construct
  source 1..21: mol_type = other RNA
tccatcttttatgtatatact

SEQ ID NO: 749 - RNA, 21 nt, synthetic construct
  source 1..21: mol_type = other RNA
ttccatcttttatgtatatac

SEQ ID NO: 750 - RNA, 21 nt, synthetic construct
  source 1..21: mol_type = other RNA
tctccatcttttatgtatata

SEQ ID NO: 751 - RNA, 21 nt, synthetic construct
  source 1..21: mol_type = other RNA
tgtctccatcttttatgtata

SEQ ID NO: 752 - RNA, 21 nt, synthetic construct
  source 1..21: mol_type = other RNA
tctggacttgagttctcatta

SEQ ID NO: 753 - RNA, 21 nt, synthetic construct
  source 1..21: mol_type = other RNA
ttctggacttgagttctcatt

SEQ ID NO: 754 - RNA, 21 nt, synthetic construct
  source 1..21: mol_type = other RNA
tttctggacttgagttctcat

SEQ ID NO: 755 - RNA, 21 nt, synthetic construct
  source 1..21: mol_type = other RNA
tgtggttattctatgaacaaa

SEQ ID NO: 756 - RNA, 21 nt, synthetic construct
  source 1..21: mol_type = other RNA
taggagtgtaatatgtactta

SEQ ID NO: 757 - RNA, 21 nt, synthetic construct
  source 1..21: mol_type = other RNA
tttattatataaatgtaattt

SEQ ID NO: 758 - RNA, 21 nt, synthetic construct
  source 1..21: mol_type = other RNA
tcttaattgaatgtatttaaa

SEQ ID NO: 759 - RNA, 21 nt, synthetic construct
  source 1..21: mol_type = other RNA
tttaatagaaaattacacacg

SEQ ID NO: 760 - RNA, 21 nt, synthetic construct
  source 1..21: mol_type = other RNA
tttaatatgacagtgccttct

SEQ ID NO: 761 - RNA, 21 nt, synthetic construct
  source 1..21: mol_type = other RNA
taaactaggtaactatcaaaa

SEQ ID NO: 762 - RNA, 21 nt, synthetic construct
  source 1..21: mol_type = other RNA
tgaaatagctatttagaactg

SEQ ID NO: 763 - RNA, 21 nt, synthetic construct
  source 1..21: mol_type = other RNA
ttgtaaagaatcctatgtaaa

SEQ ID NO: 764 - RNA, 21 nt, synthetic construct
  source 1..21: mol_type = other RNA
taacattttgagcattcatag

SEQ ID NO: 765 - RNA, 21 nt, synthetic construct
  source 1..21: mol_type = other RNA
taaacattttgagcattcata

SEQ ID NO: 766 - RNA, 21 nt, synthetic construct
  source 1..21: mol_type = other RNA
tcaaacattttgagcattcat

SEQ ID NO: 767 - RNA, 21 nt, synthetic construct
  source 1..21: mol_type = other RNA
tcaagtataactacaatataa

SEQ ID NO: 768 - RNA, 21 nt, synthetic construct
  source 1..21: mol_type = other RNA
ttcaagtataactacaatata

SEQ ID NO: 769 - RNA, 16 nt, synthetic construct
  source 1..16: mol_type = other RNA
tacaggatgaaaagaa

SEQ ID NO: 770 - RNA, 16 nt, synthetic construct
  source 1..16: mol_type = other RNA
gccctcattgaacaaa

SEQ ID NO: 771 - RNA, 16 nt, synthetic construct
  source 1..16: mol_type = other RNA
ccctcattgaacaaca

SEQ ID NO: 772 - RNA, 16 nt, synthetic construct
  source 1..16: mol_type = other RNA
cctcattgaacaacga

SEQ ID NO: 773 - RNA, 16 nt, synthetic construct
  source 1..16: mol_type = other RNA
tcattgaacaacgcaa

SEQ ID NO: 774 - RNA, 16 nt, synthetic construct
  source 1..16: mol_type = other RNA
atagtattgaacaaaa

SEQ ID NO: 775 - RNA, 16 nt, synthetic construct
  source 1..16: mol_type = other RNA
tagtattgaacaaaga

SEQ ID NO: 776 - RNA, 16 nt, synthetic construct
  source 1..16: mol_type = other RNA
gaacaaagggaaaaca

SEQ ID NO: 777 - RNA, 16 nt, synthetic construct
  source 1..16: mol_type = other RNA
aacaaagggaaaacaa

SEQ ID NO: 778 - RNA, 16 nt, synthetic construct
  source 1..16: mol_type = other RNA
aaacaatcttccgtta

SEQ ID NO: 779 - RNA, 16 nt, synthetic construct
  source 1..16: mol_type = other RNA
aacaatcttccgttta

SEQ ID NO: 780 - RNA, 16 nt, synthetic construct
  source 1..16: mol_type = other RNA
acaatcttccgtttca

SEQ ID NO: 781 - RNA, 16 nt, synthetic construct
  source 1..16: mol_type = other RNA
caatcttccgtttcaa

SEQ ID NO: 782 - RNA, 16 nt, synthetic construct
  source 1..16: mol_type = other RNA
atcttccgtttcaata

SEQ ID NO: 783 - RNA, 16 nt, synthetic construct
  source 1..16: mol_type = other RNA
tcttccgtttcaatga

SEQ ID NO: 784 - RNA, 16 nt, synthetic construct
  source 1..16: mol_type = other RNA
cttccgtttcaatgca

SEQ ID NO: 785 - RNA, 16 nt, synthetic construct
  source 1..16: mol_type = other RNA
ttccgtttcaatgcca

SEQ ID NO: 786 - RNA, 16 nt, synthetic construct
  source 1..16: mol_type = other RNA
cctttcagtcctctaa

SEQ ID NO: 787 - RNA, 16 nt, synthetic construct
  source 1..16: mol_type = other RNA
ctttcagtcctctaaa

SEQ ID NO: 788 - RNA, 16 nt, synthetic construct
  source 1..16: mol_type = other RNA
tttcagtcctctaaga

SEQ ID NO: 789 - RNA, 16 nt, synthetic construct
  source 1..16: mol_type = other RNA
ttcagtcctctaagaa

SEQ ID NO: 790 - RNA, 16 nt, synthetic construct
  source 1..16: mol_type = other RNA
agtcctctaagaagaa

SEQ ID NO: 791 - RNA, 16 nt, synthetic construct
  source 1..16: mol_type = other RNA
cctctaagaagaataa

SEQ ID NO: 792 - RNA, 16 nt, synthetic construct
  source 1..16: mol_type = other RNA
agaagaatatctatta

SEQ ID NO: 793 - RNA, 16 nt, synthetic construct
  source 1..16: mol_type = other RNA
gaagaatatctattaa

SEQ ID NO: 794 - RNA, 16 nt, synthetic construct
  source 1..16: mol_type = other RNA
aagaatatctattaaa

SEQ ID NO: 795 - RNA, 16 nt, synthetic construct
  source 1..16: mol_type = other RNA
agaatatctattaaga

SEQ ID NO: 796 - RNA, 16 nt, synthetic construct
  source 1..16: mol_type = other RNA
gaatatctattaagaa

SEQ ID NO: 797 - RNA, 16 nt, synthetic construct
  source 1..16: mol_type = other RNA
aatatctattaagata

SEQ ID NO: 798 - RNA, 16 nt, synthetic construct
  source 1..16: mol_type = other RNA
atatctattaagatta

SEQ ID NO: 799 - RNA, 16 nt, synthetic construct
  source 1..16: mol_type = other RNA
ctattaagattttaga

SEQ ID NO: 800 - RNA, 16 nt, synthetic construct
  source 1..16: mol_type = other RNA
ctgacaaactgcataa

SEQ ID NO: 801 - RNA, 16 nt, synthetic construct
  source 1..16: mol_type = other RNA
tgacaaactgcatata

SEQ ID NO: 802 - RNA, 16 nt, synthetic construct
  source 1..16: mol_type = other RNA
gtcgagtacactttta

SEQ ID NO: 803 - RNA, 16 nt, synthetic construct
  source 1..16: mol_type = other RNA
tcgagtacacttttaa

SEQ ID NO: 804 - RNA, 16 nt, synthetic construct
  source 1..16: mol_type = other RNA
cgagtacacttttaca

SEQ ID NO: 805 - RNA, 16 nt, synthetic construct
  source 1..16: mol_type = other RNA
gagtacacttttacta

SEQ ID NO: 806 - RNA, 16 nt, synthetic construct
  source 1..16: mol_type = other RNA
tacacttttactggaa

SEQ ID NO: 807 - RNA, 16 nt, synthetic construct
  source 1..16: mol_type = other RNA
acacttttactggaaa

SEQ ID NO: 808 - RNA, 16 nt, synthetic construct
  source 1..16: mol_type = other RNA
cacttttactggaata

SEQ ID NO: 809 - RNA, 16 nt, synthetic construct
  source 1..16: mol_type = other RNA
acttttactggaataa

SEQ ID NO: 810 - RNA, 16 nt, synthetic construct
  source 1..16: mol_type = other RNA
cgtatttaacagaata

SEQ ID NO: 811 - RNA, 16 nt, synthetic construct
  source 1..16: mol_type = other RNA
gtatttaacagaatta

SEQ ID NO: 812 - RNA, 16 nt, synthetic construct
  source 1..16: mol_type = other RNA
tatttaacagaattta

SEQ ID NO: 813 - RNA, 16 nt, synthetic construct
  source 1..16: mol_type = other RNA
ttaacagaatttgtaa

SEQ ID NO: 814 - RNA, 16 nt, synthetic construct
  source 1..16: mol_type = other RNA
taacagaatttgtaaa

SEQ ID NO: 815 - RNA, 16 nt, synthetic construct
  source 1..16: mol_type = other RNA
actatttctgtaatca

SEQ ID NO: 816 - RNA, 16 nt, synthetic construct
  source 1..16: mol_type = other RNA
tttctgtaatcccaga

SEQ ID NO: 817 - RNA, 16 nt, synthetic construct
  source 1..16: mol_type = other RNA
tttgcactaattggaa

SEQ ID NO: 818 - RNA, 16 nt, synthetic construct
  source 1..16: mol_type = other RNA
ttgcactaattggaca

SEQ ID NO: 819 - RNA, 16 nt, synthetic construct
  source 1..16: mol_type = other RNA
tgcactaattggacta

SEQ ID NO: 820 - RNA, 16 nt, synthetic construct
  source 1..16: mol_type = other RNA
ttggactacagctgta

SEQ ID NO: 821 - RNA, 16 nt, synthetic construct
  source 1..16: mol_type = other RNA
ataatgaaacattaga

SEQ ID NO: 822 - RNA, 16 nt, synthetic construct
  source 1..16: mol_type = other RNA
tgaaacattagaaaga

SEQ ID NO: 823 - RNA, 16 nt, synthetic construct
  source 1..16: mol_type = other RNA
aaacattagaaagcaa

SEQ ID NO: 824 - RNA, 16 nt, synthetic construct
  source 1..16: mol_type = other RNA
aacattagaaagcata

SEQ ID NO: 825 - RNA, 16 nt, synthetic construct
  source 1..16: mol_type = other RNA
gtttcagcacagatta

SEQ ID NO: 826 - RNA, 16 nt, synthetic construct
  source 1..16: mol_type = other RNA
tgattatggctacaca

SEQ ID NO: 827 - RNA, 16 nt, synthetic construct
  source 1..16: mol_type = other RNA
gattatggctacacga

SEQ ID NO: 828 - RNA, 16 nt, synthetic construct
  source 1..16: mol_type = other RNA
attatggctacacgaa

SEQ ID NO: 829 - RNA, 16 nt, synthetic construct
  source 1..16: mol_type = other RNA
ttatggctacacgaga

SEQ ID NO: 830 - RNA, 16 nt, synthetic construct
  source 1..16: mol_type = other RNA
acgagctttgacacta

SEQ ID NO: 831 - RNA, 16 nt, synthetic construct
  source 1..16: mol_type = other RNA
cgagctttgacactta

SEQ ID NO: 832 - RNA, 16 nt, synthetic construct
  source 1..16: mol_type = other RNA
gagctttgacacttta

SEQ ID NO: 833 - RNA, 16 nt, synthetic construct
  source 1..16: mol_type = other RNA
agctttgacactttca

SEQ ID NO: 834 - RNA, 16 nt, synthetic construct
  source 1..16: mol_type = other RNA
gcagcagaacaagtca

SEQ ID NO: 835 - RNA, 16 nt, synthetic construct
  source 1..16: mol_type = other RNA
cagcagaacaagtcta

SEQ ID NO: 836 - RNA, 16 nt, synthetic construct
  source 1..16: mol_type = other RNA
actgtggaagaactta

SEQ ID NO: 837 - RNA, 16 nt, synthetic construct
  source 1..16: mol_type = other RNA
gctctccatattggaa

SEQ ID NO: 838 - RNA, 16 nt, synthetic construct
  source 1..16: mol_type = other RNA
atattggataaaatta

SEQ ID NO: 839 - RNA, 16 nt, synthetic construct
  source 1..16: mol_type = other RNA
attggataaaattcaa

SEQ ID NO: 840 - RNA, 16 nt, synthetic construct
  source 1..16: mol_type = other RNA
ttggataaaattcaaa

SEQ ID NO: 841 - RNA, 16 nt, synthetic construct
  source 1..16: mol_type = other RNA
tggataaaattcaaaa

SEQ ID NO: 842 - RNA, 16 nt, synthetic construct
  source 1..16: mol_type = other RNA
tgtatctattttatta

SEQ ID NO: 843 - RNA, 16 nt, synthetic construct
  source 1..16: mol_type = other RNA
tctattttattgtaaa

SEQ ID NO: 844 - RNA, 16 nt, synthetic construct
  source 1..16: mol_type = other RNA
ctattttattgtaata

SEQ ID NO: 845 - RNA, 16 nt, synthetic construct
  source 1..16: mol_type = other RNA
attttattgtaatgga

SEQ ID NO: 846 - RNA, 16 nt, synthetic construct
  source 1..16: mol_type = other RNA
ttttattgtaatggaa

SEQ ID NO: 847 - RNA, 16 nt, synthetic construct
  source 1..16: mol_type = other RNA
tttattgtaatggata

SEQ ID NO: 848 - RNA, 16 nt, synthetic construct
  source 1..16: mol_type = other RNA
ttattgtaatggatca

SEQ ID NO: 849 - RNA, 16 nt, synthetic construct
  source 1..16: mol_type = other RNA
tattgtaatggatcca

SEQ ID NO: 850 - RNA, 16 nt, synthetic construct
  source 1..16: mol_type = other RNA
attgtaatggatccta

SEQ ID NO: 851 - RNA, 16 nt, synthetic construct
  source 1..16: mol_type = other RNA
ttgtaatggatcctta

SEQ ID NO: 852 - RNA, 16 nt, synthetic construct
  source 1..16: mol_type = other RNA
tgtaatggatccttta

SEQ ID NO: 853 - RNA, 16 nt, synthetic construct
  source 1..16: mol_type = other RNA
gtaatggatcctttta

SEQ ID NO: 854 - RNA, 16 nt, synthetic construct
  source 1..16: mol_type = other RNA
taatggatccttttga

SEQ ID NO: 855 - RNA, 16 nt, synthetic construct
  source 1..16: mol_type = other RNA
aatggatccttttgta

SEQ ID NO: 856 - RNA, 16 nt, synthetic construct
  source 1..16: mol_type = other RNA
atggatccttttgtaa

SEQ ID NO: 857 - RNA, 16 nt, synthetic construct
  source 1..16: mol_type = other RNA
tagatcttgcaattaa

SEQ ID NO: 858 - RNA, 16 nt, synthetic construct
  source 1..16: mol_type = other RNA
ccatttgcatagttta

SEQ ID NO: 859 - RNA, 16 nt, synthetic construct
  source 1..16: mol_type = other RNA
catttgcatagtttta

SEQ ID NO: 860 - RNA, 16 nt, synthetic construct
  source 1..16: mol_type = other RNA
atttgcatagttttaa

SEQ ID NO: 861 - RNA, 16 nt, synthetic construct
  source 1..16: mol_type = other RNA
tttgcatagttttaaa

SEQ ID NO: 862 - RNA, 16 nt, synthetic construct
  source 1..16: mol_type = other RNA
ttgcatagttttaaaa

SEQ ID NO: 863 - RNA, 16 nt, synthetic construct
  source 1..16: mol_type = other RNA
gcatagttttaaacaa

SEQ ID NO: 864 - RNA, 16 nt, synthetic construct
  source 1..16: mol_type = other RNA
catagttttaaacaca

SEQ ID NO: 865 - RNA, 16 nt, synthetic construct
  source 1..16: mol_type = other RNA
catatgagtatttcca

SEQ ID NO: 866 - RNA, 16 nt, synthetic construct
  source 1..16: mol_type = other RNA
atatgagtatttccaa

SEQ ID NO: 867 - RNA, 16 nt, synthetic construct
  source 1..16: mol_type = other RNA
tatgagtatttccaaa

SEQ ID NO: 868 - RNA, 16 nt, synthetic construct
  source 1..16: mol_type = other RNA
tatttccaagtaggca

SEQ ID NO: 869 - RNA, 16 nt, synthetic construct
  source 1..16: mol_type = other RNA
tatgtgccttattgta

SEQ ID NO: 870 - RNA, 16 nt, synthetic construct
  source 1..16: mol_type = other RNA
atgtgccttattgtta

SEQ ID NO: 871 - RNA, 16 nt, synthetic construct
  source 1..16: mol_type = other RNA
gcaattgaagaagaca

SEQ ID NO: 872 - RNA, 16 nt, synthetic construct
  source 1..16: mol_type = other RNA
tcccagcctcacagta

SEQ ID NO: 873 - RNA, 16 nt, synthetic construct
  source 1..16: mol_type = other RNA
acagttgataacccta

SEQ ID NO: 874 - RNA, 16 nt, synthetic construct
  source 1..16: mol_type = other RNA
gctgccaagttaacaa

SEQ ID NO: 875 - RNA, 16 nt, synthetic construct
  source 1..16: mol_type = other RNA
ctgccaagttaacata

SEQ ID NO: 876 - RNA, 16 nt, synthetic construct
  source 1..16: mol_type = other RNA
ccaagttaacatagaa

SEQ ID NO: 877 - RNA, 16 nt, synthetic construct
  source 1..16: mol_type = other RNA
gttggtttgaaagcta

SEQ ID NO: 878 - RNA, 16 nt, synthetic construct
  source 1..16: mol_type = other RNA
ttggtttgaaagctta

SEQ ID NO: 879 - RNA, 16 nt, synthetic construct
  source 1..16: mol_type = other RNA
tggtttgaaagcttca

SEQ ID NO: 880 - RNA, 16 nt, synthetic construct
  source 1..16: mol_type = other RNA
tttgaaagcttcatta

SEQ ID NO: 881 - RNA, 16 nt, synthetic construct
  source 1..16: mol_type = other RNA
ttgaaagcttcattga

SEQ ID NO: 882 - RNA, 16 nt, synthetic construct
  source 1..16: mol_type = other RNA
tgaaagcttcattgta

SEQ ID NO: 883 - RNA, 16 nt, synthetic construct
  source 1..16: mol_type = other RNA
gaaagcttcattgtca

SEQ ID NO: 884 - RNA, 16 nt, synthetic construct
  source 1..16: mol_type = other RNA
accattaagattatca

SEQ ID NO: 885 - RNA, 16 nt, synthetic construct
  source 1..16: mol_type = other RNA
tccatcatgaatgtga

SEQ ID NO: 886 - RNA, 16 nt, synthetic construct
  source 1..16: mol_type = other RNA
agttctatgagtgtaa

SEQ ID NO: 887 - RNA, 16 nt, synthetic construct
  source 1..16: mol_type = other RNA
gttctatgagtgtata

SEQ ID NO: 888 - RNA, 16 nt, synthetic construct
  source 1..16: mol_type = other RNA
ttctatgagtgtatta

SEQ ID NO: 889 - RNA, 16 nt, synthetic construct
  source 1..16: mol_type = other RNA
tcaaaatgtgcgatga

SEQ ID NO: 890 - RNA, 16 nt, synthetic construct
  source 1..16: mol_type = other RNA
caaaatgtgcgatgga

SEQ ID NO: 891 - RNA, 16 nt, synthetic construct
  source 1..16: mol_type = other RNA
aaaatgtgcgatggaa

SEQ ID NO: 892 - RNA, 16 nt, synthetic construct
  source 1..16: mol_type = other RNA
aaatgtgcgatggaaa

SEQ ID NO: 893 - RNA, 16 nt, synthetic construct
  source 1..16: mol_type = other RNA
ctctgcttcaagttga

SEQ ID NO: 894 - RNA, 16 nt, synthetic construct
  source 1..16: mol_type = other RNA
tctgcttcaagttgca

SEQ ID NO: 895 - RNA, 16 nt, synthetic construct
  source 1..16: mol_type = other RNA
ctgcttcaagttgcaa

SEQ ID NO: 896 - RNA, 16 nt, synthetic construct
  source 1..16: mol_type = other RNA
ttcaagttgcaactta

SEQ ID NO: 897 - RNA, 16 nt, synthetic construct
  source 1..16: mol_type = other RNA
tcaagttgcaacttta

SEQ ID NO: 898 - RNA, 16 nt, synthetic construct
  source 1..16: mol_type = other RNA
caagttgcaactttta

SEQ ID NO: 899 - RNA, 16 nt, synthetic construct
  source 1..16: mol_type = other RNA
tcattcttcactttga

SEQ ID NO: 900 - RNA, 16 nt, synthetic construct
  source 1..16: mol_type = other RNA
cattcttcactttgaa

SEQ ID NO: 901 - RNA, 16 nt, synthetic construct
  source 1..16: mol_type = other RNA
attcttcactttgaaa

SEQ ID NO: 902 - RNA, 16 nt, synthetic construct
  source 1..16: mol_type = other RNA
ttcttcactttgaaca

SEQ ID NO: 903 - RNA, 16 nt, synthetic construct
  source 1..16: mol_type = other RNA
ttcactttgaacttga

SEQ ID NO: 904 - RNA, 16 nt, synthetic construct
  source 1..16: mol_type = other RNA
tcactttgaacttgta

SEQ ID NO: 905 - RNA, 16 nt, synthetic construct
  source 1..16: mol_type = other RNA
ttgaacttgttcatta

SEQ ID NO: 906 - RNA, 16 nt, synthetic construct
  source 1..16: mol_type = other RNA
gaacttgttcattgga

SEQ ID NO: 907 - RNA, 16 nt, synthetic construct
  source 1..16: mol_type = other RNA
cttgttcattggtgta

SEQ ID NO: 908 - RNA, 16 nt, synthetic construct
  source 1..16: mol_type = other RNA
gataatttcaaccaaa

SEQ ID NO: 909 - RNA, 16 nt, synthetic construct
  source 1..16: mol_type = other RNA
ttcaaccaacagaaaa

SEQ ID NO: 910 - RNA, 16 nt, synthetic construct
  source 1..16: mol_type = other RNA
ccaaggatgtatatta

SEQ ID NO: 911 - RNA, 16 nt, synthetic construct
  source 1..16: mol_type = other RNA
caaggatgtatattta

SEQ ID NO: 912 - RNA, 16 nt, synthetic construct
  source 1..16: mol_type = other RNA
aggatgtatatttgaa

SEQ ID NO: 913 - RNA, 16 nt, synthetic construct
  source 1..16: mol_type = other RNA
gatgtatatttgacca

SEQ ID NO: 914 - RNA, 16 nt, synthetic construct
  source 1..16: mol_type = other RNA
atgtatatttgaccta

SEQ ID NO: 915 - RNA, 16 nt, synthetic construct
  source 1..16: mol_type = other RNA
tgtatatttgacctaa

SEQ ID NO: 916 - RNA, 16 nt, synthetic construct
  source 1..16: mol_type = other RNA
gtatatttgacctaga

SEQ ID NO: 917 - RNA, 16 nt, synthetic construct
  source 1..16: mol_type = other RNA
tcatggttcttatcta

SEQ ID NO: 918 - RNA, 16 nt, synthetic construct
  source 1..16: mol_type = other RNA
acatggtaaccatgaa

SEQ ID NO: 919 - RNA, 16 nt, synthetic construct
  source 1..16: mol_type = other RNA
catggtaaccatgata

SEQ ID NO: 920 - RNA, 16 nt, synthetic construct
  source 1..16: mol_type = other RNA
atggtaaccatgatga

SEQ ID NO: 921 - RNA, 16 nt, synthetic construct
  source 1..16: mol_type = other RNA
tggtaaccatgatgga

SEQ ID NO: 922 - RNA, 16 nt, synthetic construct
  source 1..16: mol_type = other RNA
gtaaccatgatggtaa

SEQ ID NO: 923 - RNA, 16 nt, synthetic construct
  source 1..16: mol_type = other RNA
taaccatgatggtaga

SEQ ID NO: 924 - RNA, 16 nt, synthetic construct
  source 1..16: mol_type = other RNA
atggaattaatgacaa

SEQ ID NO: 925 - RNA, 16 nt, synthetic construct
  source 1..16: mol_type = other RNA
tggaattaatgacata

SEQ ID NO: 926 - RNA, 16 nt, synthetic construct
  source 1..16: mol_type = other RNA
aatgacatgttcaata

SEQ ID NO: 927 - RNA, 16 nt, synthetic construct
  source 1..16: mol_type = other RNA
tgacatgttcaattta

SEQ ID NO: 928 - RNA, 16 nt, synthetic construct
  source 1..16: mol_type = other RNA
gacatgttcaatttta

SEQ ID NO: 929 - RNA, 16 nt, synthetic construct
  source 1..16: mol_type = other RNA
tctcttcgttcacaga

SEQ ID NO: 930 - RNA, 16 nt, synthetic construct
  source 1..16: mol_type = other RNA
ctcttcgttcacagaa

SEQ ID NO: 931 - RNA, 16 nt, synthetic construct
  source 1..16: mol_type = other RNA
tcttcgttcacagata

SEQ ID NO: 932 - RNA, 16 nt, synthetic construct
  source 1..16: mol_type = other RNA
cttcgttcacagatga

SEQ ID NO: 933 - RNA, 16 nt, synthetic construct
  source 1..16: mol_type = other RNA
gaagaaaggttcatga

SEQ ID NO: 934 - RNA, 16 nt, synthetic construct
  source 1..16: mol_type = other RNA
agaaaggttcatgtca

SEQ ID NO: 935 - RNA, 16 nt, synthetic construct
  source 1..16: mol_type = other RNA
gaaaggttcatgtcta

SEQ ID NO: 936 - RNA, 16 nt, synthetic construct
  source 1..16: mol_type = other RNA
gtatatacataaaaga

SEQ ID NO: 937 - RNA, 16 nt, synthetic construct
  source 1..16: mol_type = other RNA
tatatacataaaagaa

SEQ ID NO: 938 - RNA, 16 nt, synthetic construct
  source 1..16: mol_type = other RNA
atatacataaaagata

SEQ ID NO: 939 - RNA, 16 nt, synthetic construct
  source 1..16: mol_type = other RNA
tatacataaaagatga

SEQ ID NO: 940 - RNA, 16 nt, synthetic construct
  source 1..16: mol_type = other RNA
atacataaaagatgga

SEQ ID NO: 941 - RNA, 16 nt, synthetic construct
  source 1..16: mol_type = other RNA
tacataaaagatggaa

SEQ ID NO: 942 - RNA, 16 nt, synthetic construct
  source 1..16: mol_type = other RNA
acataaaagatggaga

SEQ ID NO: 943 - RNA, 16 nt, synthetic construct
  source 1..16: mol_type = other RNA
ataaaagatggagaca

SEQ ID NO: 944 - RNA, 16 nt, synthetic construct
  source 1..16: mol_type = other RNA
agaactcaagtccaga

SEQ ID NO: 945 - RNA, 16 nt, synthetic construct
  source 1..16: mol_type = other RNA
gaactcaagtccagaa

SEQ ID NO: 946 - RNA, 16 nt, synthetic construct
  source 1..16: mol_type = other RNA
aactcaagtccagaaa

SEQ ID NO: 947 - RNA, 16 nt, synthetic construct
  source 1..16: mol_type = other RNA
tcatagaataaccaca

SEQ ID NO: 948 - RNA, 16 nt, synthetic construct
  source 1..16: mol_type = other RNA
acatattacactccta

SEQ ID NO: 949 - RNA, 16 nt, synthetic construct
  source 1..16: mol_type = other RNA
acatttatataataaa

SEQ ID NO: 950 - RNA, 16 nt, synthetic construct
  source 1..16: mol_type = other RNA
atacattcaattaaga

SEQ ID NO: 951 - RNA, 16 nt, synthetic construct
  source 1..16: mol_type = other RNA
gtaattttctattaaa

SEQ ID NO: 952 - RNA, 16 nt, synthetic construct
  source 1..16: mol_type = other RNA
gcactgtcatattaaa

SEQ ID NO: 953 - RNA, 16 nt, synthetic construct
  source 1..16: mol_type = other RNA
atagttacctagttta

SEQ ID NO: 954 - RNA, 16 nt, synthetic construct
  source 1..16: mol_type = other RNA
ctaaatagctatttca

SEQ ID NO: 955 - RNA, 16 nt, synthetic construct
  source 1..16: mol_type = other RNA
ataggattctttacaa

SEQ ID NO: 956 - RNA, 16 nt, synthetic construct
  source 1..16: mol_type = other RNA
aatgctcaaaatgtta

SEQ ID NO: 957 - RNA, 16 nt, synthetic construct
  source 1..16: mol_type = other RNA
atgctcaaaatgttta

SEQ ID NO: 958 - RNA, 16 nt, synthetic construct
  source 1..16: mol_type = other RNA
tgctcaaaatgtttga

SEQ ID NO: 959 - RNA, 16 nt, synthetic construct
  source 1..16: mol_type = other RNA
ttgtagttatacttga

SEQ ID NO: 960 - RNA, 16 nt, synthetic construct
  source 1..16: mol_type = other RNA
tgtagttatacttgaa

SEQ ID NO: 961 - RNA, 21 nt, Homo sapiens
  source 1..21: mol_type = mRNA
aaatatacaggatgaaaagat

SEQ ID NO: 962 - RNA, 21 nt, Homo sapiens
  source 1..21: mol_type = mRNA
ctcttgccctcattgaacaac

SEQ ID NO: 963 - RNA, 21 nt, Homo sapiens
  source 1..21: mol_type = mRNA
tcttgccctcattgaacaacg

SEQ ID NO: 964 - RNA, 21 nt, Homo sapiens
  source 1..21: mol_type = mRNA
cttgccctcattgaacaacgc

SEQ ID NO: 965 - RNA, 21 nt, Homo sapiens
  source 1..21: mol_type = mRNA
tgccctcattgaacaacgcat

SEQ ID NO: 966 - RNA, 21 nt, Homo sapiens
  source 1..21: mol_type = mRNA
ctttcatagtattgaacaaag

SEQ ID NO: 967 - RNA, 21 nt, Homo sapiens
  source 1..21: mol_type = mRNA
tttcatagtattgaacaaagg

SEQ ID NO: 968 - RNA, 21 nt, Homo sapiens
  source 1..21: mol_type = mRNA
gtattgaacaaagggaaaaca

SEQ ID NO: 969 - RNA, 21 nt, Homo sapiens
  source 1..21: mol_type = mRNA
tattgaacaaagggaaaacaa

SEQ ID NO: 970 - RNA, 21 nt, Homo sapiens
  source 1..21: mol_type = mRNA
agggaaaacaatcttccgttt

SEQ ID NO: 971 - RNA, 21 nt, Homo sapiens
  source 1..21: mol_type = mRNA
gggaaaacaatcttccgtttc

SEQ ID NO: 972 - RNA, 21 nt, Homo sapiens
  source 1..21: mol_type = mRNA
ggaaaacaatcttccgtttca

SEQ ID NO: 973 - RNA, 21 nt, Homo sapiens
  source 1..21: mol_type = mRNA
gaaaacaatcttccgtttcaa

SEQ ID NO: 974 - RNA, 21 nt, Homo sapiens
  source 1..21: mol_type = mRNA
aaacaatcttccgtttcaatg

SEQ ID NO: 975 - RNA, 21 nt, Homo sapiens
  source 1..21: mol_type = mRNA
aacaatcttccgtttcaatgc

SEQ ID NO: 976 - RNA, 21 nt, Homo sapiens
  source 1..21: mol_type = mRNA
acaatcttccgtttcaatgcc

SEQ ID NO: 977 - RNA, 21 nt, Homo sapiens
  source 1..21: mol_type = mRNA
caatcttccgtttcaatgcca

SEQ ID NO: 978 - RNA, 21 nt, Homo sapiens
  source 1..21: mol_type = mRNA
tttctcctttcagtcctctaa

SEQ ID NO: 979 - RNA, 21 nt, Homo sapiens
  source 1..21: mol_type = mRNA
ttctcctttcagtcctctaag

SEQ ID NO: 980 - RNA, 21 nt, Homo sapiens
  source 1..21: mol_type = mRNA
tctcctttcagtcctctaaga

SEQ ID NO: 981 - RNA, 21 nt, Homo sapiens
  source 1..21: mol_type = mRNA
ctcctttcagtcctctaagaa

SEQ ID NO: 982 - RNA, 21 nt, Homo sapiens
  source 1..21: mol_type = mRNA
ctttcagtcctctaagaagaa

SEQ ID NO: 983 - RNA, 21 nt, Homo sapiens
  source 1..21: mol_type = mRNA
tcagtcctctaagaagaatat

SEQ ID NO: 984 - RNA, 21 nt, Homo sapiens
  source 1..21: mol_type = mRNA
ctctaagaagaatatctatta

SEQ ID NO: 985 - RNA, 21 nt, Homo sapiens
  source 1..21: mol_type = mRNA
tctaagaagaatatctattaa

SEQ ID NO: 986 - RNA, 21 nt, Homo sapiens
  source 1..21: mol_type = mRNA
ctaagaagaatatctattaag

SEQ ID NO: 987 - RNA, 21 nt, Homo sapiens
  source 1..21: mol_type = mRNA
taagaagaatatctattaaga

SEQ ID NO: 988 - RNA, 21 nt, Homo sapiens
  source 1..21: mol_type = mRNA
aagaagaatatctattaagat

SEQ ID NO: 989 - RNA, 21 nt, Homo sapiens
  source 1..21: mol_type = mRNA
agaagaatatctattaagatt

SEQ ID NO: 990 - RNA, 21 nt, Homo sapiens
  source 1..21: mol_type = mRNA
gaagaatatctattaagattt

SEQ ID NO: 991 - RNA, 21 nt, Homo sapiens
  source 1..21: mol_type = mRNA
aatatctattaagattttagt

SEQ ID NO: 992 - RNA, 21 nt, Homo sapiens
  source 1..21: mol_type = mRNA
ctattctgacaaactgcatat

SEQ ID NO: 993 - RNA, 21 nt, Homo sapiens
  source 1..21: mol_type = mRNA
tattctgacaaactgcatatt

SEQ ID NO: 994 - RNA, 21 nt, Homo sapiens
  source 1..21: mol_type = mRNA
aaaatgtcgagtacactttta

SEQ ID NO: 995 - RNA, 21 nt, Homo sapiens
  source 1..21: mol_type = mRNA
aaatgtcgagtacacttttac

SEQ ID NO: 996 - RNA, 21 nt, Homo sapiens
  source 1..21: mol_type = mRNA
aatgtcgagtacacttttact

SEQ ID NO: 997 - RNA, 21 nt, Homo sapiens
  source 1..21: mol_type = mRNA
atgtcgagtacacttttactg

SEQ ID NO: 998 - RNA, 21 nt, Homo sapiens
  source 1..21: mol_type = mRNA
tcgagtacacttttactggaa

SEQ ID NO: 999 - RNA, 21 nt, Homo sapiens
  source 1..21: mol_type = mRNA
cgagtacacttttactggaat

SEQ ID NO: 1000 - RNA, 21 nt, Homo sapiens
  source 1..21: mol_type = mRNA
gagtacacttttactggaata

SEQ ID NO: 1001 - RNA, 21 nt, Homo sapiens
  source 1..21: mol_type = mRNA
agtacacttttactggaatat

SEQ ID NO: 1002 - RNA, 21 nt, Homo sapiens
  source 1..21: mol_type = mRNA
ttttgcgtatttaacagaatt

SEQ ID NO: 1003 - RNA, 21 nt, Homo sapiens
  source 1..21: mol_type = mRNA
tttgcgtatttaacagaattt

SEQ ID NO: 1004 - RNA, 21 nt, Homo sapiens
  source 1..21: mol_type = mRNA
ttgcgtatttaacagaatttg

SEQ ID NO: 1005 - RNA, 21 nt, Homo sapiens
  source 1..21: mol_type = mRNA
cgtatttaacagaatttgtaa

SEQ ID NO: 1006 - RNA, 21 nt, Homo sapiens
  source 1..21: mol_type = mRNA
gtatttaacagaatttgtaaa

SEQ ID NO: 1007 - RNA, 21 nt, Homo sapiens
  source 1..21: mol_type = mRNA
tgaaaactatttctgtaatcc

SEQ ID NO: 1008 - RNA, 21 nt, Homo sapiens
  source 1..21: mol_type = mRNA
aactatttctgtaatcccagg

SEQ ID NO: 1009 - RNA, 21 nt, Homo sapiens
  source 1..21: mol_type = mRNA
gtgtgtttgcactaattggac

SEQ ID NO: 1010 - RNA, 21 nt, Homo sapiens
  source 1..21: mol_type = mRNA
tgtgtttgcactaattggact

SEQ ID NO: 1011 - RNA, 21 nt, Homo sapiens
  source 1..21: mol_type = mRNA
gtgtttgcactaattggacta

SEQ ID NO: 1012 - RNA, 21 nt, Homo sapiens
  source 1..21: mol_type = mRNA
actaattggactacagctgtt

SEQ ID NO: 1013 - RNA, 21 nt, Homo sapiens
  source 1..21: mol_type = mRNA
tgaaaataatgaaacattaga

SEQ ID NO: 1014 - RNA, 21 nt, Homo sapiens
  source 1..21: mol_type = mRNA
aataatgaaacattagaaagc

SEQ ID NO: 1015 - RNA, 21 nt, Homo sapiens
  source 1..21: mol_type = mRNA
taatgaaacattagaaagcat

SEQ ID NO: 1016 - RNA, 21 nt, Homo sapiens
  source 1..21: mol_type = mRNA
aatgaaacattagaaagcata

SEQ ID NO: 1017 - RNA, 21 nt, Homo sapiens
  source 1..21: mol_type = mRNA
ttgtggtttcagcacagattc

SEQ ID NO: 1018 - RNA, 21 nt, Homo sapiens
  source 1..21: mol_type = mRNA
aaccctgattatggctacacg

SEQ ID NO: 1019 - RNA, 21 nt, Homo sapiens
  source 1..21: mol_type = mRNA
accctgattatggctacacga

SEQ ID NO: 1020 - RNA, 21 nt, Homo sapiens
  source 1..21: mol_type = mRNA
ccctgattatggctacacgag

SEQ ID NO: 1021 - RNA, 21 nt, Homo sapiens
  source 1..21: mol_type = mRNA
cctgattatggctacacgagc

SEQ ID NO: 1022 - RNA, 21 nt, Homo sapiens
  source 1..21: mol_type = mRNA
gctacacgagctttgacactt

SEQ ID NO: 1023 - RNA, 21 nt, Homo sapiens
  source 1..21: mol_type = mRNA
ctacacgagctttgacacttt

SEQ ID NO: 1024 - RNA, 21 nt, Homo sapiens
  source 1..21: mol_type = mRNA
tacacgagctttgacactttc

SEQ ID NO: 1025 - RNA, 21 nt, Homo sapiens
  source 1..21: mol_type = mRNA
acacgagctttgacactttca

SEQ ID NO: 1026 - RNA, 21 nt, Homo sapiens
  source 1..21: mol_type = mRNA
gcgaagcagcagaacaagtct

SEQ ID NO: 1027 - RNA, 21 nt, Homo sapiens
  source 1..21: mol_type = mRNA
cgaagcagcagaacaagtctt

SEQ ID NO: 1028 - RNA, 21 nt, Homo sapiens
  source 1..21: mol_type = mRNA
caaacactgtggaagaacttg

SEQ ID NO: 1029 - RNA, 21 nt, Homo sapiens
  source 1..21: mol_type = mRNA
gaattgctctccatattggat

SEQ ID NO: 1030 - RNA, 21 nt, Homo sapiens
  source 1..21: mol_type = mRNA
tctccatattggataaaattc

SEQ ID NO: 1031 - RNA, 21 nt, Homo sapiens
  source 1..21: mol_type = mRNA
tccatattggataaaattcaa

SEQ ID NO: 1032 - RNA, 21 nt, Homo sapiens
  source 1..21: mol_type = mRNA
ccatattggataaaattcaaa

SEQ ID NO: 1033 - RNA, 21 nt, Homo sapiens
  source 1..21: mol_type = mRNA
catattggataaaattcaaaa

SEQ ID NO: 1034 - RNA, 21 nt, Homo sapiens
  source 1..21: mol_type = mRNA
aaaagtgtatctattttattg

SEQ ID NO: 1035 - RNA, 21 nt, Homo sapiens
  source 1..21: mol_type = mRNA
gtgtatctattttattgtaat

SEQ ID NO: 1036 - RNA, 21 nt, Homo sapiens
  source 1..21: mol_type = mRNA
tgtatctattttattgtaatg

SEQ ID NO: 1037 - RNA, 21 nt, Homo sapiens
  source 1..21: mol_type = mRNA
tatctattttattgtaatgga

SEQ ID NO: 1038 - RNA, 21 nt, Homo sapiens
  source 1..21: mol_type = mRNA
atctattttattgtaatggat

SEQ ID NO: 1039 - RNA, 21 nt, Homo sapiens
  source 1..21: mol_type = mRNA
tctattttattgtaatggatc

SEQ ID NO: 1040 - RNA, 21 nt, Homo sapiens
  source 1..21: mol_type = mRNA
ctattttattgtaatggatcc

SEQ ID NO: 1041 - RNA, 21 nt, Homo sapiens
  source 1..21: mol_type = mRNA
tattttattgtaatggatcct

SEQ ID NO: 1042 - RNA, 21 nt, Homo sapiens
  source 1..21: mol_type = mRNA
attttattgtaatggatcctt

SEQ ID NO: 1043 - RNA, 21 nt, Homo sapiens
  source 1..21: mol_type = mRNA
ttttattgtaatggatccttt

SEQ ID NO: 1044 - RNA, 21 nt, Homo sapiens
  source 1..21: mol_type = mRNA
tttattgtaatggatcctttt

SEQ ID NO: 1045 - RNA, 21 nt, Homo sapiens
  source 1..21: mol_type = mRNA
ttattgtaatggatccttttg

SEQ ID NO: 1046 - RNA, 21 nt, Homo sapiens
  source 1..21: mol_type = mRNA
tattgtaatggatccttttgt

SEQ ID NO: 1047 - RNA, 21 nt, Homo sapiens
  source 1..21: mol_type = mRNA
attgtaatggatccttttgta

SEQ ID NO: 1048 - RNA, 21 nt, Homo sapiens
  source 1..21: mol_type = mRNA
ttgtaatggatccttttgtag

SEQ ID NO: 1049 - RNA, 21 nt, Homo sapiens
  source 1..21: mol_type = mRNA
ttttgtagatcttgcaattac

SEQ ID NO: 1050 - RNA, 21 nt, Homo sapiens
  source 1..21: mol_type = mRNA
aattaccatttgcatagtttt

SEQ ID NO: 1051 - RNA, 21 nt, Homo sapiens
  source 1..21: mol_type = mRNA
attaccatttgcatagtttta

SEQ ID NO: 1052 - RNA, 21 nt, Homo sapiens
  source 1..21: mol_type = mRNA
ttaccatttgcatagttttaa

SEQ ID NO: 1053 - RNA, 21 nt, Homo sapiens
  source 1..21: mol_type = mRNA
taccatttgcatagttttaaa

SEQ ID NO: 1054 - RNA, 21 nt, Homo sapiens
  source 1..21: mol_type = mRNA
accatttgcatagttttaaac

SEQ ID NO: 1055 - RNA, 21 nt, Homo sapiens
  source 1..21: mol_type = mRNA
catttgcatagttttaaacac

SEQ ID NO: 1056 - RNA, 21 nt, Homo sapiens
  source 1..21: mol_type = mRNA
atttgcatagttttaaacaca

SEQ ID NO: 1057 - RNA, 21 nt, Homo sapiens
  source 1..21: mol_type = mRNA
ggatccatatgagtatttcca

SEQ ID NO: 1058 - RNA, 21 nt, Homo sapiens
  source 1..21: mol_type = mRNA
gatccatatgagtatttccaa

SEQ ID NO: 1059 - RNA, 21 nt, Homo sapiens
  source 1..21: mol_type = mRNA
atccatatgagtatttccaag

SEQ ID NO: 1060 - RNA, 21 nt, Homo sapiens
  source 1..21: mol_type = mRNA
atgagtatttccaagtaggct

SEQ ID NO: 1061 - RNA, 21 nt, Homo sapiens
  source 1..21: mol_type = mRNA
caagctatgtgccttattgtt

SEQ ID NO: 1062 - RNA, 21 nt, Homo sapiens
  source 1..21: mol_type = mRNA
aagctatgtgccttattgttt

SEQ ID NO: 1063 - RNA, 21 nt, Homo sapiens
  source 1..21: mol_type = mRNA
ttacagcaattgaagaagacc

SEQ ID NO: 1064 - RNA, 21 nt, Homo sapiens
  source 1..21: mol_type = mRNA
cacaatcccagcctcacagtg

SEQ ID NO: 1065 - RNA, 21 nt, Homo sapiens
  source 1..21: mol_type = mRNA
gcagcacagttgataaccctt

SEQ ID NO: 1066 - RNA, 21 nt, Homo sapiens
  source 1..21: mol_type = mRNA
ctcatgctgccaagttaacat

SEQ ID NO: 1067 - RNA, 21 nt, Homo sapiens
  source 1..21: mol_type = mRNA
tcatgctgccaagttaacata

SEQ ID NO: 1068 - RNA, 21 nt, Homo sapiens
  source 1..21: mol_type = mRNA
tgctgccaagttaacatagag

SEQ ID NO: 1069 - RNA, 21 nt, Homo sapiens
  source 1..21: mol_type = mRNA
acacagttggtttgaaagctt

SEQ ID NO: 1070 - RNA, 21 nt, Homo sapiens
  source 1..21: mol_type = mRNA
cacagttggtttgaaagcttc

SEQ ID NO: 1071 - RNA, 21 nt, Homo sapiens
  source 1..21: mol_type = mRNA
acagttggtttgaaagcttca

SEQ ID NO: 1072 - RNA, 21 nt, Homo sapiens
  source 1..21: mol_type = mRNA
gttggtttgaaagcttcattg

SEQ ID NO: 1073 - RNA, 21 nt, Homo sapiens
  source 1..21: mol_type = mRNA
ttggtttgaaagcttcattgt

SEQ ID NO: 1074 - RNA, 21 nt, Homo sapiens
  source 1..21: mol_type = mRNA
tggtttgaaagcttcattgtc

SEQ ID NO: 1075 - RNA, 21 nt, Homo sapiens
  source 1..21: mol_type = mRNA
ggtttgaaagcttcattgtcc

SEQ ID NO: 1076 - RNA, 21 nt, Homo sapiens
  source 1..21: mol_type = mRNA
aaaagaccattaagattatcc

SEQ ID NO: 1077 - RNA, 21 nt, Homo sapiens
  source 1..21: mol_type = mRNA
ttccttccatcatgaatgtgc

SEQ ID NO: 1078 - RNA, 21 nt, Homo sapiens
  source 1..21: mol_type = mRNA
tggcaagttctatgagtgtat

SEQ ID NO: 1079 - RNA, 21 nt, Homo sapiens
  source 1..21: mol_type = mRNA
ggcaagttctatgagtgtatt

SEQ ID NO: 1080 - RNA, 21 nt, Homo sapiens
  source 1..21: mol_type = mRNA
gcaagttctatgagtgtatta

SEQ ID NO: 1081 - RNA, 21 nt, Homo sapiens
  source 1..21: mol_type = mRNA
gttagtcaaaatgtgcgatgg

SEQ ID NO: 1082 - RNA, 21 nt, Homo sapiens
  source 1..21: mol_type = mRNA
ttagtcaaaatgtgcgatgga

SEQ ID NO: 1083 - RNA, 21 nt, Homo sapiens
  source 1..21: mol_type = mRNA
tagtcaaaatgtgcgatggaa

SEQ ID NO: 1084 - RNA, 21 nt, Homo sapiens
  source 1..21: mol_type = mRNA
agtcaaaatgtgcgatggaaa

SEQ ID NO: 1085 - RNA, 21 nt, Homo sapiens
  source 1..21: mol_type = mRNA
cctatctctgcttcaagttgc

SEQ ID NO: 1086 - RNA, 21 nt, Homo sapiens
  source 1..21: mol_type = mRNA
ctatctctgcttcaagttgca

SEQ ID NO: 1087 - RNA, 21 nt, Homo sapiens
  source 1..21: mol_type = mRNA
tatctctgcttcaagttgcaa

SEQ ID NO: 1088 - RNA, 21 nt, Homo sapiens
  source 1..21: mol_type = mRNA
tctgcttcaagttgcaacttt

SEQ ID NO: 1089 - RNA, 21 nt, Homo sapiens
  source 1..21: mol_type = mRNA
ctgcttcaagttgcaactttt

SEQ ID NO: 1090 - RNA, 21 nt, Homo sapiens
  source 1..21: mol_type = mRNA
tgcttcaagttgcaactttta

SEQ ID NO: 1091 - RNA, 21 nt, Homo sapiens
  source 1..21: mol_type = mRNA
ttgggtcattcttcactttga

SEQ ID NO: 1092 - RNA, 21 nt, Homo sapiens
  source 1..21: mol_type = mRNA
tgggtcattcttcactttgaa

SEQ ID NO: 1093 - RNA, 21 nt, Homo sapiens
  source 1..21: mol_type = mRNA
gggtcattcttcactttgaac

SEQ ID NO: 1094 - RNA, 21 nt, Homo sapiens
  source 1..21: mol_type = mRNA
ggtcattcttcactttgaact

SEQ ID NO: 1095 - RNA, 21 nt, Homo sapiens
  source 1..21: mol_type = mRNA
cattcttcactttgaacttgt

SEQ ID NO: 1096 - RNA, 21 nt, Homo sapiens
  source 1..21: mol_type = mRNA
attcttcactttgaacttgtt

SEQ ID NO: 1097 - RNA, 21 nt, Homo sapiens
  source 1..21: mol_type = mRNA
tcactttgaacttgttcattg

SEQ ID NO: 1098 - RNA, 21 nt, Homo sapiens
  source 1..21: mol_type = mRNA
actttgaacttgttcattggt

SEQ ID NO: 1099 - RNA, 21 nt, Homo sapiens
  source 1..21: mol_type = mRNA
ttgaacttgttcattggtgtc

SEQ ID NO: 1100 - RNA, 21 nt, Homo sapiens
  source 1..21: mol_type = mRNA
tcatagataatttcaaccaac

SEQ ID NO: 1101 - RNA, 21 nt, Homo sapiens
  source 1..21: mol_type = mRNA
ataatttcaaccaacagaaaa

SEQ ID NO: 1102 - RNA, 21 nt, Homo sapiens
  source 1..21: mol_type = mRNA
aaaatccaaggatgtatattt

SEQ ID NO: 1103 - RNA, 21 nt, Homo sapiens
  source 1..21: mol_type = mRNA
aaatccaaggatgtatatttg

SEQ ID NO: 1104 - RNA, 21 nt, Homo sapiens
  source 1..21: mol_type = mRNA
atccaaggatgtatatttgac

SEQ ID NO: 1105 - RNA, 21 nt, Homo sapiens
  source 1..21: mol_type = mRNA
ccaaggatgtatatttgacct

SEQ ID NO: 1106 - RNA, 21 nt, Homo sapiens
  source 1..21: mol_type = mRNA
caaggatgtatatttgaccta

SEQ ID NO: 1107 - RNA, 21 nt, Homo sapiens
  source 1..21: mol_type = mRNA
aaggatgtatatttgacctag

SEQ ID NO: 1108 - RNA, 21 nt, Homo sapiens
  source 1..21: mol_type = mRNA
aggatgtatatttgacctagt

SEQ ID NO: 1109 - RNA, 21 nt, Homo sapiens
  source 1..21: mol_type = mRNA
tagtatcatggttcttatctg

SEQ ID NO: 1110 - RNA, 21 nt, Homo sapiens
  source 1..21: mol_type = mRNA
tctcaacatggtaaccatgat

SEQ ID NO: 1111 - RNA, 21 nt, Homo sapiens
  source 1..21: mol_type = mRNA
ctcaacatggtaaccatgatg

SEQ ID NO: 1112 - RNA, 21 nt, Homo sapiens
  source 1..21: mol_type = mRNA
tcaacatggtaaccatgatgg

SEQ ID NO: 1113 - RNA, 21 nt, Homo sapiens
  source 1..21: mol_type = mRNA
caacatggtaaccatgatggt

SEQ ID NO: 1114 - RNA, 21 nt, Homo sapiens
  source 1..21: mol_type = mRNA
acatggtaaccatgatggtag

SEQ ID NO: 1115 - RNA, 21 nt, Homo sapiens
  source 1..21: mol_type = mRNA
catggtaaccatgatggtaga

SEQ ID NO: 1116 - RNA, 21 nt, Homo sapiens
  source 1..21: mol_type = mRNA
ggaagatggaattaatgacat

SEQ ID NO: 1117 - RNA, 21 nt, Homo sapiens
  source 1..21: mol_type = mRNA
gaagatggaattaatgacatg

SEQ ID NO: 1118 - RNA, 21 nt, Homo sapiens
  source 1..21: mol_type = mRNA
gaattaatgacatgttcaatt

SEQ ID NO: 1119 - RNA, 21 nt, Homo sapiens
  source 1..21: mol_type = mRNA
attaatgacatgttcaatttt

SEQ ID NO: 1120 - RNA, 21 nt, Homo sapiens
  source 1..21: mol_type = mRNA
ttaatgacatgttcaattttg

SEQ ID NO: 1121 - RNA, 21 nt, Homo sapiens
  source 1..21: mol_type = mRNA
tggattctcttcgttcacaga

SEQ ID NO: 1122 - RNA, 21 nt, Homo sapiens
  source 1..21: mol_type = mRNA
ggattctcttcgttcacagat

SEQ ID NO: 1123 - RNA, 21 nt, Homo sapiens
  source 1..21: mol_type = mRNA
gattctcttcgttcacagatg

SEQ ID NO: 1124 - RNA, 21 nt, Homo sapiens
  source 1..21: mol_type = mRNA
attctcttcgttcacagatgg

SEQ ID NO: 1125 - RNA, 21 nt, Homo sapiens
  source 1..21: mol_type = mRNA
agatggaagaaaggttcatgt

SEQ ID NO: 1126 - RNA, 21 nt, Homo sapiens
  source 1..21: mol_type = mRNA
atggaagaaaggttcatgtct

SEQ ID NO: 1127 - RNA, 21 nt, Homo sapiens
  source 1..21: mol_type = mRNA
tggaagaaaggttcatgtctg

SEQ ID NO: 1128 - RNA, 21 nt, Homo sapiens
  source 1..21: mol_type = mRNA
atcaagtatatacataaaaga

SEQ ID NO: 1129 - RNA, 21 nt, Homo sapiens
  source 1..21: mol_type = mRNA
tcaagtatatacataaaagat

SEQ ID NO: 1130 - RNA, 21 nt, Homo sapiens
  source 1..21: mol_type = mRNA
caagtatatacataaaagatg

SEQ ID NO: 1131 - RNA, 21 nt, Homo sapiens
  source 1..21: mol_type = mRNA
aagtatatacataaaagatgg

SEQ ID NO: 1132 - RNA, 21 nt, Homo sapiens
  source 1..21: mol_type = mRNA
agtatatacataaaagatgga

SEQ ID NO: 1133 - RNA, 21 nt, Homo sapiens
  source 1..21: mol_type = mRNA
gtatatacataaaagatggag

SEQ ID NO: 1134 - RNA, 21 nt, Homo sapiens
  source 1..21: mol_type = mRNA
tatatacataaaagatggaga

SEQ ID NO: 1135 - RNA, 21 nt, Homo sapiens
  source 1..21: mol_type = mRNA
tatacataaaagatggagaca

SEQ ID NO: 1136 - RNA, 21 nt, Homo sapiens
  source 1..21: mol_type = mRNA
taatgagaactcaagtccaga

SEQ ID NO: 1137 - RNA, 21 nt, Homo sapiens
  source 1..21: mol_type = mRNA
aatgagaactcaagtccagaa

SEQ ID NO: 1138 - RNA, 21 nt, Homo sapiens
  source 1..21: mol_type = mRNA
atgagaactcaagtccagaaa

SEQ ID NO: 1139 - RNA, 21 nt, Homo sapiens
  source 1..21: mol_type = mRNA
tttgttcatagaataaccaca

SEQ ID NO: 1140 - RNA, 21 nt, Homo sapiens
  source 1..21: mol_type = mRNA
taagtacatattacactcctc

SEQ ID NO: 1141 - RNA, 21 nt, Homo sapiens
  source 1..21: mol_type = mRNA
aaattacatttatataataaa

SEQ ID NO: 1142 - RNA, 21 nt, Homo sapiens
  source 1..21: mol_type = mRNA
tttaaatacattcaattaaga

SEQ ID NO: 1143 - RNA, 21 nt, Homo sapiens
  source 1..21: mol_type = mRNA
cgtgtgtaattttctattaat

SEQ ID NO: 1144 - RNA, 21 nt, Homo sapiens
  source 1..21: mol_type = mRNA
agaaggcactgtcatattaat

SEQ ID NO: 1145 - RNA, 21 nt, Homo sapiens
  source 1..21: mol_type = mRNA
ttttgatagttacctagtttg

SEQ ID NO: 1146 - RNA, 21 nt, Homo sapiens
  source 1..21: mol_type = mRNA
cagttctaaatagctatttca

SEQ ID NO: 1147 - RNA, 21 nt, Homo sapiens
  source 1..21: mol_type = mRNA
tttacataggattctttacaa

SEQ ID NO: 1148 - RNA, 21 nt, Homo sapiens
  source 1..21: mol_type = mRNA
ctatgaatgctcaaaatgttt

SEQ ID NO: 1149 - RNA, 21 nt, Homo sapiens
  source 1..21: mol_type = mRNA
tatgaatgctcaaaatgtttg

SEQ ID NO: 1150 - RNA, 21 nt, Homo sapiens
  source 1..21: mol_type = mRNA
atgaatgctcaaaatgtttga

SEQ ID NO: 1151 - RNA, 21 nt, Homo sapiens
  source 1..21: mol_type = mRNA
ttatattgtagttatacttga

SEQ ID NO: 1152 - RNA, 21 nt, Homo sapiens
  source 1..21: mol_type = mRNA
tatattgtagttatacttgag